(19) United States
(12) Patent Application Publication
(10) Pub. No.: US 2025/0136688 A1
(43) Pub. Date: May 1, 2025

(54) STABILIZED CD3 ANTIGEN BINDING AGENTS AND METHODS OF USE THEREOF

(71) Applicant: Janssen Biotech, Inc., Horsham, PA (US)

(72) Inventors: Adam Zwolak, Bala Cynwyd, PA (US); Jinquan Luo, Malvern, PA (US)

(21) Appl. No.: 18/795,775

(22) Filed: Aug. 6, 2024

Related U.S. Application Data
(60) Provisional application No. 63/518,049, filed on Aug. 7, 2023.

(51) Int. Cl. C07K 16/28; A61K 39/00; A61P 35/00; A61P 37/04; C07K 16/30; C07K 16/40

(52) U.S. Cl. CPC C07K 16/2809; A61P 35/00; A61P 37/04; C07K 16/2896; C07K 16/3069; C07K 16/40; A61K 2039/505; C07K 2317/31; C07K 2317/526; C07K 2317/565; C07K 2317/622; C07K 2317/92; C07K 2317/94

(57) ABSTRACT

Stabilized CD3 antigen binding agents and methods of use thereof are disclosed.

### CROSS-REFERENCE TO RELATED APPLICATIONS

This application claims priority to U.S. Provisional Application No. 63/518,049, filed on Aug. 7, 2023 the disclosure of which is herein incorporated by reference in its entirety

### SEQUENCE LISTING

The instant application contains a Sequence Listing which has been submitted electronically in XML format and is hereby incorporated by reference in its entirety. The XML copy, created on Aug. 6, 2024, is named JBI6831USNP1_SL.xml and is 161 kilobytes in size.

### TECHNICAL FIELD

The disclosure provided herein relates to stapled anti-cluster of differentiation 3 (CD3)-antigen-binding fragment, capable of specifically binding to human and non-human CD3, and in particular to bispecific antibodies comprising stapled anti-CD3 antigen-binding fragments that are cross-reactive with CD3 and a second target antigen. The disclosure also pertains to uses of such antibodies and antigen-binding fragments in the treatment of a disease or disorder.

### BACKGROUND

Bispecific antibodies and antibody fragments have been explored as a means to recruit cytolytic T cells to kill tumor cells. However, the clinical use of many T cell-recruiting bispecific antibodies has been limited by challenges including unfavorable pharmacokinetics, potential immunogenicity, and manufacturing issues. There thus exists a considerable need for bispecific antibodies that recruit cytolytic T cells to kill tumor cells that exhibit reduced toxicity and favorable manufacturing profiles.

The human CD3 T cell antigen receptor protein complex is composed of six distinct chains: a CD3γ chain (SwissProt P09693), a CD36 chain (SwissProt P04234), two CD3ε chains (SwissProt P07766), and one CD3ζ chain homodimer (SwissProt P20963) (εγ: εδ:ζζ), which is associated with the T cell receptor α and β chain. This complex plays an important role in coupling antigen recognition to several intracellular signal-transduction pathways. The CD3 complex mediates signal transduction, resulting in T cell activation and proliferation. CD3 is required for immune response.

Antigen binding single chain variable fragments (scFv) are modules that can be utilized broadly as therapeutics, imaging agents, diagnostic agents or as portions of heterologous molecules such as multispecific molecules. One of the challenges of scFvs is the low stability and tendencies to aggregate (reviewed in Worn and Pluckthun (2001) J Mol Biol 305: 989-1010; Rothlisberger et al., (2005) J Mol Biol 347: 773-789; Gross et al., (1989) Transplant Proc 21(1 Pt 1): 127-130, Porter et al., (2011) J Cancer 2: 331-332; Porter et al., (2011) N Engl J Med 365: 725-733). Therefore there is a need for improved scFv designs that may be optionally incorporated into multispecific molecules and heterologous molecules.

### SUMMARY OF THE INVENTION

In one aspect, provided herein is a binding agent comprising an antigen binding region that binds to cluster of differentiation 3ε (CD3ε), wherein the antigen binding region that binds to CD3ε comprises a stapled scFv (spFv) comprising a variable heavy chain sequence (VH) and a variable light chain sequence (VL) specific for binding to CD3ε and a Linker sequence between the VH and VL, wherein the Linker comprises at least one cysteine residue (Cys) that serves as an anchor point.

In some embodiments, the spFv comprises a disulfide bond between a surface exposed cysteine residue on at least one of the VH and VL the anchor point of the Linker.

In some embodiments, the spFv comprises two disulfide bonds, wherein the first disulfide bonds forms between a surface exposed cysteine residue on the VH and a first anchor point of the Linker, and the second disulfide bonds forms between a surface exposed cysteine residue on the VL a second anchor point of the Linker.

In some embodiments, the spFv is in the orientation of VH-Linker-VL.

In some embodiments, the spFv is in the orientation of VL-Linker-VH.

In some embodiments, the Linker is selected from the group consisting of SEQ ID NO:2, SEQ ID NO:3, SEQ ID NO:4, SEQ ID NO:5 and SEQ ID NO:6.

In some embodiments, the antigen binding region that binds to CD3ε comprises the HCDR1 of SEQ ID NO:7, the HCDR2 of SEQ ID NO:8, the HCDR3 of SEQ ID NO:9, the LCDR1 of SEQ ID NO:10, the LCDR2 of SEQ ID NO: 11, and the LCDR3 of SEQ ID NO:12.

In some embodiments, the antigen binding region that binds to CD3ε comprises the HCDR1 of SEQ ID NO:13, the HCDR2 of SEQ ID NO:14, the HCDR3 of SEQ ID NO:9, the LCDR1 of SEQ ID NO:10, the LCDR2 of SEQ ID NO: 11, and the LCDR3 of SEQ ID NO:12.

In some embodiments, the antigen binding region that binds to CD3ε comprises the HCDR1 of SEQ ID NO:15, the HCDR2 of SEQ ID NO:16, the HCDR3 of SEQ ID NO:9, the LCDR1 of SEQ ID NO:10, the LCDR2 of SEQ ID NO: 11, and the LCDR3 of SEQ ID NO:12.

In some embodiments, the antigen binding region that binds to CD3ε comprises the HCDR1 of SEQ ID NO:17, the HCDR2 of SEQ ID NO:18, the HCDR3 of SEQ ID NO:19, the LCDR1 of SEQ ID NO:20 the LCDR2 of SEQ ID NO:21, and the LCDR3 of SEQ ID NO:22.

In some embodiments, the antigen binding region that binds to CD3ε comprises the HCDR1 of SEQ ID NO:23, the HCDR2 of SEQ ID NO:24, the HCDR3 of SEQ ID NO:25, the LCDR1 of SEQ ID NO:26, the LCDR2 of DSS, and the LCDR3 of SEQ ID NO:12.

In some embodiments, the antigen binding region that binds to CD3ε comprises a VH domain as set forth in SEQ ID NO:28, and a VL domain as set forth in SEQ ID NO:29.

In some embodiments, the antigen binding region that binds to CD3ε comprises a spFv as set forth in SEQ ID NO:30.

In some embodiments, the binding agent is a bispecific antibody or a multi-specific antibody.

In some embodiments, the binding agent further comprises an immunoglobulin (Ig) constant region, or a fragment of the Ig constant region, wherein optionally the fragment of the Ig constant region is an Fc region or an CH3 domain.

In some embodiments, the Ig constant region, the fragment of the Ig constant region, the Fc region, or the CH3 domain comprises at least one mutation.

In some embodiments, at least one mutation is selected from the group consisting of L234A/L235A/D265S, F234A/L235A, L234A/L235A, V234A/G237A/P238S/H268A/V309L/A330S/P331S, F234A/L235A, S228P/F234A/L235A, N297A, V234A/G237A, K214T/E233P/L234V/L235A/G236-deleted/A327G/P331A/D365E/L358M, H268Q/V309L/A330S/P331S, S267E/L328F, L234F/L235E/D265A, L234A/L235A/G237A/P238S/H268A/A330S/P331S, S228P/F234A/L235A/G237A/P238S and S228P/F234A/L235A/G236-deleted/G237A/P238S, wherein residue numbering is according to the EU index.

In some embodiments, at least one mutation is selected from the group consisting of T366S/L368A/Y407V, T366W, T350V, L351Y, F405A, Y407V, T366Y, T366L, F405W, T394W, K392L, T394S, Y407T, Y407A, L351Y/F405A/Y407V, T366I/K392M/T394W, F405A/Y407V, T366L/K392M/T394W, T366L/K392L/T394W, L351Y/Y407A, L351Y/Y407V, T366A/K409F, T366V/K409F, T366A/K409F, T350V/L351Y/F405A/Y407V and T350V/T366L/K392L/T394W, wherein residue numbering is according to the EU index.

In some embodiments, the binding agent comprises knob-in-hole mutations, wherein the knob mutations comprise T366S/L368A/Y407V, and the hole mutation comprises T366W.

In some embodiments, the agent comprises a bispecific protein comprising an antigen binding region that binds a second antigen other than CD3R.

In some embodiments, the second antigen is a tumor antigen.

In one aspect, provided herein is a composition comprising the binding agent comprising an antigen binding region that binds to cluster of differentiation 3ε (CD3ε), wherein the antigen binding region that binds to CD3ε comprises a stapled scFv (spFv) comprising a variable heavy chain sequence (VH) and a variable light chain sequence (VL) specific for binding to CD3ε and a Linker sequence between the VH and VL, wherein the Linker comprises at least one cysteine residue (Cys) that serves as an anchor point, and a pharmaceutically acceptable carrier.

In one aspect, provided herein is a polynucleotide comprising nucleotide sequences encoding a VH, a VL, or both a VH and a VL of the binding agent comprising an antigen binding region that binds to cluster of differentiation 3ε (CD3ε), wherein the antigen binding region that binds to CD3ε comprises a stapled scFv (spFv) comprising a variable heavy chain sequence (VH) and a variable light chain sequence (VL) specific for binding to CD3ε and a Linker sequence between the VH and VL, wherein the Linker comprises at least one cysteine residue (Cys) that serves as an anchor point, and a pharmaceutically acceptable carrier.

In one aspect, provided herein is a vector comprising a polynucleotide comprising nucleotide sequences encoding a VH, a VL, or both a VH and a VL of the binding agent comprising an antigen binding region that binds to cluster of differentiation 3ε (CD3ε), wherein the antigen binding region that binds to CD3ε comprises a stapled scFv (spFv) comprising a variable heavy chain sequence (VH) and a variable light chain sequence (VL) specific for binding to CD3ε and a Linker sequence between the VH and VL, wherein the Linker comprises at least one cysteine residue (Cys) that serves as an anchor point, and a pharmaceutically acceptable carrier.

In one aspect, provided herein is a cell comprising a polynucleotide comprising nucleotide sequences encoding a VH, a VL, or both a VH and a VL of the binding agent comprising an antigen binding region that binds to cluster of differentiation 3ε (CD3ε), wherein the antigen binding region that binds to CD3ε comprises a stapled scFv (spFv) comprising a variable heavy chain sequence (VH) and a variable light chain sequence (VL) specific for binding to CD3ε and a Linker sequence between the VH and VL, wherein the Linker comprises at least one cysteine residue (Cys) that serves as an anchor point, and a pharmaceutically acceptable carrier.

In one aspect, provided herein is a kit comprising the binding agent comprising an antigen binding region that binds to cluster of differentiation 3ε (CD3ε), wherein the antigen binding region that binds to CD3ε comprises a stapled scFv (spFv) comprising a variable heavy chain sequence (VH) and a variable light chain sequence (VL) specific for binding to CD3ε and a Linker sequence between the VH and VL, wherein the Linker comprises at least one cysteine residue (Cys) that serves as an anchor point, and a pharmaceutically acceptable carrier.

In one aspect, provided herein is a method of treating or slowing the progression of a disease or disorder in a subject, the method comprising administering to the subject at least one binding agent comprising an antigen binding region that binds to cluster of differentiation 3ε (CD3ε), wherein the antigen binding region that binds to CD3ε comprises a stapled scFv (spFv) comprising a variable heavy chain sequence (VH) and a variable light chain sequence (VL) specific for binding to CD3ε and a Linker sequence between the VH and VL, wherein the Linker comprises at least one cysteine residue (Cys) that serves as an anchor point, and a pharmaceutically acceptable carrier.

In one aspect, provided herein is a method of directing a T cell to a target cell expressing a target antigen, comprising contacting the T cell with an effective amount of a binding agent comprising an antigen binding region that binds to cluster of differentiation 3ε (CD3ε), wherein the antigen binding region that binds to CD3ε comprises a stapled scFv (spFv) comprising a variable heavy chain sequence (VH) and a variable light chain sequence (VL) specific for binding to CD3ε and a Linker sequence between the VH and VL, wherein the Linker comprises at least one cysteine residue (Cys) that serves as an anchor point, and a pharmaceutically acceptable carrier or a composition comprising the binding agent and a pharmaceutically acceptable carrier, wherein the antigen binding region that binds to CD3ε binds the T cell and the antigen binding region that binds to a second antigen other than CD3ε binds to the target cell.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIG. 1 shows an exemplary design of the stapled scFv (spFv). The VL and the VH are connected by a flexible linker shown as a dashed line in the Figure containing a staple sequence CPPC (SEQ ID NO: 157) “SS” indicates disulfide bonds between the staple sequence in the linker and anchor points.

FIG. 2 shows a graphical illustration of anchor point selection for spFv in the VL-linker-VH orientation. Fv of the germline human antibody (pdb id 5I19, GLk1) was used for graphics and illustrative distance measurements. Distances shown in dashed lines are between CP atoms of the residues in A. Structurally conserved framework positions with desired distances were chosen as anchor points for mutation into Cys. Anchor points for VL-linker-VH orientation were Chothia position 42 for VL (K42 in the Figure) and position 105 for VH (Q105 in the Figure). The C-terminal VL residue (K107) and the N-terminal VH residue (Q1) are also shown.

FIG. 3 shows a graphical illustration of anchor point selection for spFv in the VH-linker-VL orientation. Fv of the germline human antibody (pdb id 5I19, GLk1) was used for graphics and illustrative distance measurements. Distances shown in dashed lines are between CP atoms of the residues in A. Structurally conserved framework positions with desired distances were chosen as anchor points for mutation into Cys. Anchor points for VH-linker-VL orientation were Chothia position 43 for VH (K43 in the Figure) and position 100 for VL (Q100 in the Figure). The C-terminal VH residue (S114) and the N-terminal VL residue (D1) are also shown.

FIG. 4 shows a graphical illustration of Cβ(Cys1)-Cβ(Cys2) distance between the two Cys residues in the mouse two heavy chain IgG2a (pdb id 1igt) hinge CPPC (SEQ ID NO: 31). The distances are shown in Ångstrom in the Figure.

FIG. 5 shows a graphical illustration of Cβ(Cys1)-Cβ(Cys2) distance between the two Cys residues in the two heavy chains of human IgG (pdb id 5dk3) hinge CPPC (SEQ ID NO: 31). The distances are shown in Ångstrom in the Figure.

FIG. 6 shows the chosen VL anchor points highlighted in grey and numbered as 1 and 2 below the amino acid alignments. The VL sequences are numbered according to the Chothia numbering scheme. The VL anchor point 1 (Chothia position 42) was used in spFv in the VL-linker-VH orientation and the VL anchor point 2 (Chothia position 100) was used in spFv in the VH-linker-VL orientation. GLk1VL: SEQ ID NO: 32, GLk2VL: SEQ ID NO: 33, CAT2200VL: SEQ ID NO: 34; CAT2200bVL: SEQ ID NO: 35.

FIG. 7 shows the chosen VH anchor points highlighted in grey and numbered as 1 and 2 below the amino acid alignments. The VH sequences are numbered according to the Chothia numbering scheme. The VH anchor point 1 (Chothia position 105) was used in spFv in the VL-linker-VH orientation and the VH anchor point 2 (Chothia position 43) was used in spFv in the VH-linker-VL orientation. GLk1VH: SEQ ID NO: 36; GLk2VH: SEQ ID NO: 37, CAT2200aVH: SEQ ID NO: 38.

FIG. 8 shows the structure of GLk1 spFv VL-VH. The formation of the staple between the VH and VL anchor points and the linker is evident from the structure.

FIG. 9 shows the structure of GLk1 spFv VH-VL. The formation of the staple between the VH and VL anchor points and the linker is evident from the structure.

FIG. 10 shows the structure of GLk2 spFv VH-VL. The formation of the staple between the VH and VL anchor points and the linker is evident from the structure.

FIG. 11 shows the structure of CAT2200b spFv VH-VL. The formation of the staple between the VH and VL anchor points and the linker is evident from the structure.

FIG. 12 shows the comparison of the unbound CAT2200b spFv VH-VL (top) compared to CAT2200a scFv VL-VH bound to IL-17A (bottom).

FIG. 13 shows the comparison of the front views of the structures of unbound CAT2200b spFv VH-VL (top) compared to CAT2200a spFv VL-VH bound to IL-17A (bottom).

FIG. 14 shows the comparison of the back views of the structures of unbound CAT2200b spFv VH-VL (top) compared to CAT2200a spFv VL-VH bound to IL-17A (bottom).

FIG. 15 shows dose dependent binding of stapled PSMA×CD3 antibody (PS3B2040) to LNCap human prostate tumor cells by flow cytometry.

FIG. 16 shows dose dependent binding of stapled PSMA×CD3 antibody (PS3B2040) to PAN-T cell by flow cytometry.

FIG. 17 shows dose dependent cytotoxicity of stapled PSMA×CD3 antibody (PS3B2040) in human PBMCs.

FIG. 18 shows dose dependent cytotoxicity of stapled PSMA×CD3 antibody (PS3B2040) in human Pan T cells.

FIG. 19 shows a schematic of NPP3B815 (NPP3B56×CD3B2030-N106A), a bispecific antibody targeting CD3 and ENPP3. AAS, L234A, L235A, D265S; CD, cluster of differentiation; Fab, fragment antigen-binding; hole, T366S, L368A, Y407V; knob, T366W; ENPP3, ectonucleotide pyrophosphatase/phosphodiesterase family member 3; Ig, immunoglobulin; spFv, stapled single-chain fragment variable.

FIG. 20 shows ENPP3 expression (receptor density) in different cancer cell lines with variable expression levels. Flow-cytometry-based membrane ENPP3 detection and receptor occupancy quantification measured using a commercial ENPP3 antibody (i.e., clone NP4D6) on a panel of endogenous cancer cell lines. Representative histogram showing ENPP3 expression in a high, medium, and negative cell line. Abbreviations: ABC, antibody binding capacity; ENPP3, ectonucleotide pyrophosphatase/phosphodiesterase family member 3; HCC, hepatocellular carcinoma; LD, Live Dead; LLOD, lower limit of detection; Med, medium; Neg, negative; RCC, renal cell carcinoma; ULOD, upper limit of detection.

FIG. 21 shows ENPP3 expression in different in vivo CDX and PDX model systems from ex vivo tumors. Flow-cytometry (using commercial antibody clone NP4D6) and IHC-based evaluation (using commercial antibody clone E5M2W) of ENPP3 expression on 2 CDX models, i.e., VMRCRCW (RCC) and HepG2 (HCC), and a RCC PDX model, i.e., RXF488. Magnification is 30× for all images. Abbreviations: ENPP3, ectonucleotide pyrophosphatase/phosphodiesterase family member 3; CDX, cell-line-derived xenograft; HCC, hepatocellular carcinoma; IHC, immunohistochemistry; LD, Live Dead; PDX, patient-derived xenograft; RCC, renal cell carcinoma.

FIGS. 22A and 22B show the binding of NPP3B815 to endogenous ENPP3-expressing tumor cell lines and isolated T cells. FIG. 22A shows the binding of ENPP3×CD3 (NPP3B815) and Null×CD3 (CD3B2533) on A704 (ENPP3-high), VMRCRCW (ENPP3-medium), and HepG2 ENPP3KO (ENPP3-negative) cell lines was evaluated by flow cytometry. FIG. 22B shows the binding of ENPP3×CD3 (NPP3B815, NPP3B815) and isotype control (79C3B613) antibodies evaluated by flow cytometry on T cells isolated from 6 different healthy human donors. Solid lines denote NPP3B815 and dotted lines (bottom of graph) denote 79C3B613 binding. Abbreviations: CD, cluster of differentiation; ENPP3, ectonucleotide pyrophosphatase/phosphodiesterase family member 3; geomean, geometric mean.

FIG. 23 shows binding of NPP3B815 to ENPP1, ENPP2, and ENPP3 overexpressing cell lines. Binding of ENPP3×CD3 (NPP3B815) and isotype control (79C3B613) on CHO parental cell line or CHO cell lines overexpressing ENPP1, ENPP2 or ENPP3 was evaluated by flow cytometry. Abbreviations: CD, cluster of differentiation; ENPP1, ectonucleotide pyrophosphatase/phosphodiesterase family member 1; ENPP2, ectonucleotide pyrophosphatase/phosphodiesterase family member 2; ENPP3, ectonucleotide pyrophosphatase/phosphodiesterase family member 3.

FIG. 24 shows NPP3B815-induced tumor cell killing of a panel of tumor cell lines with endogenous ENPP3 expression. Incucyte-based assessment of tumor cell killing (i.e., loss of Nuclight-red-positive cells) was measured upon treatment with ENPP3×CD3 (NPP3B815, NPP3B815), ENPP3×Null (NPP3B812), and Null×CD3 (79C3B615) antibodies in the presence of isolated T cells from Donor 888668965 at E:T ratio of 3:1 on a panel of cell lines. Data plotted at 72 hours post treatment. Error bars are SEM. Abbreviations: CD, cluster of differentiation; ENPP3, ectonucleotide pyrophosphatase/phosphodiesterase family member 3; E:T ratio, effector-to-target cell ratio; SEM, standard error of the mean.

FIG. 25 shows NPP3B815-induced T cell activation in a panel of tumor cell lines with endogenous ENPP3 expression. T cell activation (i.e., CD25+ T cells) was measured by flow cytometry upon treatment with ENPP3×CD3 (NPP3B815, NPP3B815), ENPP3×Null (NPP3B812), and Null×CD3 (79C3B615) antibodies for 48 hours in the presence of isolated T cells from Donor 888668965 at E:T ratio of 3:1 with A704 (ENPP3-high), VMRCRCW (ENPP3-medium), HepG2 (ENPP3-medium), and HepG2 ENPP3 KO (ENPP3-negative) cell lines. Error bars are SEM. Abbreviations: CD, cluster of differentiation; ENPP3, ectonucleotide pyrophosphatase/phosphodiesterase family member 3; E:T ratio, effector-to-target cell ratio.

FIG. 26A, FIG. 26B, FIG. 26C, FIG. 26D, FIG. 26E and FIG. 26F show NPP3B815-induced tumor cell killing and T cell activation in the presence of T cells isolated from multiple donors in ENPP3-positive and ENPP3-negative cell lines. FIG. 26A, FIG. 26B, and FIG. 26C show Incucyte-based assessment of tumor cell killing (ie, loss of Nuclight-red-positive cells) measured upon treatment with ENPP3×CD3 (NPP3B815) antibody in the presence of T cells (E:T ratio of 3:1) isolated from 6 different healthy human donors tested with A704 (ENPP3-high), VMRCRCW (ENPP3-medium), and HepG2 ENPP3 KO (ENPP3-negative) cell lines. Data plotted at 72 hours post treatment. Error bars are SEM. FIG. 26D, FIG. 26E and FIG. 26F show T cell activation (ie, CD25+ T cells) was measured by flow cytometry upon treatment with ENPP3×CD3 (NPP3B815) for 48 hours in the presence of T cells (E:T ratio of 3:1) isolated from 6 different healthy human donors tested with A704 (ENPP3-high), VMRCRCW (ENPP3-medium), and HepG2 ENPP3 KO (ENPP3-negative) cell lines. Error bars are SEM. Abbreviations: CD, cluster of differentiation; ENPP3, ectonucleotide pyrophosphatase/phosphodiesterase family member 3; E:T ratio, effector-to-target cell ratio; SEM, standard error of the mean.

FIG. 27A, FIG. 27B, FIG. 27C, FIG. 27D, FIG. 27E, FIG. 27F and FIG. 27G show NPP3B815-induced tumor cell killing in the presence of T cells isolated from multiple donors at different E:T ratios. FIG. 27A, FIG. 27B, FIG. 27C, and FIG. 27D show Incucyte-based assessment of tumor cell killing (i.e., loss of Nuclight-red-positive cells) measured upon treatment with ENPP3×CD3 (NPP3B815) in the presence of T cells isolated from 6 different healthy human donors and added at 2 different E:T ratios of 1:1 and 1:3 to the assay with the ENPP3-high cell line, A704. Data plotted at 72- and 120-hours post-treatment. Error Bars are SEM. FIG. 27E, FIG. 27F and FIG. 27G show Incucyte-based assessment of tumor cell killing (ie, loss of Nuclight-red-positive cells) measured upon treatment with ENPP3×CD3 (NPP3B815) in the presence of T cells isolated from 6 different healthy human donors and added at 2 different E:T ratios of 1:1 and 1:3 to the assay with VMRCRCW (ENPP3-medium) cells. Data plotted at 72 and 120 hours post treatment. Error Bars are SEM. Abbreviations: CD, cluster of differentiation; Conc., concentration; D, donor; ENPP3, ectonucleotide pyrophosphatase/phosphodiesterase family member 3; E:T ratio, effector-to-target cell ratio; IFN, interferon; IL, interleukin; SEM, standard error of the mean; TNF, tumor necrosis factor.

FIG. 28 shows NPP3B815-induced cytokine release in the presence of T cells isolated from multiple donors in ENPP3-high cancer cell line A704. Cytokine release was measured by flow cytometry upon treatment with ENPP3×CD3 (NPP3B815) for 48 hours in the presence of T cells (E:T ratio of 3:1) isolated from 6 different healthy human donors tested with A704 (ENPP3-high) cells. Error bars are SEM. Abbreviations: CD, cluster of differentiation; Conc., concentration; D, donor; ENPP3, ectonucleotide pyrophosphatase/phosphodiesterase family member 3; E:T ratio, effector-to-target cell ratio; IFN, interferon; IL, interleukin; SEM, standard error of the mean; TNF, tumor necrosis factor.

FIG. 29 shows NPP3B815-induced tumor cell killing with PBMC donors at different E:T ratios. Incucyte-based assessment of tumor cell killing (i.e., loss of Nuclight-red-positive cells) measured upon treatment with ENPP3×CD3 (NPP3B815) and Null×CD3 (79C3B615) antibodies in the presence of PBMCs (NPP3B815: E:T ratio of 5:1, 3:1 or 1:1; 79C3B615: E:T ratio of 5:1) isolated from 6 different healthy human donors tested with A704 (ENPP3-high) cell line. Data plotted at 68 hours or 72 hours post treatment. Error bars are SEM. Abbreviations: CD, cluster of differentiation; Conc., concentration; D, donor; ENPP3, ectonucleotide pyrophosphatase/phosphodiesterase family member 3; E:T ratio, effector-to-target cell ratio; PBMC, peripheral blood mononuclear cell; SEM, standard error of the mean.

FIGS. 30A and 30B show binding of toom molecule NPP3B847 to cynomolgus ENPP3-expressing cells and T cells. (FIG. 30A) Binding of ENPP3×CD3 tool (NPP3B847; ENPP3 binder NPP3B56 with cyno cross reactive CD3B219), and isotype control (79C3B613) on the HepG2-huENPP3-KO cell line with cyno ENPP3-OE was evaluated by flow cytometry. Error Bars are SEM. (FIG. 30B) Binding of ENPP3×CD3 tool (NPP3B847) was evaluated on human and cyno T cells. Error Bars are SEM. Abbreviations: CD, cluster of differentiation; cyno, cynomolgus monkey; ENPP3, ectonucleotide pyrophosphatase/phosphodiesterase family member 3; Geomean, geometric mean; SEM, standard error of the mean.

FIG. 31 shows human and cynomolgus monkey T-cell activation and T-cell mediated cytotoxicity. Tumor cell killing and T-cell activation (i.e., CD25⁺ T cells) was measured by flow cytometry upon treatment with ENPP3×CD3 (NPP3B815), matched Null×CD3 (79C3B615; with CD3B2030-N106A arm), ENPP3×CD3 tool (NPP3B847; ENPP3 binder NPP3B56 paired with cyno cross reactive CD3B219) and matched Null×CD3 (NPP3B41; with CD3B219 arm) antibodies for 48 hours in the presence of human and cyno T cells (E:T ratio of 3:1) tested with HepG2-huENPP3-KO cell line with cyno ENPP3-OE. Error Bars are SEM. Abbreviations: CD, cluster of differentiation; cyno, cynomolgus monkey; ENPP3, ectonucleotide pyrophosphatase/phosphodiesterase family member 3; E:T ratio, effector-to-target cell ratio; SEM, standard error of the mean.

FIGS. 32A and 32B show the effect of NPP3B815 on VMRCRCW established xenografts in mice (Study ONC2022-035). NSG mice bearing established VMRCRCW xenografts were IP dosed with NPP3B815 or Null×CD3 control antibody at the indicated doses. (FIG. 32A) Group tumor volumes are graphed as mean±SEM. Tumor cells were implanted on Day 0 and T cells were implanted on Day 10. Treatment with NPP3B815 or Null×CD3 control antibodies was on Days 11, 14, 18, 21, 25, 28, 31, and 34 (represented by line underneath X axis). (FIG. 32B) Individual tumor graphs for CD3×Null control antibody and NPP3B815 1 mg/kg treated groups. * Denotes significant difference of NPP3B815-treated groups on Day 39 (n=10/group) versus the control group. Abbreviations: CD, cluster of differentiation; IP, intraperitoneal; NSG, non-obese diabetic (NOD) severe combined immunodeficiency (scid) gamma or NOD.Cg-Prkdcscid Il2rgtm1Wj1/SzJ; SEM, standard error of the mean.

FIGS. 33A and 33B show the effect of NPP3B194 (unstapled [scFv] ENPP3×CD3) on HepG2 established xenografts in mice (Study P764Y). NSG mice bearing established VMRCRCW xenografts were IP dosed with NPP3B194 or Null×CD3 control antibodies at the indicated doses. (FIG. 33A) Group tumor volumes are graphed as mean±SEM. Tumor cells were implanted on Day 0 and T cells were implanted on Day 21. Treatment with NPP3B194 or Null×CD3 control antibody was on Days 22, 25, 29, 32, 36, and 39 (represented by line underneath X axis). (FIG. 33B) Individual tumor graphs for groups. * Denotes significant difference of NPP3B194-treated groups on Day 52 (n≥7/group) versus the control group. Abbreviations: CD, cluster of differentiation; IP, intraperitoneal; NSG, non-obese diabetic (NOD) severe combined immunodeficiency (scid) gamma or NOD.Cg-Prkdcscid Il2rgtm1Wj1/SzJ; SEM, standard error of the mean.

FIG. 34 shows binding of GUCY2C_mAb to human and cyno GUCY2C.

FIG. 35A shows binding of GUCY2C×CD3 biAb-1 and 2 to T84 cell line. FIG. 35B shows binding of GUCY2C×CD3 biAb-1 and 2 to T cells.

FIG. 36 shows competition flow cytometry between GUCY2C_biAb-1 and reference biAb.

FIG. 37 shows T cell mediated cytotoxicity (upper panel) and T-cell activation of GUCY2C×CD3_biAb-1, GUCY2C×CD3_biAb-2 and reference biAb.

FIG. 38 shows anti-tumor efficacy of GUCY2C×CD3 biAb-1 in a T-cell humanized HT55 CDX model.

FIG. 39 shows the binding of CDC3B253 to NCI-H146 cells.

FIG. 40 shows the binding of CDC3B253 to GSU cells.

FIG. 41 shows the binding of CDC3B253 to NUGC-4 cells.

FIG. 42 shows the binding of CDC3B253 to NUGC-4 cells CLDN18.2 KO cells.

### DETAILED DESCRIPTION OF ILLUSTRATIVE EMBODIMENTS

The disclosed methods may be understood more readily by reference to the following detailed description taken in connection with the accompanying figures, which form a part of this disclosure. It is to be understood that the disclosed methods are not limited to the specific methods described and/or shown herein, and that the terminology used herein is for the purpose of describing particular embodiments by way of example only and is not intended to be limiting of the claimed methods.

Unless specifically stated otherwise, any description as to a possible mechanism or mode of action or reason for improvement is meant to be illustrative only, and the disclosed methods are not to be constrained by the correctness or incorrectness of any such suggested mechanism or mode of action or reason for improvement.

Where a range of numerical values is recited or established herein, the range includes the endpoints thereof and all the individual integers and fractions within the range, and also includes each of the narrower ranges therein formed by all the various possible combinations of those endpoints and internal integers and fractions to form subgroups of the larger group of values within the stated range to the same extent as if each of those narrower ranges was explicitly recited. Where a range of numerical values is stated herein as being greater than a stated value, the range is nevertheless finite and is bounded on its upper end by a value that is operable within the context of the methods as described herein. Where a range of numerical values is stated herein as being less than a stated value, the range is nevertheless bounded on its lower end by a non-zero value. It is not intended that the scope of the methods be limited to the specific values recited when defining a range. All ranges are inclusive and combinable.

When values are expressed as approximations, by use of the antecedent “about,” it will be understood that the particular value forms another embodiment. Reference to a particular numerical value includes at least that particular value, unless the context clearly dictates otherwise.

It is to be appreciated that certain features of the disclosed methods which are, for clarity, described herein in the context of separate embodiments, may also be provided in combination in a single embodiment. Conversely, various features of the disclosed methods that are, for brevity, described in the context of a single embodiment, may also be provided separately or in any sub combination.

As used herein, the singular forms “a”, “an”, and “the” include the plural.

Various terms relating to aspects of the description are used throughout the specification and claims. Such terms are to be given their ordinary meaning in the art unless otherwise indicated. Other specifically defined terms are to be construed in a manner consistent with the definitions provided herein.

The term “about” is used to encompass variations of ±10% or less, variations of 5% or less, variations of ±1% or less, variations of ±0.5% or less, or variations of ±0.1% or less from the specified value.

The transitional terms “comprising,” “consisting essentially of,” and “consisting of” are intended to connote their generally accepted meanings in the patent vernacular; that is, (i) “comprising,” which is synonymous with “including,” “containing,” or “characterized by,” is inclusive or open-ended and does not exclude additional, unrecited elements or method steps; (ii) “consisting of” excludes any element, step, or ingredient not specified in the claim; and (iii) “consisting essentially of” limits the scope of a claim to the specified materials or steps “and those that do not materially affect the basic and novel characteristic(s)” of the claimed disclosure. Embodiments described in terms of the phrase “comprising” (or its equivalents) also provide as embodiments those independently described in terms of “consisting of” and “consisting essentially of.” Embodiments described in terms of the phrase “consisting essentially of” (or its equivalents) also provide as embodiments those independently described in terms of “consisting of.”

### Stabilized CD3 Antibody Fragments

The present disclosure provides antigen-binding antibody fragments that bind to CD3 and antigen binding molecules comprising the antigen-binding antibody fragments that bind to CD3. In certain circumstances there are advantages of using antibody fragments, rather than whole antibodies. The smaller size of the fragments allows for rapid clearance, and may lead to improved access to cells, tissues, or organs. For a review of certain antibody fragments, see Hudson et al., 2003, Nature Med. 9:129-34.

Various techniques have been developed for the production of antibody fragments. Traditionally, these fragments were derived via proteolytic digestion of intact antibodies (see, e.g., Morimoto et al., 1992, J. Biochem. Biophys. Methods 24:107-17; and Brennan et al., 1985, Science 229:81-83). However, these fragments can now be produced directly by recombinant host cells. Fab, Fv, scFv and spFv antibody fragments can all be expressed in and secreted from E. coli or yeast cells, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)₂ fragments (Carter et al., 1992, Bio/Technology 10:163-67). According to another approach, F(ab′)₂ fragments can be isolated directly from recombinant host cell culture. Fab and F(ab′)₂ fragment with increased in vivo half-life comprising salvage receptor binding epitope residues are described in, for example, U.S. Pat. No. 5,869,046. Other techniques for the production of antibody fragments will be apparent to the skilled practitioner. In certain embodiments, an antibody is a single chain Fv fragment (scFv) (see, e.g., WO 93/16185; U.S. Pat. Nos. 5,571,894 and 5,587,458). In certain embodiments, an antibody is a stapled single chain Fv fragment (spFv) (see, e.g., WO2021030657). Fv and scFv have intact combining sites that are devoid of constant regions; thus, they may be suitable for reduced nonspecific binding during in vivo use. scFv fusion proteins may be constructed to yield fusion of an effector protein at either the amino or the carboxy terminus of an scFv (See, e.g., Borrebaeck ed., supra). The antibody fragment may also be a “linear antibody,” for example, as described in the references cited above. Such linear antibodies may be monospecific or multi-specific, such as bispecific.

Smaller antibody-derived binding structures are the separate variable domains (V domains) also termed single variable domain antibodies (sdAbs). Certain types of organisms, the camelids and cartilaginous fish, possess high affinity single V-like domains mounted on an Fc equivalent domain structure as part of their immune system. (Woolven et al., 1999, Immunogenetics 50: 98-101; and Streltsov et al., 2004, Proc Natl Acad Sci USA. 101:12444-49). The V-like domains (called VhH in camelids and V-NAR in sharks) typically display long surface loops, which allow penetration of cavities of target antigens. They also stabilize isolated VH domains by masking hydrophobic surface patches.

These VhH and V-NAR domains have been used to engineer sdAbs. Human V domain variants have been designed using selection from phage libraries and other approaches that have resulted in stable, high binding VL- and VH-derived domains.

Antibodies comprising the antigen binding molecules provided herein include, but are not limited to, immunoglobulin molecules and immunologically active portions of immunoglobulin molecules, for example, molecules that contain an antigen binding site that bind to a CD3 epitope. The immunoglobulin molecules provided herein can be of any class (e.g., IgG, IgE, IgM, IgD, and IgA) or any subclass (e.g., IgG1, IgG2, IgG3, IgG4, IgA1, and IgA2) of immunoglobulin molecule.

Variants and derivatives of antibodies include antibody functional fragments that retain the ability to bind to a CD3 epitope. Exemplary functional fragments include Fab fragments (e.g., an antibody fragment that contains the antigen-binding domain and comprises a light chain and part of a heavy chain bridged by a disulfide bond); Fab′ (e.g., an antibody fragment containing a single antigen-binding domain comprising an Fab and an additional portion of the heavy chain through the hinge region); F(ab′)₂ (e.g., two Fab′ molecules joined by interchain disulfide bonds in the hinge regions of the heavy chains; the Fab′ molecules may be directed toward the same or different epitopes); a bispecific Fab (e.g., a Fab molecule having two antigen binding domains, each of which may be directed to a different epitope); a single chain comprising a variable region, also known as, scFv (e.g., the variable, antigen-binding determinative region of a single light and heavy chain of an antibody linked together by a chain of 10-25 amino acids); a disulfide-linked Fv, or dsFv (e.g., the variable, antigen-binding determinative region of a single light and heavy chain of an antibody linked together by a disulfide bond); a stapled scFv, or spFv (e.g., an scFv comprising at least one disulfide bond between the VH or VL and the linker); a camelized VH (e.g., the variable, antigen-binding determinative region of a single heavy chain of an antibody in which some amino acids at the VH interface are those found in the heavy chain of naturally occurring camel antibodies); a bispecific scFv (e.g., an scFv or a dsFv molecule having two antigen-binding domains, each of which may be directed to a different epitope); a diabody (e.g., a dimerized scFv formed when the VH domain of a first scFv assembles with the VL domain of a second scFv and the VL domain of the first scFv assembles with the VH domain of the second scFv; the two antigen-binding regions of the diabody may be directed towards the same or different epitopes); and a triabody (e.g., a trimerized scFv, formed in a manner similar to a diabody, but in which three antigen-binding domains are created in a single complex; the three antigen-binding domains may be directed towards the same or different epitopes).

In some embodiments, the CD3 binding region is stapled single chain antibody molecules (e.g., spFv). In some embodiments, the antigen binding molecules comprising the spFv CD3 binding arm are antibodies, multi-specific antibodies, nanobodies, diabodies, tribodies, tetrabodies, minibodies, dual variable domain antibodies (DVD), or single variable domain antibodies (e.g., camelid antibodies). Any VH and the VL domains that bind CD3 can be engineered into an spFv CD3 binding molecule.

“CD3” refers to an antigen which is expressed on T cells as part of the multimolecular T cell receptor (TCR) complex and which consists of a homodimer or heterodimer formed from the association of two or four receptor chains: CD3 epsilon, CD3 delta, CD3 zeta and CD3 gamma. Human CD3 epsilon comprises the amino acid sequence of SEQ ID NO: 1. The extracellular domain spans residues 23-126 of the full length CD3. All references to proteins, polypeptides and protein fragments herein are intended to refer to the human version of the respective protein, polypeptide or protein fragment unless explicitly specified as being from a non-human species. Thus, “CD3” means human CD3 unless specified as being from a non-human species, e.g., “mouse CD3” “monkey CD3,” etc.

(Human CD3 epsilon)
SEQ ID NO: 1
MQSGTHWRVLGLCLLSVGVWGQDGNEEMGGITQTPYKVSISGTTV
ILTCPQYPGSEILWQHNDKNIGGDEDDKNIGSDEDHLSLKEFSEL
EQSGYYVCYPRGSKPEDANFYLYLRARVCENCMEMDVMSVATIVI
VDICITGGLLLLVYYWSKNRKAKAKPVTRGAGAGGRQRGQNKERP
PPVPNPDYEPIRKGQRDLYSGLNQRRI

As used herein the term “antibody” is meant in a broad sense and includes immunoglobulin molecules including monoclonal antibodies including murine, human, humanized and chimeric monoclonal antibodies, multispecific antibodies, such as bispecific, trispecific, tetraspecific, dimeric, tetrameric or multimeric antibodies, single chain antibodies, domain antibodies and any other modified configuration of the immunoglobulin molecule that comprises an antigen binding site of the required specificity. The term antibody includes intact antibodies and antibody fragments.

In general, antibodies are proteins or peptide chains that exhibit binding specificity to a specific antigen. Antibody structures are well known. Immunoglobulins can be assigned to five major classes (i.e., IgA, IgD, IgE, IgG and IgM), depending on the heavy chain constant domain amino acid sequence. IgA and IgG are further sub-classified as the isotypes IgA1, IgA2, IgG1, IgG2, IgG3 and IgG4. Accordingly, the antibodies of the invention can be of any of the five major classes or corresponding sub-classes. Preferably, the antibodies of the invention are IgG1, IgG2, IgG3 or IgG4. Antibody light chains of vertebrate species can be assigned to one of two clearly distinct types, namely kappa and lambda, based on the amino acid sequences of their constant domains. Accordingly, the antibodies of the invention can contain a kappa or lambda light chain constant domain. According to some embodiments, the antibodies of the invention include heavy and/or light chain constant regions from rat or human antibodies. In addition to the heavy and light constant domains, antibodies contain an antigen-binding region that is made up of a light chain variable region and a heavy chain variable region, each of which contains three domains (i.e., complementarity determining regions 1-3; CDR1, CDR2, and CDR3). The light chain variable region domains are alternatively referred to as LCDR1, LCDR2, and LCDR3, and the heavy chain variable region domains are alternatively referred to as HCDR1, HCDR2, and HCDR3.

The term “variable region” or “variable domain” refers to the heavy or light chain domain that is involved in the binding of the antibody to the antigen. The variable domains of the heavy or light chain (VH and VL, respectively) comprise four framework regions (FR) and three complementarity determining regions (CDRs).

“Complementarity determining regions” (CDR) are antibody regions that bind an antigen. There are three CDRs in the VH (HCDR1, HCDR2, HCDR3) and three CDRs in the VL (LCDR1, LCDR2, LCDR3). CDRs may be defined using various delineations such as Kabat (Wu et al. (1970) J Exp Med 132: 211-50; Kabat et al., Sequences of Proteins of Immunological Interest, 5th Ed. Public Health Service, National Institutes of Health, Bethesda, Md., 1991), Chothia (Chothia et al. (1987) J Mol Biol 196: 901-17), IMGT (Lefranc et al. (2003) DevComp Immunol 27: 55-77) and AbM (Martin et al. (1996) J Mol Biol 263: 800-15,). The correspondence between the various delineations and variable region numbering is described (see e.g., Lefranc et al. (2003) Dev Comp Immunol 27: 55-77; Honegger and Pluckthun (2001), J Mol Biol 309:657-70; International ImMunoGeneTics (IMGT) database; Web resources, http://www_imgt_org). Available programs such as abYsis by UCL Business PLC may be used to delineate CDRs. The terms “CDR”, “HCDR1”, “HCDR2”, “HCDR3”, “LCDR1”, “LCDR2” and “LCDR3” as used herein include CDRs defined by any of the methods described supra, Kabat, Chothia, IMGT or AbM, unless otherwise explicitly stated in the specification.

Correspondence between the numbering system, including, for example, the Kabat numbering and the IMGT unique numbering system, is well known to one skilled in the art (see, e.g., Kabat, supra; Chothia, supra; Martin, supra; Lefranc et al., supra).

TABLE 1

| | IMGT | Kabat | AbM | Chothia |
|---|---|---|---|---|
| V_{H} CDR1 | 27-38 | 31-35 | 26-35 | 26-32 |
| V_{H} CDR2 | 56-65 | 50-65 | 50-58 | 53-55 |
| V_{H} CDR3 | 105-117 | 95-102 | 95-102 | 96-101 |
| V_{L} CDR1 | 27-38 | 24-34 | 24-34 | 26-32 |
| V_{L} CDR2 | 56-65 | 50-56 | 50-56 | 50-52 |
| V_{L} CDR3 | 105-117 | 89-97 | 89-97 | 91-96 |

“Specifically binds,” “specific binding,” “specifically binding” or “binds” refer to a proteinaceous molecule binding to an antigen or an epitope within the antigen with greater affinity than for other antigens. Typically, the proteinaceous molecule binds to the antigen or the epitope within the antigen with an equilibrium dissociation constant (KD) of about 1×10⁻⁷ M or less, for example about 5×10⁻⁸ M or less, about 1×10⁻⁸ M or less, about 1×10⁻⁹ M or less, about 1×10⁻¹⁰ M or less, about 1×10⁻¹¹ M or less, or about 1×10⁻¹² M or less, typically with the KD that is at least one hundred fold less than its KD for binding to a non-specific antigen (e.g., BSA, casein). The term “KD” refers to the dissociation constant, which is obtained from the ratio of KD to Ka (i.e., Kd/Ka) and is expressed as a molar concentration (M). KD values for antibodies can be determined using methods in the art in view of the present disclosure. For example, the KD of an antibody can be determined by using surface plasmon resonance, such as by using a biosensor system, e.g., a Biacore® system, or by using bio-layer interferometry technology, such as an Octet RED96 system. The smaller the value of the KD of an antibody, the higher affinity that the antibody binds to a target antigen.

As used herein, an antibody that “binds to PSMA” or that “specifically binds to PSMA” refers to an antibody that binds to PSMA, preferably human PSMA, with a KD of 1×10⁻⁷ M or less, preferably 1×10⁻⁸ M or less, more preferably 5×10⁻⁹ M or less, 1×10⁻⁹ M or less, 5×10⁻¹⁰ M or less, or 1×10⁻¹⁰ M, 5×10⁻¹¹ M, 1×10⁻¹¹ M, 5×10⁻¹² M, or 1×10⁻¹² M or less.

As used herein, an antibody that “binds to CD3” or that “specifically binds to CD3” refers to an antibody that binds to CD3, preferably human CD3, with a KD of 1×10⁻⁷ M or less, preferably 1×10⁻⁸ M or less, more preferably 5×10⁻⁹ M or less, 1×10⁻⁹ M or less, 5×10⁻¹⁰ M or less, or 1×10⁻¹⁰ M, 5×10⁻¹¹ M, 1×10⁻¹¹ M, 5×10⁻¹² M, or 1×10⁻¹² M or less.

“Bispecific” refers to an antibody that specifically binds two distinct antigens or two distinct epitopes within the same antigen. The bispecific antibody may have cross-reactivity to other related antigens, for example to the same antigen from other species (homologs), such as human or monkey, for example Macaca cynomolgus (cynomolgus, cyno) or Pan troglodytes, or may bind an epitope that is shared between two or more distinct antigens.

“Bispecific CD3 antibody” refers to an antibody with a first binding arm that binds a first target antigen of interest and a second binding arm that binds CD3.

In some embodiments, one or more antigen binding arm of the bispecific CD3 antibody include Fv fragments, single chain scFv fragments (scFv), single chain disulfide bond stabilized scFv fragments or stapled scFv fragment (spFv), Fab, F(ab)2, or single chain antibodies.

The term “intact antibodies” refers to an antibody having a structure similar to a native antibody. “Intact antibodies” are comprised of two heavy chains (HC) and two light chains (LC) inter-connected by disulfide bonds as well as multimers thereof (e.g. IgM). Each heavy chain is comprised of a heavy chain variable region (VH) and a heavy chain constant region (comprised of domains CH1, hinge, CH2 and CH3). Each light chain is comprised of a light chain variable region (VL) and a light chain constant region (CL). Antibody light chains of any vertebrate species may be assigned to one of two clearly distinct types, namely kappa (κ) and lambda (λ), based on the amino acid sequences of their constant domains. The VH and the VL regions may be further subdivided into regions of hypervariability, termed complementarity determining regions (CDR), interspersed with framework regions (FR). Each VH and VL is composed of three CDRs and four FR segments, arranged from amino-to-carboxy-terminus in the following order: FR1, CDR1, FR2, CDR2, FR3, CDR3 and FR4.

As used herein, the term “antibody fragment” refers to a molecule other than an intact antibody. Antibody fragments may be synthetic, enzymatically obtainable or genetically engineered polypeptides and include portions of an immunoglobulin that bind an antigen, such as a VH, a VL, a VH and a VL, a Fab, a Fab′, a F(ab′)2, a Fd and a Fv fragments, a disulfide stabilized Fv fragment (dsFv), a (dsFv)2, a bispecific dsFv (dsFv-dsFv′), a disulfide stabilized diabody (ds diabody), a single-chain antibody molecule (scFv), or a disulfide stabilized single-chain antibody molecule (spFv or stapled scFv), a single domain antibody (sdab) an scFv dimer (bivalent diabody), a multispecific antibody formed from a portion of an antibody comprising one or more CDRs, a camelized single domain antibody, a nanobody, a domain antibody, a domain antibody (dAb) consisting of one VH domain or one VL domain, a shark variable IgNAR domain, a camelized VH domain, a VHH domain, a minimal recognition unit consisting of the amino acid residues that mimic the CDRs of an antibody, such as a FR3-CDR3-FR4 portion, the HCDR1, the HCDR2 and/or the HCDR3 and the LCDR1, the LCDR2 and/or the LCDR3, an alternative scaffold that bind an antigen, a bivalent domain antibody, a multispecific protein comprising the antibody or any other antibody fragment that binds to an antigen but does not comprise a complete antibody structure.

In some embodiments, the method of the disclosure is practiced by administering a bispecific CD3 antibody comprising a heavy chain (HC), a light chain (LC) and a stapled single chain Fv (spFv).

“Single chain Fv” or “scFv” are fusion proteins comprising at least one antibody fragment comprising a light chain variable region (VL) and at least one antibody fragment comprising a heavy chain variable region (VH), wherein the VL and the VH are contiguously linked via a polypeptide linker, and capable of being expressed as a single chain polypeptide. A

scFv may have the VL and VH variable regions in either order, e.g., with respect to the N-terminal and C-terminal ends of the polypeptide, the scFv may comprise VL-linker-VH or may comprise VH-linker-VL. An scFv can comprise a linker peptide, such as two to about eight glycine or other amino acid residues, which connects the variable region of the heavy chain and the variable region of the light chain. Exemplary amino acids that may be included into the linker are Gly, Ser Pro, Thr, Glu, Lys, Arg, Ile, Leu, His and The. The linker should have a length that is adequate to link the VH and the VL in such a way that they form the correct conformation relative to one another so that they retain the desired activity, such as binding to PSMA or binding to CD3.

“Stapled single chain Fv”, “stapled scFv” or “spFv” refers to a scFv that comprises one or more disulfide bonds between the VH and the linker or the VL and the linker. Typically, the spFv may comprise one disulfide bond between the VH and the linker, and one disulfide bond between the VL and the linker, or two disulfide bonds between the VH and the linker and the VL and the linker.

“Staple” refers to the spFv linker that contains one or two Cys residues capable of forming a disulfide bond with the anchor point Cys.

“Anchor point” refers to a VH or a VL framework residue in the scFv that can be mutated into Cys without adverse effect to the overall scFv structure and is capable of forming a disulfide bond with a Cys residing in the scFv linker.

“VH Cysteine” or “VH Cys” refers to a Cys residue that resides in the VH framework.

“VL Cysteine” or “VL Cys” refers to a Cys residue that resides in the VL framework.

The linker may be about 5-50 amino acids long. In some embodiments, the linker is about 10-40 amino acids long. In some embodiments, the linker is about 10-35 amino acids long. In some embodiments, the linker is about 10-30 amino acids long. In some embodiments, the linker is about 10-25 amino acids long. In some embodiments, the linker is about 10-20 amino acids long. In some embodiments, the linker is about 15-20 amino acids long. In some embodiments, the linker is 6 amino acids long. In some embodiments, the linker is 7 amino acids long. In some embodiments, the linker is 8 amino acids long. In some embodiments, the linker is 9 amino acids long. In some embodiments, the linker is 10 amino acids long. In some embodiments, the linker is 11 amino acids long. In some embodiments, the linker is 12 amino acids long. In some embodiments, the linker is 13 amino acids long. In some embodiments, the linker is 14 amino acids long. In some embodiments, the linker is 15 amino acids long. In some embodiments, the linker is 16 amino acids long. In some embodiments, the linker is 17 amino acids long. In some embodiments, the linker is 18 amino acids long. In some embodiments, the linker is 19 amino acids long. In some embodiments, the linker is 20 amino acids long. In some embodiments, the linker is 21 amino acids long. In some embodiments, the linker is 22 amino acids long. In some embodiments, the linker is 23 amino acids long. In some embodiments, the linker is 24 amino acids long. In some embodiments, the linker is 25 amino acids long. In some embodiments, the linker is 26 amino acids long. In some embodiments, the linker is 27 amino acids long. In some embodiments, the linker is 28 amino acids long. In some embodiments, the linker is 29 amino acids long. In some embodiments, the linker is 30 amino acids long. In some embodiments, the linker is 31 amino acids long. In some embodiments, the linker is 32 amino acids long. In some embodiments, the linker is 33 amino acids long. In some embodiments, the linker is 34 amino acids long. In some embodiments, the linker is 35 amino acids long. In some embodiments, the linker is 36 amino acids long. In some embodiments, the linker is 37 amino acids long. In some embodiments, the linker is 38 amino acids long. In some embodiments, the linker is 39 amino acids long. In some embodiments, the linker is 40 amino acids long.

Exemplary linkers that contain two Cys residues are listed in Table 2.

TABLE 2
The amino acid sequences of spFv linkers
with two Cys residues.

| Linker | Linker amino | Linker |
|---|---|---|
| name | acid sequence | SEQ ID NO: |
| Linker 35 | GGGSGGSGGCPPCGGSGG | 2 |
| Linker 36 | GGGSGGCPPCGGGSGG | 3 |
| Linker 37 | GGSGGSGGCPPCGSGG | 4 |
| Linker 38 | GGGSGGSGGCPPCGSGG | 5 |
| Linker 39 | GGGSGGGSGCPPCGGGG | 6 |

In some embodiments, the method of the disclosure is practiced by administering a bispecific CD3 antibody comprising, consisting of and/or consisting essentially of a first binding domain that binds a tumor antigen of interest and a second binding domain that binds CD3, wherein the second binding domain that binds CD3 comprises, consists of and/or consists essentially of a spFv comprising a Linker of Table 2.

In some embodiments, the method of the disclosure is practiced by administering a bispecific CD3 antibody comprising, consisting of and/or consisting essentially of a first binding domain that binds a tumor antigen of interest and a second binding domain that binds CD3, wherein the second binding domain that binds CD3 comprises, consists of and/or consists essentially of a spFv comprising a VL, a Linker of Table 2 and a VH (VL-Linker-VH).

In some embodiments, the method of the disclosure is practiced by administering a bispecific CD3 antibody comprising, consisting of and/or consisting essentially of a first binding domain that binds a tumor antigen of interest and a binding domain that binds CD3, wherein the binding domain that binds CD3 comprises, consists of and/or consists essentially of a spFv comprising a VH, a Linker of Table 2 and a VL (VH-Linker-VL).

In some embodiments, the spFv comprises a first disulfide bond between a structurally conserved surface exposed VH position that is mutated to cysteine (Cys) and a first Linker Cys; and a second disulfide bond between a structurally conserved surface exposed VL position that is mutated to Cys and a second Linker Cys.

In some embodiments, the VH Cys of the spFv is at position H3, H5, H40, H43, H46 or H105, wherein residue numbering is according to Chothia.

In some embodiments, the VL Cys of the spFv is at position L3, L5, L39, L42, L43, L45, L100 or L102, wherein residue numbering is according to Chothia.

In some embodiments, the VH Cys is at H105 and the VL Cys is at L42; the VH Cys is at H43 and the VL Cys is at a L100; the VH Cys is at H3 and the VL Cys is at L3; the VH Cys is at H3 and the VL Cys is at L5; the VH Cys is at H3 and the VL Cys is at L39; the VH Cys is at H3 and the VL Cys is at L42; the VH Cys is at H3 and the VL Cys is at L45; the VH Cys is at H3 and the VL Cys is at L100; the VH Cys is at H3 and the VL Cys is at L102; the VH Cys is at H5 and the VL Cys is at L3; the VH Cys is at H5 and the VL Cys is at L5; the VH Cys is at H5 and the VL Cys is at L39; the VH Cys is at H5 and the VL Cys is at L42; the VH Cys is at H5 and the VL Cys is at L45; the VH Cys is at H5 and the VL Cys is at L100; the VH Cys is at H5 and the VL Cys is at L102; the VH Cys is at H40 and the VL Cys is at L3; the VH Cys is at H40 and the VL Cys is at L5; the VH Cys is at H40 and the VL Cys is at L39; the VH Cys is at H40 and the VL Cys is at L42; the VH Cys is at H40 and the VL Cys is at L45; the VH Cys is at H40 and the VL Cys is at L100; the VH Cys is at H40 and the VL Cys is at L102; the VH Cys is at H43 and the VL Cys is at L3; the VH Cys is at H43 and the VL Cys is at L5; the VH Cys is at H43 and the VL Cys is at L39; the VH Cys is at H43 and the VL Cys is at L42; the VH Cys is at H43 and the VL Cys is at L45; the VH Cys is at H43 and the VL Cys is at L102; the VH Cys is at H46 and the VL Cys is at L3; the VH Cys is at H46 and the VL Cys is at L5; the VH Cys is at H46 and the VL Cys is at L39; the VH Cys is at H46 and the VL Cys is at L42; the VH Cys is at H46 and the VL Cys is at L45; the VH Cys is at H46 and the VL Cys is at L100; the VH Cys is at H46 and the VL Cys is at L102; the VH Cys is at H105 and the VL Cys is at L3; the VH Cys is at H105 and the VL Cys is at L5; the VH Cys is at H105 and the VL Cys is at L39; the VH Cys is at H105 and the VL Cys is at L43; the VH Cys is at H105 and the VL Cys is at L45; the VH Cys is at H105 and the VL Cys is at L100; or the VH Cys is at H105 and the VL Cys is at L102, wherein residue numbering is according to Chothia.

In some embodiments, the spFv comprises, consists of and/or consists essentially of a Linker from Table 2.

In some embodiments, the spFv Linker comprises, consists of and/or consists essentially of the amino acid sequence of SEQ ID NO: 2. In some embodiments, the spFv Linker comprises, consists of and/or consists essentially of the amino acid sequence of SEQ ID NO: 3. In some embodiments, the spFv Linker comprises, consists of and/or consists essentially of the amino acid sequence of SEQ ID NO: 4. In some embodiments, the spFv Linker comprises, consists of and/or consists essentially of the amino acid sequence of SEQ ID NO: 5. In some embodiments, the spFv Linker comprises, consists of and/or consists essentially of the amino acid sequence of SEQ ID NO: 6.

In some embodiments, the spFv linker is the linker of sequence GGGSGGSGGCPPCGGSGG (SEQ ID NO: 2). In some embodiments, the spFv linker is the linker of sequence GGGSGGCPPCGGGSGG (SEQ ID NO: 3). In some embodiments, the spFv linker is the linker of sequence GGSGGSGGCPPCGSGG (SEQ ID NO: 4). In some embodiments, the spFv linker is the linker of sequence GGGSGGSGGCPPCGSGG (SEQ ID NO: 5). In some embodiments, the spFv linker is the linker of sequence GGGSGGGSGCPPCGGGG (SEQ ID NO: 6).

In some embodiments, the spFv comprises, consists of and/or consists essentially of a VH comprising, consisting of and/or consisting essentially of a Cys at H105; a VL comprising a Cys at L42; and a Linker comprising, consisting of and/or consisting essentially of an amino acid sequence of SEQ ID NOs: 2, 3, 4, 5, or 6; wherein the spFv is in the VL-L-VH orientation.

In some embodiments, the spFv comprises, consists of and/or consists essentially of a VH comprising, consisting of and/or consisting essentially of a Cys at H105; a VL comprising a Cys at L42; and a Linker comprising, consisting of and/or consisting essentially of an amino acid sequence of SEQ ID NOs: 2, 3, 4, 5, or 6; wherein the spFv is in the VL-L-VH orientation.

In some embodiments, the spFv comprises, consists of and/or consists essentially of a VH comprising a Cys at H105; a VL comprising, consisting of and/or consisting essentially of a Cys at L45; and a Linker comprising, consisting of and/or consisting essentially of an amino acid sequence of SEQ ID NOs: 2, 3, 4, 5, or 6; wherein the spFv is in the VL-L-VH orientation.

In some embodiments, the spFv comprises, consists of and/or consists essentially of a VH comprising, consisting of and/or consisting essentially of a Cys at H105; a VL comprising, consisting of and/or consisting essentially of a Cys at L39; and a Linker comprising an amino acid sequence of SEQ ID NOs: 2, 3, 4, 5, or 6; wherein the spFv is in the VL-L-VH orientation.

In some embodiments, the spFv comprises, consists of and/or consists essentially of a VH comprising, consisting of and/or consisting essentially of a Cys at H5; a VL comprising, consisting of and/or consisting essentially of a Cys at L42; and a Linker comprising, consisting of and/or consisting essentially of an amino acid sequence of SEQ ID NOs: 2, 3, 4, 5, or 6; wherein the spFv is in the VL-L-VH orientation.

In some embodiments, the spFv comprises, consists of and/or consists essentially of a VH comprising, consisting of and/or consisting essentially of a Cys at H5; a VL comprising, consisting of and/or consisting essentially of a Cys at L45; and a Linker comprising, consisting of and/or consisting essentially of an amino acid sequence of SEQ ID NOs: 2, 3, 4, 5, or 6; wherein the spFv is in the VL-L-VH orientation.

In some embodiments, the spFv comprises, consists of and/or consists essentially of a VH comprising, consisting of and/or consisting essentially of a Cys at H5; a VL comprising, consisting of and/or consisting essentially of a Cys at L39; and a Linker comprising, consisting of and/or consisting essentially of an amino acid sequence of SEQ ID NOs: 2, 3, 4, 5, or 6; wherein the spFv is in the VL-L-VH orientation.

In some embodiments, the spFv comprises, consists of and/or consists essentially of a VH comprising, consisting of and/or consisting essentially of a Cys at H3; a VL comprising, consisting of and/or consisting essentially of a Cys at L42; and a Linker comprising, consisting of and/or consisting essentially of an amino acid sequence of SEQ ID NOs: 2, 3, 4, 5, or 6; wherein the spFv is in the VL-L-VH orientation.

In some embodiments, the spFv comprises, consists of and/or consists essentially of a VH comprising a Cys at H3; a VL comprising, consisting of and/or consisting essentially of a Cys at L45; and a Linker comprising, consisting of and/or consisting essentially of an amino acid sequence of SEQ ID NOs: 2, 3, 4, 5, or 6; wherein the spFv is in the VL-L-VH orientation.

In some embodiments, the spFv comprises, consists of and/or consists essentially of a VH comprising, consisting of and/or consisting essentially of a Cys at H3; a VL comprising, consisting of and/or consisting essentially of a Cys at L39; and a Linker comprising, consisting of and/or consisting essentially of an amino acid sequence of SEQ ID NOs: 2, 3, 4, 5, or 6; wherein the spFv is in the VL-L-VH orientation.

In some embodiments, the spFv comprises, consists of and/or consists essentially of a VH comprising, consisting of and/or consisting essentially of a Cys at H43; a VL comprising a Cys at L100; and a Linker comprising, consisting of and/or consisting essentially of an amino acid sequence of SEQ ID NOs: 2, 3, 4, 5, or 6; wherein the spFv is in the VH-L-VL orientation.

In some embodiments, the spFv comprises, consists of and/or consists essentially of a VH comprising, consisting of and/or consisting essentially of a Cys at H43; a VL comprising, consisting of and/or consisting essentially of a Cys at L102; and a Linker comprising, consisting of and/or consisting essentially of an amino acid sequence of SEQ ID NOs: 2, 3, 4, 5, or 6; wherein the spFv is in the VH-L-VL orientation.

In some embodiments, the spFv comprises, consists of and/or consists essentially of a VH comprising, consisting of and/or consisting essentially of a Cys at H43; a VL comprising a Cys at L5; and a Linker comprising, consisting of and/or consisting essentially of an amino acid sequence of SEQ ID NOs: 2, 3, 4, 5, or 6; wherein the spFv is in the VH-L-VL orientation.

In some embodiments, the spFv comprises, consists of and/or consists essentially of a VH comprising, consisting of and/or consisting essentially of a Cys at H43; a VL comprising, consisting of and/or consisting essentially of a Cys at L3; and a Linker comprising, consisting of and/or consisting essentially of an amino acid sequence of SEQ ID NOs: 2, 3, 4, 5, or 6; wherein the spFv is in the VH-L-VL orientation.

In some embodiments, the spFv comprises, consists of and/or consists essentially of a VH comprising, consisting of and/or consisting essentially of a Cys at H40; a VL comprising, consisting of and/or consisting essentially of a Cys at L100; and a Linker comprising, consisting of and/or consisting essentially of an amino acid sequence of SEQ ID NOs: 2, 3, 4, 5, or 6; wherein the spFv is in the VH-L-VL orientation.

In some embodiments, the spFv comprises, consists of and/or consists essentially of a VH comprising, consisting of and/or consisting essentially of a Cys at H40; a VL comprising, consisting of and/or consisting essentially of a Cys at L102; and a Linker comprising, consisting of and/or consisting essentially of an amino acid sequence of SEQ ID NOs: 2, 3, 4, 5, or 6; wherein the spFv is in the VH-L-VL orientation.

In some embodiments, the spFv comprises, consists of and/or consists essentially of a VH comprising a Cys at H40; a VL comprising, consisting of and/or consisting essentially of a Cys at L5; and a Linker comprising, consisting of and/or consisting essentially of an amino acid sequence of SEQ ID NOs: 2, 3, 4, 5, or 6; wherein the spFv is in the VH-L-VL orientation.

In some embodiments, the spFv comprises, consists of and/or consists essentially of a VH comprising, consisting of and/or consisting essentially of a Cys at H40; a VL comprising, consisting of and/or consisting essentially of a Cys at L3; and a Linker comprising, consisting of and/or consisting essentially of an amino acid sequence of SEQ ID NOs: 2, 3, 4, 5, or 6; wherein the spFv is in the VH-L-VL orientation.

In some embodiments, the spFv comprises, consists of and/or consists essentially of a VH comprising, consisting of and/or consisting essentially of a Cys at H46; a VL comprising, consisting of and/or consisting essentially of a Cys at L100; and a Linker comprising, consisting of and/or consisting essentially of an amino acid sequence of SEQ ID NOs: 2, 3, 4, 5, or 6; wherein the spFv is in the VH-L-VL orientation.

In some embodiments, the spFv comprises, consists of and/or consists essentially of a VH comprising a Cys at H46; a VL comprising, consisting of and/or consisting essentially of a Cys at L102; and a Linker comprising, consisting of and/or consisting essentially of an amino acid sequence of SEQ ID NOs: 2, 3, 4, 5, or 6; wherein the spFv is in the VH-L-VL orientation.

In some embodiments, the spFv comprises, consists of and/or consists essentially of a VH comprising, consisting of and/or consisting essentially of a Cys at H46; a VL comprising, consisting of and/or consisting essentially of a Cys at L5; and a Linker comprising, consisting of and/or consisting essentially of an amino acid sequence of SEQ ID NOs: 2, 3, 4, 5, or 6; wherein the spFv is in the VH-L-VL orientation.

In some embodiments, the spFv comprises, consists of and/or consists essentially of a VH comprising, consisting of and/or consisting essentially of a Cys at H46; a VL comprising, consisting of and/or consisting essentially of a Cys at L3; and a Linker comprising, consisting of and/or consisting essentially of an amino acid sequence of SEQ ID NOs: 2, 3, 4, 5, or 6; wherein the spFv is in the VH-L-VL orientation.

While the specific examples disclose spFv with two disulfide bonds, it is readily envisioned that spFv with one disulfide bond, formed between the linker Cys and either the VH Cys or the VL Cys can be made and utilized, generating “half-anchored” molecules. The anchor positions are the same as in spFv having one or two disulfide bonds. The linker Cys position may vary in the half-anchored molecule as long as it satisfies distance and geometry requirements for disulfide bond formation with the anchor point. It is expected that the half-anchored spFv will restrain VL/VH relative movement similar to the VL/VH pair stabilized with two disulfide bonds, and thus will also be stabilizing.

In some embodiments, the multi-specific CD3 antibodies used in the method of treatment of the disclosure include chimeric, humanized or fully human antibodies that specifically bind to CD3.

“Human antibody” refers to an antibody that is optimized to have minimal immune response when administered to a human subject. Variable regions of human antibody are derived from human immunoglobulin sequences. If human antibody contains a constant region or a portion of the constant region, the constant region is also derived from human immunoglobulin sequences. Human antibody comprises heavy and light chain variable regions that are “derived from” sequences of human origin if the variable regions of the human antibody are obtained from a system that uses human germline immunoglobulin or rearranged immunoglobulin genes. Such exemplary systems are human immunoglobulin gene libraries displayed on phage, and transgenic non-human animals such as mice or rats carrying human immunoglobulin loci. “Human antibody” typically contains amino acid differences when compared to the immunoglobulins expressed in humans due to differences between the systems used to obtain the human antibody and human immunoglobulin loci, introduction of somatic mutations or intentional introduction of substitutions into the frameworks or CDRs, or both.

Typically, a “human antibody” is at least about 80%, 81%, 82%, 83%, 84%, 85%, 86%, 87%, 88%, 89%, 90%, 91%, 92%, 93%, 94%, 95%, 96%, 97%, 98% or 99% identical in amino acid sequence to an amino acid sequence encoded by human germline immunoglobulin or rearranged immunoglobulin genes. In some cases, “human antibody” may contain consensus framework sequences derived from human framework sequence analyses, for example as described in Knappik et al., (2000) J Mol Biol 296:57-86, or a synthetic HCDR3 incorporated into human immunoglobulin gene libraries displayed on phage, for example as described in Shi et al., (2010) J Mol Biol 397:385-96, and in Int. Patent Publ. No. WO2009/085462. Antibodies in which at least one CDR is derived from a non-human species are not included in the definition of “human antibody”.

Transgenic animals, such as mice, rat or chicken carrying human immunoglobulin (Ig) loci in their genome may be used to generate the antibodies used in the method of the disclosure, and are described in for example U.S. Pat. No. 6,150,584, Int. Patent Publ. No. WO1999/45962, Int. Patent Publ. Nos. WO2002/066630, WO2002/43478, WO2002/043478 and WO1990/04036. The endogenous immunoglobulin loci in such animal may be disrupted or deleted, and at least one complete or partial human immunoglobulin locus may be inserted into the genome of the animal using homologous or non-homologous recombination, using transchromosomes, or using minigenes. Companies such as Regeneron (World Wide Web: regeneron.com), Harbour Antibodies (World Wide Web: harbourantibodies.com), Open Monoclonal Technology, Inc. (OMT) (World Wide Web: omtinc.net), KyMab (World Wide Web: kymab.com), Trianni (World Wide Web: trianni.com) and Ablexis (World Wide Web: ablexis.com) can be engaged to provide human antibodies directed against a selected antigen.

The antibodies generated by immunizing non-human animals may be humanized using methods well known in the art. Generally, a humanized or engineered antibody has one or more amino acid residues from a source that is non-human, e.g., but not limited to, mouse, rat, rabbit, non-human primate or other mammal. Exemplary humanization techniques including selection of human acceptor frameworks include CDR grafting (U.S. Pat. No. 5,225,539), SDR grafting (U.S. Pat. No. 6,818,749), Resurfacing (Padlan, (1991) Mol Immunol 28:489-499), Specificity Determining Residues Resurfacing (U.S. Patent Publ. No. 2010/0261620), human framework adaptation (U.S. Pat. No. 8,748,356) or superhumanization (U.S. Pat. No. 7,709,226). In these methods, CDRs or a subset of CDR residues of parental antibodies are transferred onto human frameworks that may be selected based on their overall homology to the parental frameworks, based on similarity in CDR length, or canonical structure identity, or a combination thereof.

Humanized antigen binding domains may be further optimized to improve their selectivity or affinity to a desired antigen by incorporating altered framework support residues to preserve binding affinity (backmutations) by techniques such as those described in Int. Patent Publ. Nos. WO1090/007861 and WO1992/22653, or by introducing variation at any of the CDRs for example to improve affinity of the antigen binding domain.

The bispecific CD3 spFv antibody used in accordance with the present disclosure can be produced by recombinant means, including from mammalian cell or transgenic preparations, or can be purified from other biological sources, as described herein or as known in the art.

Antibodies used in the method of the present disclosure can be produced by a cell line, a mixed cell line, an immortalized cell or clonal population of immortalized cells, as well known in the art. Cell lines can be engineered to express the antibodies of the disclosure and the antibody can be produced intracellularly, in the periplasmic space, or directly secreted into the medium.

Cell lysate or supernatant comprising the spFv CD3 antibody can be purified using, for example, hydroxylapatite chromatography, gel electrophoresis, dialysis, and affinity chromatography. Other techniques for protein purification such as fractionation on an ion-exchange column, ethanol precipitation, Reverse Phase HPLC, chromatography on silica), chromatography on an anion or cation exchange resin are also available.

In some embodiments, the CD3ε binding region provided herein comprises an HCDR1 comprising an amino acid sequence of any of SEQ ID NO: 7, 13, 15, 17, or 23; (ii) an HCDR2 comprising an amino acid sequence of any of SEQ ID NO: 8, 14, 16, 18 or 24, (iii) an HCDR3 comprising an amino acid sequence SEQ ID NO:9, 19 or 25; (iv) a LCDR1 comprising an amino acid sequence of SEQ ID NO: 10, 20 or 26; (v) a LCDR2 comprising an amino acid sequence of SEQ ID NO: 11, 21 or DSS; and/or (vi) a LCDR3 comprising an amino acid sequence of SEQ ID NO:12 or 22.

In some specific embodiments, in the CD3ε binding region provided herein, the HCDR1 comprises the amino acid sequence of SEQ ID NO:7, the HCDR2 comprises the amino acid sequence of SEQ ID NO:8, the HCDR3 comprises the amino acid sequence of SEQ ID NO:9, the LCDR1 comprises the amino acid sequence of SEQ ID NO:10, the LCDR2 comprises the amino acid sequence of SEQ ID NO:11, and the LCDR3 comprises the amino acid sequence of SEQ ID NO:12.

In some specific embodiments, in the CD3ε binding region provided herein, the HCDR1 comprises the amino acid sequence of SEQ ID NO:13, the HCDR2 comprises the amino acid sequence of SEQ ID NO:14, the HCDR3 comprises the amino acid sequence of SEQ ID NO:9, the LCDR1 comprises the amino acid sequence of SEQ ID NO:10 the LCDR2 comprises the amino acid sequence of SEQ ID NO:11, and the LCDR3 comprises the amino acid sequence of SEQ ID NO:12.

In some specific embodiments, in the CD3ε binding region provided herein, the HCDR1 comprises the amino acid sequence of SEQ ID NO:15, the HCDR2 comprises the amino acid sequence of SEQ ID NO:16, the HCDR3 comprises the amino acid sequence of SEQ ID NO:9, the LCDR1 comprises the amino acid sequence of SEQ ID NO:10, the LCDR2 comprises the amino acid sequence of SEQ ID NO:11, and the LCDR3 comprises the amino acid sequence of SEQ ID NO:12.

In some specific embodiments, in the CD3ε binding region provided herein, the HCDR1 comprises the amino acid sequence of SEQ ID NO:17, the HCDR2 comprises the amino acid sequence of SEQ ID NO:18, the HCDR3 comprises the amino acid sequence of SEQ ID NO:19, the LCDR1 comprises the amino acid sequence of SEQ ID NO:20, the LCDR2 comprises the amino acid sequence of SEQ ID NO:21, and the LCDR3 comprises the amino acid sequence of SEQ ID NO:22.

In some specific embodiments, in the CD3ε binding region provided herein, the HCDR1 comprises the amino acid sequence of SEQ ID NO:23, the HCDR2 comprises the amino acid sequence of SEQ ID NO:24, the HCDR3 comprises the amino acid sequence of SEQ ID NO:25, the LCDR1 comprises the amino acid sequence of SEQ ID NO:26, the LCDR2 comprises the amino acid sequence of DSS and the LCDR3 comprises the amino acid sequence of SEQ ID NO:12.

In one embodiment, provided herein is a binding region that binds CD3R, comprising a VH comprising a HCDR1, a HCDR2, and a HCDR3 having an amino acid sequence of SEQ ID NOs:7, 8 and 9, respectively. In another embodiment, provided herein is a binding region that binds CD3R, comprising a VL comprising a LCDR1, a LCDR2, and a LCDR3 having an amino acid sequence of SEQ ID NOs:10, 11 and 12, respectively. In another embodiment, provided herein is a binding region that binds CD3R, comprising: (i) a VH comprising a HCDR1, a HCDR2, and a HCDR3 having an amino acid sequence of SEQ ID NOs:7, 8 and 9, respectively, and (ii) a VL comprising a LCDR1, a LCDR2, and a LCDR3 having an amino acid sequence of SEQ ID NOs:10, 11 and 12, respectively.

In one embodiment, provided herein is a binding region that binds CD3R, comprising a VH comprising a HCDR1, a HCDR2, and a HCDR3 having an amino acid sequence of SEQ ID NOs:13, 14 and 9, respectively. In another embodiment, provided herein is a binding region that binds CD3R, comprising a VL comprising a LCDR1, a LCDR2, and a LCDR3 having an amino acid sequence of SEQ ID NOs:10, 11 and 12, respectively. In another embodiment, provided herein is a binding region that binds CD3R, comprising: (i) a VH comprising a HCDR1, a HCDR2, and a HCDR3 having an amino acid sequence of SEQ ID NOs:13, 14 and 9, respectively, and (ii) a VL comprising a LCDR1, a LCDR2, and a LCDR3 having an amino acid sequence of SEQ ID NOs:10, 11 and 12, respectively.

In one embodiment, provided herein is a binding region that binds CD3R, comprising a VH comprising a HCDR1, a HCDR2, and a HCDR3 having an amino acid sequence of SEQ ID NOs:15, 16 and 9, respectively. In another embodiment, provided herein is a binding region that binds CD3R, comprising a VL comprising a LCDR1, a LCDR2, and a LCDR3 having an amino acid sequence of SEQ ID NOs:10, 11 and 12, respectively. In another embodiment, provided herein is a binding region that binds CD3R, comprising: (i) a VH comprising a HCDR1, a HCDR2, and a HCDR3 having an amino acid sequence of SEQ ID NOs:15, 16 and 9, respectively, and (ii) a VL comprising a LCDR1, a LCDR2, and a LCDR3 having an amino acid sequence of SEQ ID NOs:10, 11 and 12, respectively.

In one embodiment, provided herein is a binding region that binds CD3R, comprising a VH comprising a HCDR1, a HCDR2, and a HCDR3 having an amino acid sequence of SEQ ID NOs:17, 18, and 19, respectively. In another embodiment, provided herein is a binding region that binds CD3R, comprising a VL comprising a LCDR1, a LCDR2, and a LCDR3 having an amino acid sequence of SEQ ID NOs:20, 21 and 22, respectively. In another embodiment, provided herein is a binding region that binds CD3R, comprising: (i) a VH comprising a HCDR1, a HCDR2, and a HCDR3 having an amino acid sequence of SEQ ID NOs:17, 18, and 19, respectively, and (ii) a VL comprising a LCDR1, a LCDR2, and a LCDR3 having an amino acid sequence of SEQ ID NOs:20, 21 and 22, respectively.

In one embodiment, provided herein is a binding region that binds CD3R, comprising a VH comprising a HCDR1, a HCDR2, and a HCDR3 having an amino acid sequence of SEQ ID NOs:23, 24 and 25, respectively. In another embodiment, provided herein is a binding region that binds CD3R, comprising a VL comprising a LCDR1, a LCDR2, and a LCDR3 having an amino acid sequence of SEQ ID NO:26, DSS and SEQ ID NO:12, respectively. In another embodiment, provided herein is a binding region that binds CD3R, comprising: (i) a VH comprising a HCDR1, a HCDR2, and a HCDR3 having an amino acid sequence of SEQ ID NOs:23, 24 and 25, respectively, and (ii) a VL comprising a LCDR1, a LCDR2, and a LCDR3 having an amino acid sequence of SEQ ID NO:26, DSS and SEQ ID NO:12, respectively.

In some embodiments, the CD3ε binding region further comprises one or more framework regions. Framework regions described herein are determined based upon the boundaries of the CDR numbering system. In other words, if the CDRs are determined by, e.g., Kabat, IMGT, or Chothia, then the framework regions are the amino acid residues surrounding the CDRs in the variable region in the format, from the N-terminus to C-terminus: FR1-CDR1-FR2-CDR2-FR3-CDR3-FR4. For example, FR1 is defined as the amino acid residues N-terminal to the CDR1 amino acid residues as defined by, e.g., the Kabat numbering system, the IMGT numbering system, or the Chothia numbering system, FR2 is defined as the amino acid residues between CDR1 and CDR2 amino acid residues as defined by, e.g., the Kabat numbering system, the IMGT numbering system, or the Chothia numbering system, FR3 is defined as the amino acid residues between CDR2 and CDR3 amino acid residues as defined by, e.g., the Kabat numbering system, the IMGT numbering system, or the Chothia numbering system, and FR4 is defined as the amino acid residues C-terminal to the CDR3 amino acid residues as defined by, e.g., the Kabat numbering system, the IMGT numbering system, or the Chothia numbering system. In some embodiments, the CD3ε binding region further comprises one or more framework regions of SEQ ID NO:28, 29, 202 or 203.

In some embodiments, the CD3ε binding region provided herein comprises a VH comprising the amino acid sequence of SEQ ID NO:28, and a VL comprising the amino acid sequence of SEQ ID NO:29. In some embodiments, the CD3ε binding region provided herein comprises a VH comprising the amino acid sequence of SEQ ID NO:202, and a VL comprising the amino acid sequence of SEQ ID NO:203.

In certain embodiments, the CD3ε binding region provided herein comprises amino acid sequences with certain percent identity relative to any CD3ε binding region provided herein. The determination of percent identity can be accomplished using mathematical algorithms known in the art or described herein.

In some embodiments, the CD3ε binding region provided herein contains substitutions (e.g., conservative substitutions), insertions, or deletions relative to the reference sequence, but the CD3ε binding region comprising that sequence retains the ability to bind to CD3P. In some embodiments, a total of 1 to 10 amino acids have been substituted, inserted and/or deleted in a reference amino acid sequence. In some embodiments, substitutions, insertions, or deletions occur in regions outside the CDRs (i.e., in the FRs). Optionally, the CD3ε binding region provided herein includes post-translational modifications of a reference sequence.

TABLE 3
CD3B2030-N106A Binding Region Sequences

| | Kabat | Chothia | AbM | Contact | IMGT |
|---|---|---|---|---|---|
| Heavy Chain | RSTMH | GYTFTRS | GYTFTRSTMH | TRSTMH | GYTFTRST |
| variable region | (SEQ ID NO: 7) | (SEQ ID NO: 13) | (SEQ ID NO: 15) | (SEQ ID NO: 17) | (SEQ ID |
| CDR1 | | | | | NO: 23) |
| Heavy Chain | YINPSSAYTNY | NPSSAY | YINPSSAYTN | WIGYINPSSAYTN | INPSSAYT |
| variable region | NQKFQG | (SEQ ID NO: 14) | (SEQ ID NO: 16) | (SEQ ID NO: 18) | (SEQ ID |
| CDR2 | (SEQ ID NO: 8) | | | | NO: 24) |
| Heavy Chain | PQVHYDYAGF | PQVHYDYAGFPY | PQVHYDYAGFPY | ASPQVHYDYAGF | ASPQVHYDY |
| variable region | PY | (SEQ ID NO: 9) | (SEQ ID NO: 9) | P | AGFPY |
| CDR3 | (SEQ ID NO: 9) | | | (SEQ ID NO: 19) | (SEQ ID |
| | | | | | NO: 25) |
| Light Chain | SASSSVSYMN | SASSSVSYMN | SASSSVSYMN | SYMNWY | SSVSY |
| variable region | (SEQ ID NO: 10) | (SEQ ID NO: 10) | (SEQ ID NO: 10) | (SEQ ID NO: 20) | (SEQ ID |
| CDR1 | | | | | NO: 26) |
| Light Chain | DSSKLAS | DSSKLAS | DSSKLAS | RWIYDSSKLA | DSS |
| variable region | (SEQ ID NO: 11) | (SEQ ID NO: 11) | (SEQ ID NO: 11) | (SEQ ID NO: 21) | |
| CDR2 | | | | | |
| Light Chain | QQWSRNPPT | QQWSRNPPT | QQWSRNPPT | QQWSRNPP | QQWSRNPPT |
| variable region | (SEQ ID NO: 12) | (SEQ ID NO: 12) | (SEQ ID NO: 12) | (SEQ ID NO: 22) | (SEQ ID |
| CDR3 | | | | | NO: 12) |

VH amino acid sequence of CD3B2030-N106A.
SEQ ID NO: 28
QVQLVQSGAEVKKPGSSVKVSCKASGYTFTRSTMHWVKQAPGQGL
EWIGYINPSSAYTNYNQKFQGRVTLTADKSTSTAYMELSSLRSED
TAVYYCASPQVHYDYAGFPYWGCGTLVTVSS
VL amino acid sequence of CD3B2030-N106A.
SEQ ID NO: 29
EIVLTQSPATLSASPGERVTLSCSASSSVSYMNWYQQKPGCAPRR
WIYDSSKLASGVPARFSGSGSGRDYTLTISSLEPEDFAVYYCQQW
SRNPPTFGGGTKVEIK
SpFv (VL-linker-VH) amino acid sequence of
CD3B2030-N106A;
SEQ ID NO: 30
EIVLTQSPATLSASPGERVTLSCSASSSVSYMNWYQQKPGCAPRR
WIYDSSKLASGVPARFSGSGSGRDYTLTISSLEPEDFAVYYCQQW
SRNPPTFGGGTKVEIKGGGSGGSGGCPPCGGSGGQVQLVQSGAEV
KKPGSSVKVSCKASGYTFTRSTMHWVKQAPGQGLEWIGYINPSSA
YTNYNQKFQGRVTLTADKSTSTAYMELSSLRSEDTAVYYCASPQV
HYDYAGFPYWGCGTLVTVSS
DNA sequence encoding VH
(SEQ ID NO: 68)
CAGGTTCAGCTGGTTCAGTCTGGCGCCGAAGTGAAGAAACCTGGC
TCCTCCGTGAAAGTGTCCTGCAAGGCTTCCGGCTACACCTTTACC
AGATCCACCATGCACTGGGTCAAGCAGGCCCCTGGACAAGGCTTG
GAGTGGATCGGCTACATCAACCCCAGCTCCGCCTACACCAACTAC
AACCAGAAATTCCAGGGCAGAGTGACCCTGACCGCCGACAAGTCT
ACCTCCACCGCCTACATGGAACTGTCCAGCCTGAGATCTGAGGAC
ACCGCCGTGTACTACTGCGCCTCTCCTCAGGTTCACTACGACTAC
GCCGGCTTTCCTTATTGGGGCTGTGGCACCCTGGTCACCGTTTCT
TCT
DNA sequence encoding VL
(SEQ ID NO: 69)
GAGATCGTGCTGACCCAGTCTCCTGCCACACTGAGTGCTTCTCCA
GGCGAGAGAGTGACCCTGTCCTGCTCCGCTTCCTCCTCCGTGTCC
TACATGAACTGGTATCAGCAGAAGCCCGGCTGCGCCCCTAGAAGA
TGGATCTACGACTCCTCCAAGCTGGCCTCTGGCGTGCCTGCTAGA
TTTTCCGGCTCTGGCTCTGGCAGAGACTATACCCTGACAATCTCC
AGCCTGGAACCTGAGGACTTCGCCGTGTACTACTGCCAGCAGTGG
TCTAGGAACCCTCCTACCTTTGGCGGAGGCACCAAGGTGGAAATC
AAG
DNA sequence encoding SpFv (VL-linker-VH)
amino acid sequence of CD3B2030-N106A
(SEQ ID NO: 70)
GAGATCGTGCTGACCCAGTCTCCTGCCACACTGAGTGCTTCTCCA
GGCGAGAGAGTGACCCTGTCCTGCTCCGCTTCCTCCTCCGTGTCC
TACATGAACTGGTATCAGCAGAAGCCCGGCTGCGCCCCTAGAAGA
TGGATCTACGACTCCTCCAAGCTGGCCTCTGGCGTGCCTGCTAGA
TTTTCCGGCTCTGGCTCTGGCAGAGACTATACCCTGACAATCTCC
AGCCTGGAACCTGAGGACTTCGCCGTGTACTACTGCCAGCAGTGG
TCTAGGAACCCTCCTACCTTTGGCGGAGGCACCAAGGTGGAAATC
AAGGGCGGAGGTTCTGGTGGATCTGGCGGATGTCCTCCTTGTGGT
GGAAGCGGCGGACAGGTTCAGCTGGTTCAGTCTGGCGCCGAAGTG
AAGAAACCTGGCTCCTCCGTGAAAGTGTCCTGCAAGGCTTCCGGC
TACACCTTTACCAGATCCACCATGCACTGGGTCAAGCAGGCCCCT
GGACAAGGCTTGGAGTGGATCGGCTACATCAACCCCAGCTCCGCC
TACACCAACTACAACCAGAAATTCCAGGGCAGAGTGACCCTGACC
GCCGACAAGTCTACCTCCACCGCCTACATGGAACTGTCCAGCCTG
AGATCTGAGGACACCGCCGTGTACTACTGCGCCTCTCCTCAGGTT
CACTACGACTACGCCGGCTTTCCTTATTGGGGCTGTGGCACCCTG
GTCACCGTTTCTTCTGAGCCCAAATCTAGCGACAAAACTCACACA
TGCCCACCGTGCCCAGCACCTGAAGCCGCCGGGGGACCGTCAGTC
TTCCTCTTCCCCCCAAAACCCAAGGACACCCTCATGATCTCCCGG
ACCCCTGAGGTCACATGCGTGGTGGTGAGCGTGAGCCACGAAGAC
CCTGAGGTCAAGTTCAACTGGTACGTGGACGGCGTGGAGGTGCAT
AATGCCAAGACAAAGCCGCGGGAGGAGCAGTACAACAGCACGTAC
CGTGTGGTCAGCGTCCTCACCGTCCTGCACCAGGACTGGCTGAAT
GGCAAGGAGTACAAGTGCAAGGTGTCGAACAAAGCCCTCCCAGCC
CCCATCGAGAAAACCATCTCCAAAGCCAAAGGGCAGCCCCGAGAA
CCACAGGTGTACACCCTGCCCCCATCCCGGGAGGAGATGACCAAG
AACCAGGTCAGCCTGTGGTGCCTGGTCAAAGGCTTCTATCCCAGC
GACATCGCCGTGGAGTGGGAGAGCAATGGGCAGCCGGAGAACAAC
TACAAGACCACGCCTCCCGTGCTGGACTCCGACGGCTCCTTCTTC
CTCTACAGCAAGCTCACCGTGGACAAGAGCAGATGGCAGCAGGGG
AACGTCTTCTCATGCTCCGTGATGCATGAGGCTCTGCACAACCAC
TACACGCAGAAGTCTCTCTCCCTGTCTCCGGGAAAA

### Fc Engineering

In some embodiments, the CD3 spFv of the disclosure are conjugated to an Ig constant region or a fragment of the Ig constant region to impart antibody-like properties, including Fc effector functions C1q binding, complement dependent cytotoxicity (CDC), Fc receptor binding, antibody-dependent cell-mediated cytotoxicity (ADCC), phagocytosis or down regulation of cell surface receptors (e.g., B cell receptor; BCR). In some embodiments, the CD3 spFv of the disclosure are used to make a fusion protein, wherein the fusion protein comprises the CD3 spFv and an Ig constant region or a fragment of the Ig constant region to impart antibody-like properties, including Fc effector functions C1q binding, complement dependent cytotoxicity (CDC), Fc receptor binding, antibody-dependent cell-mediated cytotoxicity (ADCC), phagocytosis or down regulation of cell surface receptors (e.g., B cell receptor; BCR). The Ig constant region or the fragment of the Ig constant region functions also as a half-life extending moiety as discussed herein. The CD3 spFv may further be engineered as described herein.

Immunoglobulin heavy chain constant region comprised of subdomains CH1, hinge, CH2 and CH3. The CH1 domain spans residues A118-V215, the CH2 domain residues A231-K340 and the CH3 domain residues G341-K447 on the heavy chain, residue numbering according to the EU Index. In some instances, G341 is referred as a CH2 domain residue. Hinge is generally defined as including E216 and terminating at P230 of human IgG1. In some embodiments, the Ig Fc region comprises at least the CH2 and the CH3 domains of the Ig constant region, and therefore comprises at least a region from about A231 to K447 of Ig heavy chain constant region.

In some embodiments, the C-terminal lysine (CTL) is removed from the Ig constant region. Accordingly, in some embodiments, the Ig Fc region comprises at least a region from about A231 to G446 of Ig heavy chain constant region. In specific embodiments, the CTL is removed from the Ig constant region by endogenous circulating carboxypeptidases in the blood stream (Cai et al., (2011) Biotechnol Bioeng 108:404-412). In some embodiments, during manufacturing, CTL removal may be controlled to less than the maximum level by control of concentration of extracellular Zn2+, EDTA or EDTA-Fe3+ as described in U.S. Patent Publ. No. US20140273092. CTL content of proteins may be measured using known methods.

In other embodiments, the CD3 spFv fused to the Ig constant region has a C-terminal lysine content from about 10% to about 90%. In other embodiments, the C-terminal lysine content is from about 20% to about 80%. In other embodiments, the C-terminal lysine content is from about 40% to about 70%. In other embodiments, the C-terminal lysine content is from about 55% to about 70%. In other embodiments, the C-terminal lysine content is about 60%.

The present disclosure also provides a CD3 spFv fused or conjugated to an immunoglobulin (Ig) constant region or a fragment of the Ig constant region. In some embodiments, the Ig constant region is a heavy chain constant region. In some embodiments, the Ig constant region is a light chain constant region. In some embodiments, the fragment of the Ig constant region comprises a Fc region. In some embodiments, the fragment of the Ig constant region comprises a CH2 domain. In some embodiments, the fragment of the Ig constant region comprises a CH3 domain. In some embodiments, the fragment of the Ig constant region comprises the CH2 domain and the CH3 domain. In some embodiments, the fragment of the Ig constant region comprises at least portion of a hinge, the CH2 domain and the CH3 domain. Portion of the hinge refers to one or more amino acid residues of the Ig hinge. In some embodiments, the fragment of the Ig constant region comprises the hinge, the CH2 domain and the CH3 domain.

In some embodiments, the CD3 spFv is fused or conjugated to the N-terminus of the Ig constant region or the fragment of the Ig constant region. In some embodiments, the CD3 spFv is fused or conjugated to the C-terminus of the Ig constant region or the fragment of the Ig constant region. In some embodiments, the CD3 spFv is fused or conjugated to the Ig constant region or the fragment of the Ig constant region via a second linker.

The CD3 spFv of the disclosure fused or conjugated to Ig constant region or the fragment of the Ig constant region may be assessed for their functionality using several known assays. Binding to CD3 can be assessed using methods described herein. Altered properties imparted by the Ig constant domain or the fragment of the Ig constant region such as Fc region may be assayed in Fc receptor binding assays using soluble forms of the receptors, such as the FcγRI, FcγRII, FcγRIII or FcRn receptors, or using cell-based assays measuring for example ADCC, CDC or ADCβ.

ADCC can be assessed using an in vitro assay using NK cells as effector cells. Cytolysis may be detected by the release of label (e.g. radioactive substrates, fluorescent dyes or natural intracellular proteins) from the lysed cells. In an exemplary assay, target cells are used with a ratio of 1 target cell to 4 effector cells. Target cells are pre-labeled with BATDA and combined with effector cells and the test antibody. The samples are incubated for 2 hours and cell lysis measured by measuring released BATDA into the supernatant. Data is normalized to maximal cytotoxicity with 0.67% Triton X-100 (Sigma Aldrich) and minimal control determined by spontaneous release of BATDA from target cells in the absence of any antibody.

ADCP can be evaluated by using monocyte-derived macrophages as effector cells and target cells which are engineered to express GFP or other labeled molecule. In an exemplary assay, effector:target cell ratio may be for example 4:1. Effector cells may be incubated with target cells for 4 hours with or without the antibody of the invention. After incubation, cells may be detached using accutase. Macrophages may be identified with anti-CD11b and anti-CD14 antibodies coupled to a fluorescent label, and percent phagocytosis may be determined based on % GFP fluorescence in the CD11+CD14+ macrophages using standard methods.

CDC of cells may be measured for example by plating Daudi cells at 1×105 cells/well (50 μL/well) in RPMI-B (RPMI supplemented with 1% BSA), adding 50 μL of test protein to the wells at final concentration between 0-100 μg/mL, incubating the reaction for 15 min at room temperature, adding 11 μL of pooled human serum to the wells, and incubation the reaction for 45 min at 37° C. Percentage (%) lysed cells may be detected as % propidium iodide stained cells in FACS assay using standard methods.

In some embodiments, it may be desirable to modify an anti-CD3 antibody provided herein by Fc engineering. In certain embodiments, the modification to the Fc region of the antibody results in the decrease or elimination of an effector function of the antibody. In certain embodiments, the effector function is ADCC, ADCβ, and/or CDC. In some embodiments, the effector function is ADCC. In other embodiments, the effector function is ADCβ. In other embodiments, the effector function is CDC. In one embodiment, the effector function is ADCC and ADCβ. In one embodiment, the effector function is ADCC and CDC. In one embodiment, the effector function is ADCP and CDC. In one embodiment, the effector function is ADCC, ADCP and CDC. This may be achieved by introducing one or more amino acid substitutions in an Fc region of the antibody. For example, substitutions into human IgG1 using IgG2 residues at positions 233-236 and IgG4 residues at positions 327, 330, and 331 were shown to greatly reduce ADCC and CDC (see, e.g., Armour et al., 1999, Eur. J. Immunol. 29(8):2613-24; and Shields et al., 2001, J. Biol. Chem. 276(9): 6591-604). Other Fc variants are provided elsewhere herein.

To increase the serum half-life of the antibody, one may incorporate a salvage receptor binding epitope into the antibody (especially an antibody fragment), for example, as described in U.S. Pat. No. 5,739,277. Term “salvage receptor binding epitope” refers to an epitope of the Fc region of an IgG molecule (e.g., IgG1, IgG2, IgG3, or IgG4) that is responsible for increasing the in vivo serum half-life of the IgG molecule.

In some embodiments, the Ig constant region or the fragment of the Ig constant region comprises at least one mutation that modulates a half-life of the CD3 spFv. In some embodiments, the at least one mutation that modulates the half-life of the CD3 spFv is selected from the group consisting of H435A, P257I/N434H, D376V/N434H, M252Y/S254T/T256E/H433K/N434F, T308P/N434A, and H435R, wherein residue numbering is according to the EU index. In some embodiments, the CD3 spFv comprises a first Ig constant region or a fragment thereof and a second Ig constant region or a fragment thereof. In some embodiments, one or both of the first Ig constant region or a fragment thereof and a second Ig constant region or a fragment thereof comprises at least one mutation that modulates a half-life of the CD3 spFv independently selected from the group consisting of H435A, P257I/N434H, D376V/N434H, M252Y/S254T/T256E/H433K/N434F, T308P/N434A, and H435R, wherein residue numbering is according to the EU index.

In some embodiments, the Ig constant region or the fragment of the Ig constant region comprises at least one mutation that results in reduced binding of the CD3 spFv to a FcγR.

In some embodiments, the at least one mutation that results in reduced binding of the CD3 spFv to the FcγR is selected from the group consisting of F234A/L235A, L234A/L235A, L234A/L235A/D265S, V234A/G237A/P238S/H268A/V309L/A330S/P331S, F234A/L235A, S228P/F234A/L235A, N297A, V234A/G237A, K214T/E233P/L234V/L235A/G236-deleted/A327G/P331A/D365E/L358M, H268Q/V309L/A330S/P331S, S267E/L328F, L234F/L235E/D265A, L234A/L235A/G237A/P238S/H268A/A330S/P331S, S228P/F234A/L235A/G237A/P238S and S228P/F234A/L235A/G236-deleted/G237A/P238S, wherein residue numbering is according to the EU index. In some embodiments, the CD3 spFv comprises a first Ig constant region or a fragment thereof and a second Ig constant region or a fragment thereof. In some embodiments, one or both of the first Ig constant region or a fragment thereof and a second Ig constant region or a fragment thereof comprises at least one mutation that modulates a half-life of the CD3 spFv independently selected from the group consisting of F234A/L235A, L234A/L235A, L234A/L235A/D265S, V234A/G237A/P238S/H268A/V309L/A330S/P331S, F234A/L235A, S228P/F234A/L235A, N297A, V234A/G237A, K214T/E233P/L234V/L235A/G236-deleted/A327G/P331A/D365E/L358M, H268Q/V309L/A330S/P331S, S267E/L328F, L234F/L235E/D265A, L234A/L235A/G237A/P238S/H268A/A330S/P331S, S228P/F234A/L235A/G237A/P238S and S228P/F234A/L235A/G236-deleted/G237A/P238S, wherein residue numbering is according to the EU index.

In some embodiments, the Ig constant region or the fragment of the Ig constant region comprises at least one mutation that results in enhanced binding of the CD3 spFv to a FcγR.

In some embodiments, the at least one mutation that results in enhanced binding of the CD3 spFv to the FcγR is selected from the group consisting of S239D/I332E, S298A/E333A/K334A, F243L/R292P/Y300L, F243L/R292P/Y300L/P396L, F243L/R292P/Y300L/V305I/P396L and G236A/S239D/I332E, wherein residue numbering is according to the EU index. In some embodiments, the FcγR is FcγRI, FcγRIIA, FcγRIIB or FcγRIII, or any combination thereof. In some embodiments, the CD3 spFv comprises a first Ig constant region or a fragment thereof and a second Ig constant region or a fragment thereof. In some embodiments, one or both of the first Ig constant region or a fragment thereof and a second Ig constant region or a fragment thereof comprises at least one mutation that modulates a half-life of the CD3 spFv independently selected from the group consisting of S239D/I332E, S298A/E333A/K334A, F243L/R292P/Y300L, F243L/R292P/Y300L/P396L, F243L/R292P/Y300L/V305I/P396L and G236A/S239D/I332E, wherein residue numbering is according to the EU index.

In some embodiments, the binding agent comprises at least one mutation in a CH3 domain of a first Ig constant region or in a CH3 domain of the fragment of the first Ig constant region and/or at least one mutation in a CH3 domain of a second Ig constant region or in a CH3 domain of the fragment of the second Ig constant region. In some embodiments, the at least one mutation in a CH3 domain of the first Ig constant region or in a CH3 domain of the fragment of the first Ig constant region and/or at least one mutation in a CH3 domain of the second Ig constant region or in a CH3 domain of the fragment of the second Ig constant region is selected from the group consisting of T350V, L351Y, F405A, Y407V, T366Y, T366W, T366L, F405W, K392L, T394W, T394S, Y407T, Y407A, H435R, Y436F, T366S/L368A/Y407V, L351Y/F405A/Y407V, T366I/K392M/T394W, T366L/K392L/T394W, F405A/Y407V, T366L/K392M/T394W, L351Y/Y407A, L351Y/Y407V, T366A/K409F, L351Y/Y407A, T366V/K409F, T366A/K409F, T350V/L351Y/F405A/Y407V, T350V/T366L/K392L/T394W, and H435R/L436F wherein residue numbering is according to the EU index. In some embodiments, at least one mutation in the CH3 domain is selected from the group consisting of H435R, Y436F and H435R/L436F, wherein residue numbering is according to the EU index

In some embodiments, the first Ig constant region or the fragment of the first Ig constant region and the second Ig constant region or the fragment of the second Ig constant region comprise the following mutations L234A_L235A_D265S_T350V_L351Y_F405A_Y407V in the first Ig constant region and L234A_L235A_D265S_T350V_T366L_K392L_T394W in the second Ig constant region; or L234A_L235A_D265S_T350V_T366L_K392L_T394W in the first Ig constant region and L234A_L235A_D265S_T350V_L351Y_F405A_Y407V in the second Ig constant region.

### Alternative Binding Agents

The present disclosure encompasses non-immunoglobulin binding agents comprising the CD3 spFv disclosed herein. These alternative binding agents may include, for example, any of the engineered protein scaffolds known in the art. Such scaffolds may comprise one or more CDRs as shown in Table 3. Such scaffolds include, for example, anticalins, which are based upon the lipocalin scaffold, a protein structure characterized by a rigid beta-barrel that supports four hypervariable loops which form the ligand binding site. Novel binding specificities may be engineered by targeted random mutagenesis in the loop regions, in combination with functional display and guided selection (see, e.g., Skerra, 2008, FEBS J. 275:2677-83). Other suitable scaffolds may include, for example, adnectins, or monobodies, based on the tenth extracellular domain of human fibronectin III (see, e.g., Koide and Koide, 2007, Methods Mol. Biol. 352: 95-109); affibodies, based on the Z domain of staphylococcal protein A (see, e.g., Nygren et al., 2008, FEBS J. 275:2668-76); DARPins, based on ankyrin repeat proteins (see, e.g., Stumpp et al., 2008, Drug. Discov. Today 13:695-701); fynomers, based on the SH3 domain of the human Fyn protein kinase (see, e.g., Grabulovski et al., 2007, J. Biol. Chem. 282:3196-204); affitins, based on Sac7d from Sulfolobus acidolarius (see, e.g., Krehenbrink et al., 2008, J. Mol. Biol. 383:1058-68); affilins, based on human y-B-crystallin (see, e.g., Ebersbach et al., 2007, J. Mol. Biol. 372:172-85); avimers, based on the A domain of membrane receptor proteins (see, e.g., Silverman et al., 2005, Biotechnol. 23:1556-61); cysteine-rich knottin peptides (see, e.g., Kolmar, 2008, FEBS J. 275:2684-90); and engineered Kunitz-type inhibitors (see, e.g., Nixon and Wood, 2006, Curr. Opin. Drug. Discov. Dev. 9:261-68). For a review, see, for example, Gebauer and Skerra, 2009, Curr. Opin. Chem. Biol. 13:245-55.

### Multispecific Binding Proteins

In one aspect of the present disclosure, the CD3 spFv described herein are incorporated into multispecific (e.g., bispecific or trispecific) binding molecules that can bind to one or more other antigens in addition to CD3.

Methods for making multispecific antibodies are known in the art, such as, by co-expression of two immunoglobulin heavy chain-light chain pairs, where the two heavy chains have different specificities (see, e.g., Milstein and Cuello, 1983, Nature 305:537-40). For further details of generating bispecific antibodies, see, for example, Bispecific Antibodies (Kontermann ed., 2011).

In some embodiments, the multispecific protein is bispecific. In some embodiments, the multispecific protein is trispecific. In some embodiments, the multispecific protein is tetraspecific.

In specific embodiments, the binding agents described herein are bispecific antibodies. In some embodiments, bispecific antibodies described herein are monoclonal antibodies that have binding specificities for at least two different antigens. In certain embodiments, bispecific antibodies are human or humanized antibodies. In certain embodiments, one of the binding specificities is for CD3 and the other is for any other antigen. In some embodiments, one of the binding specificities is for CD3, and the other is for a surface antigen expressed on cells. Bispecific antibodies can be prepared as full length antibodies or antibody fragments (e.g., F(ab′)2 bispecific antibodies).

In other embodiments, the multispecific protein is monovalent for binding to CD3. In other embodiments, the multispecific protein is bivalent for binding to CD3. In other embodiments, the multispecific protein is monovalent for binding to the second antigen. In other embodiments, the multispecific protein is bivalent for binding to second antigen. In other embodiments, the multispecific protein is monovalent for binding to CD3, and is monovalent for binding to the second antigen. In other embodiments, the multispecific protein is bivalent for binding to CD3, and is monovalent for binding to the second antigen. In other embodiments, the multispecific protein is monovalent for binding to CD3, and is bivalent for binding to the second antigen. In other embodiments, the multispecific protein is bivalent for binding to CD3, and is bivalent for binding to the second antigen.

In some embodiments, the second antigen binding domain that binds the second antigen is selected from a scFv, a spFv, a (scFv)2, a Fv, a Fab, a F(ab′)2, a Fd, a dAb or a VHH. In some embodiments, second antigen binding domain that binds the second antigen comprises a Fab. In some embodiments, the second antigen binding domain that binds the second antigen comprises a F(ab′)2. In some embodiments, the second antigen binding domain that binds the second antigen comprises a VHH. In some embodiments, the second antigen binding domain that binds the second antigen comprises a Fv. In some embodiments, the second antigen binding domain that binds the second antigen comprises a Fd. In some embodiments, the second antigen binding domain that binds the second antigen comprises a scFv. In some embodiments, the second antigen binding domain that binds the second antigen comprises a spFv.

In some embodiments, comprises a is fused or conjugated to a first immunoglobulin (Ig) constant region or a fragment of the first Ig constant region and/or the second antigen binding domain that binds the tumor antigen is fused or conjugated to a second immunoglobulin (Ig) constant region or a fragment of the second Ig constant region.

In some embodiments, the fragment of the first Ig constant region and/or the fragment of the second Ig constant region comprises a Fc region. In some embodiments, the fragment of the first Ig constant region and/or the fragment of the second Ig constant region comprises a CH2 domain. In some embodiments, the fragment of the first Ig constant region and/or the fragment of the second Ig constant region comprises a CH3 domain. In some embodiments, the fragment of the first Ig constant region and/or the fragment of the second Ig constant region comprises the CH2 domain and the CH3 domain.

In some embodiments, the fragment of the first Ig constant region and/or the fragment of the second Ig constant region comprises at least portion of a hinge, the CH2 domain and the CH3 domain. In some embodiments, the fragment of the Ig constant region comprises the hinge, the CH2 domain and the CH3 domain.

In some embodiments, the multispecific protein further comprises a second linker (L2) between the first antigen binding domain that binds CD3 and the first Ig constant region or the fragment of the first Ig constant region and the second antigen binding domain that binds the second antigen and the second Ig constant region or the fragment of the second Ig constant region.

In some embodiments, the first Ig constant region or the fragment of the first Ig constant region and the second Ig constant region or the fragment of the second Ig constant region is an IgG1, an IgG2, and IgG3 or an IgG4 isotype. In some embodiments, the first Ig constant region or the fragment of the first Ig constant region and the second Ig constant region or the fragment of the second Ig constant region is an IgG1 isotype. In some embodiments, the first Ig constant region or the fragment of the first Ig constant region and the second Ig constant region or the fragment of the second Ig constant region is an IgG2 isotype. In some embodiments, the first Ig constant region or the fragment of the first Ig constant region and the second Ig constant region or the fragment of the second Ig constant region is an IgG3 isotype. In some embodiments, the first Ig constant region or the fragment of the first Ig constant region and the second Ig constant region or the fragment of the second Ig constant region is an IgG4 isotype.

The first Ig constant region or the fragment of the first Ig constant region and the second Ig constant region or the fragment of the second Ig constant region can further be engineered as described herein. In some embodiments, the first Ig constant region or the fragment of the first Ig constant region and the second Ig constant region or the fragment of the second Ig constant region comprises at least one mutation that results in reduced binding of the multispecific protein to a FcγR.

In some embodiments, the at least one mutation that results in reduced binding of the multispecific protein to the FcγR is selected from the group consisting of F234A/L235A, L234A/L235A, L234A/L235A/D265S, V234A/G237A/P238S/H268A/V309L/A330S/P331S, F234A/L235A, S228P/F234A/L235A, N297A, V234A/G237A, K214T/E233P/L234V/L235A/G236-deleted/A327G/P331A/D365E/L358M, H268Q/V309L/A330S/P331S, S267E/L328F, L234F/L235E/D265A, L234A/L235A/G237A/P238S/H268A/A330S/P331S, S228P/F234A/L235A/G237A/P238S and S228P/F234A/L235A/G236-deleted/G237A/P238S, wherein residue numbering is according to the EU index.

In some embodiments, the first Ig constant region or the fragment of the first Ig constant region and the second Ig constant region or the fragment of the second Ig constant region comprises at least one mutation that results in enhanced binding of the multispecific protein to a Fcγ receptor (FcγR).

In some embodiments, the at least one mutation that results in enhanced binding of the multispecific protein to the FcγR is selected from the group consisting of S239D/I332E, S298A/E333A/K334A, F243L/R292P/Y300L, F243L/R292P/Y300L/P396L, F243L/R292P/Y300L/V305I/P396L and G236A/S239D/I332E, wherein residue numbering is according to the EU index. In some embodiments, the FcγR is FcγRI, FcγRIIA, FcγRIIB or FcγRIII, or any combination thereof.

In some embodiments, the first Ig constant region or the fragment of the first Ig constant region and the second Ig constant region or the fragment of the second Ig constant region comprises at least one mutation that modulates a half-life of the multispecific protein. In some embodiments, the at least one mutation that modulates the half-life of the multispecific protein is selected from the group consisting of H435A, P257I/N434H, D376V/N434H, M252Y/S254T/T256E/H433K/N434F, T308P/N434A, and H435R, wherein residue numbering is according to the EU index.

In some embodiments, the multispecific protein comprises at least one mutation in a CH3 domain of the first Ig constant region or in a CH3 domain of the fragment of the first Ig constant region and/or at least one mutation in a CH3 domain of the second Ig constant region or in a CH3 domain of the fragment of the second Ig constant region. In some embodiments, the at least one mutation in a CH3 domain of the first Ig constant region or in a CH3 domain of the fragment of the first Ig constant region and/or at least one mutation in a CH3 domain of the second Ig constant region or in a CH3 domain of the fragment of the second Ig constant region is selected from the group consisting of T350V, L351Y, F405A, Y407V, T366Y, T366W, T366L, F405W, K392L, T394W, T394S, Y407T, Y407A, T366S/L368A/Y407V, L351Y/F405A/Y407V, T366I/K392M/T394W, T366L/K392L/T394W, F405A/Y407V, T366L/K392M/T394W, L351Y/Y407A, L351Y/Y407V, T366A/K409F, L351Y/Y407A, T366V/K409F, T366A/K409F, T350V/L351Y/F405A/Y407V and T350V/T366L/K392L/T394W, wherein residue numbering is according to the EU index.

In some embodiments, the first Ig constant region or the fragment of the first Ig constant region and the second Ig constant region or the fragment of the second Ig constant region comprise the following mutations L234A_L235A_D265S_T350V_L351Y_F405A_Y407V in the first Ig constant region and L234A_L235A_D265S_T350V_T366L_K392L_T394W in the second Ig constant region; or L234A_L235A_D265S_T350V_T366L_K392L_T394W in the first Ig constant region and L234A_L235A_D265S_T350V_L351Y_F405A_Y407V in the second Ig constant region.

In some embodiments, the multispecific binding proteins provided herein are antibodies having a full-length antibody structure. “Full length antibody” refers to an antibody having two full length antibody heavy chains and two full length antibody light chains. A full-length antibody heavy chain (HC) consists of well-known heavy chain variable and constant domains VH, CH1, hinge, CH2, and CH3. A full-length antibody light chain (LC) consists of well-known light chain variable and constant domains VL and CL. The full-length antibody can be lacking the C-terminal lysine (K) in either one or both heavy chains. “Fab-arm” or “half molecule” refers to one heavy chain-light chain pair that specifically binds an antigen.

Full length bispecific antibodies can be generated for example using Fab arm exchange (or half molecule exchange) between two monospecific bivalent antibodies by introducing substitutions at the heavy chain CH3 interface in each half molecule to favor heterodimer formation of two antibody half molecules having distinct specificity either in vitro in cell-free environment or using co-expression. The Fab arm exchange reaction is the result of a disulfide-bond isomerization reaction and dissociation-association of CH3 domains. The heavy chain disulfide bonds in the hinge regions of the parental monospecific antibodies are reduced. The resulting free cysteines of one of the parental monospecific antibodies form an inter heavy-chain disulfide bond with cysteine residues of a second parental monospecific antibody molecule and simultaneously CH3 domains of the parental antibodies release and reform by dissociation-association. The CH3 domains of the Fab arms can be engineered to favor heterodimerization over homodimerization. The resulting product is a bispecific antibody having two Fab arms or half molecules which each bind a distinct epitope, i.e. an epitope on a target antigen of interest and an epitope on CD3.

“Homodimerization” refers to an interaction of two heavy chains having identical CH3 amino acid sequences. “Homodimer” refers to an antibody having two heavy chains with identical CH3 amino acid sequences. “Heterodimerization” refers to an interaction of two heavy chains having non-identical CH3 amino acid sequences. “Heterodimer” refers to an antibody having two heavy chains with non-identical CH3 amino acid sequences.

In some embodiments, the binding proteins provided herein include designs such as the Triomab/Quadroma (Trion Pharma/Fresenius Biotech), Knob-in-Hole (Genentech), CrossMAbs (Roche) and the electrostatically-matched (Chugai, Amgen, NovoNordisk, Oncomed), the LUZ-Y (Genentech), the Strand Exchange Engineered Domain body (SEEDbody) (EMD Serono), the Biclonic (Merus) and the DuoBody (Genmab A/S).

In some embodiments, a multispecific binding protein provided herein is in the knob-and-hole format. In some embodiments, a multispecific binding protein provided herein is in a DuoBody format.

The Triomab quadroma technology can be used to generate full length bispecific antibodies provided herein. Triomab technology promotes Fab arm exchange between two parental chimeric antibodies, one parental mAb having IgG2a and the second parental mAb having rat IgG2b constant regions, yielding chimeric bispecific antibodies.

The “knob-in-hole” strategy (see, e.g., International Publication No. WO 2006/028936) can be used to generate full length bispecific antibodies. Briefly, selected amino acids forming the interface of the CH3 domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob.” Exemplary CH3 substitution pairs forming a knob and a hole are (expressed as modified position in the first CH3 domain of the first heavy chain/modified position in the second CH3 domain of the second heavy chain): T366Y/F405A, T366W/F405W, F405W/Y407A, T394W/Y407T, T394S/Y407A, T366W/T394S, F405W/T394S and T366W/T366S_L368A_Y407V.

The CrossMAb technology can be used to generate full length bispecific antibodies provided herein. CrossMAbs, in addition to utilizing the “knob-in-hole” strategy to promoter Fab arm exchange, have in one of the half arms the CH1 and the CL domains exchanged to ensure correct light chain pairing of the resulting bispecific antibody (see e.g. U.S. Pat. No. 8,242,247).

Other cross-over strategies can be used to generate full length bispecific antibodies provided herein by exchanging variable or constant, or both domains between the heavy chain and the light chain or within the heavy chain in the bispecific antibodies, either in one or both arms. These exchanges include for example VH-CH1 with VL-CL, VH with VL, CH3 with CL and CH3 with CH1 as described in International Publication Nos. WO 2009/080254, WO 2009/080251, WO 2009/018386 and WO 2009/080252.

Other strategies such as promoting heavy chain heterodimerization using electrostatic interactions by substituting positively charged residues at one CH3 surface and negatively charged residues at a second CH3 surface can be used, as described in US Pat. Publ. No. US2010/0015133; US Pat. Publ. No. US2009/0182127; US Pat. Publ. No. US2010/028637; or US Pat. Publ. No. US2011/0123532. In other strategies, heterodimerization can be promoted by the following substitutions (expressed as modified position in the first CH3 domain of the first heavy chain/modified position in the second CH3 domain of the second heavy chain): L351Y_F405AY407V/T394W, T366I_K392M_T394W/F405A_Y407V, T366L_K392M_T394W/F405A_Y407V, L351Y_Y407A/T366A_K409F, L351Y_Y407A/T366V K409F Y407A/T366A_K409F, or T350V_L351Y_F405A Y407V/T350V_T366L_K392L_T394W as described in U.S. Pat. Publ. No. US2012/0149876 or U.S. Pat. Publ. No. US2013/0195849. Other strategies that can facilitate the removal of homodimer and/or half-molecule species include incorporating both substitutions H435R and Y436F in a heavy chain CH3 domain.

LUZ-Y technology can be utilized to generate bispecific antibodies provided herein. In this technology, a leucine zipper is added into the C terminus of the CH3 domains to drive the heterodimer assembly from parental mAbs that is removed post-purification as described in Wranik et al., (2012) J Biol Chem 287(52): 42221-9.

SEEDbody technology can be utilized to generate bispecific antibodies provided herein. SEEDbodies have, in their constant domains, select IgG residues substituted with IgA residues to promote heterodimerization as described in U.S. Patent No. US20070287170.

In addition to methods described above, binding agents provided herein can be generated in vitro in a cell-free environment by introducing asymmetrical mutations in the CH3 regions of two mono specific homodimeric antibodies and forming the bispecific heterodimeric antibody from two parent monospecific homodimeric antibodies in reducing conditions to allow disulfide bond isomerization according to methods described in PCT Pat. Publ. No. WO 2011/131746.

In some embodiments described herein, the bispecific CD3 binding agent described herein comprises a first binding region comprising a CD3 spFv and a second binding region binding a second antigen, and comprises at least one substitution in an antibody CH3 constant domain. Substitutions are typically made at the DNA level to a molecule such as the constant domain of the antibody using standard methods.

The antibodies provided herein can be engineered into various well-known antibody forms.

In some embodiments, the bispecific antibody is a diabody or a cross-body.

In some embodiments, the bispecific antibody includes IgG-like molecules with complementary CH3 domains that promote heterodimerization; recombinant IgG-like dual targeting molecules, wherein the two sides of the molecule each contain the Fab fragment or part of the Fab fragment of at least two different antibodies; IgG fusion molecules, wherein full length IgG antibodies are fused to an extra Fab fragment or parts of Fab fragment; Fc fusion molecules, wherein single chain Fv molecules or stabilized diabodies are fused to heavy-chain constant-domains, Fc-regions or parts thereof; Fab fusion molecules, wherein different Fab-fragments are fused together; ScFv- and diabody-based and heavy chain antibodies (e.g., domain antibodies, nanobodies) wherein different single chain Fv molecules or different diabodies or different heavy-chain antibodies (e.g. domain antibodies, nanobodies) are fused to each other or to another protein or carrier molecule.

In some embodiments, recombinant IgG-like dual targeting molecules include Dual Targeting (DT)-Ig (GSK/Domantis), Two-in-one Antibody (Genentech), Cross-linked Mabs (Karmanos Cancer Center), mAb2 (F-Star) and CovX-body (CovX/Pfizer).

In some embodiments, IgG fusion molecules include Dual Variable Domain (DVD)-Ig (Abbott), IgG-like Bispecific (ImClone/Eli Lilly), Ts2Ab (MedImmune/AZ) and BsAb (Zymogenetics), HERCULES (Biogen Idec) and TvAb (Roche).

In some embodiments, Fc fusion molecules can include ScFv/Fc Fusions (Academic Institution), SCORPION (Emergent BioSolutions/Trubion, Zymogenetics/BMS), Dual Affinity Retargeting Technology (Fc-DART) (MacroGenics) and Dual(ScFv)2-Fab (National Research Center for Antibody Medicine—China).

In some embodiments, Fab fusion bispecific antibodies include F(ab)2 (Medarex/AMGEN), Dual-Action or Bis-Fab (Genentech), Dock-and-Lock (DNL) (ImmunoMedics), Bivalent Bispecific (Biotecnol) and Fab-Fv (UCB-Celltech). ScFv-, diabody-based, and domain antibodies, include but are not limited to, Bispecific T Cell Engager (BiTE) (Micromet), Tandem Diabody (Tandab) (Affimed), Dual Affinity Retargeting Technology (DART) (MacroGenics), Single-chain Diabody (Academic), TCR-like Antibodies (AIT, ReceptorLogics), Human Serum Albumin ScFv Fusion (Merrimack) and COMBODY (Epigen Biotech), dual targeting nanobodies (Ablynx), dual targeting heavy chain only domain antibodies. Various formats of bispecific antibodies have been described, for example in Chames and Baty (2009) Curr Opin Drug Disc Dev 12: 276 and in Nunez-Prado et al., (2015) Drug Discovery Today 20(5):588-594.

Bispecific spFv CD3 Binding Proteins

In some embodiments, the bispecific spFv CD3 antibody includes a first variable domain that specifically binds a target antigen of interest and a second variable domain that specifically binds CD3, wherein the first variable domain that specifically binds a target antigen of interest includes a VH and a VL; and the second variable domain that specifically binds CD3 comprises an HCDR1, HCDR2, HCDR3, LCDR1, LCDR2, and LCDR3 from Table 3 and a Linker from Table 2.

In some embodiments, the bispecific spFv CD3 antibody includes a first variable domain that specifically bind a target antigen of interest and a second variable domain that specifically binds CD3, wherein the first variable domain that specifically binds a target antigen of interest includes a VH and a VL; and the second variable domain that specifically binds CD3 comprises a VH of SEQ ID NO: 28 and a VL of SEQ ID NO: 29.

In some embodiments, the bispecific spFv CD3 antibody includes a first variable domain that specifically bind a target antigen of interest and a second variable domain that specifically binds CD3, wherein the first variable domain that specifically binds a tumor antigen of interest includes a VH and a VL; and the second variable domain that specifically binds CD3 comprises a VH of SEQ ID NO: 28, a VL of SEQ ID NO: 29, and a Linker of Table 2.

In some embodiments, the bispecific spFv CD3 antibody includes a first variable domain that specifically bind a target antigen of interest and a second variable domain that specifically binds CD3, wherein the first variable domain that specifically binds a tumor antigen of interest includes a VH and a VL; and the second variable domain that specifically binds CD3 includes a spFv of SEQ ID NO: 30.

As described herein, any antigen other than CD3 can be selected as the second antigen target for the present multispecific CD3 binding agent. In specific embodiments, the multispecific binding agent is a multispecific binding protein.

In some embodiments, the bispecific spFv CD3 antibody used in the method of treatment of the disclosure include antibodies that specifically bind to CD3 and one or more additional target antigen. Exemplary target antigens include, but are not limited to bacterial antigens, viral antigens, tumor antigens, antigens associated with an autoimmune disease or disorder, and antigens associated with an inflammatory disease or disorder.

In one embodiment, one or more additional antigens are tumor antigens. Exemplary tumor antigens include, but are not limited to, prostate specific membrane antigen (PSMA) and ectonucleotide pyrophosphatase/phosphodiesterase family member 3 (ENPP3), claudin, and GUCY2C.

### Stabilized PSMA×CD3 Bispecific Antibodies

“PSMA” refers to prostate specific membrane antigen. The extracellular domain spans residues 44-750 of the full length PSMA. All references to proteins, polypeptides and protein fragments herein are intended to refer to the human version of the respective protein, polypeptide or protein fragment unless explicitly specified as being from a non-human species. Thus, “PSMA” means human PSMA unless specified as being from a non-human species, e.g., “mouse PSMA” or “monkey PSMA” etc.

In some embodiments, the bispecific antibody comprises a PSMA×CD3 antibody comprising, consisting of and/or consisting essentially of a first variable domain that binds PSMA and a second variable domain that binds CD3, wherein the first variable domain that binds PSMA or the second variable domain that binds CD3 is a disulfide stabilized scFv (spFv).

In some embodiments, the bispecific antibody comprises a PSMA×CD3 antibody comprising, consisting of and/or consisting essentially of a first variable domain that binds PSMA and a second variable domain that binds CD3, wherein the first variable domain that binds PSMA is a Fab and wherein the second variable domain that binds CD3 is a disulfide stabilized scFv (spFv) comprising, consisting of and/or consisting essentially of from the N- to C-terminus, a VL comprising a VL Cysteine, a Linker of Table 2 and a VH comprising a VH Cysteine (VL-Linker-VH).

In some embodiments, the bispecific antibody comprises a PSMA×CD3 antibody comprising, consisting of and/or consisting essentially of a first variable domain that binds PSMA and a second variable domain that binds CD3, wherein the first variable domain that binds PSMA is a Fab and wherein the second variable domain that binds CD3 is a disulfide stabilized scFv (spFv) comprising, consisting of and/or consisting essentially of from the N- to C-terminus, a VH comprising, consisting of and/or consisting essentially of a VH Cysteine, a Linker of Table 2 and a VL comprising a VL Cysteine (VH-Linker-VL).

Any PSMA binding molecule can be incorporated into a bispecific binding molecule of the disclosure.

In some embodiments, the bispecific antibody comprises a PSMA×CD3 antibody having a first variable domain that specifically binds to PSMA and a second variable domain that specifically binds to CD3, wherein the second variable domain that specifically binds CD3 includes a VH of SEQ ID NO: 28 and a VL of SEQ ID NO: 29 and a Linker of Table 2.

In some embodiments, the bispecific antibody comprises a PSMA×CD3 antibody includes a first variable domain that specifically bind PSMA, and a second variable domain that specifically binds to CD3, wherein the second variable domain that specifically binds CD3 includes a spFv of SEQ ID NO: 30.

### Stabilized ENPP3×CD3 Bispecific Antibodies

ENPP3 (CD203c) is a member of the cell surface ectonucleotide pyrophosphatase/phosphodiesterase (ENPP) family consisting of 7 structurally related molecules with ATPase and ATP pyrophosphatase activities involved in hydrolysis of extracellular nucleotides (Stefan et al., 2005, Trends Biochem Sci, 30(10):542-550). ENPP3 is a 150 kDa single-pass Type II transmembrane protein, with an extracellular region containing nuclease-like domain, a catalytic domain, and a somatomedin B-like domain (Borza et al., 2022, J Biol Chem, 298(2):101526; Hausmann et al., 2013, Adv Biol Regul, 53(1):112-117).

In some embodiments, the bispecific antibody comprises a ENPP3×CD3 antibody comprising, consisting of and/or consisting essentially of a first variable domain that binds PSMA and a second variable domain that binds CD3, wherein the first variable domain that binds PSMA or the second variable domain that binds CD3 is a disulfide stabilized scFv (spFv).

In some embodiments, the bispecific antibody comprises a ENPP3×CD3 antibody comprising, consisting of and/or consisting essentially of a first variable domain that binds ENPP3 and a second variable domain that binds CD3, wherein the first variable domain that binds ENPP3 is a Fab and wherein the second variable domain that binds CD3 is a disulfide stabilized scFv (spFv) comprising, consisting of and/or consisting essentially of from the N- to C-terminus, a VL comprising a VL Cysteine, a Linker of Table 2 and a VH comprising a VH Cysteine (VL-Linker-VH).

In some embodiments, the bispecific antibody comprises a ENPP3×CD3 antibody comprising, consisting of and/or consisting essentially of a first variable domain that binds ENPP3 and a second variable domain that binds CD3, wherein the first variable domain that binds ENPP3 is a Fab and wherein the second variable domain that binds CD3 is a disulfide stabilized scFv (spFv) comprising, consisting of and/or consisting essentially of from the N- to C-terminus, a VH comprising, consisting of and/or consisting essentially of a VH Cysteine, a Linker of Table 2 and a VL comprising a VL Cysteine (VH-Linker-VL).

Any ENPP3 binding molecule can be incorporated into a bispecific binding molecule of the disclosure.

In some embodiments, the bispecific antibody comprises a ENPP3×CD3 antibody having a first variable domain that specifically binds to ENPP3 and a second variable domain that specifically binds to CD3, wherein the second variable domain that specifically binds CD3 includes a VH of SEQ ID NO: 28 and a VL of SEQ ID NO: 29 and a Linker of Table 2.

In some embodiments, the bispecific antibody comprises a ENPP3×CD3 antibody includes a first variable domain that specifically binds to ENPP3, and a second variable domain that specifically binds to CD3, wherein the second variable domain that specifically binds CD3 includes a spFv of SEQ ID NO: 30.

### Stabilized Claudin×CD3 Bispecific Antibodies

Claudin 18.2 (CLDN18.2) is a 4-transmembrane domain tight junction protein that plays a role in cell-cell adhesion and regulation of paracellular ion transport in the gastric epithelium. In normal tissues, CLDN18.2 expression is strictly confined to differentiated epithelial cells of the gastric mucosa. CLDN18.2 is highly expressed in cancers of the digestive system including gastric and pancreatic adenocarcinomas, gastroesophageal junction (GEJ) tumors, esophageal adenocarcinomas, and metastatic lesions.

In some embodiments, the bispecific antibody comprises a CLDN18.2×CD3 antibody comprising, consisting of and/or consisting essentially of a first variable domain that binds CLDN18.2 and a second variable domain that binds CD3, wherein the first variable domain that binds GUCY2C or the second variable domain that binds CD3 is a disulfide stabilized scFv (spFv).

In some embodiments, the bispecific antibody comprises a CLDN18.2×CD3 antibody comprising, consisting of and/or consisting essentially of a first variable domain that binds CLDN18.2 and a second variable domain that binds CD3, wherein the first variable domain that binds CLDN18.2 is a Fab and wherein the second variable domain that binds CD3 is a disulfide stabilized scFv (spFv) comprising, consisting of and/or consisting essentially of from the N- to C-terminus, a VL comprising a VL Cysteine, a Linker of Table 2 and a VH comprising a VH Cysteine (VL-Linker-VH).

In some embodiments, the bispecific antibody comprises a CLDN18.2×CD3 antibody comprising, consisting of and/or consisting essentially of a first variable domain that binds CLDN18.2 and a second variable domain that binds CD3, wherein the first variable domain that binds CLDN18.2 is a Fab and wherein the second variable domain that binds CD3 is a disulfide stabilized scFv (spFv) comprising, consisting of and/or consisting essentially of from the N- to C-terminus, a VH comprising, consisting of and/or consisting essentially of a VH Cysteine, a Linker of Table 2 and a VL comprising a VL Cysteine (VH-Linker-VL).

Any CLDN18.2 binding molecule can be incorporated into a bispecific binding molecule of the disclosure.

In some embodiments, the bispecific antibody comprises a CLDN18.2×CD3 antibody having a first variable domain that specifically binds to CLDN18.2 and a second variable domain that specifically binds to CD3, wherein the second variable domain that specifically binds CD3 includes a VH of SEQ ID NO: 28 and a VL of SEQ ID NO: 29 and a Linker of Table 2.

In some embodiments, the bispecific antibody comprises a CLDN18.2×CD3 antibody includes a first variable domain that specifically binds CLDN18.2, and a second variable domain that specifically binds to CD3, wherein the second variable domain that specifically binds CD3 includes a spFv of SEQ ID NO: 30.

### Stabilized GUCY2C×CD3 Bispecific Antibodies

“GUCY2C” refers to guanylate cyclase 2C, also known as guanylyl cyclase C (GC-C), intestinal guanylate cyclase, guanylate cyclase-C receptor, or the heat-stable enterotoxin receptor (hSTAR). GUCY2C is an enzyme found in the luminal aspect of intestinal epithelium and dopamine neurons in the brain

In some embodiments, the bispecific antibody comprises a GUCY2C×CD3 antibody comprising, consisting of and/or consisting essentially of a first variable domain that binds GUCY2C and a second variable domain that binds CD3, wherein the first variable domain that binds GUCY2C or the second variable domain that binds CD3 is a disulfide stabilized scFv (spFv).

In some embodiments, the bispecific antibody comprises a GUCY2C×CD3 antibody comprising, consisting of and/or consisting essentially of a first variable domain that binds GUCY2C and a second variable domain that binds CD3, wherein the first variable domain that binds GUCY2C is a Fab and wherein the second variable domain that binds CD3 is a disulfide stabilized scFv (spFv) comprising, consisting of and/or consisting essentially of from the N- to C-terminus, a VL comprising a VL Cysteine, a Linker of Table 2 and a VH comprising a VH Cysteine (VL-Linker-VH).

In some embodiments, the bispecific antibody comprises a GUCY2C×CD3 antibody comprising, consisting of and/or consisting essentially of a first variable domain that binds GUCY2C and a second variable domain that binds CD3, wherein the first variable domain that binds GUCY2C is a Fab and wherein the second variable domain that binds CD3 is a disulfide stabilized scFv (spFv) comprising, consisting of and/or consisting essentially of from the N- to C-terminus, a VH comprising, consisting of and/or consisting essentially of a VH Cysteine, a Linker of Table 2 and a VL comprising a VL Cysteine (VH-Linker-VL).

Any GUCY2C binding molecule can be incorporated into a bispecific binding molecule of the disclosure.

In some embodiments, the bispecific antibody comprises a GUCY2C×CD3 antibody having a first variable domain that specifically binds to GUCY2C and a second variable domain that specifically binds to CD3, wherein the second variable domain that specifically binds CD3 includes a VH of SEQ ID NO: 28 and a VL of SEQ ID NO: 29 and a Linker of Table 2.

In some embodiments, the bispecific antibody comprises a GUCY2C×CD3 antibody includes a first variable domain that specifically binds GUCY2C, and a second variable domain that specifically binds to CD3, wherein the second variable domain that specifically binds CD3 includes a spFv of SEQ ID NO: 30.

### CD3 Antibody Fragment Variants

The disclosure also encompasses variants of the spFv CD3 binding molecules and bispecific antibodies comprising the spFv CD3 binding molecules described above.

The term “variants” refers to antibodies comprising one or more mutations, substitutions, deletions and/or additions of one or more amino acid residues. Such an addition, substitution or deletion can be located at any position in the molecule. In the case where several amino acids have been added, substituted or deleted, any combination of addition, substitution or deletion can be considered, on condition that the resulting antibody still has at least the advantageous properties of the antibody of the invention.

Sequences of the spFv CD3 antibodies may comprise amino acid sequences with at least 80% identity or homology to the sequences described above. In some embodiments, the sequence identity may be about 80%, 81%, 82%, 83%, 84%, 85%, 86%, 87%, 88%, 89%, 90%, 91%, 92%, 93%, 94%, 95%, 96%, 97%, 98% or 99% to the antigen binding domains that bind CD3 described above. Use of variants of the antigen binding domains that bind CD3 includes 1, 2, 3, 4, 5, 6, 7, 8, 9, 10, 11, 12, 13, 14, 15, 16, 17, 18, 19, 20, 21, 22, 23, 24, 25, 26, 27, 28 or 29 amino acid substitutions in the antigen binding domain that bind CD3 are within the scope of the disclosure, as long as they retain or have improved functional properties when compared to the parent antigen binding domains.

Variants of the antigen binding domains that bind CD3 include one or more deletions and/or additions of one or more amino acid residues. Such an addition, substitution or deletion can be located at any position in the molecule. In the case where several amino acids have been added, substituted or deleted, any combination of addition, substitution or deletion can be considered, on condition that the resulting antibody still has at least the advantageous properties of the antibody of the invention.

In some embodiments, the bispecific spFv CD3 antibody includes a first variable domain that specifically binds a target antigen of interest and a second variable domain that specifically binds CD3, wherein the second variable domain that specifically binds CD3 includes a VH which is at least 80% identical to the VH of SEQ ID NO: 28 and a VL which is at least 80% identical to the VL of SEQ ID NO: 29.

In some embodiments, the bispecific spFv CD3 antibody includes a first variable domain that specifically binds a target antigen of interest and a second variable domain that specifically binds CD3, wherein the second variable domain that specifically binds CD3 includes a spFv which is at least 80% identical to the spFv of SEQ ID NO: 30.

The terms “identical” or percent “identity,” in the context of two or more nucleic acids or polypeptide sequences (e.g., spFv CD3 antibodies and polynucleotides that encode them), refer to two or more sequences or subsequences that are the same or have a specified percentage of amino acid residues or nucleotides that are the same, when compared and aligned for maximum correspondence, as measured using one of the following sequence comparison algorithms or by visual inspection. The percent (%) amino acid sequence identity with respect to a reference polypeptide is defined as the percentage of amino acid residues in a given sequence that are identical to the amino acid residues in the reference polypeptide sequence. The percent (%) identity between the two sequences is a function of the number of identical positions shared by the sequences (i.e., % identity=number of identical positions/total number of positions ×100), taking into account the number of gaps, and the length of each gap, which need to be introduced for optimal alignment of the two sequences. The percent identity between two amino acid sequences can be determined using various the algorithms that are within the skill in the art, using publicly available software such as BLAS, BLAST-2, ALIGN. Megalin (DNASTAR) or the GAP program available in the GCG software package.

A polypeptide is typically substantially identical to a second polypeptide, for example, where the two peptides differ only by conservative substitutions. The antibodies of the present disclosure also include those for which binding characteristics, functional or physical properties have been improved by direct mutations. In some embodiments, variant antigen binding domains that bind CD3 comprise one or two conservative substitutions in any of the CDR regions, while retaining desired functional properties of the parent antibody that bind CD3.

“Conservative modifications” refer to amino acid modifications that do not significantly affect or alter the binding characteristics of the antibody containing the amino acid modifications. Conservative modifications include amino acid substitutions, additions and deletions. Conservative amino acid substitutions are those in which the amino acid is replaced with an amino acid residue having a similar side chain. The families of amino acid residues having similar side chains are well defined and include amino acids with acidic side chains (e.g., aspartic acid, glutamic acid), basic side chains (e.g., lysine, arginine, histidine), nonpolar side chains (e.g., alanine, valine, leucine, isoleucine, proline, phenylalanine, methionine), uncharged polar side chains (e.g., glycine, asparagine, glutamine, cysteine, serine, threonine, tyrosine, tryptophan), aromatic side chains (e.g., phenylalanine, tryptophan, histidine, tyrosine), aliphatic side chains (e.g., glycine, alanine, valine, leucine, isoleucine, serine, threonine), amide (e.g., asparagine, glutamine), beta-branched side chains (e.g., threonine, valine, isoleucine) and sulfur-containing side chains (cysteine, methionine). Furthermore, any native residue in the polypeptide may also be substituted with alanine, as has been previously described for alanine scanning mutagenesis (MacLennan et al., (1988) Acta Physiol Scand Suppl 643:55-67; Sasaki et al., (1988) Adv Biophys 35:1-24). Amino acid substitutions to the antibodies of the application can be made by known methods for example by PCR mutagenesis (U.S. Pat. No. 4,683,195).

Alternatively, libraries of variants can be generated for example using random (NNK) or non-random codons, for example DVK codons, which encode 11 amino acids (Ala, Cys, Asp, Glu, Gly, Lys, Asn, Arg, Ser, Tyr, Trp). The resulting variants can be tested for their characteristics using assays described herein.

### Polynucleotides, Vectors, and Host Cells

Also disclosed are isolated polynucleotides that encode the spFv CD3 binding molecules of the disclosure. The isolated polynucleotides capable of encoding the spFv CD3 binding molecules of the disclosure may be included on the same, or different, vectors to produce antibodies or antigen-binding fragments of the invention.

In some embodiments, the polynucleotides of the invention include a polynucleotide encoding a leader sequence. Any leader sequence known in the art may be employed. The polynucleotide encoding the leader sequence may include a restriction endonuclease cleavage site or a translation initiation site.

Also provided are vectors comprising the polynucleotides of the invention. The vectors can be expression vectors. The expression vector may contain one or more additional sequences such as but not limited to regulatory sequences (e.g., promoter, enhancer), a selection marker, and a polyadenylation signal.

Recombinant expression vectors within the scope of the description include synthetic, or cDNA-derived nucleic acid fragments that encode at least one recombinant protein which may be operably linked to suitable regulatory elements. Such regulatory elements may include a transcriptional promoter, sequences encoding suitable mRNA ribosomal binding sites, and sequences that control the termination of transcription and translation. Expression vectors, especially mammalian expression vectors, may also include one or more nontranscribed elements such as an origin of replication, a suitable promoter and enhancer linked to the gene to be expressed, other 5′ or 3′ flanking nontranscribed sequences, 5′ or 3′ nontranslated sequences (such as necessary ribosome binding sites), a polyadenylation site, splice donor and acceptor sites, or transcriptional termination sequences. An origin of replication that confers the ability to replicate in a host may also be incorporated.

The transcriptional and translational control sequences in expression vectors to be used in transforming vertebrate cells may be provided by viral sources. Exemplary vectors may be constructed as described by Okayama and Berg, 3 Mol. Cell. Biol. 280 (1983).

In some embodiments, the antibody- or antigen-binding fragment-coding sequence is placed under control of a powerful constitutive promoter. In addition, many viral promoters function constitutively in eukaryotic cells and are suitable for use with the described embodiments. In one embodiment, the coding sequence of the bispecific antibody of the invention or an antigen-binding fragment thereof is placed under control of an inducible promoter.

Vectors described herein may contain one or more Internal Ribosome Entry Site(s) (IRES). Inclusion of an IRES sequence into fusion vectors may be beneficial for enhancing expression of some proteins. In some embodiments the vector system will include one or more polyadenylation sites, which may be upstream or downstream of any of the aforementioned nucleic acid sequences.

The vectors may comprise selection markers. A nucleic acid sequence encoding a selection marker or the cloning site may be upstream or downstream of a nucleic acid sequence encoding a polypeptide of interest or cloning site.

The vectors described herein may be used to transform various cells with the genes encoding the described antibodies or antigen-binding fragments. For example, the vectors may be used to generate bispecific spFv CD3 binding molecules of the disclosure. Thus, the invention also provides a host cell comprising the vectors of the invention.

Numerous techniques are known in the art for the introduction of foreign genes into cells and may be used to construct the recombinant cells for purposes of carrying out the described methods, in accordance with the various embodiments described and exemplified herein. The technique used should provide for the stable transfer of the heterologous gene sequence to the host cell, such that the heterologous gene sequence is heritable and expressible by the cell progeny, and so that the necessary development and physiological functions of the recipient cells are not disrupted. Techniques which may be used include but are not limited to chromosome transfer (e.g., cell fusion, chromosome mediated gene transfer, micro cell mediated gene transfer), physical methods (e.g., transfection, spheroplast fusion, microinjection, electroporation, liposome carrier), viral vector transfer (e.g., recombinant DNA viruses, recombinant RNA viruses) and the like (described in Cline, 29 Pharmac. Ther. 69-92 (1985)). Calcium phosphate precipitation and polyethylene glycol (PEG)-induced fusion of bacterial protoplasts with mammalian cells may also be used to transform cells.

Cells suitable for use in the expression of the antibodies or antigen-binding fragments described herein are preferably eukaryotic cells, more preferably cells of plant, rodent, or human origin. In addition, expression of antibodies may be accomplished using hybridoma cells. Methods for producing hybridomas are well established in the art.

Cells transformed with expression vectors of the invention may be selected or screened for recombinant expression of the antibodies or antigen-binding fragments of the invention. Recombinant-positive cells are expanded and screened for subclones exhibiting a desired phenotype, such as high-level expression, enhanced growth properties, or the ability to yield proteins with desired biochemical characteristics, for example, due to protein modification or altered post-translational modifications. These phenotypes may be due to inherent properties of a given subclone or to mutation. Mutations may be effected through the use of chemicals, UV-wavelength light, radiation, viruses, insertional mutagens, inhibition of DNA mismatch repair, or a combination of such methods.

### Methods of Treatment

Provided herein are methods of treating a subject with a disease or disorder by administering to the subject at least an effective amount of a binding molecule comprising the spFv CD3 binding molecule, antibody comprising the spFv CD3 binding molecule or a variant of an spFv CD3 binding molecule or any pharmaceutical composition thereof. In some embodiments, the binding molecule comprises a bispecific antibody comprising a spFv CD3 binding arm and an antigen binding arm. In some embodiments, the antigen binding arm is specific for binding an antigen associated with the disease or disorder being treated.

“Treat”, “treating” or “treatment” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, delaying the progression of condition, relieving a condition caused by the disease or condition, or stopping the symptoms of the disease or condition either prophylactically and/or therapeutically.

As used herein, the terms “effective amount” refers to an amount sufficient to achieve a concentration of compound that is capable of treating a disease or condition. Such concentrations can be routinely determined by those of skilled in the art. The amount of the antibody of the present invention actually administered will typically be determined by a physician, in the light of the relevant circumstances, including the condition to be treated, the chosen route of administration, the actual compound administered, the age, weight, and response of the subject, the severity of the subject's symptoms, and the like. It will also be appreciated by those of skilled in the art that the dosage may be dependent on the stability of the administered antibody.

An effective amount may vary depending on factors such as the disease state, age, sex, and weight of the individual, the physical condition of the subject, the duration of the treatment, the nature of concurrent therapy (if any), the specific formulations employed, the structure of the antibody or of its variants and the ability of a therapeutic or a combination of therapeutics to elicit a desired response in the individual. An effective amount of the administered antibody may depend on the type and severity of the disease being treated, and the route of administration of the antibody polypeptide or the pharmaceutical composition of the antibody.

In case of conflict, the specification, including definitions, will control. As used herein and in the appended claims, the singular forms “a,” “an,” and “the” include plural referents unless the context clearly dictates otherwise. Thus, for example, reference to “a peptide sequence” or “a treatment,” includes a plurality of such sequences, treatments, and so forth. It is further noted that the claims can be drafted to exclude any optional element. As such, this statement is intended to serve as antecedent basis for use of such exclusive terminology such as “solely,” “only” and the like in connection with the recitation of claim elements, or use of a “negative” limitation.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges can independently be included in the smaller ranges, and are also encompassed within the invention, subject to any specifically excluded limit in the stated range. Where the stated range includes one or both of the limits, ranges excluding either or both of those included limits are also included in the invention.

As used herein, numerical values are often presented in a range format throughout this document. The use of a range format is merely for convenience and brevity and should not be construed as an inflexible limitation on the scope of the invention unless the context clearly indicates otherwise. Accordingly, the use of a range expressly includes all possible subranges, all individual numerical values within that range, and all numerical values or numerical ranges including integers within such ranges and fractions of the values or the integers within ranges, unless the context clearly indicates otherwise. This construction applies regardless of the breadth of the range and in all contexts throughout this patent document. Thus, for example, reference to a range of 90-100% includes 91-99%, 92-98%, 93-95%, 91-98%, 91-97%, 91-96%, 91-95%, 91-94%, 91-93%, and so forth. Reference to a range of 90-100% also includes 91%, 92%, 93%, 94%, 95%, 96%, 97%, etc., as well as 91.1%, 91.2%, 91.3%, 91.4%, 91.5%, etc., 92.1%, 92.2%, 92.3%, 92.4%, 92.5%, etc., and so forth. In addition, reference to a range of 1-3, 3-5, 5-10, 10-20, 20-30, 30-40, 40-50, 50-60, 60-70, 70-80, 80-90, 90-100, 100-110, 110-120, 120-130, 130-140, 140-150, 150-160, 160-170, 170-180, 180-190, 190-200, 200-225, 225-250 includes 1, 2, 3, 4, 5, 6, 7, 8, 9, 10, 11, 12, 13, 14, 15, 16, 17, 18, 19, 20, etc. In a further example, reference to a range of 25-250, 250-500, 500-1000, 1000-2500, 2500-5000, 5000-25,000, or 5000-50,000 includes any numerical value or range within or encompassing such values, e.g., 25, 26, 27, 28, 29 . . . 250, 251, 252, 253, 254 . . . 500, 501, 502, 503, 504 . . . , etc. The use of a series of ranges includes combinations of the upper and lower ranges to provide another range. This construction applies regardless of the breadth of the range and in all contexts throughout this patent document. Thus, for example, reference to a series of ranges such as 5-10, 10-20, 20-30, 30-40, 40-50, 50-75, 75-100, 100-150, includes ranges such as 5-20, 5-30, 5-40, 5-50, 5-75, 5-100, 5-150, and 10-30, 10-40, 10-50, 10-75, 10-100, 10-150, and 20-40, 20-50, 20-75, 20-100, 20-150, and so forth.

For the sake of conciseness, certain abbreviations are used herein. One example is the single letter abbreviation to represent amino acid residues. The amino acids and their corresponding three letter and single letter abbreviations are as follows:

| | | |
|---|---|---|
| alanine | Ala | (A) |
| arginine | Arg | (R) |
| asparagine | Asn | (N) |
| aspartic acid | Asp | (D) |
| cysteine | Cys | (C) |
| glutamic acid | Glu | (E) |
| glutamine | Gln | (Q) |
| glycine | Gly | (G) |
| histidine | His | (H) |
| isoleucine | Ile | (I) |
| leucine | Leu | (L) |
| lysine | Lys | (K) |
| methionine | Met | (M) |
| phenylalanine | Phe | (F) |
| proline | Pro | (P) |
| serine | Ser | (S) |
| threonine | Thr | (T) |
| tryptophan | Trp | (W) |
| tyrosine | Tyr | (Y) |
| valine | Val | (V) |

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include, aspects that are not expressly included in the invention are nevertheless disclosed herein.

Particular embodiments of this invention are described herein. Upon reading the foregoing description, variations of the disclosed embodiments may become apparent to individuals working in the art, and it is expected that those skilled artisans may employ such variations as appropriate. Accordingly, it is intended that the invention be practiced otherwise than as specifically described herein, and that the invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All publications, patent applications, accession numbers, and other references cited in this specification are herein incorporated by reference in its entirety as if each individual publication or patent application were specifically and individually indicated to be incorporated by reference. The publications discussed herein are provided solely for their disclosure prior to the filing date of the present application. Nothing herein is to be construed as an admission that the present invention is not entitled to antedate such publication by virtue of prior invention. Further, the dates of publication provided can be different from the actual publication dates which can need to be independently confirmed.

A number of embodiments of the invention have been described. Nevertheless, it will be understood that various modifications may be made without departing from the spirit and scope of the invention. Accordingly, the descriptions in the Experimental section are intended to illustrate but not limit the scope of invention described in the claims.

### EMBODIMENTS

A binding agent comprising an antigen binding region that binds to cluster of differentiation 3ε (CD3ε), wherein the antigen binding region that binds to CD3ε comprises a stapled scFv (spFv) comprising a variable heavy chain sequence (VH) and a variable light chain sequence (VL) specific for binding to CD3ε and a Linker sequence between the VH and VL, wherein the Linker comprises at least one cysteine residue (Cys) that serves as an anchor point.

The binding agent of embodiment 1, wherein the spFv comprises a disulfide bond between a surface exposed cysteine residue on at least one of the VH and VL the anchor point of the Linker.

The binding agent of embodiment 1, wherein the spFv comprises two disulfide bonds, wherein the first disulfide bonds forms between a surface exposed cysteine residue on the VH and a first anchor point of the Linker, and the second disulfide bonds forms between a surface exposed cysteine residue on the VL a second anchor point of the Linker.

The binding agent of embodiment 1, wherein the spFv is in the orientation of VH-Linker-VL.

The binding agent of embodiment 1, wherein the spFv is in the orientation of VL-Linker-VH.

The binding agent of any one of embodiments 1-5, wherein the Linker is selected from the group consisting of SEQ ID NO:2, SEQ ID NO:3, SEQ ID NO:4, SEQ ID NO:5 and SEQ ID NO:6.

The binding agent of any one of embodiments 1-6, wherein the antigen binding region that binds to CD3ε comprises CDR sequences selected from the group consisting of:
a) the HCDR1 comprises the amino acid sequence of SEQ ID NO:7, the HCDR2 comprises the amino acid sequence of SEQ ID NO:8, the HCDR3 comprises the amino acid sequence of SEQ ID NO:9, the LCDR1 comprises the amino acid sequence of SEQ ID NO:10, the LCDR2 comprises the amino acid sequence of SEQ ID NO:11, and the LCDR3 comprises the amino acid sequence of SEQ ID NO:12;
b) the HCDR1 comprises the amino acid sequence of SEQ ID NO:13, the HCDR2 comprises the amino acid sequence of SEQ ID NO:14, the HCDR3 comprises the amino acid sequence of SEQ ID NO:9, the LCDR1 comprises the amino acid sequence of SEQ ID NO:10, the LCDR2 comprises the amino acid sequence of SEQ ID NO:11, and the LCDR3 comprises the amino acid sequence of SEQ ID NO:12;
c) the HCDR1 comprises the amino acid sequence of SEQ ID NO:15, the HCDR2 comprises the amino acid sequence of SEQ ID NO:16, the HCDR3 comprises the amino acid sequence of SEQ ID NO:9, the LCDR1 comprises the amino acid sequence of SEQ ID NO:10, the LCDR2 comprises the amino acid sequence of SEQ ID NO:11, and the LCDR3 comprises the amino acid sequence of SEQ ID NO:12;
d) the HCDR1 comprises the amino acid sequence of SEQ ID NO:17, the HCDR2 comprises the amino acid sequence of SEQ ID NO:18, the HCDR3 comprises the amino acid sequence of SEQ ID NO:19, the LCDR1 comprises the amino acid sequence of SEQ ID NO:20 the LCDR2 comprises the amino acid sequence of SEQ ID NO:21, and the LCDR3 comprises the amino acid sequence of SEQ ID NO:22;
e) the HCDR1 comprises the amino acid sequence of SEQ ID NO:23, the HCDR2 comprises the amino acid sequence of SEQ ID NO:24, the HCDR3 comprises the amino acid sequence of SEQ ID NO:25, the LCDR1 comprises the amino acid sequence of SEQ ID NO:26, the LCDR2 comprises the amino acid sequence of DSS, and the LCDR3 comprises the amino acid sequence of SEQ ID NO:12.

The binding agent of embodiment 7, wherein in the antigen binding region that binds to CD3ε comprises a VH domain as set forth in SEQ ID NO:28, and a VL domain as set forth in SEQ ID NO:29.

The binding agent of embodiment 7, wherein in the antigen binding region that binds to CD3ε comprises a spFv as set forth in SEQ ID NO:30.

The binding agent of any one of embodiments 1 to 9, wherein the binding agent is a bispecific antibody or a multi-specific antibody.

The binding agent of any one of embodiments 1 to 10, further comprising an immunoglobulin (Ig) constant region, or a fragment of the Ig constant region, wherein optionally the fragment of the Ig constant region is an Fc region or an CH3 domain.

The binding agent of embodiment 11, wherein the Ig constant region, the fragment of the Ig constant region, the Fc region, or the CH3 domain comprises at least one mutation.

The binding agent of embodiment 12, wherein the at least one mutation is selected from the group consisting of L234A/L235A/D265S, F234A/L235A, L234A/L235A, V234A/G237A/P238S/H268A/V309L/A330S/P331S, F234A/L235A, S228P/F234A/L235A, N297A, V234A/G237A, K214T/E233P/L234V/L235A/G236-deleted/A327G/P331A/D365E/L358M, H268Q/V309L/A330S/P331S, S267E/L328F, L234F/L235E/D265A, L234A/L235A/G237A/P238S/H268A/A330S/P331S, S228P/F234A/L235A/G237A/P238S and S228P/F234A/L235A/G236-deleted/G237A/P238S, wherein residue numbering is according to the EU index.

The binding agent of embodiment 12, wherein the at least one mutation is selected from the group consisting of T366S/L368A/Y407V, T366W, T350V, L351Y, F405A, Y407V, T366Y, T366L, F405W, T394W, K392L, T394S, Y407T, Y407A, L351Y/F405A/Y407V, T366I/K392M/T394W, F405A/Y407V, T366L/K392M/T394W, T366L/K392L/T394W, L351Y/Y407A, L351Y/Y407V, T366A/K409F, T366V/K409F, T366A/K409F, T350V/L351Y/F405A/Y407V and T350V/T366L/K392L/T394W, wherein residue numbering is according to the EU index.

The binding agent of embodiment 12, wherein the binding agent comprises knob-in-hole mutations, wherein the knob mutations comprise T366S/L368A/Y407V, and the hole mutation comprises T366W.

The binding agent of any one of embodiments 10 to 15, wherein the agent comprises a bispecific protein comprising an antigen binding region that binds a second antigen other than CD3R.

The binding agent of embodiment 16, wherein the second antigen is a tumor antigen.

A composition comprising the binding agent of any one of embodiments 1 to 17, and a pharmaceutically acceptable carrier.

A polynucleotide comprising nucleotide sequences encoding a VH, a VL, or both a VH and a VL of the binding agent of any one of embodiments 1 to 17.

A vector comprising the polynucleotide of embodiment 19.

A cell comprising the polynucleotide of embodiment 19.

A kit comprising the binding agent of any one of embodiments 1 to 17.

A method of treating or slowing the progression of a disease or disorder in a subject, the method comprising administering to the subject at least one binding molecule of any one of embodiments 1-17.

A method of directing a T cell to a target cell expressing a target antigen, comprising contacting the T cell with an effective amount of the binding agent of embodiment 16 or 17 or a composition comprising the binding agent and a pharmaceutically acceptable carrier, wherein the antigen binding region that binds to CD3ε binds the T cell and the antigen binding region that binds to a second antigen other than CD3ε binds to the target cell.

### EXAMPLES

### Example 1: Design of Stabilized ScFvs

A monoclonal antibody (mAb) recognizes its target antigen through the two variable domains VL and VH. A single chain Fv (scFv) was first designed by Bird et al. (1988) Science 242:423-426 (1988) as a genetic fusion of VL and VH with a flexible linker in either VL-linker-VH or VH-linker-VL orientations. The flexible linker is typically three or four repeats of glycine-serine linker. A scFv recapitulates the antigen binding specificity and largely the affinity of its parental mAb. These scFv molecules have found wide applications as detection/diagnostics reagents or as building blocks for making more elaborate molecules such as bi-, multi-specific therapeutics (Brinkmann and Kontermann (2017) MAbs 9: 182-212) or in CAR-T therapeutics (Gross et al., (1989), Transplant Proc 21(1 Pt 1): 127-130; Porter et al., (2011) J Cancer 2: 331-332; Porter et al., (2011) N Engl J Med 365: 725-733).

One of the challenges of scFv molecules is the low stability and tendencies to aggregate (reviewed in Worn and Pluckthun (2001) J Mol Biol 305: 989-1010; Rothlisberger et al., (2005) J Mol Biol 347: 773-789). A number of strategies have been attempted to improve their properties (Arnd et al., (2001) J Mol Biol 312: 221-228; Monsellier et al., (2006) J Mol Biol 362: 580-593; Zhao et al., (2010) Int J Mol Sci 12: 1-11; Perchiacca and Tessier (2012) Annu Rev Chem Biomol Eng 3: 263-286; Asial et al., (2013) Nat Commmun 4: 2901; Gil and Schrum (2013) Adv Biosci Biteccchnol 4: 73-84; Tiller and Tessier (2015) Annu rev Biomed Eng 17: 191-216) These strategies include introducing disulfide bonds between VL/VH domains, improving VL/VH domain stability and/or interface interactions using different experimental methods, using additional dimerization motifs and others. A key difficulty is that most of these strategies are often specific to the VH/VL pair and cannot be readily transferred to other VH/VL pairs. Sometimes, engineering may have negative impact on the VL/VH structure and the scFv property. Recently, Zhang et al. introduced a disulfide between position 44 of VH and position 100 of VL of a an anti-aflatoxin Bi scFv (H4) and successfully achieved significant stabilization of the scFv while preserving its binding affinity (Zhao et al., (2010) Int J Mol Sci 12: 1-11). However, because of the distance and angle restraints between the chosen two positions, the inter-VL/VH disulfide, if applied to other VL/VH pairs, may restrict/distort the relative orientation between the two domains, which is often required for binding.

The interface between the heavy and light chains of the Fab fragment comprises VH/VL and CH1/CL interactions. The two independent sets of interactions provide synergistic stabilization effects. In addition, the V/C junction also contributes some stabilization effects. In comparison, in a scFv the VH/VL interface is maintained by the VH/VL interactions only. The linker, being designed to be flexible and non-restrictive except in cases where the length is designed to be so short to promote inter-scFv interactions for dimer and oligomer formation, only loosely couples the two together. It is known that the length and nature of the linker, when long enough, contributes little to the stability of the scFv.

### 1.1 “Stapling” Design

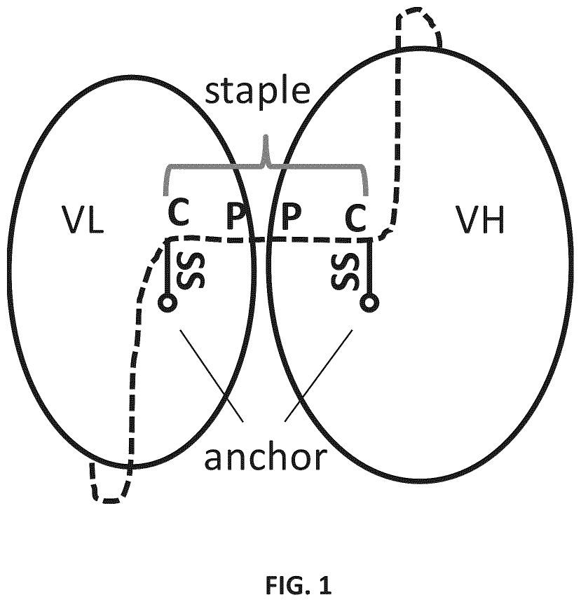

The purpose of the work was to design and generate stabilized scFvs by restraining but not negatively impacting the relative movements between the VH and the VL forming the scFv. This was accomplished by stabilizing the scFv by engineering disulfide bonds between the VH and the linker and between the VL and the linker. The restraints (i.e., disulfide bonds), when properly positioned, would then play the role of the synergistic effects afforded by the CH1/CL and V/C interactions discussed above. To this end, two structurally conserved surface exposed framework positions (anchor points) were identified, one each on VH and VL, which were non-overlapping with the typical predicted antigen binding site, and mutated into cysteine (Cys) residues. Two positions were subsequently chosen in the flexible linker for Cys positions. When the distances and locations between the linker Cys residues were designed in a manner that facilitated formation of disulfide bonds between the linker Cys and each anchor point, the VH and the VL would be tethered more tightly together when compared to tethering in the absence of the disulfide bonds. This scheme is depicted in FIG. 1 with an exemplary linker containing CPPC sequence (SEQ ID NO: 31). The concept of forming disulfide bonds between the flexible linker and anchor points is herein referred to as “stapling”. The resulting “stapled” scFv molecules are herein referred as spFv (“stapled Fv”).

### 1.2 Choice of the Anchor Points, Design of Staple Sequences and Linkers

For the stapling scheme to be widely applicable, it is important that the anchor points be structurally conserved, exposed on surface of both VL and VH and whose mutation to Cys residue will not impact folding of VL and VH or binding to antigens. The distances and geometry of the anchor points and the N and C termini of the VL and VH domains are also important considerations for proper disulfide formation.

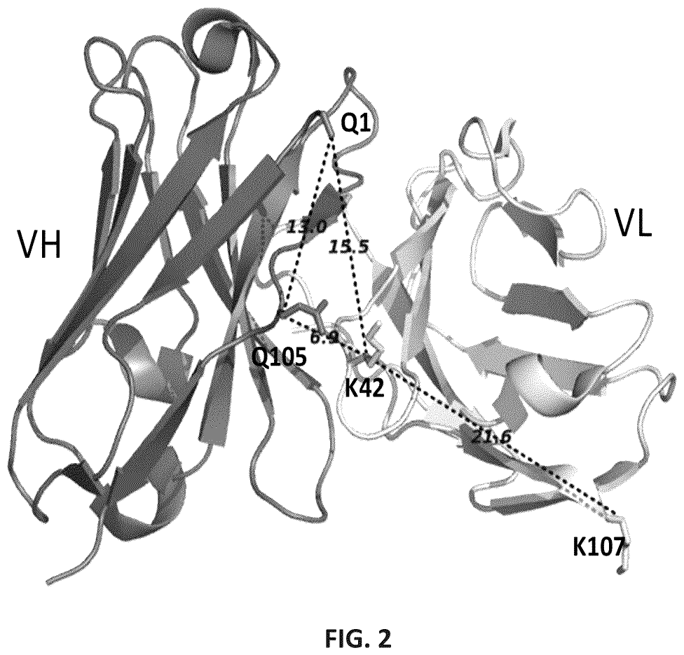

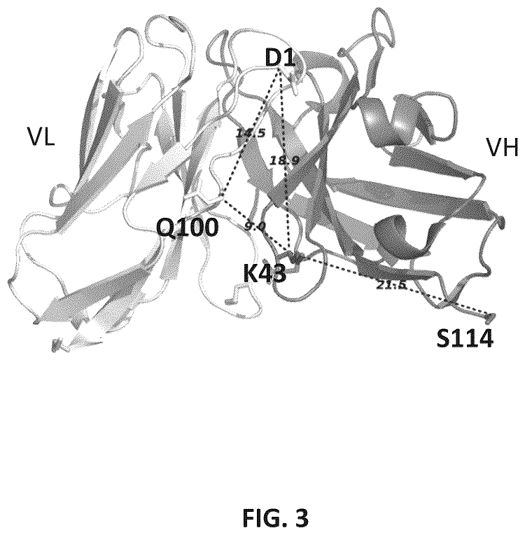

The anchor points were chosen separately for spFv in the VL-linker-VH and VH-linker-VL orientation. For the VL-linker-VH orientation, Chothia position 42 in the VL and Chothia position 105 in the VH were chosen as anchor points. A graphical illustration of the chosen anchor points for the spFv in the VL-linker-VH orientation is shown in FIG. 2 within the Fv of a human germline antibody (pdb ID 5I19, GLk1 hereafter). In GLk1, VL Chothia position 42 is lysine (K) and VH Chothia position 105 glutamine (Q). For the VH-linker-VL orientation, Chothia position 100 in the VL and Chothia position 43 in the VH were chosen as anchor points. FIG. 3 shows the graphical illustration of the chosen anchor points for the spFv in the VH-linker-VL orientation within the Fv of a human germline antibody (pdb ID 5I19, GLk1). In GLk1, VL Chothia position 100 is glutamine (Q) and VH Chothia position 43 is lysine (K). The chosen anchor points were structurally conserved, and the geometry was very similar in antibodies containing either kappa or lambda light chains. The distances between the pairs of the anchor points ranged from approximately 7 Å (for the VL-linker-VH orientation) to approximately 9 Å (for the VH-linker-VL orientation).

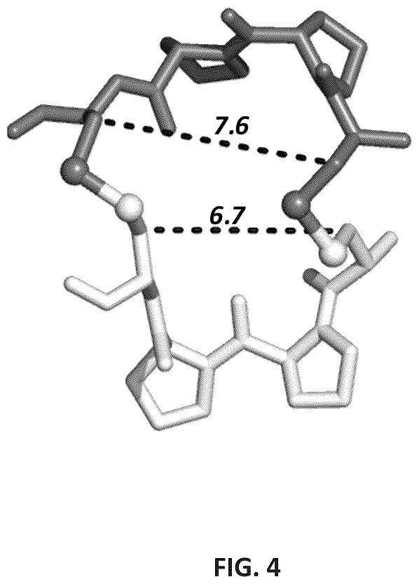

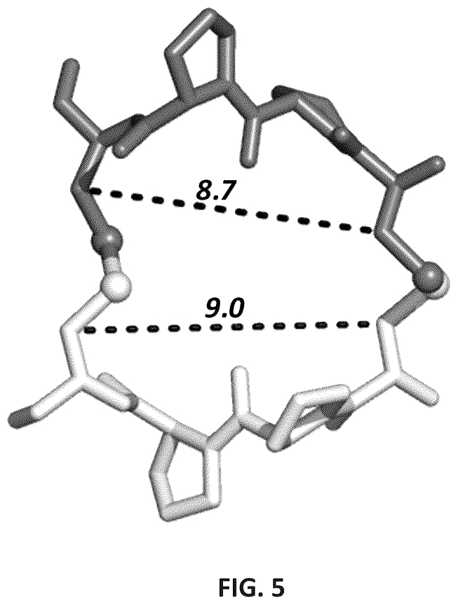

The staple sequences embedded within the linker connecting the VH and the VL were designed to be of similar length with the distances between the anchor points in the spFv. As an initial example of the staple sequence, CPPC (SEQ ID NO: 31) was chosen as a possible staple sequence, partly because this sequence occurs natively in human IgG1 hinge as well as in some rodent IgGs. The structures of the hinges of human and mouse IgG molecules demonstrated that the Cβ(cys1)-Cβ(cys2) distances in a mouse IgG hinge (FIG. 4) and a human IgG (FIG. 5) ranged from about 7 Å to 9 Å. As this range was very similar to the distances between the two anchor points in both VL-linker-VH and VH-linker-VL orientations, the CPPC (SEQ ID NO: 31) staple sequence had the potential to provide correct geometry for stapling, i.e., forming proper disulfide bonds efficiently and correctly to the anchor points. In general, the staple sequences were designed to have two Cys residues. For proper stapling, the N-terminal Cys of the staple sequence formed disulfide bond with the spFv N-terminal domain anchor point and the C-terminal Cys of the staple sequence formed a disulfide bond with the spFv C-terminal domain anchor point.

The linker connecting the VH and the VL was thus designed to comprises the staple sequence and connecting sequences both N-terminal and C-terminal to the staple sequence to extend the linker to provide sufficient linker length to allow intrachain folding of the VH and the VL and to facilitate proper positioning of the staple sequence.

In the VL-linker-VH design, the distances between the VL anchor point (K42), VH anchor point (Q105), C-terminus of the VL (K107) and the N-terminus of the VH (Q1) are shown in FIG. 2. In the VH-linker-VL design, the distances between the VL anchor point (Q100), the VH anchor point (K43), the C-terminus of the VH (S114) and the N-terminus of the VL (D1) are shown in FIG. 3. Modeling suggested that these distances can be spanned by linker lengths of about 14-19 residues, in which the staple sequence of 4 residues is flanked by a N-terminal linker extension of about 6-9 residues and a C-terminal linker extension of about 4-6 residues. The designed linker length could thus be expressed as n+4+m, in which n=6-9 residues and m=4-6 residues, and 4 indicates the length of the CPPC (SEQ ID NO: 31) staple sequence. The n and m residues could be glycine or serine, or other amino acid residues. These linker lengths are expected to be long and flexible enough to allow stapling but too short to allow scrambling.

### Example 2: Generation and Characterization of spFvs

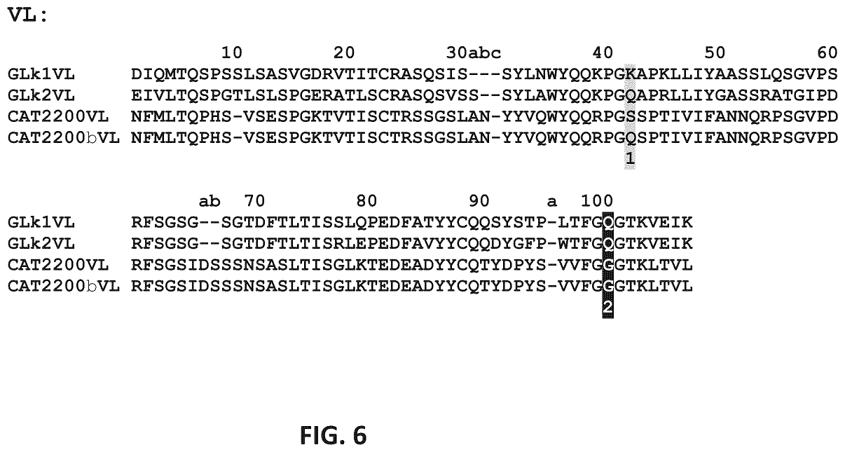

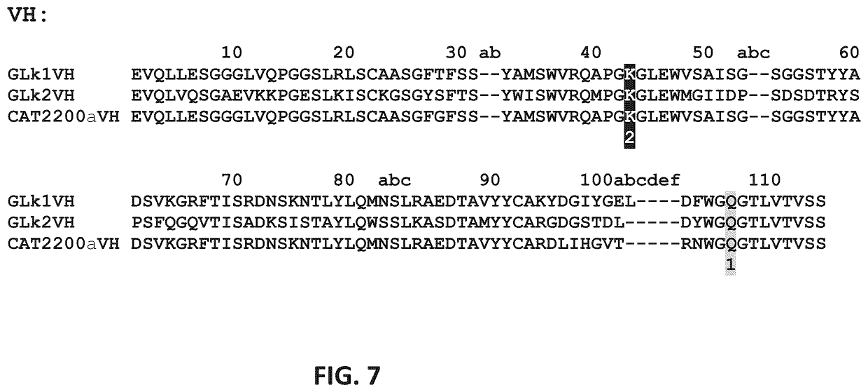

In order to assess the stapling designs, three human antibodies were chosen to generate scFv and corresponding spFvs: two antibodies with kappa light chains (GLk1 and GLk2) from the synthetic phage antibody libraries (Shi et al., (2010) J Mol Biol 397:385-396) and a lambda-containing antibody (CAT2200) obtained from a publication (Gerhardt et al. (2009) J Mol Biol 394:905-921). For CAT2200, a T28G mutation was introduced in the parental VH to generate a variant (CAT2200a) to reduce some of its interactions with its target, IL-17. In addition, a S42Q mutation (Chothia) was engineered into the parental CAT2200 VL and paired with the T28G VH to generate CAT2200b. The amino acid sequences of the VL and the VH domains of GLk1, GLk2, CAT2200a and CAT2200b are shown in FIG. 6 and FIG. 7, respectively. The VH domain amino acid sequence is identical between BAT2200a and CAT2200b. GLk1VH is closest to human IGHV2-23*01 GLk2VH to human IGHV5-51, CAT2200VH to human IGHV2-23*01. GLk1VL is closest human IGKV1-39*01, GLk2VL to human IGKV3-20*01, and CAT2200VL to human IGLV6-57*01.

All scFv and spFv molecules were generated and expressed in both VL-linker-VH and VH-linker-VL orientations. For the scFv constructs, a standard linker was used (SEQ ID NO:39). For the spFv, different linker lengths within the n and m ranges above were used. For GLk1 spFv, 9-4-5 linkers were used for both orientations. For GLk2 spFv, the 9-4-5 and 6-4-6 linker lengths were used for the VL-VH and VH-VL orientations, respectively. For CAT2200a spFv, VL-VH molecules were made with the 8-4-4 and 9-4-4 linkers, respectively, and CAT2200b spFv VH-VL was made with the 9-4-4 linker. Table 4 shows the generated molecules and their linker sequences. Table 5 shows the amino acid sequences or the generated molecules.

TABLE 4

| | | | Linker |
|---|---|---|---|
| | Linker | Linker amino acid | SEQ |
| Molecule Name | type | sequence | ID NO: |
| GLk1 scFv VL-VH | 4x G4S | GGGGSGGGGSGGGGSGGGGS | 39 |
| GLk1 spFv VL-VH | 9 + 4 + 5 | GGGSGGSGGCPPCGGSGG | 2 |
| GLk1 scFv VH-VL | 4x G4S | AGGGGSGGGGSGGGGSGGG | 156 |
| | | GS | |
| GLk1 spFv VH-VL | 9 + 4 + 5 | GGGSGGSGGCPPCGGSGG | 2 |
| GLk2 scFv VL-VH | 4x G4S | GGGGSGGGGSGGGGSGGGGS | 39 |
| GLk2 spFv VL-VH | 9 + 4 + 5 | GGGSGGSGGCPPCGGSGG | 2 |
| GLk2 scFv VH-VL | 4x G4S | GGGGSGGGGSGGGGSGGGGS | 39 |
| GLK2 spFv VH-VL | 6 + 4 + 6 | GGGSGGCPPCGGGSGG | 3 |
| CAT2200a scFv VL-VH | 4x G4S | GGGGSGGGGSGGGGSGGGGS | 39 |
| CAT2200a spFv VL-VH | 8 + 4 + 4 | GGSGGSGGCPPCGSGG | 4 |
| CAT2200b scFv VL-VH | 4x G4S | GGGGSGGGGSGGGGSGGGGS | 39 |
| CAT2200a spFv VL-VH | 9 + 4 + 4 | GGGSGGSGGCPPCGSGG | 5 |
| CAT2200a scFv VH-VL | 4x G4S | GGGGSGGGGSGGGGSGGGGS | 39 |
| CAT2200b spFv VH-VL | 9 + 4 + 4v2 | GGGSGGGSGCPPCGGGG | 6 |

TABLE 5

| | | SEQ ID |
|---|---|---|
| Molecule name | Protein sequence | NO: |
| GLk1 scFv VL-VH | DIQMTQSPSSLSASVGDRVTITCRASQSISSYLNWYQQKPGK | 40 |
| | APKLLIYAASSLQSGVPSRFSGSGSGTDFTLTISSLQPEDFAT | |
| | YYCQQSYSTPLTFGQGTKVEIKRGGGGSGGGGGGGGSGG | |
| | GGSEVQLLESGGGLVQPGGSLRLSCAASGFTFSSYAMSWVR | |
| | QAPGKGLEWVSAISGSGGSTYYADSVKGRFTISRDNSKNTL | |
| | YLQMNSLRAEDTAVYYCAKYDGIYGELDFWGQGTLVTVSS | |
| | GHHHHHH | |
| GLk1 spFv VL-VH | DIQMTQSPSSLSASVGDRVTITCRASQSISSYLNWYQQKPGC | 41 |
| | APKLLIYAASSLQSGVPSRFSGSGSGTDFTLTISSLQPEDFAT | |
| | YYCQQSYSTPLTFGQGTKVEIKRGGGSGGSGGCPPCGGSGG | |
| | EVQLLESGGGLVQPGGSLRLSCAASGFTFSSYAMSWVRQAP | |
| | GKGLEWVSAISGSGGSTYYADSVKGRFTISRDNSKNTLYLQ | |
| | MNSLRAEDTAVYYCAKYDGIYGELDFWGCGTLVTVSSGHH | |
| | HHHH | |
| GLk1 scFv VH-VL | EVQLLESGGGLVQPGGSLRLSCAASGFTFSSYAMSWVRQAP | 42 |
| | GKGLEWVSAISGSGGSTYYADSVKGRFTISRDNSKNTLYLQ | |
| | MNSLRAEDTAVYYCAKYDGIYGELDFWGQGTLVTVSSAGG | |
| | GGSGGGGSGGGGSGGGGSDIQMTQSPSSLSASVGDRVTITC | |
| | RASQSISSYLNWYQQKPGKAPKLLIYAASSLQSGVPSRFSGS | |
| | GSGTDFTLTISSLQPEDFATYYCQQSYSTPLTFGQGTKVEIKR | |
| | GHHHHHH | |
| GLk1 spFv VH-VL | EVQLLESGGGLVQPGGSLRLSCAASGFTFSSYAMSWVRQAP | 43 |
| | GCGLEWVSAISGSGGSTYYADSVKGRFTISRDNSKNTLYLQ | |
| | MNSLRAEDTAVYYCAKYDGIYGELDFWGQGTLVTVSSGGG | |
| | SGGSGGCPPCGGSGGDIQMTQSPSSLSASVGDRVTITCRASQ | |
| | SISSYLNWYQQKPGKAPKLLIYAASSLQSGVPSRFSGSGSGT | |
| | DFTLTISSLQPEDFATYYCQQSYSTPLTFGCGTKVEIKRGHH | |
| | HHHH | |
| GLk2 scFv VL-VH | EIVLTQSPGTLSLSPGERATLSCRASQSVSSSYLAWYQQKPG | 44 |
| | QAPRLLIYGASSRATGIPDRFSGSGSGTDFTLTISRLEPEDFA | |
| | VYYCQQDYGFPWTFGQGTKVEIKGGGGSGGGGSGGGGSG | |
| | GGGSEVQLVQSGAEVKKPGESLKISCKGSGYSFTSYWISWV | |
| | RQMPGKGLEWMGIIDPSDSDTRYSPSFQGQVTISADKSISTA | |
| | YLQWSSLKASDTAMYYCARGDGSTDLDYWGQGTLVTVSS | |
| | GHHHHHH | |
| GLk2 spFv VL-VH | EIVLTQSPGTLSLSPGERATLSCRASQSVSSSYLAWYQQKPG | 45 |
| | CAPRLLIYGASSRATGIPDRFSGSGSGTDFTLTISRLEPEDFAV | |
| | YYCQQDYGFPWTFGQGTKVEIKGGGSGGSGGCPPCGGSGG | |
| | EVQLVQSGAEVKKPGESLKISCKGSGYSFTSYWISWVRQMP | |
| | GKGLEWMGIIDPSDSDTRYSPSFQGQVTISADKSISTAYLQW | |
| | SSLKASDTAMYYCARGDGSTDLDYWGCGTLVTVSSGHHH | |
| | HHH | |
| GLk2 scFv VH-VL | EVQLVQSGAEVKKPGESLKISCKGSGYSFTSYWISWVRQMP | 46 |
| | GKGLEWMGIIDPSDSDTRYSPSFQGQVTISADKSISTAYLQW | |
| | SSLKASDTAMYYCARGDGSTDLDYWGQGTLVTVSSGGGGS | |
| | GGGGSGGGGSGGGGSEIVLTQSPGTLSLSPGERATLSCRASQ | |
| | SVSSSYLAWYQQKPGQAPRLLIYGASSRATGIPDRFSGSGSG | |
| | TDFTLTISRLEPEDFAVYYCQQDYGFPWTFGQGTKVEIKGH | |
| | HHHHH | |
| GLk2 spFv VH-VL | EVQLVQSGAEVKKPGESLKISCKGSGYSFTSYWISWVRQMP | 47 |
| | GCGLEWMGIIDPSDSDTRYSPSFQGQVTISADKSISTAYLQW | |
| | SSLKASDTAMYYCARGDGSTDLDYWGQGTLVTVSSGGGSG | |
| | GCPPCGGGSGGEIVLTQSPGTLSLSPGERATLSCRASQSVSSS | |
| | YLAWYQQKPGQAPRLLIYGASSRATGIPDRFSGSGSGTDFTL | |
| | TISRLEPEDFAVYYCQQDYGFPWTFGCGTKVEIKGHHHHHH | |
| CAT2200a scFv | NFMLTQPHSVSESPGKTVTISCTRSSGSLANYYVQWYQQRP | 48 |
| VL-VH | GSSPTIVIFANNQRPSGVPDRFSGSIDSSSNSASLTISGLKT | |
| | EDEADYYCQTYDPYSVVFGGGTKLTVLGGGGSGGGGSGGGGS | |
| | GGGGSEVQLLESGGGLVQPGGSLRLSCAASGFGFSSYAMS | |
| | WVRQAPGKGLEWVSAISGSGGSTYYADSVKGRFTISRDNSK | |
| | NTLYLQMNSLRAEDTAVYYCARDLIHGVTRNWGQGTLVT | |
| | VSSGHHHHHH | |
| CAT2200a spFv | NFMLTQPHSVSESPGKTVTISCTRSSGSLANYYVQWYQQRP | 49 |
| VL-VH | GCSPTIVIFANNQRPSGVPDRFSGSIDSSSNSASLTISGLKT | |
| | EDEADYYCQTYDPYSVVFGGGTKLTVLGGSGGSGGCPPCGSG | |
| | GEVQLLESGGGLVQPGGSLRLSCAASGFGFSSYAMSWVRQ | |
| | APGKGLEWVSAISGSGGSTYYADSVKGRFTISRDNSKNTLY | |
| | LQMNSLRAEDTAVYYCARDLIHGVTRNWGCGTLVTVSSGH | |
| | HHHHIH | |
| CAT2200b scFv | NFMLTQPHSVSESPGKTVTISCTRSSGSLANYYVQWYQQRP | 50 |
| VL-VH | GQSPTIVIFANNQRPSGVPDRFSGSIDSSSNSASLTISGLKT | |
| | EDEADYYCQTYDPYSVVFGGGTKLTVLGGGGSGGGGSGGGGS | |
| | GGGGSEVQLLESGGGLVQPGGSLRLSCAASGFGFSSYAMS | |
| | WVRQAPGKGLEWVSAISGSGGSTYYADSVKGRFTISRDNSK | |
| | NTLYLQMNSLRAEDTAVYYCARDLIHGVTRNWGQGTLVT | |
| | VSSGHHHHHH | |
| CAT2200a spFv | NFMLTQPHSVSESPGKTVTISCTRSSGSLANYYVQWYQQRP | 51 |
| VL-VH | GCSPTIVIFANNQRPSGVPDRFSGSIDSSSNSASLTISGLKT | |
| | EDEADYYCQTYDPYSVVFGGGTKLTVLGGGSGGSGGCPPCGS | |
| | GGEVQLLESGGGLVQPGGSLRLSCAASGFGFSSYAMSWVR | |
| | QAPGKGLEWVSAISGSGGSTYYADSVKGRFTISRDNSKNTL | |
| | YLQMNSLRAEDTAVYYCARDLIHGVTRNWGCGTLVTVSSG | |
| | HHHHIHIH | |
| CAT2200a scFv | EVQLLESGGGLVQPGGSLRLSCAASGFGFSSYAMSWVRQAP | 52 |
| VH-VL | GKGLEWVSAISGSGGSTYYADSVKGRFTISRDNSKNTLYLQ | |
| | MNSLRAEDTAVYYCARDLIHGVTRNWGQGTLVTVSSGGG | |
| | GSGGGGSGGGGSGGGGSNFMLTQPHSVSESPGKTVTISCTR | |
| | SSGSLANYYVQWYQQRPGSSPTIVIFANNQRPSGVPDRFSGS | |
| | IDSSSNSASLTISGLKTEDEADYYCQTYDPYSVVFGGGTKLT | |
| | VLGHHHHHH | |
| CAT2200b spFv | EVQLLESGGGLVQPGGSLRLSCAASGFGFSSYAMSWVRQAP | 53 |
| VH-VL | GCGLEWVSAISGSGGSTYYADSVKGRFTISRDNSKNTLYLQ | |
| | MNSLRAEDTAVYYCARDLIHGVTRNWGQGTLVTVSSGGGS | |
| | GGGSGCPPCGGGGNFMLTQPHSVSESPGKTVTISCTRSSGSL | |
| | ANYYVQWYQQRPGQSPTIVIFANNQRPSGVPDRFSGSIDSSS | |
| | NSASLTISGLKTEDEADYYCQTYDPYSVVFGCGTKLTVLGH | |
| | HHHHH | |

All scFv and spFv molecules except CAT2200a scFv VL-VH were cloned into a CMV promoter driven mammalian expression vector. These constructs were transfected into Expi293 cells using manufacturer protocols and cells were cultured for 5 days. Each Protein was purified from the clarified supernatant on 1 ml His-TRAP HP columns (GE Healthcare) via an AKTAXPRESS system (GE Healthcare). The column was prepared with a gradient of 0-100% Elution Buffer (Wash Buffer: 50 mM Tris, pH 7.5, 500 mM NaCl, 20 mM Imidazole; Elution Buffer: 50 mM Tris, pH 7.5, 500 mM NaCl, 500 mM Imidazole) to remove loosely bound nickel and then re-equilibration in DPBS. The cleared supernatant was first adjusted to 50 mM Tris, pH 7.5 and 20 mM imidazole and then loaded over 1 mL HisTRAP HP column at 4° C. 0.8 mL/min. The column was then washed with PBS until stable baseline was obtained. Then the column was further washed with 20 CV of Wash Buffer, eluted with Elution buffer into a single injection loop and desalted in 1×DPBS over 26/10 HiPrep Desalting Column and fractions collected. Fractions containing the purified protein were then pooled and concentrated. The Glk2 scFv and spFv proteins were dialyzed into DPBS for thermal stability measurements (DSC and NanoDSF) and 25 mM Tris, pH 7.5 and 100 mM NaCl for other studies. The other scFv and spFv proteins were dialyzed in 25 mM IES, pH 6.0 and 100 mM NaCl.

CAT2200a scFv VL-VH was purchased from a vendor. Concentration was 0.77 mg/mL in DPBS, pH 7.2. A mutant of IL-17 (12-132 with K70Q A132Q C106S mutations, IL-17 hereafter for simplicity (SEQ ID NO: 22) was purchased from Accelagen (CA). The protein was refolded from E. coli inclusion body following their proprietary refolding protocol and provided at 1.50 mg/mL in 20 mM NaCl, 20 mM MES, pH 6.0.

2.1 Thermal Stability of the Generated scFv and spFv Molecules

The thermal stability of the scFv and spFv molecules was investigated by differential thermal calorimetry (DSC). The scFv and spFv proteins were dialyzed overnight against 1×DPBS (Gibco) for GLk1 and CAT2200a/CAT2200b or MES (25 mM MES, pH 6.0, 100 mM NaCl) for GLk2. Dialysis buffer was then 0.22 micron filtered and used as the reference solution and for buffer-buffer blanks in the DSC experiment. Proteins were diluted to ˜0.5 mg/mL in the filtered buffer and 400 μL of each protein or buffer sample was loaded into a 96-deepwell plate (MicroLiter Analytical Supplies, 07-2100) and kept at 4° C. in the autosampler drawer over the course of the experiment. A MicroCal Capillary DSC with Autosampler (Malvern) was used to perform the DSC experiments. DSC scans were performed from 25-95° C. at a 60° C./h scan rate with no sample rescans. No feedback was selected and the filtering period was set at 15 s. After each sample, cells were cleaned with a 10% Contrad-70 solution and a buffer-buffer blank was run. Data analysis was performed using Origin 7.0 with the MicroCal VP-Capillary DSC Automated Analysis add-on (Malvern). The baseline range and type were manually chosen and then subtracted. The previous buffer blank was subtracted from the sample curve followed by concentration-dependent normalization. The thermal melting profiles were analyzed using both 2-state and non-2-state transitions. Two-state fits (one transition) agreed poorly with the experimental curves. Thus, with two transitions (Tm1 and Tm2) were calculated by manually performing non-2-state fits. The Tm data are reported in Table 6. The DSC profiles of all scFv and spFv proteins exhibited a skewness that could only be fitted with non-2-state transitions. Thus, for each scFv or spFv, two transitions (Tm1 and Tm2) were reported (Table 6). Most likely, these two transitions correspond to the melting Tm of the VL and VH domains, respectively. In general, upon comparison, the differences between scFv and spFv for either Tm1 or Tm2, there is a roughly 10° C. increase by stapling, regardless of the Tm of the starting scFv. There is only one exception, i.e., the case of GLk2 scFv and spFv (VH-VL orientation) difference, at ˜7° C. This is likely due to the shorter 6+4+6 linker which may have caused slight strain in the stapling geometry. The fact that ΔTm1 (VL) and ΔTm2 (VH) were nearly identical suggests that stapling lead to stabilization of the domains themselves in addition to strengthening the VL/VH interactions. Alternatively, stronger VH/VL interactions transmits the stabilization effects into stabilization of the VL/VH domains. In summary, stapling as described in this work significantly increases the stability of scFv.

TABLE 6

| | | DSC Stability | | | | | | |
|---|---|---|---|---|---|---|---|---|
| | Linker | Tm1 | SD(Tm1) | Tm2 | SD(Tm2) | ΔTm1 | ΔTm2 | Δ(Tm1 − |
| Molecule name | type | (° C.) | (° C.) | (° C.) | (° C.) | (° C.) | (° C.) | Tm2) |
| GLk1 scFv VL-VH | 4x G4S | 68.9 | 0.2 | 72.1 | 0.0 | 9.7 | 9.0 | 3.3 |
| GLk1 spFv VL-VH | 9 + 4 + 5 | 78.5 | 0.2 | 81.1 | 0.0 | | | 2.6 |
| GLk1 scFv VH-VL | 4x G4S | 67.7 | 0.3 | 70.9 | 0.0 | 10.0 | 9.2 | 3.3 |
| GLk1 spFv VH-VL | 9 + 4 + 5 | 77.7 | 0.2 | 80.1 | 0.1 | | | 2.4 |
| GLk2 scFv VL-VH | 4x G4S | 56.6 | 0.2 | 58.6 | 0.1 | 10.9 | 10.5 | 2.0 |
| GLk2 spFv VL-VH | 9 + 4 + 5 | 67.6 | 0.3 | 69.2 | 0.1 | | | 1.6 |
| GLk2 scFv VH-VL | 4x G4S | 56.4 | 0.1 | 58.3 | 0.1 | 7.3 | 7.1 | 1.9 |
| GLk2 spFv VH-VL | 6 + 4 + 6 | 63.7 | 0.3 | 65.4 | 0.1 | | | 1.7 |
| CAT2200a scFv VL-VH | 4x G4S | | | | | | | |
| CAT2200a spFv VL-VH | 8 + 4 + 4 | | | | | | | |
| CAT2200b scFv VL-VH | 4x G4S | 54.7 | 0.3 | 57.6 | 0.0 | 12.0 | 11.7 | 2.9 |
| CAT2200a spFv VL-VH | 9 + 4 + 4 | 66.7 | 0.3 | 69.3 | 0.0 | | | 2.6 |
| CAT2200a scFv VH-VL | 4x G4S | 52.6 | 0.3 | 56.4 | 0.0 | 12.9 | 11.4 | 3.8 |
| CAT2200b spFv VH-VL | 9 + 4 + 4 | 65.5 | 0.3 | 67.9 | 0.0 | | | 2.4 |

ΔTm1 (° C.): Difference in Tm1 (spFv) and Tm1 (scFv)
ΔTm2 (° C.): Difference in Tm2 (spFv) and Tm2 (scFv)
Δ(Tm1 − Tm2): Difference of Tm2 and Tm1 of an scFv or spFv

### Example 3: Verification of Correct Stapling by Crystallization of Generated Scfv and SpFv Molecules

Proteins were concentrated in their respective buffers: GLk1 spFv VL-VH to 8.67 mg/mi in 25 mM MES, pH 6.0, 100 mM NaCl; GLk1 spFv VH-VL to 5 mg/mi in 25 mM MES, pH 6.0, 100 mM NaCl; GLk2 spFv VH-VL to 8.66 mg/mi in 25 mM Tris, pH17.5, 100 mM NaCl; cat2200b spFv VH-VL to 25 mM MES, pH 6.0, 100 mM NaCl. Crystallization was set up for each protein in sitting drop format in Corning 3550 crystal trays using a Mosquito robot. Each well contains 100 nl of protein and 100 nl of reservoir solution and incubated against 70 μl of reservoir at 20° C. The reservoir solutions are IH1 and IH12 custom conditions as well as PEG Jon Screen UT (Hampton Research). Some initial conditions were refined by varying reservoir components in optimization attempts. Diffraction quality crystals were obtained for some of scFv and spFv proteins. Table 7 shows the summary of the conditions used. Crystals were soaked for a few seconds in the mother liquor supplemented with 2000 glycerol and flash frozen in liquid N2. X-ray data were collected at IMCA-CAT Beamline 171D at Argonne National Lab.

TABLE 7

| Molecule | | | | Resolution |
|---|---|---|---|---|
| name | Protein | crystallization Condition | Disulfides? | (Å) |
| GLk1 spFv | 8.67 mg/mL, 25 mM MES, | 0.1M Na Acet 4.5 pH, | yes | 1.65 |
| VL-VH | pH 6.0, 100 mM NaCl | 18% w/v PEG 3350, 1M | | |
| | | LiCl | | |
| Glk1 spFv | 5 mg/mL, 25 mM MES, | 0.1M MES 6.5 pH, 5% | yes | 2.10 |
| VH-VL | pH 6.0, 100 mM NaCl | v/v PEG 400, 0.75M | | |
| | | (NH4)2SO4 | | |
| GLK2 spFv | 8.66 mg/mL , 25 mM Tris, | 0.1M Na Acet 4.5 pH, | yes | 1.50 |
| VH-VL | pH 7.5, 100 mM NaCl | 0.75M (NH4)2SO4 | | |
| CAT2200b | 4.91 mg/mL, 25 mM MES, | 0.1M Na Acet 4.5 pH, | yes | 2.40 |
| spFv | pH 6.0 100 mM NaCl | 1.5M (NH4)2SO4 | | |
| VH-VL | | | | |
| CAT2200a | 2.69 mg/mL, 20 mM | 0.1M Tris, pH 8.5, | non-stapled | 2.30 |
| scFv VL-VH/ | HEPES, pH 7.5, 50 mM | 18% PEG3K, 0.2M | | |
| IL-17 | NaCl | LiSO4 | | |
| complex | | | | |
| CAT2200a | 6.0 mg/ml, 20 mM | 15.5% PEG 3350, 0.4M | yes | 2.00 |
| spFv VL-VH/ | HEPES, pH 7.5, 50 mM | NaH₂PO₄ | | |
| IL-17 | NaCl | | | |
| complex | | | | |

3.1 Crystallization of CAT2200a scFv VL-VH and CAT2200a spFv VL-VH in Complex with IL-17

The IL-17/CAT2200a scFv VL-VH complex was generated by mixing 333 μL of IL17 (1.5 mg/ml) with 1.74 ml of Cat2200a scFv (0.69 mg/mL) and incubating for 3 hours at 4° C. The mixture was concentrated with 10 kDa cutoff Amicon Ultra concentrator to about 400 μL and loaded onto a Superdex75 column equilibrated in 250 mM NaCl, 20 mM HEPES, pH 7.5. The fractions corresponding to the complex were pooled and concentrated to a volume of 150 μL. The sample was diluted and concentrated 4 times: addition of 350 μL 50 mM NaCl, 20 mM HEPES, pH 7.5 and concentration to just under 150 μL. The volume was brought to ˜105 μL and concentration determined to be 2.69 mg/mL. Crystallization was set up in a sitting drop format using a Mosquito crystallization robot with 150 nL protein+150 nL reservoir in Corning3550 plates against 80 μL reservoir, which is a set of buffer and precipitant conditions pre-formulated in-house. The plates were incubated at 20° C. One of conditions (Na Acetate, pH 4.5, 25% PEG3K, 0.2M Am Acetate) produced very small crystals. These were harvested and turned into crystallization seeds using Hampton Seed Bead in 100 μL 27% PEG 3350, 200 mM ammonium acetate, 100 mM sodium acetate, pH 4.5 in a Hampton Seed Bead tube.

Diffraction quality crystals were obtained by the same procedure except with the addition of the seeds above: 150 nL protein+100 nL reservoir+50 μL seeds. Crystals grew from 0.1 M Tris 8.5, 18% PEG3K, 0.2M LiSO4 and were transferred to a synthetic mother liquor (0.1 M Tris, pH 8.5, 10% PEG 3350, 0.2 M LiSO4 and 20% glycerol) and flash frozen in liquid nitrogen. X-ray diffraction data were collected at IMCA-CAT ID17 at Argonne National Laboratory.

The IL-17/CAT2200a spFv VL-VH complex were generated by mixing 167 μl of IL-17 (250 μg) with 154 μl MSCW274 (467 μg in 250 mM NaCl, 20 mM MES, pH 6.5) and incubating at 4° C. overnight. The mixture was concentrated in a 10 kDa MWCO Amicon Ultra 0.5 mL concentrator to ˜100 μL, then repeatedly diluted and concentrated 5 times: concentrate to ˜150 μL and added 350 μL 50 mM NaCl, 20 mM HEPES, pH 7.5. The final volume was 100 μL and the concentration of the complex was determined to be 6.0 mg/ml. Crystallization was set up similarly as for scFv/IL-17 complex in sitting drops using the Mosquito robot. The sitting drop are composed of 150 nL protein+120 nL reservoir+30 nL seeds (scFv/IL-17 above). The reservoir solution were a set of conditions varying PEG 3350 concentration and salts. The crystallization plates were incubated at 20° C. Small crystals were obtained from 15.5% PEG 3350, 0.4 M NaH₂PO₄. Crystals were transferred into 16% PEG 3350, 0.2 M NaH₂PO₄, 20% Glycerol, and flash frozen LN2. X-ray diffraction data were collected at IMCA-CAT ID17 at Argonne National Laboratory.

All X-ray diffraction data were processed with XDS (Kabsch et al. (2010) Acta Crystallogr D Biol Crystallogr 66(Pt. 2):125-132; Monsellier and Bedouelle (2006) J Mol Biol 362:580-593) and CCP4 (Collaborative Computational Project, N. (1994) Acta Crystallogr D Biol Crystallogr 53:240-255). All crystal structures were solved by molecular replacement (MR) using Phaser (Read (2001) Acta Crystallogr D Biol Crystallogr 57(Pt 10):1373-1382) with homology models generated in MOE (Montreal, Canada) except for scFv CAT2200a scFv VL-VH/IL-17 complex, for which the structure of pdb id 2vxs (Gerhardt et al. (2009) J Mol Biol 394:905-921) was used as search models. The structural models were refined in PHENIX (Adams et al. (2004) J Synchrotron Radiat 11(Pt 1):53-55) and manually adjusted in Coot (Emsley et al. (2010) Acta Crystallogr D Biol Crystallogr 66(Pt 4):486-501). Molecular graphics figures were generated in PyMol (www_schrodinger_com).

### 3.1.1 the Structures

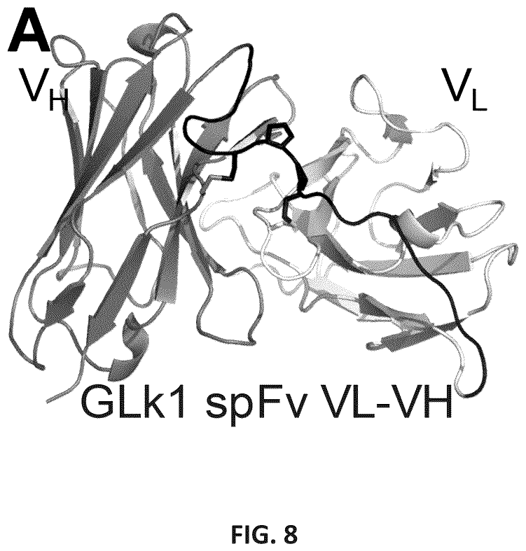

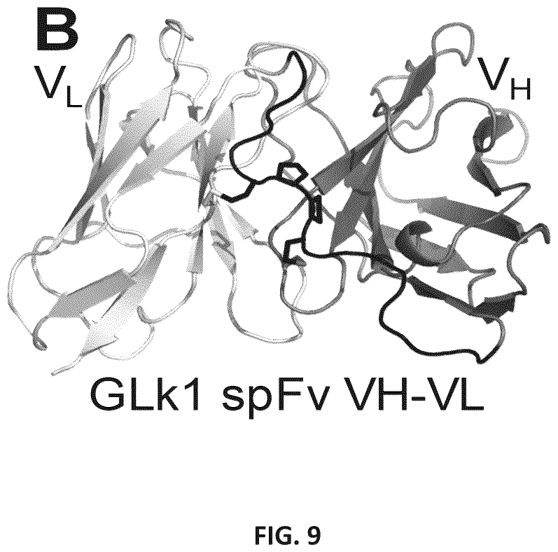

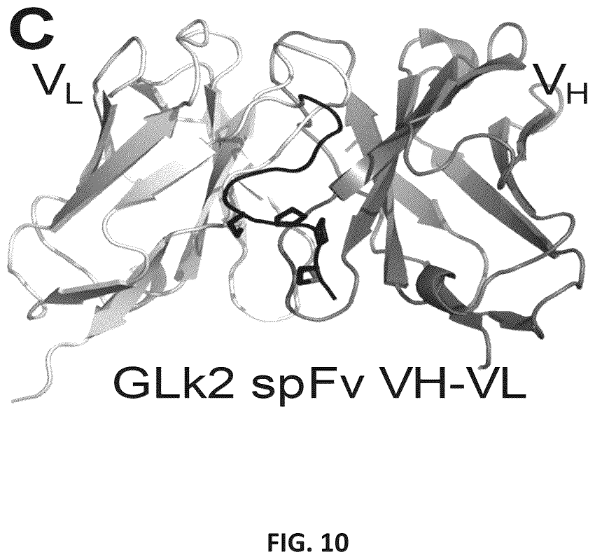

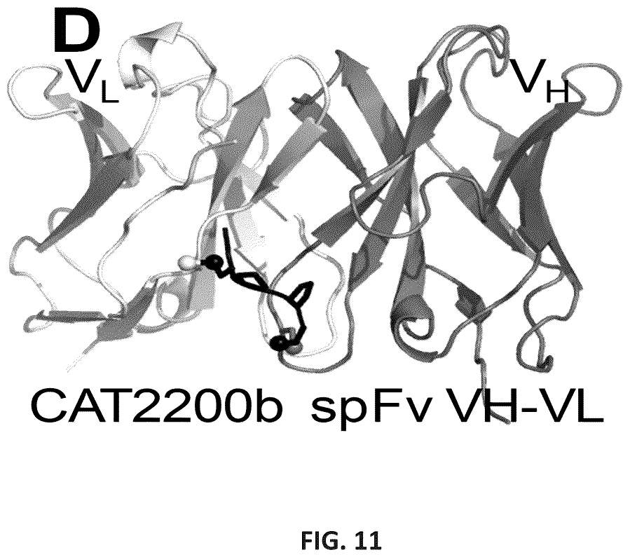

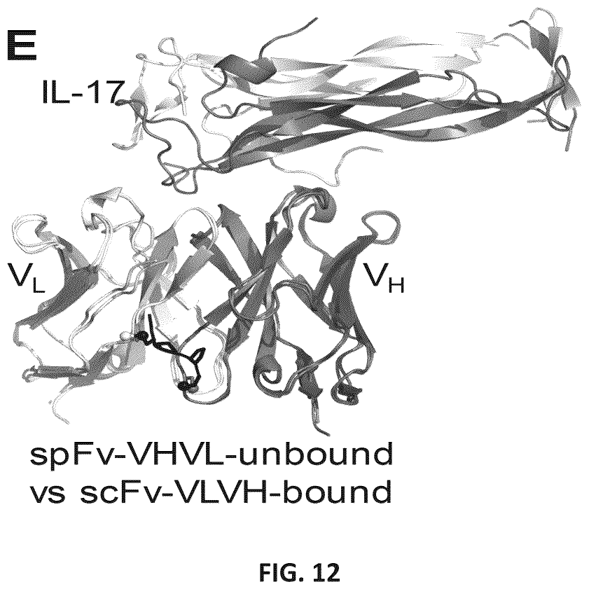

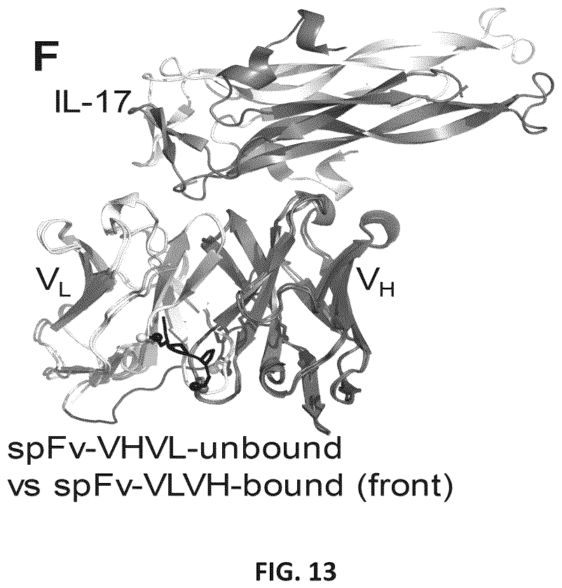

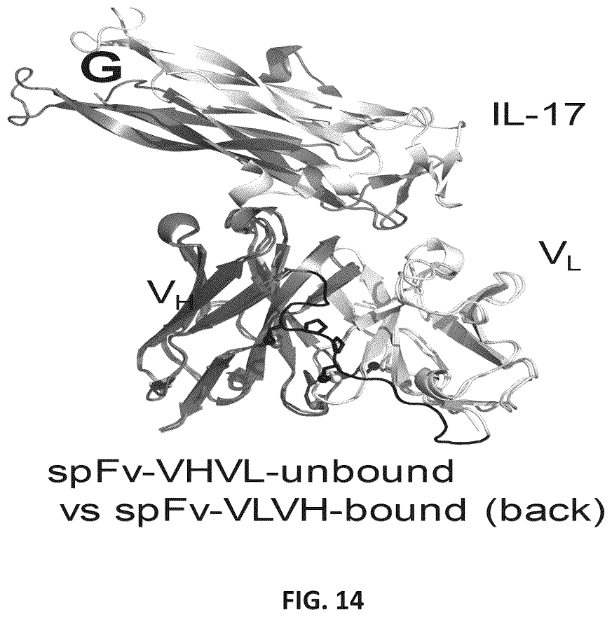

The structures of the unbound scFv and spFv molecules are shown in FIG. 8, FIG. 9, FIG. 10 and FIG. 11. FIG. 8 shows the structure of GLk1 spFv VL-VH. FIG. 9 shows the structure of GLk1 spFv VH-VL. FIG. 10 shows the structure of GLk2 spFv VH-VL. FIG. 11 shows the structure of CAT2200b spFv VH-VL. The structures were consistent with the typical Fv structures with both VL and VH domains packing against each other. In general, most of the linker residues were ordered and resolved in the electron density maps. The disulfide bonds between the staple and the anchor points were generally well ordered in both VL-VH orientations. In addition to the unbound scFv and spFv structures, we also attempted to reveal any structural impact on antigen binding. CAT2200 scFv and spFv variant molecules were crystallized in complex with its cognate target, IL-17. For the scFv and spFv of CAT2200 variants crystallized, the structures were nearly identical with and without a bound target (FIG. 12, FIG. 13, FIG. 14). FIG. 12 shows the comparison of the unbound CAT2200b spFv VH-VL compared with CAT2200a scFv VL-VH bound to IL-17. FIG. 13 shows the comparison of the front views of the structures of unbound CAT2200b spFv VH-VL compared with CAT2200a spFv VL-VH bound to IL-17. FIG. 14 shows the comparison of the back views of the structures of unbound CAT2200b spFv VH-VL compared with CAT2200a spFv VL-VH bound to IL-17. The structures were also identical regardless of orientation or presence or absence of the staple. The rmsd for all matching Cα atoms between pairs of structures were very small: 0.41 Å between unbound spFv-VH-VL and antigen-bound scFv-VL-VH (FIG. 12), 0.46 Å between unbound spFv-VH-VL and spFv-VL-VH (FIG. 13 and FIG. 14, respectively), and 0.37 Å between bound scFv and bound spFv. The structural evidence shows that the stapling works as designed. Also, stapling does not impact the domain structures of VL and VH or relative VL/VH packing.

### Example 4: Design of Additional Anchor Points

The approach described in Example 1 was used to identify any additional anchor points for stapling. The following anchor points were identified:

For VL-linker-VH orientation: VL Chothia position 42, 45 and 39 and VH Chothia positions 105, 5 and 3. In FIG. 6, the VL residues on GLk1VL are K42, K45, K39 and the VH residues on GLk1 are Q105, L5 and Q3. The staple forms between any of the positions indicated.

For VH-linker-VL orientation: VH Chothia positions 43, 40 and 46, VL Chothia positions 102, 5 and 3, the staple forms between any of the positions.

### Example 5: Generation of Stapled PSMA×CD3 Antibody (PS3B2040)

PS3B1396 is a fully human IgG1 antibody directed against the CD3 and PSMA receptors. The generation of PS3B1396 is described in U.S. Application No. 63/142,921 filed Jan. 28, 2021 and U.S. application 63/165,448 filed Mar. 24, 2021 each of which is incorporated herein by reference in its entirety. PS3B1396 comprises the CD3 binding arm of CD3B2030 N106A LH scFv and the PSMA binding arm of PSMB896-G100A-Fab.

Assays to evaluate the functional activities of PS3B1396 and structural properties such as, e.g., amino acid sequences are also described in detail in U.S. Application No. 63/142,921 filed Jan. 28, 2021 and U.S. application 63/165,448 filed Mar. 24, 2021 each of which is incorporated herein by reference in its entirety.

The scFv of the CD3 binding arm of PS3B1396 was engineered into a stabilized scFv, herein referred to as spFv (or “stapled scFv) by restraining the scFv structure, without negatively impacting, the relative movements between the VH and the VL. The stabilized scFv was generated by engineering disulfide bonds between the VH and the linker and between the VL and the linker. Two structurally conserved surface exposed framework positions (anchor points) that are not involved in antigen binding, were identified, one on the VH at position H105 and one on the VL at position L42, and mutated into cysteine (Cys) residues to generate the VH of SEQ ID NO: 28 and VL of SEQ ID NO: 29. A flexible linker of sequence GGGSGGSGGCPPCGGSGG (SEQ ID NO: 2) comprising two Cys residues was used to conjugate the VL and the VH in the VL-linker-VH (LH) format. The distance and location of the Cys residues of the linker and the Cys residues of the VH and the VL is critical for the formation of the disulfide bonds between the Cys residues of the Linker and each anchor point of the VH and the VL.

The anti-CD3 stapled scFv (anti-CD3 spFv) in the VL-Linker-VH format was then paired with anti-PSMA Fab (PSMB896-G100A-Fab) using knob-in-hole Fc homodimerization to generate the stapled PSMA×CD3 bispecific (PS3B2040). Generation characterization of PSMB896-G100A-Fab is described in U.S. Application No. 63/142,921 filed Jan. 28, 2021, and in U.S. application 63/165,448 filed Mar. 24, 2021 each of which is incorporated herein by reference in its entirety. Sequences of the PSMA and CD3 binding domains of PS3B2040 are described below.

The PSMA binding domain of PS3B2040 comprises HCDR1 of amino acid sequence SYAMS (SEQ ID NO: 54), the HCDR2 of amino acid sequence AISGGIGSTYYADSVKG (SEQ ID NO: 55), and the HCDR3 of amino acid sequence DAVGATPYYFDY (SEQ ID NO: 56) and the LCDR1 of amino acid sequence SGSSSNIGINYVS (SEQ ID NO: 57), the LCDR2 of amino acid sequence DNNKRPS (SEQ ID NO: 58), and the LCDR3 of amino acid sequence GTWDSSLSAVV (SEQ ID NO: 59) using the Kabat delineation.

### VH, VL, HC, LC of PS3B2040 PSMA Binding Domain

VH amino acid sequence of PS3B2040
PSMA binding domain;
SEQ ID NO: 60
EVQLVESGGGLVQPGGSLRLSCAASGFTFSSYAMSWVRQAPGKGL
EWVSAISGGIGSTYYADSVKGRFTISRDNSKNTLWLQMNSLRAED
TAVYYCAKDAVGATPYYFDYWGQGTLVTVSS
VL amino acid sequence of PS3B2040
PSMA binding domain;
SEQ ID NO: 61
QSVLTQPPSVSAAPGQKVTISCSGSSSNIGINYVSWYQQLPGTAP
KLLIYDNNKRPSGIPDRFSGSKSGTSATLGITGLQTGDEADYYCG
TWDSSLSAVVFGGGTKLTVL
HC1 amino acid sequence of PS3B2040
PSMA binding domain;
SEQ ID NO: 62
EVQLVESGGGLVQPGGSLRLSCAASGFTFSSYAMSWVRQAPGKGL
EWVSAISGGIGSTYYADSVKGRFTISRDNSKNTLWLQMNSLRAED
TAVYYCAKDAVGATPYYFDYWGQGTLVTVSSASTKGPSVFPLAPS
SKSTSGGTAALGCLVKDYFPEPVTVSWNSGALTSGVHTFPAVLQS
SGLYSLSSVVTVPSSSLGTQTYICNVNHKPSNTKVDKKVEPKSCD
KTHTCPPCPAPEAAGGPSVFLFPPKPKDTLMISRTPEVTCVVVSV
SHEDPEVKFNWYVDGVEVHNAKTKPREEQYNSTYRVVSVLTVLHQ
DWLNGKEYKCKVSNKALPAPIEKTISKAKGQPREPQVYTLPPSRE
EMTKNQVSLSCAVKGFYPSDIAVEWESNGQPENNYKTTPPVLDSD
GSFFLVSKLTVDKSRWQQGNVFSCSVMHEALHNRFTQKSLSLSPG
K
LC1 amino acid sequence of PS3B2040
PSMA binding domain;
SEQ ID NO: 63
QSVLTQPPSVSAAPGQKVTISCSGSSSNIGINYVSWYQQLPGTAP
KLLIYDNNKRPSGIPDRFSGSKSGTSATLGITGLQTGDEADYYCG
TWDSSLSAVVFGGGTKLTVLGQPKAAPSVTLFPPSSEELQANKAT
LVCLISDFYPGAVTVAWKADSSPVKAGVETTTPSKQSNNKYAASS
YLSLTPEQWKSHRSYSCQVTHEGSTVEKTVAPTECS
HC1 nucleic acid sequence of PS3B2040
PSMA binding domain;
SEQ ID NO: 64
GAGGTGCAGCTGGTTGAATCTGGCGGAGGATTGGTTCAGCCTGGC
GGTTCTCTGAGACTGTCTTGTGCCGCTTCCGGCTTCACCTTCTCC
AGCTACGCTATGTCCTGGGTCCGACAGGCTCCTGGCAAAGGACTG
GAATGGGTGTCCGCTATCTCTGGCGGCATCGGCTCTACCTACTAC
GCCGACTCTGTGAAGGGCAGATTCACCATCAGCCGGGACAACTCC
AAGAACACCCTGTGGCTGCAGATGAACTCCCTGAGAGCCGAGGAT
ACCGCCGTGTACTACTGTGCCAAGGATGCCGTGGGCGCTACCCCT
TACTACTTCGATTATTGGGGCCAGGGCACCCTGGTCACCGTTTCT
TCTGCCTCCACCAAGGGCCCATCGGTCTTCCCCCTGGCACCCTCC
TCCAAGAGCACCTCTGGGGGCACAGCGGCCCTGGGCTGCCTGGTC
AAGGACTACTTCCCCGAACCGGTGACGGTGTCGTGGAACTCAGGC
GCCCTGACCAGCGGCGTGCACACCTTCCCGGCTGTCCTACAGTCC
TCAGGACTCTACTCCCTCAGCAGCGTGGTGACCGTGCCCTCCAGC
AGCTTGGGCACCCAGACCTACATCTGCAACGTGAATCACAAGCCC
AGCAACACCAAGGTGGACAAGAAAGTTGAGCCCAAATCTTGTGAC
AAAACTCACACATGCCCACCGTGCCCAGCACCTGAAGCCGCCGGG
GGACCGTCAGTCTTCCTCTTCCCCCCAAAACCCAAGGACACCCTC
ATGATCTCCCGGACCCCTGAGGTCACATGCGTGGTGGTGAGCGTG
AGCCACGAAGACCCTGAGGTCAAGTTCAACTGGTACGTGGACGGC
GTGGAGGTGCATAATGCCAAGACAAAGCCGCGGGAGGAGCAGTAC
AACAGCACGTACCGTGTGGTCAGCGTCCTCACCGTCCTGCACCAG
GACTGGCTGAATGGCAAGGAGTACAAGTGCAAGGTGTCGAACAAA
GCCCTCCCAGCCCCCATCGAGAAAACCATCTCCAAAGCCAAAGGG
CAGCCCCGAGAACCACAGGTGTACACCCTGCCCCCATCCCGGGAG
GAGATGACCAAGAACCAGGTCAGCCTGTCCTGCGCCGTCAAAGGC
TTCTATCCCAGCGACATCGCCGTGGAGTGGGAGAGCAATGGGCAG
CCGGAGAACAACTACAAGACCACGCCTCCCGTGCTGGACTCCGAC
GGCTCCTTCTTCCTCGTGAGCAAGCTCACCGTGGACAAGAGCAGA
TGGCAGCAGGGGAACGTCTTCTCATGCTCCGTGATGCATGAGGCT
CTGCACAACCGGTTCACGCAGAAGTCTCTCTCCCTGTCTCCGGGA
AAA
LC1 nucleic acid sequence of PS3B2040
PSMA binding domain;
SEQ ID NO: 65
CAGTCTGTGCTGACCCAGCCTCCTTCTGTGTCTGCTGCTCCTGGC
CAGAAAGTGACCATCTCCTGCTCCGGCTCCTCCTCCAACATCGGC
ATCAACTACGTGTCCTGGTATCAGCAGCTGCCCGGCACAGCTCCC
AAACTGCTGATCTACGACAACAACAAGCGGCCCAGCGGCATCCCT
GACAGATTCTCCGGATCTAAGTCCGGCACCTCTGCTACCCTGGGC
ATCACAGGATTGCAGACAGGCGACGAGGCCGACTACTATTGCGGC
ACCTGGGACTCTTCTCTGTCCGCCGTTGTTTTTGGCGGTGGCACC
AAGCTGACAGTGCTGGGTCAGCCCAAGGCTGCACCCAGTGTCACT
CTGTTCCCGCCCTCCTCTGAGGAGCTTCAAGCCAACAAGGCCACA
CTGGTGTGTCTCATAAGTGACTTCTACCCGGGAGCCGTGACAGTG
GCCTGGAAGGCCGATAGCAGCCCCGTCAAGGCGGGAGTCGAAACC
ACCACACCCTCCAAACAAAGCAACAACAAGTACGCGGCCAGCAGC
TATCTGAGCCTGACGCCTGAGCAGTGGAAGTCCCACAGAAGCTAC
AGCTGCCAGGTCACGCATGAAGGGAGCACCGTGGAGAAGACAGTG
GCCCCTACAGAATGTTCA

### VH, VL, VL-LINKER-VH, HC of PS3B2040 CD3 Binding Domain

VH amino acid sequence of PS3B2040
CD3 binding domain.
SEQ ID NO: 28
QVQLVQSGAEVKKPGSSVKVSCKASGYTFTRSTMHWVKQAPGQGL
EWIGYINPSSAYTNYNQKFQGRVTLTADKSTSTAYMELSSLRSED
TAVYYCASPQVHYDYAGFPYWGCGTLVTVSS
VL amino acid sequence of CD3 binding
domain.
SEQ ID NO: 29
EIVLTQSPATLSASPGERVTLSCSASSSVSYMNWYQQKPGCAPRR
WIYDSSKLASGVPARFSGSGSGRDYTLTISSLEPEDFAVYYCQQW
SRNPPTFGGGTKVEIK
SpFv (VL-linker-VH) amino acid sequence
of PS3B2040 CD3 binding domain;
SEQ ID NO: 30
EIVLTQSPATLSASPGERVTLSCSASSSVSYMNWYQQKPGCAPRR
WIYDSSKLASGVPARFSGSGSGRDYTLTISSLEPEDFAVYYCQQW
SRNPPTFGGGTKVEIKGGGSGGSGGCPPCGGSGGQVQLVQSGAEV
KKPGSSVKVSCKASGYTFTRSTMHWVKQAPGQGLEWIGYINPSSA
YTNYNQKFQGRVTLTADKSTSTAYMELSSLRSEDTAVYYCASPQV
HYDYAGFPYWGCGTLVTVSS
HC2 (spFv-Fc) amino acid sequence of
PS3B2040 CD3 binding domain;
SEQ ID NO: 66
EIVLTQSPATLSASPGERVTLSCSASSSVSYMNWYQQKPGCAPRR
WIYDSSKLASGVPARFSGSGSGRDYTLTISSLEPEDFAVYYCQQW
SRNPPTFGGGTKVEIKGGGSGGSGGCPPCGGSGGQVQLVQSGAEV
KKPGSSVKVSCKASGYTFTRSTMHWVKQAPGQGLEWIGYINPSSA
YTNYNQKFQGRVTLTADKSTSTAYMELSSLRSEDTAVYYCASPQV
HYDYAGFPYWGCGTLVTVSSEPKSSDKTHTCPPCPAPEAAGGPSV
FLFPPKPKDTLMISRTPEVTCVVVSVSHEDPEVKFNWYVDGVEVH
NAKTKPREEQYNSTYRVVSVLTVLHQDWLNGKEYKCKVSNKALPA
PIEKTISKAKGQPREPQVYTLPPSREEMTKNQVSLWCLVKGFYPS
DIAVEWESNGQPENNYKTTPPVLDSDGSFFLYSKLTVDKSRWQQG
NVFSCSVMHEALHNHYTQKSLSLSPGK
HC2 (scFv-Fc) nucleic acid sequence of
PS3B2040 CD3 binding domain;
SEQ ID NO: 67
GAGATCGTGCTGACCCAGTCTCCTGCCACACTGAGTGCTTCTCCA
GGCGAGAGAGTGACCCTGTCCTGCTCCGCTTCCTCCTCCGTGTCC
TACATGAACTGGTATCAGCAGAAGCCCGGCTGCGCCCCTAGAAGA
TGGATCTACGACTCCTCCAAGCTGGCCTCTGGCGTGCCTGCTAGA
TTTTCCGGCTCTGGCTCTGGCAGAGACTATACCCTGACAATCTCC
AGCCTGGAACCTGAGGACTTCGCCGTGTACTACTGCCAGCAGTGG
TCTAGGAACCCTCCTACCTTTGGCGGAGGCACCAAGGTGGAAATC
AAGGGCGGAGGTTCTGGTGGATCTGGCGGATGTCCTCCTTGTGGT
GGAAGCGGCGGACAGGTTCAGCTGGTTCAGTCTGGCGCCGAAGTG
AAGAAACCTGGCTCCTCCGTGAAAGTGTCCTGCAAGGCTTCCGGC
TACACCTTTACCAGATCCACCATGCACTGGGTCAAGCAGGCCCCT
GGACAAGGCTTGGAGTGGATCGGCTACATCAACCCCAGCTCCGCC
TACACCAACTACAACCAGAAATTCCAGGGCAGAGTGACCCTGACC
GCCGACAAGTCTACCTCCACCGCCTACATGGAACTGTCCAGCCTG
AGATCTGAGGACACCGCCGTGTACTACTGCGCCTCTCCTCAGGTT
CACTACGACTACGCCGGCTTTCCTTATTGGGGCTGTGGCACCCTG
GTCACCGTTTCTTCTGAGCCCAAATCTAGCGACAAAACTCACACA
TGCCCACCGTGCCCAGCACCTGAAGCCGCCGGGGGACCGTCAGTC
TTCCTCTTCCCCCCAAAACCCAAGGACACCCTCATGATCTCCCGG
ACCCCTGAGGTCACATGCGTGGTGGTGAGCGTGAGCCACGAAGAC
CCTGAGGTCAAGTTCAACTGGTACGTGGACGGCGTGGAGGTGCAT
AATGCCAAGACAAAGCCGCGGGAGGAGCAGTACAACAGCACGTAC
CGTGTGGTCAGCGTCCTCACCGTCCTGCACCAGGACTGGCTGAAT
GGCAAGGAGTACAAGTGCAAGGTGTCGAACAAAGCCCTCCCAGCC
CCCATCGAGAAAACCATCTCCAAAGCCAAAGGGCAGCCCCGAGAA
CCACAGGTGTACACCCTGCCCCCATCCCGGGAGGAGATGACCAAG
AACCAGGTCAGCCTGTGGTGCCTGGTCAAAGGCTTCTATCCCAGC
GACATCGCCGTGGAGTGGGAGAGCAATGGGCAGCCGGAGAACAAC
TACAAGACCACGCCTCCCGTGCTGGACTCCGACGGCTCCTTCTTC
CTCTACAGCAAGCTCACCGTGGACAAGAGCAGATGGCAGCAGGGG
AACGTCTTCTCATGCTCCGTGATGCATGAGGCTCTGCACAACCAC
TACACGCAGAAGTCTCTCTCCCTGTCTCCGGGAAAA

### 5.1 Expression of PS3B2040

PS3B2040 was expressed in ExpiCHO™ cells by transient transfection with purified plasmid DNA following the manufacturer's recommendations. Briefly, ExpiCHO™ cells were maintained in suspension in ExpiCHO™ expression medium (ThermoFisher Scientific, Cat #A29100) in an orbital shaking incubator set at 37° C., 8% CO2 and 125 RPM. The cells were passaged and diluted prior to transfection to 6.0×106 cells per ml, maintaining cell viability at 99.0% or better. Transient transfections were done using the ExpiFectamine™ CHO transfection kit (e.g. ThermoFisher Scientific, Cat #A29131). For each ml of diluted cells to be transfected, 0.25 microgram of plasmid comprising one copy each of the PSMA HC and LC, and 0.25 microgram of a plasmid comprising one copy each of the CD3 HC and PSMA LC and 0.5 microgram of pAdVAntage DNA (Promega, Cat #E1711) was used and diluted into OptiPRO™ SFM complexation medium. For each liter of cells, 2.56 mL of ExpiFectamine™ CHO reagent was diluted into 8 mL of OptiPRO™. The diluted DNA and transfection reagent were combined for one minute, allowing DNA/lipid complex formation, and then added to the cells. After overnight incubation, ExpiCHO™ feed and ExpiFectamine™ CHO enhancers were added to the cells as per the manufacturer's Standard protocol. Cells were incubated with orbital shaking (125 rpm) at 37° C. for seven days prior to harvesting the culture broth. The culture supernatant from the transiently transfected ExpiCHO-S™ cells was clarified by centrifugation (30 min, 3000rcf) followed by filtration (0.2 μm PES membrane, Corning; Corning, NY).

### 5.2 Purification of PS3B2040

The filtered cell culture supernatant was loaded onto a pre-equilibrated (1×DPBS, pH 7.2) custom 120 mL MabSelect SuRe Protein A column (GE Healthcare) using an AKTA Avant 150 chromatography system. After loading, the column was washed with 5 column volumes (CV) of 1×DPBS, pH7.2. The protein was eluted with 8 CV of 0.1 M sodium (Na)-Acetate, pH 3.5. Protein fractions were completely neutralized by the addition of 2.5 M Tris HCl, pH 7.2 to 15% (v/v) of the final volume and syringe filtered (0.2 μm). Next, the neutralized protein solution was dialyzed against 20 mM MES, pH 5.5 prior to loading onto a pre-equilibrated (20 mM MES, pH 6.5) custom Capto S ImpAct (170 mL) column. The column was washed at 19 mL/min for 1.5 CV with 20 mM MES, pH 6.5. The protein elution gradient started at 0-5% over 1 CV at 15 mL/min. Then the gradient was increased to 30% over 15 CV (buffer A: 20 mM MES, pH 6.5; buffer B: 20 mM MES, 1M NaCl, pH 6.5). The major peak fractions were pooled, dialyzed into 1×DPBS, pH7.2 and filtered (0.2 m).

### 5.3 Binding of Stapled PSMA×CD3 Antibody (PS3B2040) on Target Cells Via Flow Cytometry.

PS3B2040 was evaluated for binding to commercial prostate cancer cell line expressing PSMA. LNCap human prostate tumor cells were washed with DPBS and 0.25% trypsin was added to allow cells to detach. Media was added to neutralize trypsin and the cells were transferred to a 15 mL conical with DPBS. The cells were centrifuged 1300 rpm for 5 minutes. DPBS was aspirated and cells were re-suspended in DPBS. The cells were counted using the Vi-cell XR cell viability analyzer and were plated at 100K/well in 100 μL DPBS. The plate was centrifuged at 1200 rpm for 3 minutes and washed 2× with DPBS. Cells were stained with Violet Live/Dead stain (Thermo-Fisher) and incubated at room temperature in the dark for 15 min. The cells were centrifuged and washed 2× with FACS staining buffer (BD Pharmingen).

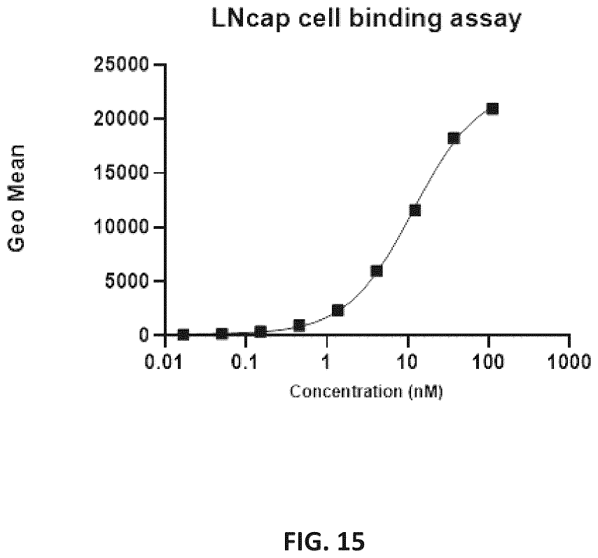

PS3B2040 was diluted to a final starting concentration of 100 nM in FACS staining buffer and 3-fold serial dilutions were prepared from the starting concentration for a total of 10 dilution points. Serially diluted PS3B2040 (100 μL/well) was added to the cells and incubated for 30 min at 37° C. Cells were washed 2× with FACS staining buffer and AlexaFluor 647-conjugated Goat anti-human secondary antibody (Jackson Immunoresearch) was added and allowed to incubate with the cells for 30 min at 4° C. Cells were washed 2× with FACS staining buffer and re-suspended in 200 μL FACS Buffer. Cells were run on BD CELESTA using FACS Diva software and analyzed using FLOWJO. FIG. 15 shows that stapled PSMA×CD3 antibody (PS3B2040) displays dose dependent PSMA cell binding with an EC50 of 11.62 nM.

### 5.4 Binding of Stapled PSMA×CD3 Antibody (PS3B2040) on PAN-T Cells Via Flow.

Human PAN-T Cells (Biological Specialty Corporation, Colmar, PA) were thawed and transferred to a 15 mL conical with DPBS. The cells were centrifuged 1300 rpm for 5 minutes. DPBS was aspirated and cells were re-suspended in DPBS. The cells were counted using the Vi-cell XR cell viability analyzer and were plated at 100K/well in 100 uL DPBS. The plate was centrifuged 1200 rpm for 3 minutes and washed 2× with DPBS. Cells were stained with Violet Live/Dead stain (Thermo-Fisher) and incubated at room temperature in the dark for 15 min. The cells were centrifuged and washed 2× with FACS staining buffer (BD Pharmingen). PS3B2040 was diluted to a final starting concentration of 1 μM in FACS staining buffer and 3-fold serial dilutions were prepared from the starting concentration for a total of 10 dilution points.

Serially diluted PS3B2040 (100 μL/well) was added to the cells and incubated for 60 min at 4° C.

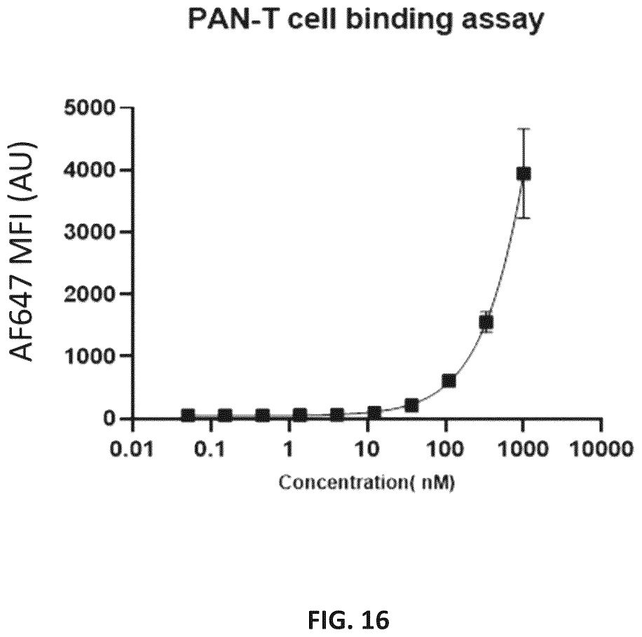

Cells were washed 2× with FACS staining buffer and AlexaFluor 647-conjugated Goat anti-human secondary antibody (Jackson Immunoresearch) was added and allowed to incubate with the cells for 30 min at 4° C. Cells were washed 2× with FACS staining buffer and re-suspended in 200 μL FACS Buffer. Cells were run on BD Celesta using FACS Diva software and analyzed using FLOWJO. FIG. 16 shows that the stapled PSMA×CD3 antibody (PS3B2040) displays dose dependent CD3 cell binding by flow cytometry.

### 5.5 Cytotoxicity of Stapled PSMA×CD3 Antibody (PS3B2040) on Target Cells Using Human PBMCs Via Incucyte

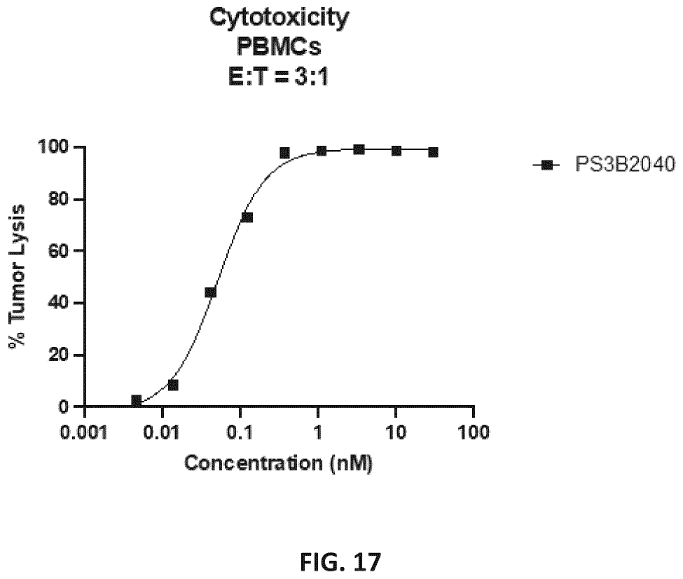

LNCaP human prostate tumor cells were washed with DPBS and 0.05% trypsin was added to allow cells to detach. Media was added to neutralize trypsin and the cells were transferred to a 15 mL conical with DPBS. The cells were centrifuged 1200 rpm for 3 minutes. DPBS was aspirated and cells were re-suspended in DPBS. The cells were counted using the Vi-cell XR cell viability analyzer and were plated at 15K/well in 100 μL DPBS. The plates were incubated at 37° C., 5% CO2 overnight. Human PBMC Cells (Discovery Life Services) were thawed and transferred to a 15 mL conical with DPBS. The cells were centrifuged 1500 rpm for 3 minutes. DPBS was aspirated and cells were re-suspended in DPBS. The cells were counted using the Vi-cell XR cell viability analyzer and were plated at an E:T of 3:1 (normalized to % CD3) in 50 μL of RPMI-10/well. PS3B2040 was prepared in a dilution block with a starting concentration of 30 nM, diluted 3-fold for a total of 10 dilution points including an untreated control. PS3B2040 was added at 50 μL/well in RPMI-10 and the plates were placed into an Incucyte SX5 imaging system (Essen) and scanned every 6 hrs for 120 hrs. The data was analyzed by normalizing to time 0 and % tumor lysis was determined and graphed (GraphPad Prism) using a non-linear fit, log(agonist) vs. response-variable slope (4-parameters). FIG. 17 shows dose dependent cytotoxicity of stapled PSMA×CD3 antibody (PS3B2040) in human PBMCs with an EC50 of 0.051 nM.

### 5.6 Cytotoxicity of Stapled PSMA×CD3 Antibody (PS3B2040) on Target Using Pan T Cells Via Incucyte

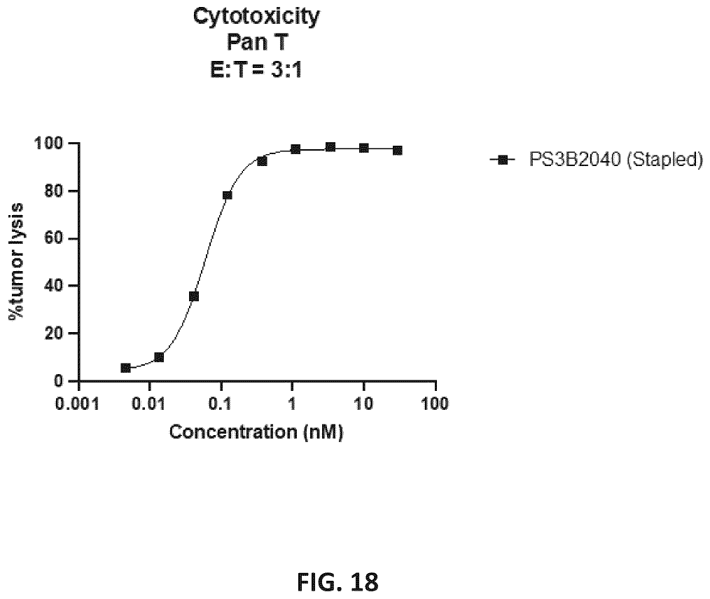

LNCaP human prostate tumor cells were washed with DPBS and 0.05% trypsin was added to allow cells to detach. Media was added to neutralize trypsin and the cells were transferred to a 15 mL conical with DPBS. The cells were centrifuged 1200 rpm for 3 minutes. DPBS was aspirated and cells were re-suspended in DPBS. The cells were counted using the Vi-cell XR cell viability analyzer and were plated at 15K/well in 100 μL RPMI-10 (***phenol red free). The plates were incubated at 37° C., 5% C02 overnight. Human Pan T Cells (Discovery Life Sciences) were thawed and transferred to a 15 mL conical with DPBS. The cells were centrifuged 1500 rpm for 3 minutes. DPBS was aspirated and cells were re-suspended in DPBS. The cells were counted using the Vi-cell XR cell viability analyzer and were plated at an E:T of 3:1 in 50 μL of RPMI-10/well. PS3B2040 was prepared in a dilution block with a starting concentration of 30 nM, diluted 3-fold for a total of 10 dilution points including an untreated control. PS3B2040 was added at 50 uL/well in RPMI-10 and the plates were placed into an Incucyte SX5 (Essen) and scanned every 6 hrs for 120 hrs. The data was analyzed by normalizing to time 0 and % tumor lysis was determined and graphed (GraphPad Prism) using a non-linear fit, log(agonist) vs. response-variable slope (4-parameter). FIG. 18 shows dose dependent cytotoxicity of stapled PSMA×CD3 antibody (PS3B2040) in human Pan T cells with an EC50 of 0.06 nM.

### Example 6: Generation of Stapled CD3×ENPP3 Antibody (NPP3B815)

NPP3B815 is a bispecific antibody (BsAb) that consists of NPP3B56 heavy chain (HC)2×CD3B2030 N106A-LH. NPP3B56 is the binder specific for ENPP3. NPP3B815 was assessed for, among other things, purity, binding affinity, thermal stability, solubility, serum stability, nonspecific binding, viscosity, and aggregation potential. NPP3B815 maintained high purity, showed no conformational changes after 2 weeks at 37° C. stress test, exhibited high solubility with acceptable viscosity, stability, and high percent monomer (at 150 mg/mL) at 40° C., maintained target binding in human serum, and showed no evidence for nonspecific binding. In summary, NPP3B815 met the ideal or acceptable criteria as demonstrated by the results from different biophysical assays, and the overall intrinsic properties were in favor of manufacturability.

### 6.1 NPP3B815

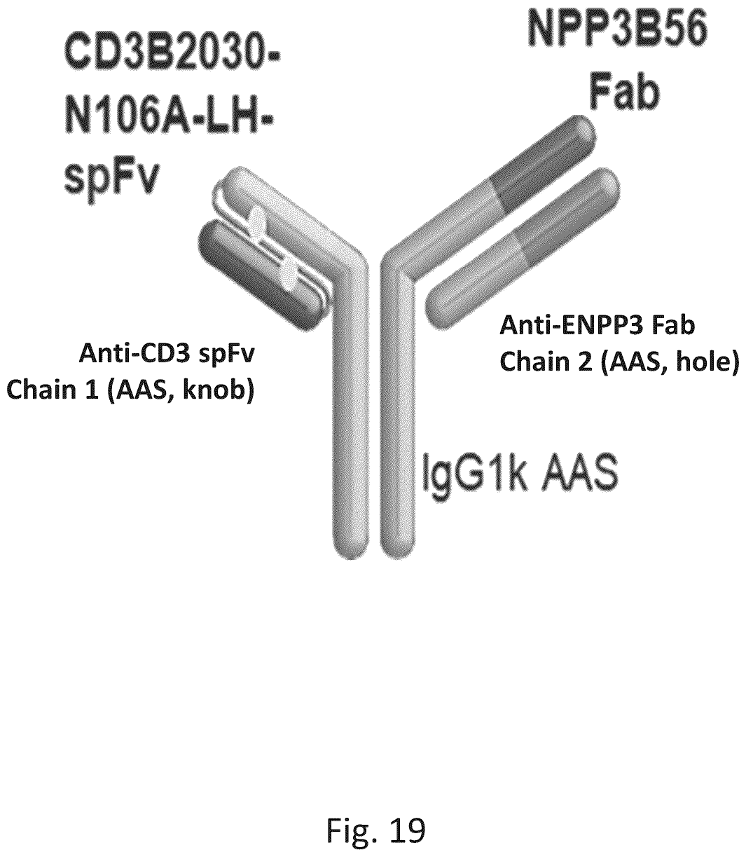

NPP3B815 (NPP3B56×CD3B2030-N106A-spFv) is an IgG1 BsAb that simultaneously binds to CD3R (Uniprot ID: P07766) on T cells and to ENPP3 (Uniprot ID: 014638) on tumor cells (FIG. 19). The antibody features mutations of L234A, L235A, and D265S (AAS; EU numbering) in the Fc region to abolish interaction with Fc receptors, and heterodimerization is enhanced using the knobs-into-holes mutations. The molecule comprises an anti-CD3 spFv on HCl.

ScFv generated by a genetic fusion of variable region light chain (VL) and variable region heavy chain (VH) with a flexible linker in either orientation recapitulates the antigen binding specificity and largely the affinity of a parental Fv. The scFv have been utilized as building block for a variety of multi-specific therapeutic biologics. However, scFv-containing molecules are prone to aggregation due to low thermal stability and transient separation and intermolecular VL/VH reassociation (‘breathing’).

These liabilities were addressed by scFv ‘stapling’, abbreviated as spFv. To this end, 2 disulfide bonds were engineered between the flexible linker and anchor positions of the VL and VH domains (one on each). This novel strategy is compatible in both VL-VH and VH-VL orientations for almost all Fv domains. Extensive characterization of several molecules demonstrates that spFv molecules not only retain the same binding and function with improved biophysical properties but stapling significantly improved protein quality and aggregation of therapeutics observed in scFv.

The spFv consists of a linker having a central ‘C1PPC2’ motif (SEQ ID NO: 157) wherein C1 forms a disulfide bond with (in the case of the spFv in the ‘light-heavy’ orientation) an engineered cysteine at Position 43 (Chothia numbering) in the VL domain and C2 forms a disulfide bond with an engineered cysteine at Position 100 in the VH domain. HC1 features the ‘knob’ mutation T366W. HC2 features the anti-ENPP3 Fab region, the ‘hole’ mutations T366S, L368A, and Y407V.

The BsAb was developed to evaluate the therapeutic potential of targeting ENPP3 for T-cell redirection. NPP3B815 was developed for the treatment of advanced solid tumors where ENPP3 is highly expressed on the cell surface.

NPP3B815 was generated by co-expression of the anti-CD3c spFv ‘knob’ HC with the anti-ENPP3 Fab HC containing the ‘hole’ and paired with the light chain (LC). The anti-CD3c variable region was derived from Cris7, identified in wild-type mice and humanized in scFv format to identify humanized variants with higher thermal stability than parental Cris7 in scFv format with a range of CD3c affinities. Briefly, the murine complementarity determining region (CDR) regions were grafted into the IGHV1-69*02-IGHJ1-01 and IGKV3-11*02-IGKJ4-01 human germlines followed by human framework adaption as described previously. The parental HC contained an NG sequence in CDR3 at amino acid Positions 106-107 (Positions 100B-100C in Kabat numbering), representing a potential risk. This risk was eliminated by mutation N106A. Although several mutations could eliminate this risk, N106A was selected based on weaker affinity of the N106A variant compared to the parental v-region, since weaker affinity towards CD3c may be associated with lower toxicity. The anti-CD3 v-region was then formatted into a spFv in the final molecule NPP3B815. The anti-ENPP3 variable region was discovered by immunizing transgenic mice (Ablexis) with a plasmid expressing full-length ENPP3 (Genedata DNA batch ID VB000066101). The parental anti-ENPP3 variable region, featured in the NPP3B56 mAb was not modified and was formatted as a Fab in the final molecule NPP3B815.

The sequences of the ENPP3 binding domain are shown in Table 8. The amino acid sequences of the CD3 binding arm are shown in SEQ ID NO: 28 (VH), SEQ ID NO: 29 (VL), SEQ ID NO: 30 (SpFv (VL-linker-VH)), and SEQ ID NO: 96 (Heavy chain spFv-Fc). Nucleotide sequences encoding the CD3 binding arm are shown in SEQ ID NO: 68 (VH), SEQ ID NO: 69 (VL), SEQ ID NO: 70 (SpFv (VL-linker-VH)), and SEQ ID NO: 99 (Heavy chain spFv-Fc). The nucleic acid sequences are shown in Table 10.

TABLE 8
NPP3B56 Binding Region Sequences

| | Kabat | Chothia | AbM | Contact | IMGT |
|---|---|---|---|---|---|
| Heavy Chain | DYSMN | GFTFSDY | GFTFSDYSMN | SDYSMN | GFTFSDYS |
| variable region | (SEQ ID | (SEQ ID | (SEQ ID | (SEQ ID | (SEQ ID |
| CDR1 | NO: 71) | NO: 77) | NO: 79) | NO: 81) | NO: 87) |
| Heavy Chain | SISSISSYVKY | SSISSY | SISSISSYVK | WVSSISSISSYVK | ISSISSYV |
| variable region | ADSVKG | (SEQ ID | (SEQ ID | (SEQ ID | (SEQ ID |
| CDR2 | (SEQ ID | NO: 78) | NO: 80) | NO: 82) | NO: 88) |
| | NO: 72) | | | | |
| Heavy Chain | GHYFDY | GHYFDY | GHYFDY | ARGHYFD | ARGHYFDY |
| variable region | (SEQ ID | (SEQ ID | (SEQ ID | (SEQ ID | (SEQ ID |
| CDR3 | NO: 73) | NO: 73) | NO: 73) | NO: 83) | NO: 89) |
| Light Chain | RASQSVSSNL | RASQSVSSNLA | RASQSVSSNLA | SSNLAWY | QSVSSN |
| variable region | A | (SEQ ID | (SEQ ID | (SEQ ID | (SEQ ID |
| CDR1 | (SEQ ID | NO: 74) | NO: 74) | NO: 84) | NO: 90) |
| | NO: 74) | | | | |
| Light Chain | GASTRAT | GASTRAT | GASTRAT | LLIYGASTRA | GAS |
| variable region | (SEQ ID | (SEQ ID | (SEQ ID | (SEQ ID | |
| CDR2 | NO: 75) | NO: 75) | NO: 75) | NO: 85) | |
| Light Chain | QQYNNWPRT | QQYNNWPRT | QQYNNWPRT | QQYNNWPR | QQYNNWPR |
| variable | (SEQ ID | (SEQ ID | (SEQ ID | (SEQ ID | T |
| region | NO: 76) | NO: 76) | NO: 76) | NO: 86) | (SEQ ID |
| CDR3 | | | | | NO: 76) |

VH
(SEQ ID NO: 92):
EVQLVESGGGRVKPGGSLRLSCAASGFTFSDYSMNWVRQAPGKGLEWVSSISSISSYVKYADSVKGRFTM
SRDNAKNSLFLQMNSLRDEDMAVYYCARGHYFDYWGQGTLVTVSS
DNA Sequence encoding VH
(SEQ ID NO: 93)
GAGGTGCAGCTGGTTGAATCTGGCGGCGGAAGAGTGAAGCCTGGCGGATCTCTGAGACTGTCTTGTGCCG
CCTCTGGCTTCACCTTCTCCGACTACTCCATGAACTGGGTCCGACAGGCTCCTGGCAAAGGCCTGGAATG
GGTGTCCTCTATCTCCTCCATCTCCAGCTACGTGAAGTACGCCGACTCCGTGAAGGGCAGATTCACCATG
TCCAGAGACAACGCCAAGAACTCCCTGTTCCTGCAGATGAACAGCCTGCGCGACGAGGACATGGCCGTGT
ACTATTGTGCCAGAGGCCACTACTTCGACTACTGGGGACAGGGCACACTGGTCACAGTCTCTTCT
VL
(SEQ ID NO: 94):
EIVMTQSPATLSVSPGERATLSCRASQSVSSNLAWYQQKPGLAPRLLIYGASTRATGIPARFSGSGSGTE
FTLTISSLQSEDFAVYYCQQYNNWPRTFGQGTKVEIK
DNA Sequence encoding VL
(SEQ ID NO: 95)
GAGATCGTGATGACCCAGTCTCCTGCCACACTGTCTGTGTCTCCCGGCGAGAGAGCTACCCTGTCTTGTA
GAGCCTCTCAGTCCGTGTCCTCCAACCTGGCCTGGTATCAGCAGAAGCCTGGACTGGCTCCCCGGCTGTT
GATCTATGGCGCTTCTACCAGAGCCACAGGCATCCCCGCTAGATTCTCCGGCTCTGGCTCTGGCACAGAG
TTTACCCTGACCATCTCCAGCCTGCAGTCCGAGGATTTCGCCGTGTACTACTGCCAGCAGTACAACAACT
GGCCCCGGACCTTTGGCCAGGGCACCAAGGTGGAAATCAAG

TABLE 9
Sequences of NPP3B815

| | Amino Acids |
|---|---|
| Heavy Chain 1 | EIVLTQSPATLSASPGERVTLSCSASSSVSYMNWYQQKPGCAPRRW |
| (1^{st} polypeptide) | IYDSSKLASGVPARFSGSGSGRDYTLTISSLEPEDFAVYYCQQWSR |
| | NPPTFGGGTKVEIKGGGSGGSGGCPPCGGSGGQVQLVQSGAEVKK |
| | PGSSVKVSCKASGYTFTRSTMHWVKQAPGQGLEWIGYINPSSAYT |
| | NYNQKFQGRVTLTADKSTSTAYMELSSLRSEDTAVYYCASPQVHY |
| | DYAGFPYWGCGTLVTVSSEPKSSDKTHTCPPCPAPEAAGGPSVFLF |
| | PPKPKDTLMISRTPEVTCVVVSVSHEDPEVKFNWYVDGVEVHNAK |
| | TKPREEQYNSTYRVVSVLTVLHQDWLNGKEYKCKVSNKALPAPIE |
| | KTISKAKGQPREPQVYTLPPSREEMTKNQVSLWCLVKGFYPSDIAV |
| | EWESNGQPENNYKTTPPVLDSDGSFFLYSKLTVDKSRWQQGNVFS |
| | CSVMHEALHNHYTQKSLSLSPGK |
| | (SEQ ID NO: 96) |
| Heavy Chain 2 | EVQLVESGGGRVKPGGSLRLSCAASGFTFSDYSMNWVRQAPGKG |
| (2^{nd} polypeptide) | LEWVSSISSISSYVKYADSVKGRFTMSRDNAKNSLFLQMNSLRDED |
| | MAVYYCARGHYFDYWGQGTLVTVSSASTKGPSVFPLAPSSKSTSG |
| | GTAALGCLVKDYFPEPVTVSWNSGALTSGVHTFPAVLQSSGLYSLS |
| | SVVTVPSSSLGTQTYICNVNHKPSNTKVDKKVEPKSCDKTHTCPPC |
| | PAPEAAGGPSVFLFPPKPKDTLMISRTPEVTCVVVSVSHEDPEVKFN |
| | WYVDGVEVHNAKTKPREEQYNSTYRVVSVLTVLHQDWLNGKEY |
| | KCKVSNKALPAPIEKTISKAKGQPREPQVYTLPPSREEMTKNQVSL |
| | SCAVKGFYPSDIAVEWESNGQPENNYKTTPPVLDSDGSFFLVSKLT |
| | VDKSRWQQGNVFSCSVMHEALHNRFTQKSLSLSPGK |
| | (SEQ ID NO: 97) |
| Light Chain (3^{rd} | EIVMTQSPATLSVSPGERATLSCRASQSVSSNLAWYQQKPGLAPRL |
| polypeptide) | LIYGASTRATGIPARFSGSGSGTEFTLTISSLQSEDFAVYYCQQYNN |
| | WPRTFGQGTKVEIKRTVAAPSVFIFPPSDEQLKSGTASVVCLLNNF |
| | YPREAKVQWKVDNALQSGNSQESVTEQDSKDSTYSLSSTLTLSKA |
| | DYEKHKVYACEVTHQGLSSPVTKSFNRGEC |
| | (SEQ ID NO: 98) |

TABLE 10
DNA Sequences Encoding NPP3B815

| | Amino Acids |
|---|---|
| Heavy Chain 1 | GAGATCGTGCTGACCCAGTCTCCTGCCACA |
| (1^{st} poly- | CTGAGTGCTTCTCCAGGCGAGAGAGTGACC |
| nucleotide) | CTGTCCTGCTCCGCTTCCTCCTCCGTGTCC |
| | TACATGAACTGGTATCAGCAGAAGCCCGGC |
| | TGCGCCCCTAGAAGATGGATCTACGACTCC |
| | TCCAAGCTGGCCTCTGGCGTGCCTGCTAGA |
| | TTTTCCGGCTCTGGCTCTGGCAGAGACTAT |
| | ACCCTGACAATCTCCAGCCTGGAACCTGAG |
| | GACTTCGCCGTGTACTACTGCCAGCAGTGG |
| | TCTAGGAACCCTCCTACCTTTGGCGGAGGC |
| | ACCAAGGTGGAAATCAAGGGCGGAGGTTCT |
| | GGTGGATCTGGCGGATGTCCTCCTTGTGGT |
| | GGAAGCGGCGGACAGGTTCAGCTGGTTCAG |
| | TCTGGCGCCGAAGTGAAGAAACCTGGCTCC |
| | TCCGTGAAAGTGTCCTGCAAGGCTTCCGGC |
| | TACACCTTTACCAGATCCACCATGCACTGG |
| | GTCAAGCAGGCCCCTGGACAAGGCTTGGAG |
| | TGGATCGGCTACATCAACCCCAGCTCCGCC |
| | TACACCAACTACAACCAGAAATTCCAGGGC |
| | AGAGTGACCCTGACCGCCGACAAGTCTACC |
| | TCCACCGCCTACATGGAACTGTCCAGCCTG |
| | AGATCTGAGGACACCGCCGTGTACTACTGC |
| | GCCTCTCCTCAGGTTCACTACGACTACGCC |
| | GGCTTTCCTTATTGGGGCTGTGGCACCCTG |
| | GTCACCGTTTCTTCTGAGCCCAAATCTAGC |
| | GACAAAACTCACACATGCCCACCGTGCCCA |
| | GCACCTGAAGCCGCCGGGGGACCGTCAGTC |
| | TTCCTCTTCCCCCCAAAACCCAAGGACACC |
| | CTCATGATCTCCCGGACCCCTGAGGTCACA |
| | TGCGTGGTGGTGAGCGTGAGCCACGAAGAC |
| | CCTGAGGTCAAGTTCAACTGGTACGTGGAC |
| | GGCGTGGAGGTGCATAATGCCAAGACAAAG |
| | CCGCGGGAGGAGCAGTACAACAGCACGTAC |
| | CGTGTGGTCAGCGTCCTCACCGTCCTGCAC |
| | CAGGACTGGCTGAATGGCAAGGAGTACAAG |
| | TGCAAGGTGTCGAACAAAGCCCTCCCAGCC |
| | CCCATCGAGAAAACCATCTCCAAAGCCAAA |
| | GGGCAGCCCCGAGAACCACAGGTGTACACC |
| | CTGCCCCCATCCCGGGAGGAGATGACCAAG |
| | AACCAGGTCAGCCTGTGGTGCCTGGTCAAA |
| | GGCTTCTATCCCAGCGACATCGCCGTGGAG |
| | TGGGAGAGCAATGGGCAGCCGGAGAACAAC |
| | TACAAGACCACGCCTCCCGTGCTGGACTCC |
| | GACGGCTCCTTCTTCCTCTACAGCAAGCTC |
| | ACCGTGGACAAGAGCAGATGGCAGCAGGGG |
| | AACGTCTTCTCATGCTCCGTGATGCATGAG |
| | GCTCTGCACAACCACTACACGCAGAAGTCT |
| | CTCTCCCTGTCTCCGGGAAAA |
| | (SEQ ID NO: 99) |
| Heavy Chain 2 | GAGGTGCAGCTGGTTGAATCTGGCGGCGGA |
| (2^{nd} poly- | AGAGTGAAGCCTGGCGGATCTCTGAGACTG |
| nucleotide) | TCTTGTGCCGCCTCTGGCTTCACCTTCTCC |
| | GACTACTCCATGAACTGGGTCCGACAGGCT |
| | CCTGGCAAAGGCCTGGAATGGGTGTCCTCT |
| | ATCTCCTCCATCTCCAGCTACGTGAAGTAC |
| | GCCGACTCCGTGAAGGGCAGATTCACCATG |
| | TCCAGAGACAACGCCAAGAACTCCCTGTTC |
| | CTGCAGATGAACAGCCTGCGCGACGAGGAC |
| | ATGGCCGTGTACTATTGTGCCAGAGGCCAC |
| | TACTTCGACTACTGGGGACAGGGCACACTG |
| | GTCACAGTCTCTTCTGCCTCCACCAAGGGC |
| | CCATCGGTCTTCCCCCTGGCACCCTCCTCC |
| | AAGAGCACCTCTGGGGGCACAGCGGCCCTG |
| | GGCTGCCTGGTCAAGGACTACTTCCCCGAA |
| | CCGGTGACGGTGTCGTGGAACTCAGGCGCC |
| | CTGACCAGCGGCGTGCACACCTTCCCGGCT |
| | GTCCTACAGTCCTCAGGACTCTACTCCCTC |
| | AGCAGCGTGGTGACCGTGCCCTCCAGCAGC |
| | TTGGGCACCCAGACCTACATCTGCAACGTG |
| | AATCACAAGCCCAGCAACACCAAGGTGGAC |
| | AAGAAAGTTGAGCCCAAATCTTGTGACAAA |
| | ACTCACACATGCCCACCGTGCCCAGCACCT |
| | GAAGCCGCCGGGGGACCGTCAGTCTTCCTC |
| | TTCCCCCCAAAACCCAAGGACACCCTCATG |
| | ATCTCCCGGACCCCTGAGGTCACATGCGTG |
| | GTGGTGAGCGTGAGCCACGAAGACCCTGAG |
| | GTCAAGTTCAACTGGTACGTGGACGGCGTG |
| | GAGGTGCATAATGCCAAGACAAAGCCGCGG |
| | GAGGAGCAGTACAACAGCACGTACCGTGTG |
| | GTCAGCGTCCTCACCGTCCTGCACCAGGAC |
| | TGGCTGAATGGCAAGGAGTACAAGTGCAAG |
| | GTGTCGAACAAAGCCCTCCCAGCCCCCATC |
| | GAGAAAACCATCTCCAAAGCCAAAGGGCAG |
| | CCCCGAGAACCACAGGTGTACACCCTGCCC |
| | CCATCCCGGGAGGAGATGACCAAGAACCAG |
| | GTCAGCCTGTCCTGCGCCGTCAAAGGCTTC |
| | TATCCCAGCGACATCGCCGTGGAGTGGGAG |
| | AGCAATGGGCAGCCGGAGAACAACTACAAG |
| | ACCACGCCTCCCGTGCTGGACTCCGACGGC |
| | TCCTTCTTCCTCGTGAGCAAGCTCACCGTG |
| | GACAAGAGCAGATGGCAGCAGGGGAACGTC |
| | TTCTCATGCTCCGTGATGCATGAGGCTCTG |
| | CACAACCGGTTCACGCAGAAGTCTCTCTCC |
| | CTGTCTCCGGGAAAA |
| | (SEQ ID NO: 100) |
| Light Chain | GAGATCGTGATGACCCAGTCTCCTGCCACA |
| (3^{rd} poly- | CTGTCTGTGTCTCCCGGCGAGAGAGCTACC |
| nucleotide) | CTGTCTTGTAGAGCCTCTCAGTCCGTGTCC |
| | TCCAACCTGGCCTGGTATCAGCAGAAGCCT |
| | GGACTGGCTCCCCGGCTGTTGATCTATGGC |
| | GCTTCTACCAGAGCCACAGGCATCCCCGCT |
| | AGATTCTCCGGCTCTGGCTCTGGCACAGAG |
| | TTTACCCTGACCATCTCCAGCCTGCAGTCC |
| | GAGGATTTCGCCGTGTACTACTGCCAGCAG |
| | TACAACAACTGGCCCCGGACCTTTGGCCAG |
| | GGCACCAAGGTGGAAATCAAGCGTACTGTG |
| | GCTGCACCATCTGTCTTCATCTTCCCGCCA |
| | TCTGATGAGCAGTTGAAATCTGGAACTGCC |
| | TCTGTTGTGTGCCTGCTGAATAACTTCTAT |
| | CCCAGAGAGGCCAAAGTACAGTGGAAGGTG |
| | GATAACGCCCTCCAATCGGGTAACTCCCAG |
| | GAGAGTGTCACAGAGCAGGACAGCAAGGAC |
| | AGCACCTACAGCCTCAGCAGCACCCTGACG |
| | CTGAGCAAAGCAGACTACGAGAAACACAAA |
| | GTCTACGCCTGCGAAGTCACCCATCAGGGC |
| | CTGAGCTCGCCCGTCACAAAGAGCTTCAAC |
| | AGGGGAGAGTGT |
| | (SEQ ID NO: 101) |

### 6.2 Selection of ENPP3-Expressing Cancer Cell Lines for NPP31B815 In Vitro and In Vivo Assessment

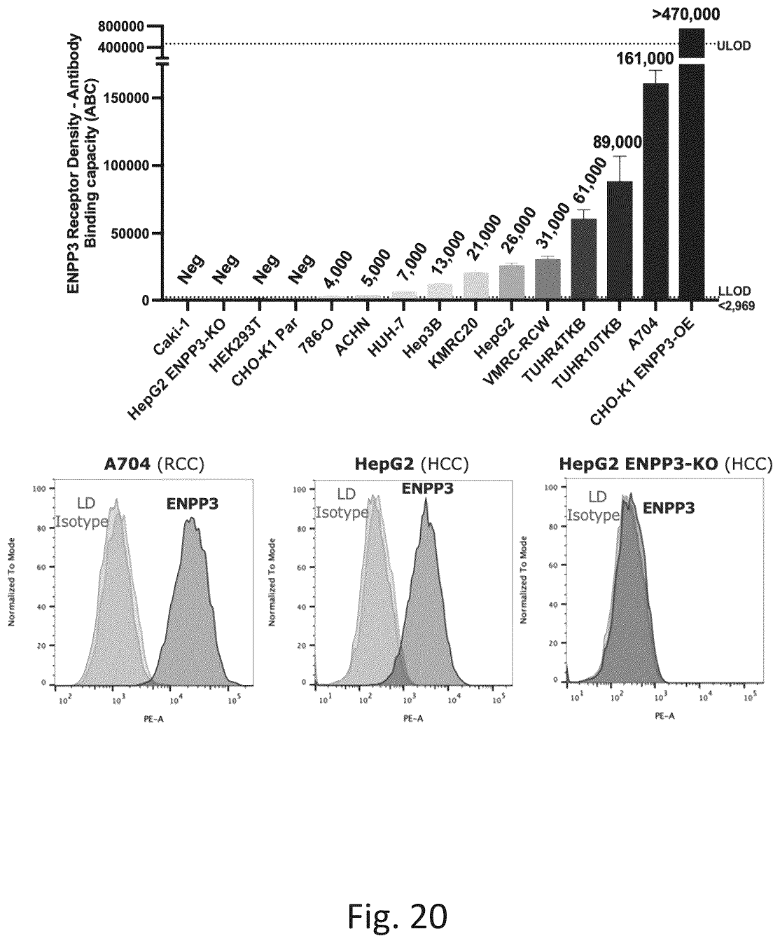

ENPP3 surface expression and receptor density were initially evaluated on a panel of 13 in vitro established RCC and HCC cell lines by flow cytometry using a commercial ENPP3 antibody (clone NP4D36) binding to a similar epitope as the ENPP3 binder arm within NPP31B815. As represented in FIG. 20, ENPP3 endogenous expression ranged from negative (determined as <lower limit of detection [LLOD], 2,969 receptors/cell) in Caki1, HEK293T and RXF393 (PDX) cell lines, low (<15,000 receptors/cell) in 786-O, ACHN, Huh7 and Hep3B, medium (15,000-40,000 receptors/cell) in KMRC20, HepG2 and VMRCRCW, medium-high (50,000-100,000 receptors/cell) in TUHR4TKB and TUHR10TKB and high (>100,000 receptors/cell) in A704 cell lines. Endogenous ENPP3 was knocked out (using clustered regularly interspaced short palindromic repeats [CRISPR]) in HepG2 cell line, thus generating the negative cell line HepG2 ENPP3-KO. Conversely, ENPP3 was overexpressed in the CHO-K1 cell line to generate the overexpression cell line CHO-K1 ENPP30E.

To confirm if similar ENPP3 expression was detectable by NPP3B815 on these cell lines, direct-labeled ENPP3×Null BsAb NPP3B812, which has the same ENPP3 binder NPP3B56 as NPP3B815, was used for flow cytometry-based receptor density studies. The level of ENPP3 expression with NPP3B812 was observed to be comparable to that seen with the commercial antibody in the cell lines tested. While these cell lines represent a range of ENPP3 expression, most of them having lower expression than the ccRCC tumors. Among these cell lines, A704, VMRCRCW, and HepG2 ENPP3-KO cell lines were used for most of the key in vitro functional studies as they represent high, medium, and negative ENPP3 expression levels, respectively.

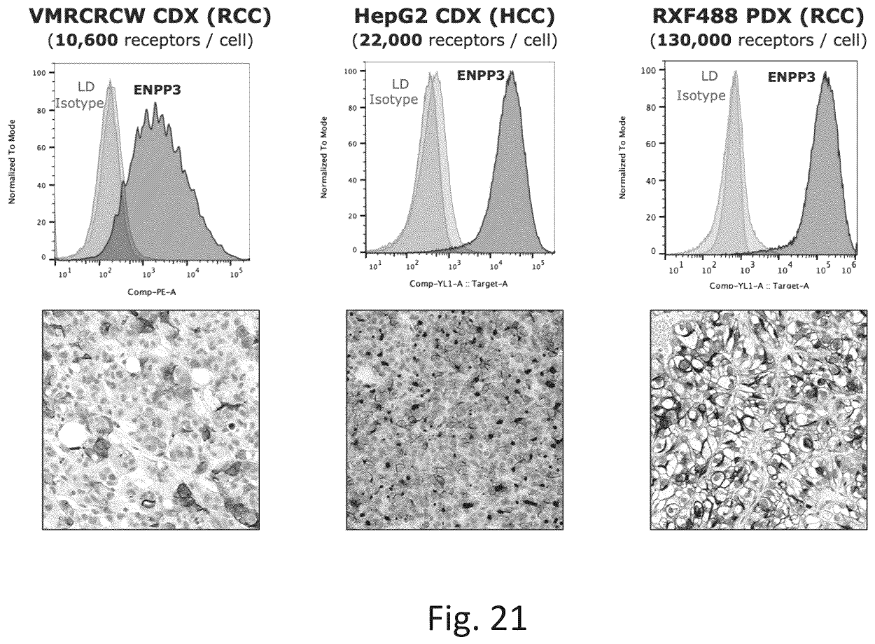

Two of these cell lines (i.e., VMRCRCW and HepG2) have also been established as in vivo CDX models to use for NPP3B815 efficacy studies. Ex vivo ENPP3 expression was measured by IHC and flow cytometry (dissociated tumors) at a tumor volume equivalent to the tumor at randomization in these 2 CDX tumor models. IHC showed ENPP3 positivity in both CDX tumors and receptor density was found to be 10,600 (VMRCRCW) and 22,000 (HepG2) ENPP3 receptors per cell (FIG. 21). To explore a higher ENPP3 expressing tumor model for in vivo studies, several positive ccRCC PDX models were identified by IHC; ENPP3 expression was evaluated ex vivo in the ccRCC PDX model RXF488, which showed high ENPP3 expression by IHC and receptor density measurements of 130,000 receptors/cell.

### 6.3 In Vitro Binding of NPP3B815 to Tumor Cells and T Cells

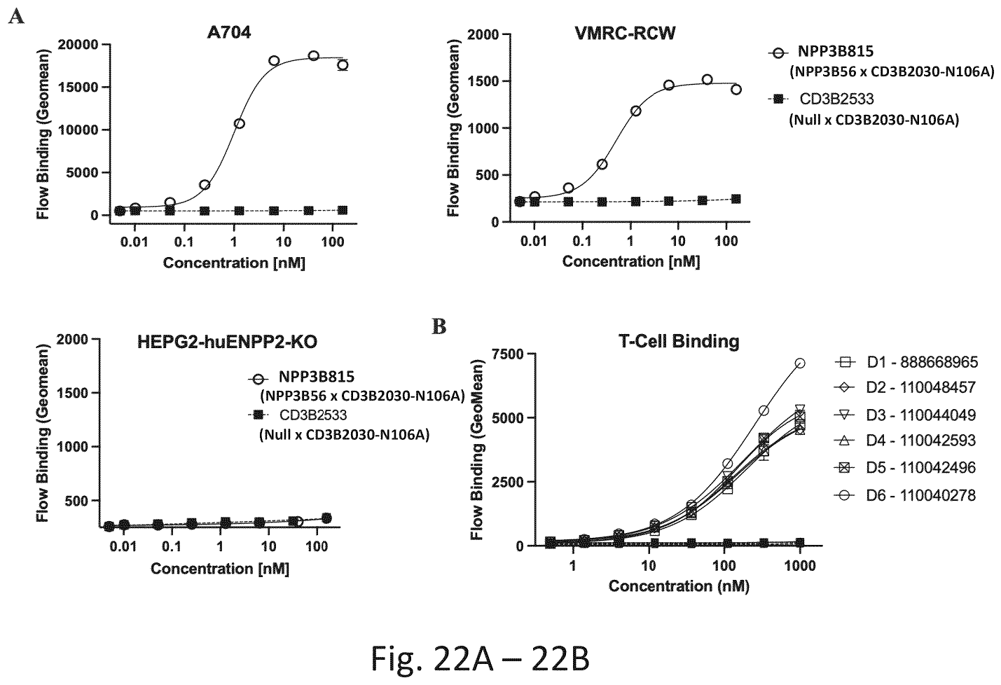

In vitro cell binding of NPP3B815 (NPP3B56×CD3B2030-N106A-spFv) was assessed on high (A704), medium (VMRCRCW), and negative endogenous ENPP3-expressing cell lines (Table 11). NPP3B815 exhibited a dose-dependent binding to both positive cell lines, A704 and VMRCRCW, with EC50 values of 1.01 nM and 0.5 nM respectively (FIG. 22 and Table 12). NPP3B815 did not exhibit binding to the ENPP3-negative cell line HepG2 ENPP3-KO. No binding was seen to any of the cell lines with the Null×CD3 (79C3B615) or isotype control (79C3B613) antibodies.

TABLE 11
ENPP3 expression (receptor density) in different cancer cell lines

| Tumor Cells | Receptor Density (ABC) |
|---|---|
| A704 | 161,000 |
| TUHR10TKB | 89,000 |
| TUHR10TKB | 61,000 |
| VMRCRCW | 31,000 |
| HepG2 | 26,000 |
| HepG2 ENPP3 KO | Negative |

Further, binding of NPP3B815 to human T cells isolated from 6 different healthy donors was assessed. A dose-dependent binding was observed to T cells from all 6 donors, with an average EC50 (±standard error of the mean) of 177±18.5 nM (FIG. 22 and Table 12). As expected, no binding was seen with isotype control antibody 79C3B613 to any of the T-cell donors.

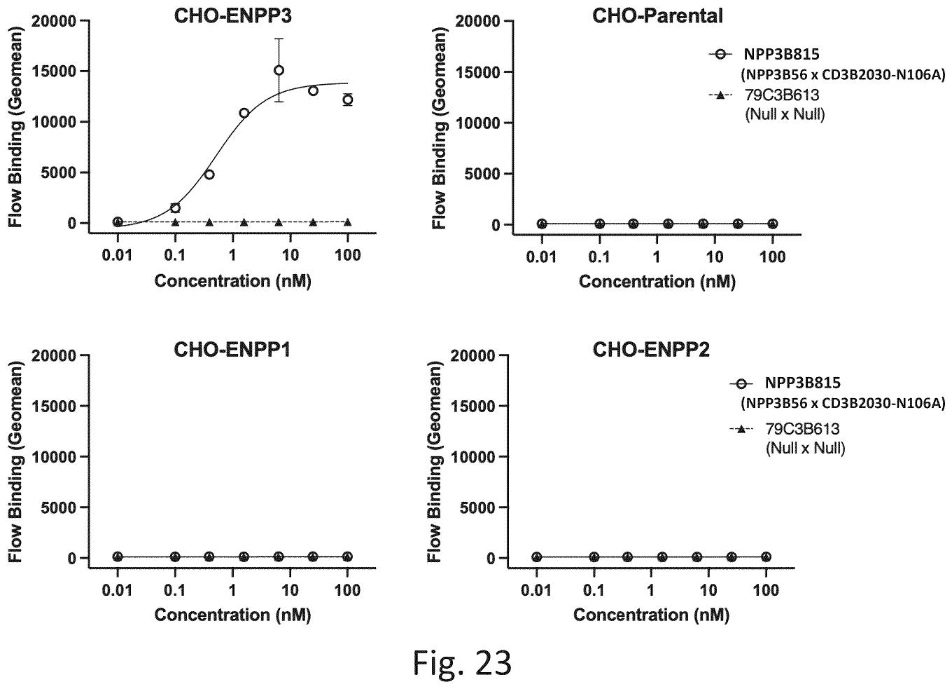

To evaluate the specificity of NPP3B815 to ENPP3 compared to other ENPP family members, cell binding was assessed on CHOK1 cells overexpressing ENPP1, ENPP2, or ENPP3 (FIG. 23). NPP3B815 exhibited a dose-dependent binding to only the CHOK1 cells overexpressing ENPP3, with a binding affinity (EC50) of 0.5 nM. NPP3B815 did not exhibit any binding to the ENPP3-negative cell line CHOK1 Parental or CHOK1 cells overexpressing ENPP1 or ENPP2 (FIG. 23 and Table 12). No binding was seen to any of the cell lines with the isotype control (79C3B613) antibody.

TABLE 12
Cell Binding EC50 values for NPP3B815 across target cells and T-cells.

| | EC50 (nM) |
|---|---|
| Target Cells | |
| A704 | 1.01 |
| VMRCRCW | 0.5 |
| HepG2 ENPP3 KO | ND |
| CHO Parental | ND |
| CHO ENPP1 OE | ND |
| CHO ENPP2 OE | ND |
| CHO ENPP3 OE | 0.51 |
| T-Cells | |
| 888668965 | 197.3 |
| 110048457 | 132 |
| 110044049 | 188.5 |
| 110042593 | 145.6 |
| 110042496 | 147.2 |
| 110040278 | 253.3 |

ECx, x % effective concentration;
ND, not determined.
EC50 values were not determined if the dose-response curve did not satisfy one or both of the following criteria: R2 has to be >0.9 and the log (95% confidence interval) difference needs to be <1.2.

6.4 NPP3B815-Induced Cytotoxicity and T-Cell Activation on a Panel of Cancer Cell Lines with Different Levels of Endogenous ENPP3 Expression

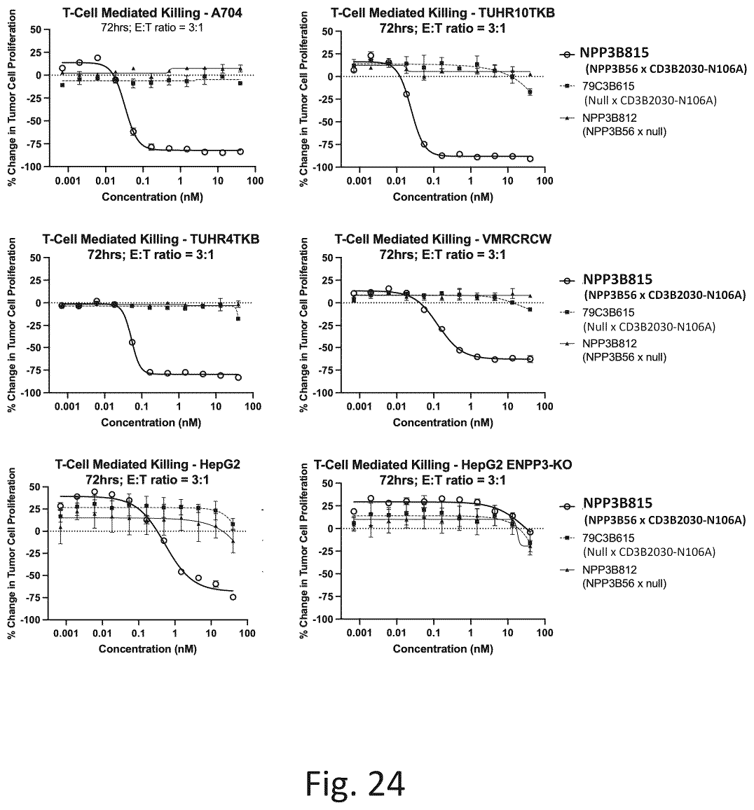

The ability of NPP3B815 (NPP3B56×CD3B2030-N106A) to induce T-cell-mediated tumor cell killing was assessed using an Incucyte (live cell time-lapse) instrument against a panel of cancer cell lines (Table 13) with different ENPP3 expression levels: high (A704), medium-high (TUHR10TKB, TUHR4TKB), medium (VMRCRCW, HepG2), and negative (HepG2 ENPP3-KO). NPP3B815-induced dose-dependent T-cell-mediated cytotoxicity at an Effector to Target cell (E:T) ratio of 3:1 on all the ENPP3-positive cell lines (A704, VMRCRCW, HepG2, TUHR4TKB, and TUHR10TKB), while no killing was seen against the negative cell line (HepG2 ENPP3-KO) (FIG. 24). As expected, no T-cell-mediated tumor cell killing was seen with either Null×CD3 (79C3B615) or ENPP3×Null (NPP3B812) control antibodies in any of the cell lines tested. The EC50 and maximum activity values for tumor cell killing are shown in Table 13, as the cell lines with higher ENPP3 expression had lower EC50 values with high max tumor cell killing.

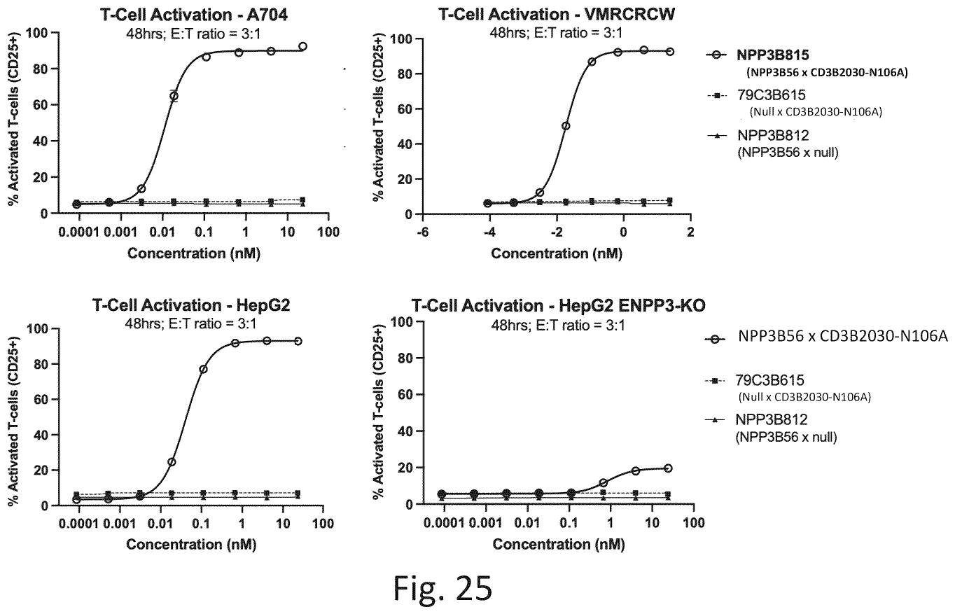

To assess the level of T cell activation induced by the ENPP3×CD3 bispecific antibody, CD25 expression was measured on T cells by flow cytometry at 48 hours post antibody treatment (FIG. 25). A dose-dependent increase in T cell activation (E:T ratio=3:1) was seen with NPP3B815 on all the ENPP3-positive cell lines tested (A704, VMRCRCW, and HepG2, while no T cell activation was seen with NPP3B815 in the negative cell line (HepG2 ENPP3-KO) or with the control antibodies Null×CD3 and ENPP3×Null in any of the cell lines tested. The EC50 and maximum activity values for T-cell activation are shown in Table 13

### Table.

TABLE 13
EC50 and maximum activity values for tumor cell
killing and T-cell activation in a panel of tumor
cell lines with endogenous ENPP3 expression.

| | Tumor cell killing | | | T-cell activation | | |
|---|---|---|---|---|---|---|
| | (Incucyte) | | | (Flow Cytometry) | | |
| | | | Max | | | Max |
| | EC50 | EC90 | killing | EC50 | EC90 | killing |
| Cell line | (nM) | (nM) | (%) | (nM) | (nM) | (%) |
| A704 | 0.03 | 0.08 | 82 | 0.011 | 0.042 | 90 |
| TUHR10TKB | 0.02 | 0.06 | 88 | — | — | — |
| TUHR4TKB | 0.05 | 0.09 | 80 | — | — | — |
| VMRCRCW | 0.13 | 0.65 | 63 | 0.018 | 0.085 | 93 |
| HepG2 | 0.53 | 3.68 | 68 | 0.041 | 0.175 | 93 |
| HepG2 | ND | ND | ND | 0.842 | 3.834 | 20 |
| ENPP3-KO | | | | | | |

ECx, x % effective concentration; Max, maximal; ND, not determined.
EC50, EC90, and Max killing values were not determined if the dose-response curve did not satisfy one or both of the following criteria: R2 has to be >0.9 and the log (95% confidence interval) difference needs to be <1.2.

### 6.5 NPP3B815-Induced Cytotoxicity and T Cell Activation Across Multiple T Cell Donors and E:T Ratios

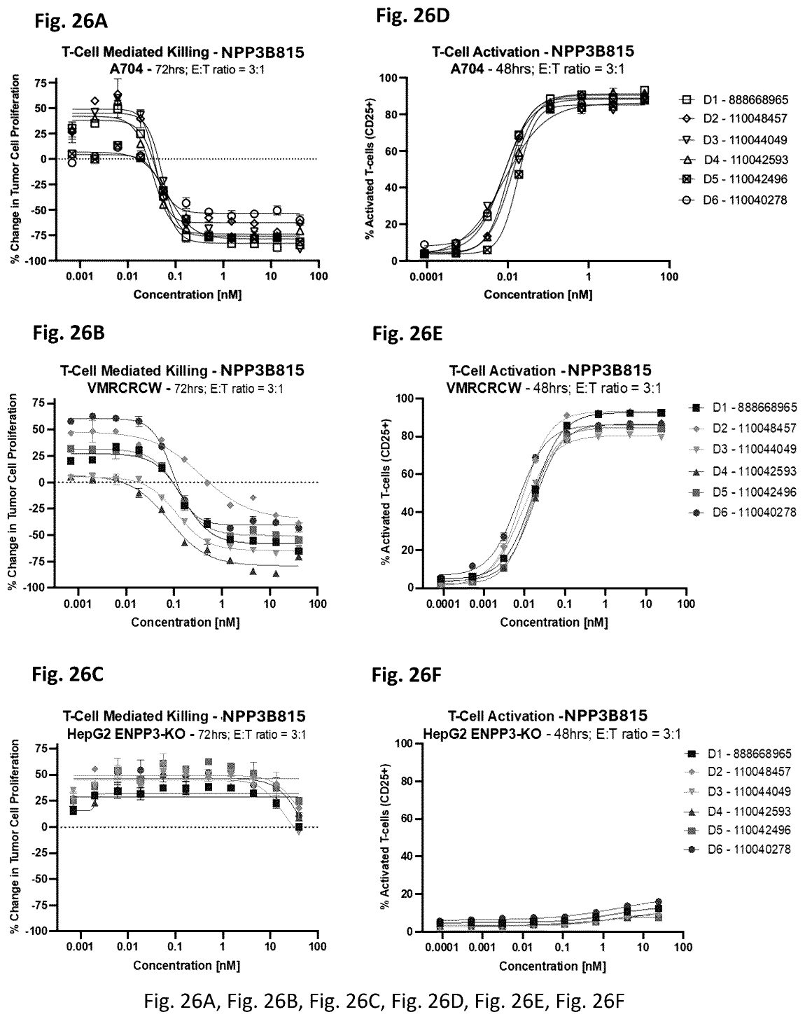

NPP3B815-induced T-cell-mediated tumor cell killing of ENPP3-positive cell lines A704 (high) and VMRCRCW (medium) and of ENPP3-negative cell line HepG2 ENPP3-KO was evaluated in the presence of T cells isolated from 6 different healthy human donors (tested at E:T ratio=3:1) (FIG. 26A). Dose-dependent T-cell-mediated cytotoxicity was seen with NPP3B815 across all 6 donors, with some variability seen in EC50 values and maximum cell killing between the different donors (Table 14). As expected, no T-cell-mediated tumor cell killing was seen with Null×CD3 control antibody 79C3B615 against all cell lines and T-cell donors tested. Like A704, dose-dependent T-cell-mediated cytotoxicity was seen with NPP3B815 across all donors in the ENPP3-medium cell line, VMRCRCW, while no killing was observed against the negative cell line (HepG2 ENPP3-KO). EC50 and maximum killing values are shown in Table 14.

In addition to the cytotoxicity assessment described above, NPP3B815-induced T-cell activation (CD25 expression by flow cytometry at 48 hours) was evaluated with the same 6 T-cell donors at an E:T ratio of 3:1 (FIG. 26B). A dose-dependent increase in T-cell activation was seen with NPP3B815 across all 6 T-cell donors in both ENPP3-positive cell lines (A704 and VMRCRCW). No T-cell activation was seen with NPP3B815 in the negative cell line (HepG2 ENPP3-KO) or with the Null×CD3 control antibody 79C3B615 against all cell lines tested. The EC50 and maximum values for T-cell activation are shown in Table 15.

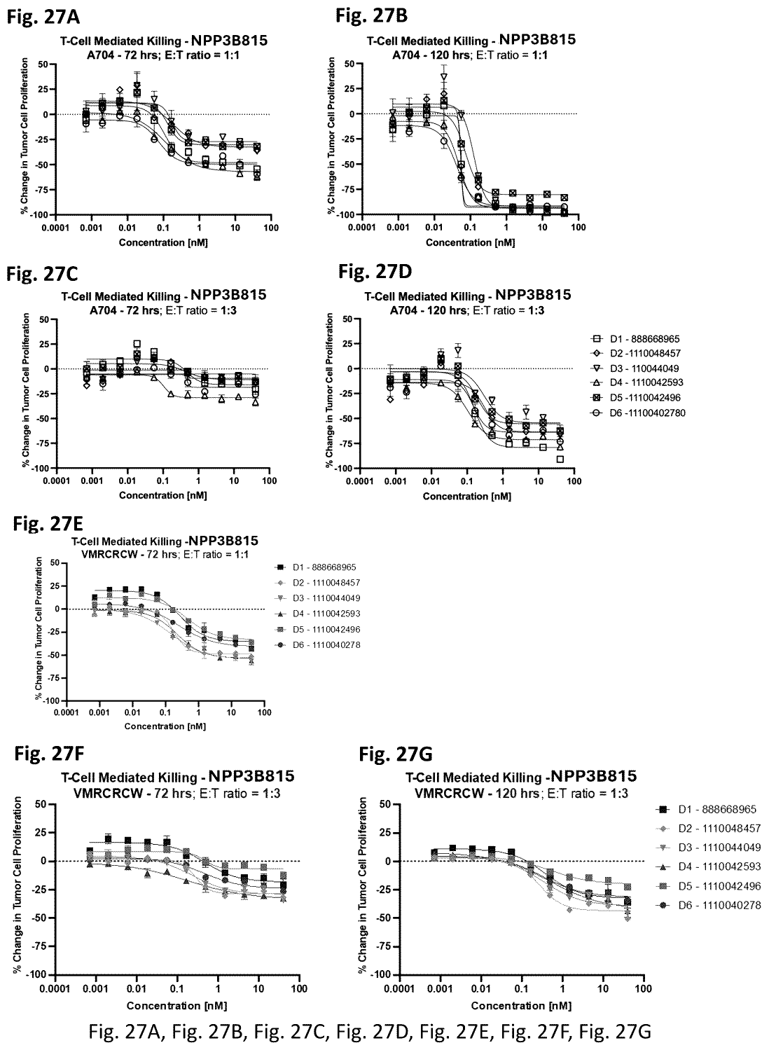

NPP3B815-induced cytotoxicity was also evaluated at lower, more physiologically relevant E:T ratios of 1:1 and 1:3, and a dose-dependent killing was observed in the ENPP3-high cell line, A704 at these E:T ratios (FIG. 27). A modest decrease in the maximum killing activity of NPP3B815 was seen at the lower E:T ratios of 1:1 (41%) and 1:3 (17%), compared to that observed in the ENPP3-high cell line A704 at an E:T ratio of 3:1 (71%), when measured at 72 hours (Table 14). NPP3B815 Interestingly, with longer incubation time (5 days vs 3 days), the maximum killing in A704 at the lower E:T ratios 1:3 increased from 41% to 91% for E:T=1:1 and from 17% to 65% 35% for E:T=1:3, leading to similar max killing as with higher E:T ratios. Similar cytotoxicity assays at lower E:T ratios were also performed in the ENPP3-medium cell line VMRCRCW and the negative cell line, HepG2 ENPP3-KO. A dose-dependent killing was observed in VMRCRCW at the lower E:T ratios, while no killing was observed against the negative cell line HepG2 ENPP3-KO (FIG. 27). As expected, no T-cell-mediated tumor cell killing was seen with the Null×CD3 control antibody 79C3B615 at any of the E:T ratios or cell lines tested. The EC50 and maximum activity values for tumor cell killing are shown in Table 14.

TABLE 14
EC50 values for tumor cell cytotoxicity across T-cell donors and E:T ratios.

| | E:T = 3:1 | | E:T = 1:1 | | | | E:T = 1:3 | | | |
|---|---|---|---|---|---|---|---|---|---|---|
| | | Max | | Max | | Max | | Max | | Max |
| | EC50 | killing | EC50 | killing | EC50 | killing | EC50 | killing | EC50 | killing |
| T-cell | (nM) @ | (%) @ | (nM) @ | (%) @ | (nM) @ | (%) @ | (nM) @ | (%) @ | (nM) @ | (%) @ |
| donors | 72 hrs | 72 hrs | 72 hrs | 72 hrs | 120 hrs | 120 hrs | 72 hrs | 72 hrs | 120 hrs | 120 hrs |

A704

| | | | | | | | | | | |
|---|---|---|---|---|---|---|---|---|---|---|
| 888668965 | 0.04545 | 83 | ND | 48 | ND | 93 | ND | 16 | 0.163 | 79 |
| 110048457 | ND | 63 | ND | 33 | ND | 92 | ND | ND | ND | 64 |
| 110044049 | 0.05244 | 76 | ND | 27 | 0.125 | 94 | ND | 10 | ND | 54 |
| 110042593 | 0.03358 | 74 | 0.114 | 57 | 0.048 | 94 | ND | 29 | 0.106 | 71 |
| 110042496 | 0.06961 | 79 | 0.132 | 30 | 0.073 | 80 | ND | 11 | 0.191 | 55 |
| 110040278 | 0.04837 | 53 | 0.075 | 50 | 0.041 | 92 | ND | 19 | 0.149 | 64 |

VMRCRCW

| | | | | | | | | | | |
|---|---|---|---|---|---|---|---|---|---|---|
| 888668965 | 0.1561 | 58 | 0.273 | 35 | 0.345 | 59 | 0.469 | 18 | 0.379 | 32 |
| 110048457 | 0.3716 | 35 | 0.177 | 48 | 0.21 | 66 | 0.276 | 29 | 0.267 | 43 |
| 110044049 | 0.1148 | 65 | 0.151 | 54 | 0.216 | 66 | 0.226 | 29 | 0.337 | 39 |
| 110042593 | 0.07951 | 79 | 0.225 | 53 | 0.218 | 68 | ND | ND | 0.485 | 43 |
| 110042496 | 0.1278 | 50 | 0.465 | 33 | 0.454 | 44 | ND | ND | 0.5 | 20 |
| 110040278 | 0.09229 | 40 | 0.243 | 41 | 0.264 | 53 | 0.456 | 24 | 0.332 | 31 |

HepG2 ENPP3-KO

| | | | | | | | | | | |
|---|---|---|---|---|---|---|---|---|---|---|
| 888668965 | ND | ND | ND | ND | ND | ND | ND | ND | ND | ND |
| 110048457 | ND | ND | ND | ND | ND | ND | ND | ND | ND | ND |
| 110044049 | ND | ND | ND | ND | ND | ND | ND | ND | ND | ND |
| 110042593 | ND | ND | ND | ND | ND | ND | ND | ND | ND | ND |
| 110042496 | ND | ND | ND | ND | ND | ND | ND | ND | ND | ND |
| 110040278 | ND | ND | ND | ND | ND | ND | ND | ND | ND | ND |

ECx, x % effective concentration; Max, maximal; ND, not determined.
EC50 and Max killing values were not determined if the dose-response curve did not satisfy one or both of the following criteria: R2 has to be >0.9 and the log (95% confidence interval) difference needs to be <1.2.

TABLE 15
EC50 values for T-cell activation across donors (E:T ratio = 3:1).

| | A704 | | VMRCRCW | | HepG2 ENPP3-KO | |
|---|---|---|---|---|---|---|
| | | Max | | Max | | Max |
| T-cell | EC50 | activity | EC50 | activity | EC50 | activity |
| donors | (nM) | (%) | (nM) | (%) | (nM) | (%) |
| 888668965 | 0.008 | 91 | 0.016 | 92 | ND | ND |
| 110048457 | 0.01 | 89 | 0.009 | 93 | 1.142 | 9.414 |
| 110044049 | 0.008 | 86 | 0.009 | 80 | ND | ND |
| 110042593 | 0.013 | 90 | 0.017 | 86 | ND | ND |
| 110042496 | 0.017 | 85 | 0.015 | 85 | ND | ND |
| 110040278 | 0.008 | 88 | 0.007 | 86 | ND | ND |

EC50 and Max activity values were not determined if the dose-response curve did not satisfy one or both of the following criteria: R2 has to be >0.9 and the log (95% confidence interval) difference needs to be <1.2.

### 6.6 NPP3B815-Induced Cytokine Release in Presence of T Cells and Tumor Cells

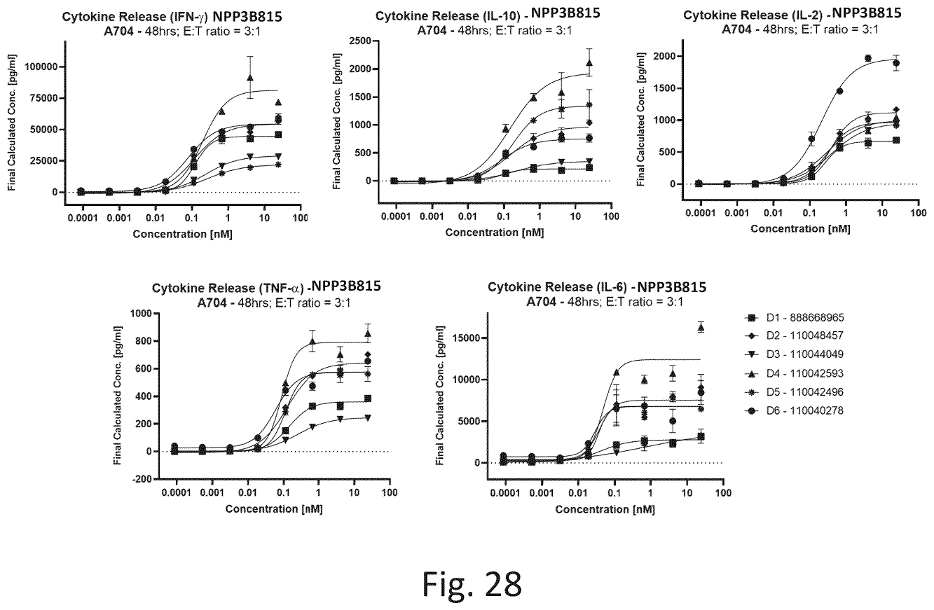

To further characterize T cell activation by NPP3B815, cytokine release profiles of 10 established inflammatory cytokines were measured using the Human Vplex Proinflammatory Panel (Meso Scale Discovery [MSD]) in the ENPP3-high cell line A704 at 48 hours post incubation with T cells from 6 different healthy human donors at an E:T ratio of 3:1. A dose-dependent increase in all pro-inflammatory cytokines tested was seen with NPP3B815 across all 6 T cell donors with some variability in response seen between donors (FIG. 28). Some cytokines such as interferon (IFN)-γ, interleukin (IL)-2, IL-10, IL-6, and tumor necrosis factor (TNF)-α exhibited a strong dose-dependent induction with NPP3B815 treatment (FIG. 28 and Table 16), while others (IL-10, IL-12p70, IL-4, IL-13, IL-8) had a modest to low dose-dependent induction. Concentrations of induced cytokines widely vary upon NPP3B815 treatment from the lowest induced cytokine (IL-1β) in the single-digit pg/mL to the highest (IFN-γ) in the 3-digit ng/mL range. The Null×CD3 control antibody 79C3B615 induced low/no cytokine response as expected, except in the case of IL-8, which showed high background signal.

TABLE 16
EC50 values for cytokine release with T-cell donors.

| T-cell donors | IFN-g | IL-10 | IL-2 | TNF-a | IL-6 |
|---|---|---|---|---|---|
| 888668965 | 0.122 | 0.085 | 0.268 | 0.143 | 0.041 |
| 110048457 | 0.117 | 0.156 | 0.390 | 0.124 | 0.037 |
| 110044049 | 0.240 | 0.210 | 0.395 | 0.241 | 0.782 |
| 110042593 | 0.198 | 0.151 | 0.295 | 0.087 | 0.047 |
| 110042496 | 0.264 | 0.177 | 0.305 | 0.112 | 0.036 |
| 110040278 | 0.078 | 0.084 | 0.211 | 0.059 | 0.029 |

ECx, x % effective concentration;
Max, maximal;
ND, not determined;
PBMC, peripheral blood mononuclear cell.
EC50, EC90, and Max killing values were not determined if the dose-response curve did not satisfy one or both of the following criteria: R2 has to be >0.9 and the log (95% confidence interval) difference needs to be <1.2.

6.7 NPP3B815-Induced Cytotoxicity from PBMC Donors

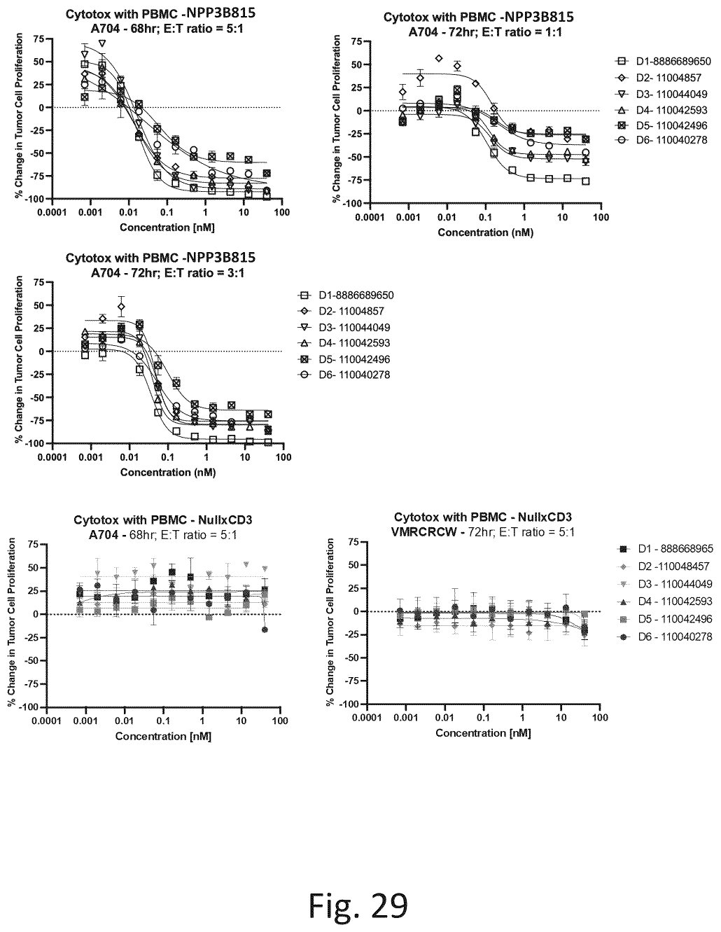

NPP3B815-induced tumor cell killing was evaluated in the presence of PBMCs isolated from corresponding 6 donors from which human T-cell were previously isolated. Dose-dependent cytotoxicity was seen with NPP3B815 treatment in the presence of all 6 PBMC donors in the ENPP3-high cell line A704 at an E:T ratio of 5:1 (FIG. 29). Dose-dependent cytotoxicity was also seen with NPP3B815 treatment in the presence of all 6 PBMC donors in the ENPP3-medium cell line VMRCRCW, while no killing was observed against the negative cell line HepG2 ENPP3-KO.

Further, NPP3B815-induced cytotoxicity in the presence of PBMCs was also evaluated at lower, more physiologically relevant E:T ratios of 3:1 and 1:1. A dose-dependent killing of ENPP3-positive cell line A704 was observed at the lower E:T ratios in the presence of all 6 PBMC donors (FIG. 29). A moderate decrease in the maximum killing activity of NPP3B815 was seen at the lowest E:T ratio of 1:1 (44%), when compared to that observed at an E:T ratio of 5:1 (82%) and 3:1 (78%). As expected, no tumor cell killing was seen with the Null×CD3 control antibody 79C3B615 in the presence of any of the PBMC donors and cell lines tested (FIG. 29). EC50 and maximum killing values are shown in Error! Reference source not found.

TABLE 17
EC50 values for tumor cell cytotoxicity with PBMC donors.

| | E:T = 5:1 | | E:T = 3:1 | | E:T = 1:1 | |
|---|---|---|---|---|---|---|
| | | Max | | Max | | Max |
| | EC50 | killing | EC50 | killing | EC50 | killing |
| T-cell donors | (nM) | (%) | (nM) | (%) | (nM) | (%) |

A704

| | | | | | | |
|---|---|---|---|---|---|---|
| 888668965 | 0.015 | 92 | 0.036 | 95 | 0.107 | 73 |
| 110048457 | 0.013 | 78 | 0.046 | 76 | ND | 26 |
| 110044049 | 0.016 | 89 | 0.052 | 79 | 0.131 | 52 |
| 110042593 | 0.015 | 83 | 0.039 | 80 | 0.124 | 47 |
| 110042496 | 0.061 | 60 | 0.097 | 64 | ND | 25 |
| 110040278 | 0.028 | 89 | 0.052 | 75 | ND | 37 |

VMRCRCW

| | | | | | | |
|---|---|---|---|---|---|---|
| 888668965 | 0.062 | 73 | — | — | — | — |
| 110048457 | 0.063 | 69 | — | — | — | — |
| 110044049 | 0.051 | 70 | — | — | — | — |
| 110042593 | 0.033 | 73 | — | — | — | — |
| 110042496 | 0.065 | 52 | — | — | — | — |
| 110040278 | 0.033 | 64 | — | — | — | — |

HepG2 ENPP3 KO

| | | | | | | |
|---|---|---|---|---|---|---|
| 888668965 | ND | ND | — | — | — | — |
| 110048457 | ND | ND | — | — | — | — |
| 110044049 | ND | ND | — | — | — | — |
| 110042593 | ND | ND | — | — | — | — |
| 110042496 | ND | ND | — | — | — | — |
| 110040278 | ND | ND | — | — | — | — |

ECx, x % effective concentration; Max, maximal; ND, not determined; PBMC, peripheral blood mononuclear cell.
EC50, EC90, and Max killing values were not determined if the dose-response curve did not satisfy one or both of the following criteria: R2 has to be >0.9 and the log (95% confidence interval) difference needs to be <1.2.

6.8 Cell Binding and Cytotoxicity with Cyno ENPP3 Expressing Cells.

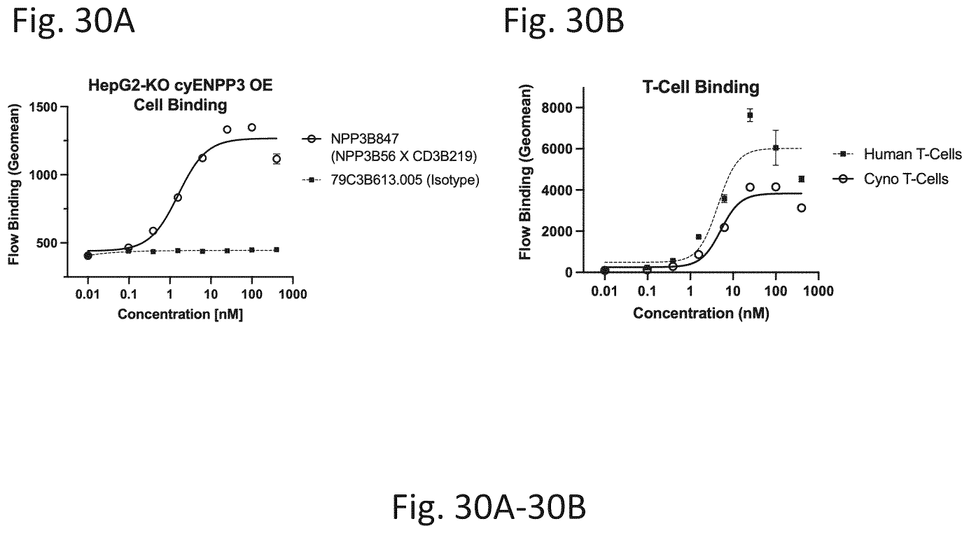

NPP3B815's ENPP3-binding arm is cynomolgus monkey cross-reactive, but its CD3-binding arm is human specific. Therefore, a tool molecule was generated (i.e., NPP3B847) containing the same ENPP3-binding arm as NPP3B815 but including a cynomolgus cross-reactive CD3-binding arm, CD3B219 (unstapled, scFv). In vitro cell binding of the tool molecule NPP3B847 was assessed on human and cyno T-cells as well as a cyno ENPP3 cell line, HepG2-KO cyENPP3 OE. NPP3B847 exhibited a dose-dependent binding to the cyno ENPP3 overexpressing cell line HepG2-KO cyENPP3 OE with an EC50 value of 1.6 nM, with no binding seen with the isotype control (79C3B613) antibody (FIG. 30A and Table 18). Further, comparable dose-dependent binding of NPP3B847 was observed with both human and cynomolgus monkey T cells, with binding affinity (EC50) values being 4.5 nM and 5.2 nM, respectively (FIG. 30B and Table 18).

TABLE 18
Summary of molecular characteristics of NPP3B815 and NPP3B847

| | | Tool molecule |
|---|---|---|
| | Species | NPP3B847 |
| CD3 Binder | | CD3B219 |
| ENPP3 (cell-based) binding | NHP | 1.6 nM |
| CD3 binding (cell-based) | Human | 4.5 nM |
| | NHP | 5.2 nM |

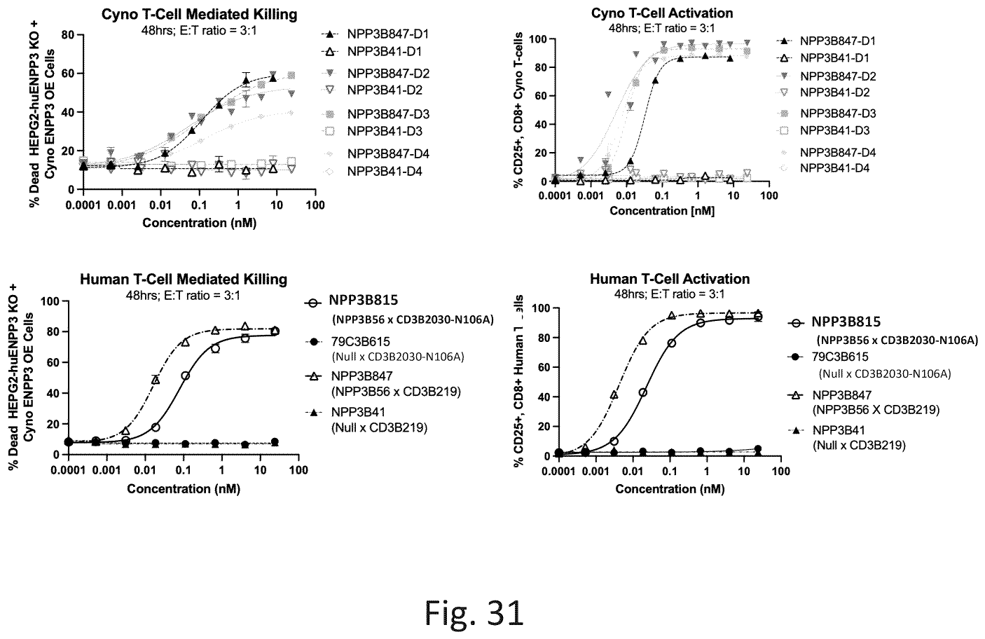

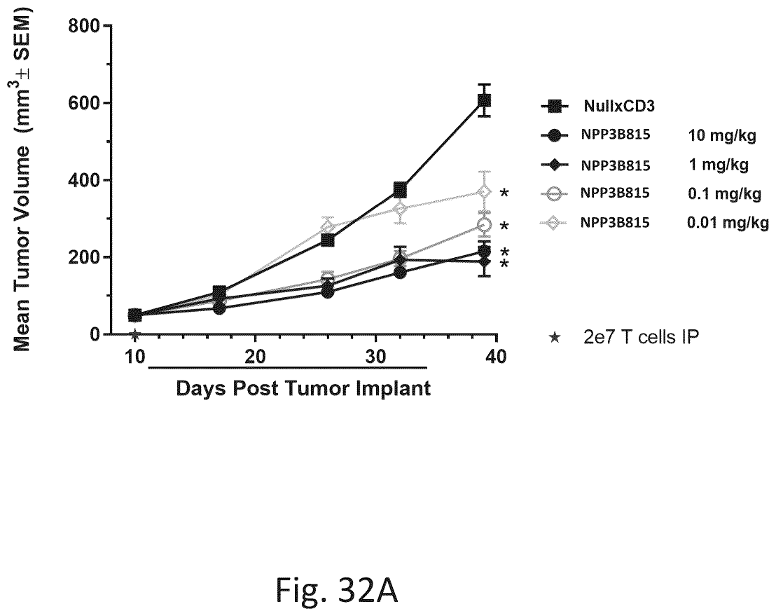

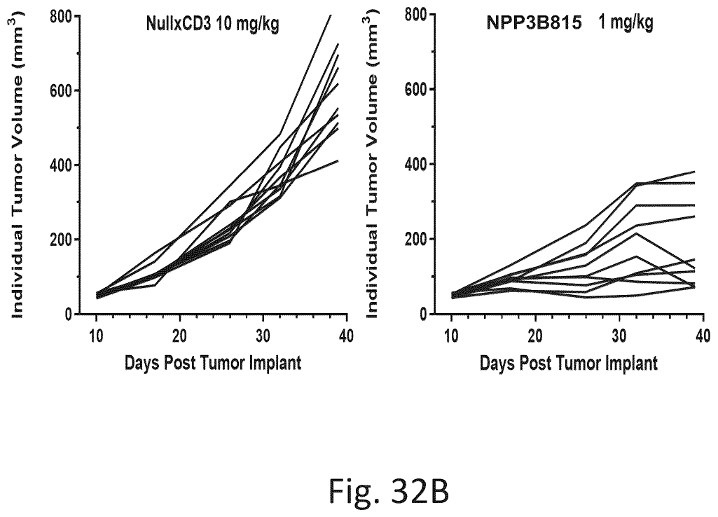

An assessment of the in vitro functional activity of the tool molecule NPP3B847 was conducted by assessing T-cell activation and T-cell-mediated tumor cell cytotoxicity in the presence of human or cynomolgus monkey T cells. These readouts were measured by flow cytometry in HepG2 ENPP3-KO cells overexpressing cyno ENPP3 (FIG. 31 and Table 19) and compared to the activity of the clinical candidate NPP3B815 in the presence of human T-cells.

NPP3B847-induced T-cell-mediated cell killing of HepG3-KO cyno ENPP3-OE cell line was evaluated in the presence of cyno T cells isolated from 4 different donors (tested at E:T ratio=3:1) (FIG. 31). Dose-dependent T-cell-mediated cytotoxicity was seen with NPP3B847 across all 4 donors, with some variability seen in EC50 values (0.05 nM-0.14 nM) between the different donors (Table 19). As expected, no T-cell-mediated tumor cell killing was seen with Null×CD3 control antibody NPP3B41 with cyno T-cell donors tested. Further, NPP3B847 induced T-cell-mediated cell killing of HepG2-KO cyno ENPP3-OE cell line was compared to that of NPP3B815 using human T-cells as effector cells at an E:T ratio=3:1 (FIG. 31). Dose-dependent T-cell-mediated cytotoxicity was seen with both NPP3B847 and NPP3B815 at EC50 values of 0.016 nM and 0.079 nM respectively, with no killing seen with Null×CD3 control antibody NPP3B41 with human T-cells (Table 19).

In addition to assessing cytotoxicity, NPP3B847-induced T-cell activation (CD25 expression by flow cytometry at 48 hours) was evaluated with the same 4 cyno T-cell donors at an E:T ratio of 3:1 (FIG. 31). A dose-dependent increase in T-cell activation (EC50=0.005 nM-0.033 nM) was seen with NPP3B847 across all 4 T-cell donors tested in the HepG3-KO cyno ENPP3-OE cell line and no activity was seen with Null×CD3 control antibody NPP3B41. Further, human T-cell activation was evaluated with both NPP3B847 and NPP3B815 using HepG2-KO cyno ENPP3-OE cell line at an E:T ratio=3:1 (FIG. 31). Dose-dependent human T-cell-activation was seen with both NPP3B847 and NPP3B815 at an EC50 value of 0.004 nM and 0.023 nM, respectively, with no activation seen with Null×CD3 control antibody NPP3B41. The EC50 values for cyno and human T-cell activation with NPP3B815 and NPP3B41 are shown in Table 19.

TABLE 19
Summary of EC50 values from cytotoxicity and T-cell activation assays using NPP3B815 and NPP3B847.

| | | | | | | | | | Human T cells | |
|---|---|---|---|---|---|---|---|---|---|---|
| | Cyno T cells | | | | | | | | | T-cell |
| | Cytotoxicity EC50 (nM) | | | | T-cell Activation EC50 (nM) | | | | Cytotox. | activation |
| | Donor | Donor | Donor | Donor | Donor | Donor | Donor | Donor | EC50 | EC50 |
| Molecule | 1 | 2 | 3 | 4 | 1 | 2 | 3 | 4 | (nM) | (nM) |
| NPP3B847 | 0.114 | 0.053 | 0.1115 | 0.137 | 0.033 | 0.005 | 0.011 | 0.008 | 0.016 | 0.004 |

| | | | | |
|---|---|---|---|---|
| NPP3B815 | N/A | N/A | 0.079 | 0.023 |

cyno, cynomolgus monkey; cytotox., cytotoxicity; EC50, 50% effective concentration; N/A, not applicable.

### 6.9 In Vivo Efficacy

The antitumor activity of the ENPP3×CD3 stapled (spFv) BsAb NPP3B815 or the unstapled (scFv) version NPP3B194 compared with Null×CD3 control antibody was evaluated in the established ENPP3-low (˜10,600 receptors/cell) human RCC CDX model VMRCRCW or the ENPP3-medium (˜22,000 receptors/cell) human HCC HepG2 model, respectively. Efficacy studies were performed in female immune-compromised NSG mice humanized with donor CD3+ pan T cells. Twice-weekly treatment with NPP3B815 or NPP3B194 administered intraperitoneally (IP) was initiated after SC tumors were established and 1 day post IP T cell engraftment. Engraftment of human T cells can lead to body weight loss due to eventual graft-versus-host disease (GvHD); however, treatment with NPP3B815 did not result in significant body weight loss as compared to the Null×CD3-treated control group. In the HepG2 tumor model, body weight loss due to tumor-induced cachexia was observed across all groups.

In Study ONC2022-035 [ELN ENPP3-00163], mice bearing established SC VMRCRCW xenografts were IP dosed with NPP3B815 twice weekly at 10, 1, 0.1, and 0.01 mg/kg or Null×CD3 control antibody (10 mg/kg) for a total of 8 doses (n=10/group). Significant antitumor efficacy was observed with NPP3B815 treatment at 10 and 1 mg/kg over time (p<0.05) with 70% and 75% A tumor growth inhibition (TGI), respectively, as compared to Null×CD3-antibody-treated control mice on Day 39 post tumor implantation (Error! Reference source not found. A). Treatment with NPP3B815 at 0.1 and 0.01 mg/kg resulted in 58% and 42% ΔTGI, respectively, as compared to the Null×CD3-antibody-treated animals on Day 39. While statistically significant (p<0.05) the data at these lower NPP3B815 doses are not biologically significant (Johnson et al., 2001, Br J Cancer, 84(10):1424-1431). The lack of biologically significant efficacy (i.e., >60% TGI) observed from treatment with NPP3B815 at 0.1 and 0.01 mg/kg demonstrated that 1 mg/kg is the minimally efficacious dose against low ENPP3 target level model.

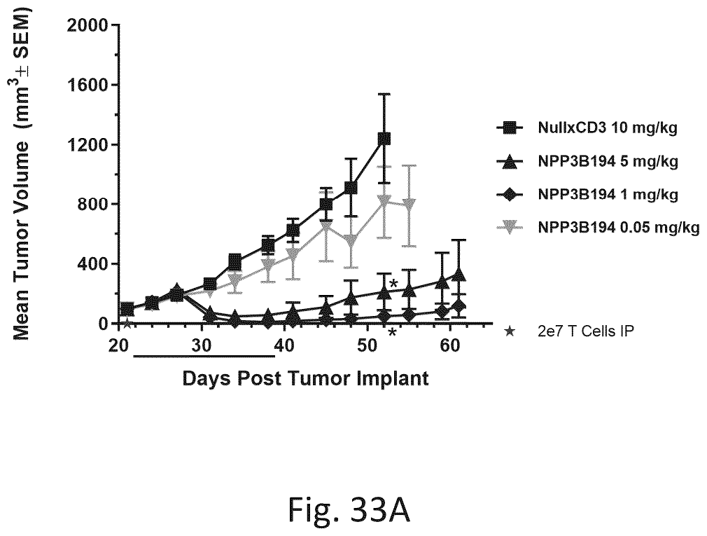

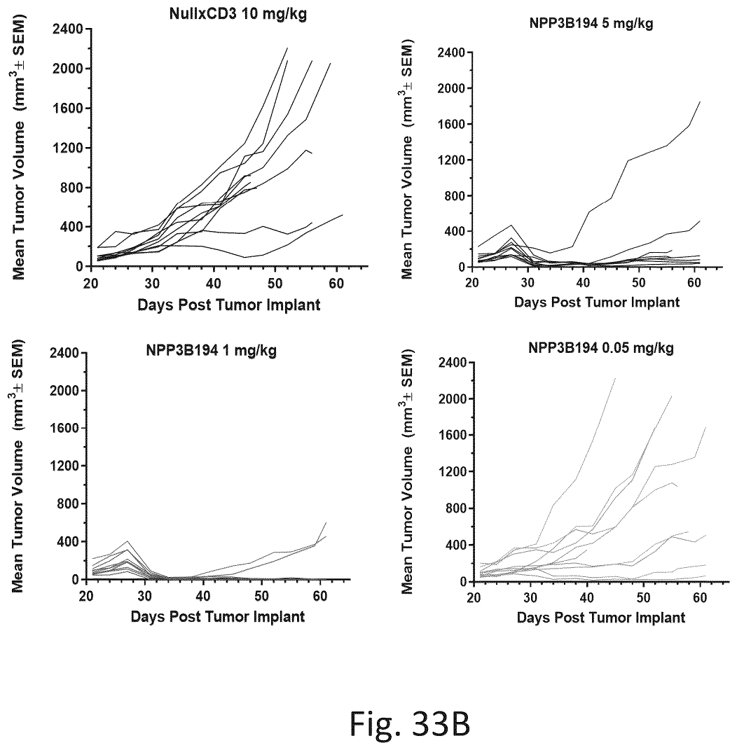

In Study P764Y [ELN ENPP3-00166], mice bearing established SC HepG2 xenografts were IP dosed with NPP3B194 (ScFv version of NPP3B815) twice weekly at 5, 1, and 0.05 mg/kg or with Null×CD3 control antibody for a total of 6 doses (n=10/group). Significant antitumor efficacy was observed with NPP3B194 treatment at 5 and 1 mg/kg over time (p<0.05) with 90% and 104% ΔTGI, respectively, as compared to Null×CD3-antibody-treated control mice on Day 52 post tumor implantation (FIG. 33). Treatment with NPP3B194 at 0.05 mg/kg was not statistically significant as compared to the Null×CD3-antibody-treated animals on Day 52. Treatment with NPP3B194 at 5 and 1 mg/kg resulted in 2 of 8 and 7 of 9 complete tumor regressions on the last day of study (Day 61), respectively. All efficacy results are summarized in Table 20.

TABLE 20
Summary of efficacy results for NPP3B815 or NPP3B194.

| | | | Animals | | |
|---|---|---|---|---|---|
| Study type | Species/test | Treatment | per group | Dose groups and key | |
| [reference] | system | duration | (M/F) | results | ELN |
| VMRCRCW | NSG mice/ | 4 weeks | 10 (F) | 42% ΔTGI at 0.01 mg/kg; | ENPP3- |
| SC established | T-cell Donor | (NPP3B815) | | 58% ΔTGI at 0.1 mg/kg; | 00163 |
| [Study | D204071 Lot | | | 75% ΔTGI at 1 mg/kg; | |
| ONC2022- | 19055100, | | | 70% ΔTGI at 10 mg/kg; | |
| 035] | HemaCare | | | q3d-q4d IP for 8 doses | |
| HepG2 SC | NSG mice/ | 3 weeks | 10 (F) | 104% ΔTGI at 1 mg/kg, 7 | ENPP3- |
| established | T-cell Donor | (NPP3B194) | | of 9 CRs; 90% ΔTGI at | 00166 |
| [Study P764Y] | D204071 Lot | | | 5 mg/kg, 2 of 8 CRs; 38% | |
| | 19057273, | | | ΔTGI at 0.05 mg/kg (ns); | |
| | HemaCare | | | q3d-q4d IP for 6 doses | |

CR, complete response;
F, female;
IP, intraperitoneal;
M, male;
NSG, non-obese diabetic (NOD) severe combined immunodeficiency (scid) gamma or NOD.Cg-Prkdescid Il2rgtm1Wj1/SzJ; q3d-q4d, twice a week;
SC, subcutaneous;
TGI, tumor growth inhibition.
p < 0.05 versus control except where noted as not significant (ns).

### Example 7. Generation of Anti-GUCY2C Antibodies

### 7.1 Immunization of Ablexis Kappa Mice

Ablexis Kappa mice were immunized with alternating boosts of 10 μg of cyno GUCY2C extracellular domain (ECD) (day 0, 14, 28) or human GUCY2C ECD (day 7, 21). Zinc chitosan nanoparticles were used in conjunction in some instances. The mice received 5 weekly boosts and a final boost at day 44 with human GUCY2C ECD (with zinc chitosan nanoparticles in some instances) and 50 μg of anti-mouse CD40 (R&D Systems MAB440).

Immune sera samples were collected once during the immunization (Day 28) and tested for binding via flow cytometry to T84 (GUCY2C+) and T84 GUCY2C KO (GUCY2C−) cells. Protein serology was done on human and cyno GUCY2C ECD proteins by MSD. Quality of Immune Response analysis was done by SPR on human and cyno GUCY2C ECD coated chips.

### 7.2 Single B-Cell Recovery

On Day 51 of the immunization schedule, spleens and inguinal lymph nodes were harvested from each mouse of group 4. All lymph nodes were pooled and homogenized into a single-cell suspension, sorted on double positive AF647-labeled cyno GUCY2C and biotinylated human GUCY2C (with streptavidin-PE detection). Cells were then plated and cultured for 7 days.

SBC supernatants from the sorted B cells were screened by protein MSD on biotinylated human and cyno GUCY2C. Primary hits were sent for V gene recovery. Selected recovered hits were expressed as human IgG1.

### 7.3 Expi293 Small Scale Transfection and Purification

Antibodies identified from the immunization campaign were cloned and expressed as human IgG1 at 2 ml scale and purified. Expi293 cells were cultured in Expi293 Expression Medium at 37° C., 7% CO2. Cells were sub-cultured when density reached the log phase growth at 2.5-3×10\hat{ }6 viable cells/mL with a 98-99% viability. On the day of transfection, the viable cell density and percent viability was determined. Cells were transfected at a density of 2.5-3×10\hat{ }6 viable cells/mL following manufacturer's Transfection protocol (ThermoFisher ExpiCHO Expression System Protocols for 24 and 96 deep well blocks and mini bioreactor tubes). Culture supernatants were harvested on Day 5 post-transfection by centrifugation at 2000 RPM for 15 minutes prior to purification. Antibodies were purified from the clarified supernatants using Capture Select CH1-XL resin slurry (Thermo Cat 2943452050). Antibodies were eluted with 0.1 M Na-Acetate, pH 3.5 and each elution fraction was neutralized with 2.5 M Tris HC1, pH 7.5 before dialyzes into DPBS. Protein concentrations were determined by measurement of absorbance at 280 nm on the filtrate using a DropSense Instrument (Trinean NV/SA).

### 7.4 Recombinant Antibody Screening

The antibodies were screened for binding on T84 cells, CHO-huGUCY2C and EL4-huGUCY2C and counter screened on untransfected EL4 in dose response titrations. The best binders were then rescreened in dose response titrations on CHO-huGUCY2C, CHO-cynoGUCY2C, untransfected CHO and EL-4 huGUCY2C. They were also tested for competition against index GUCY2C binders and a known GUCY2C binder (reference binder) for binding to CHO-huGUCY2C cells. The variable regions of selected antibodies were sequenced. A mAb called GUCYC2_mAb was selected for further studies.

### 7.5 Sequences of GUCY2C_mAb

Sequences of representative GUCY2C_mAb are provided in Tables 21-24. Table 21 shows the HCDR1, HCDR2, HCDR3, LCDR1, LCDR2 and LCDR3 sequences of GUCY2C_mAb under the Kabat delineation. Table 22 shows the HCDR1, HCDR2, HCDR3, LCDR1, LCDR2 and LCDR3 sequences of GUCY2C_mAb under the Chothia delineation. Table 23 shows the HCDR1, HCDR2, HCDR3, LCDR1, LCDR2 and LCDR3 sequences of GUCY2C_mAb under the ABM delineation. Table 24 shows the HCDR1, HCDR2, HCDR3, LCDR1, LCDR2 and LCDR3 sequences of GUCY2C_mAb under the Contact delineation. Table 25 shows the HCDR1, HCDR2, HCDR3, LCDR1, LCDR2 and LCDR3 sequences of GUCY2C_mAb under the IMGT delineation.

TABLE 21
HCDRs and LCDRs of GUYC2C_mAb under
the Kabat delineation.

| CDR | Sequence | SEQ ID NO |
|---|---|---|
| HCDR1 | HYYWS | 102 |
| HCDR2 | RIYTSGSTNYNPSLKS | 103 |
| HCDR3 | DRYTGYFDL | 104 |
| LCDR1 | RASQSVSSSYLA | 105 |
| LCDR2 | GASSRAT | 106 |
| LCDR3 | QQYGSSPPT | 107 |

TABLE 22
HCDRs and LCDRs of GUYC2C_mAb
under the Chothia delineation.

| CDR | Sequence | SEQ ID NO |
|---|---|---|
| HCDR1 | GASISHY | 108 |
| HCDR2 | YTSGS | 109 |
| HCDR3 | DRYTGYFDL | 104 |
| LCDR1 | RASQSVSSSYLA | 105 |
| LCDR2 | GASSRAT | 106 |
| LCDR3 | QQYGSSPPT | 107 |

TABLE 23
HCDRs and LCDRs of GUYC2C_mAb
under the ABM delineation.

| CDR | Sequence | SEQ ID NO |
|---|---|---|
| HCDR1 | GASISHYYWS | 110 |
| HCDR2 | RIYTSGSTN | 111 |
| HCDR3 | DRYTGYFDL | 104 |
| LCDR1 | RASQSVSSSYLA | 105 |
| LCDR2 | GASSRAT | 106 |
| LCDR3 | QQYGSSPPT | 107 |

TABLE 24
HCDRs and LCDRs of GUYC2C_mAb
under the Contact delineation.

| CDR | Sequence | SEQ ID NO |
|---|---|---|
| HCDR1 | SHYYWS | 112 |
| HCDR2 | WIGRIYTSGSTN | 113 |
| HCDR3 | ARDRYTGYFD | 114 |
| LCDR1 | SSSYLAWY | 115 |
| LCDR2 | LLIYGASSRA | 116 |
| LCDR3 | QQYGSSPP | 117 |

TABLE 25
HCDRs and LCDRs of GUYC2C_mAb
under the IMGT delineation.

| CDR | Sequence | SEQ ID NO |
|---|---|---|
| HCDR1 | GASISHYY | 118 |
| HCDR2 | IYTSGST | 119 |
| HCDR3 | ARDRYTGYFDL | 120 |
| LCDR1 | QSVSSSY | 121 |
| LCDR2 | GAS | NA |
| LCDR3 | QQYGSSPPT | 107 |

Table 26 shows the VH1 and VL1 amino acid sequences of GUCY2C_mAb. Table 27 shows the VH1 and VL1 nucleic acid sequences of GUCY2C_mAb.

TABLE 26
Amino acid sequences of the VH1
and VL1 of GUCY2C_mAb

| | Sequence | SEQ ID NO: |
|---|---|---|
| VH1 | QVQLQESGPGLVKPSETLSLT | 122 |
| | CTVSGASISHYYWSWIRRPAG | |
| | KGLEWIGRIYTSGSTNYNPSL | |
| | KSRVTVSVDTSKNQFSLKLSS | |
| | VTAADTAVYYCARDRYTGYFD | |
| | LWGRGTLVTVSS | |
| VL1 | EIVLTQSPGTLSLSPGERATL | 123 |
| | SCRASQSVSSSYLAWYQQKPG | |
| | QAPRLLIYGASSRATGIPDRF | |
| | SGSGSGTDFTLTISRLEPEDF | |
| | AVYYCQQYGSSPPTFGGGTKL | |
| | EIK | |

TABLE 27
Nucleic acid sequences of the VH1
and VL1 of GUCY2C_mAb

| | Sequence | SEQ ID NO: |
|---|---|---|
| VH1 | CAGGTGCAGCTGCAGGAGTCGGGCCCAGGACTGGTGAAG | 124 |
| | CCTTCGGAGACCCTGTCCCTCACCTGCACTGTCTCTGGTGC | |
| | CTCCATCAGTCATTACTACTGGAGCTGGATCCGGCGGCCC | |
| | GCCGGGAAGGGACTGGAGTGGATTGGGCGTATCTATACCA | |
| | GTGGGAGCACCAACTACAACCCCTCCCTCAAGAGTCGAGT | |
| | CACCGTGTCAGTAGACACGTCCAAGAACCAGTTCTCCCTG | |
| | AAGCTGAGCTCTGTGACCGCCGCGGACACGGCCGTATATT | |
| | ACTGTGCGAGAGATCGATATACGGGGTACTTCGATCTCTG | |
| | GGGCCGTGGCACCCTGGTCACTGTCTCCTCA | |
| VL1 | GAAATTGTGTTGACGCAGTCTCCAGGCACCCTGTCTTTGTC | 125 |
| | TCCAGGGGAAAGAGCCACCCTCTCCTGCAGGGCCAGTCAG | |
| | AGTGTTAGCAGCAGCTACTTAGCCTGGTACCAGCAGAAAC | |
| | CTGGCCAGGCTCCCAGGCTCCTCATCTATGGTGCATCCAG | |
| | CAGGGCCACTGGCATCCCAGACAGGTTCAGTGGCAGTGGG | |
| | TCTGGGACAGACTTCACTCTCACCATCAGCAGACTGGAGC | |
| | CTGAAGATTTTGCAGTTTATTACTGTCAGCAGTATGGTAGC | |
| | TCACCTCCCACTTTCGGCGGAGGGACCAAGCTGGAGATCA | |
| | AA | |

### Characterization of GUCY2C_mAb

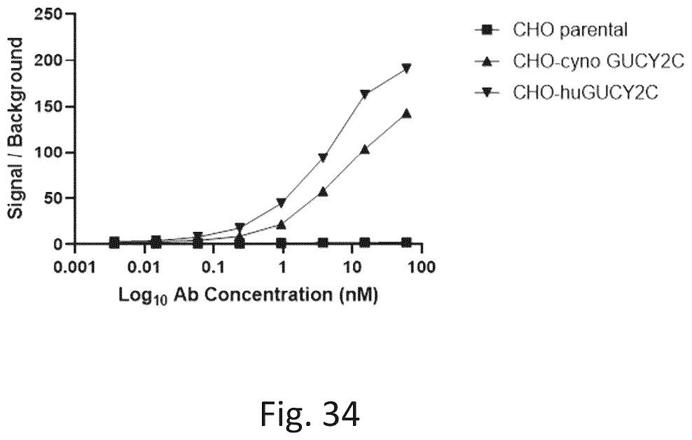

Three (3) cell lines, Chinese hamster ovary (CHO) cell line, CHO transfected with cyno GUCY2C, and CHO transfected with human GUCY2C, were incubated with a titrating dose of GUCY2C_mAb for 60 min at 37° C., washed twice, then incubated with AF647 labeled anti human secondary for 30 minutes at 4° C. Cells were washed twice again, resuspended and acquired in a flow cytometer. Specific antibody binding was assessed in the RL1H channel for each cell line. Data presented as signal/background where background is secondary only. As shown in FIG. 34, GUCY2C_mAb as generated herein binds to both human and cyno GUCY2C, with higher binding affinity to human CUCY2C.

EC50 values for binding of GUCY2C_mAb to CHO cells transfected with human or cyno GUCY2C is shown in Table 28.

TABLE 28

| Cells | EC50 |
|---|---|
| CHO-huGUCY2C | 4.6 nM |
| CHO-cynoGUCY2C | 8.7 nM |

### Generation of GUCY2C×CD3 BISPECIFIC ANTIBODIES

### 7.6 Engineering of GUCY2C×CD3 Bispecific Antibodies

In this example, two (2) GUCY2C×CD3 bispecific antibodies were generated (GUCY2C×CD3 biAb-1 and GUCY2C×CD3_biAb-2). GUCY2C×CD3_biAb-1 and GUCY2C×CD3_biAb-2 comprise the same GUCY2C binding arm, which corresponds to the GUCY2C_mAb described above and a medium and high affinity CD3 binding arm, respectively.

### 7.7 Engineering of GUCY2C×CD3_biAb-1

Briefly, the GUCY2C binding arm of GUCY2C×CD3 biAb-1 comprises an anti-GUCY2C Fab (GUCY2C_Fab) derived from GUCY2C_mAb describe in Example 1. To prepare the bispecific antibody, the VH1 and VL1 of GUCY2C_mAb were engineered in VH1-CH1-hinge-CH2-CH3 (Heavy Chain 1, HC1) and VL1-CL (Light Chain 1, LC1) formats respectively and expressed as IgG1. The Fc silencing mutation L234A/L235A/D265S were introduced in the Fc region of HC1. Mutations designed to promote selective heterodimerization (“hole” mutation T366S, L368A and Y407V) were also engineered in the Fc domain of HC1.

The CD3 binding arm of GUCY2C×CD3_biAb-1 comprises a stabilized (or stapled) scFv domain herein described as spFv-1. The CD3 arm of GUCY2C×CD3_biAb-1 is a low-medium affinity CD3 binding arm which will herein be referred to as CD3_spFv-1. The CD3_spFv-1 is derived from an anti-CD3 scFv fragment described in WO2022/201053, which is incorporated herein by reference in its entirety.

The anti-CD3 scFv as disclosed in WO2022/201053 was engineered into a stabilized scFv (herein referred to “stapled scFv” or spFv) by restraining the scFv structure, without negatively impacting, the relative movements between the VH2 and the VL2. The stabilized scFv was generated by engineering disulfide bonds between the VH2 and the linker and between the VL2 and the linker. Two structurally conserved surface exposed framework positions (anchor points) that are not involved in antigen binding, were identified, one on the VH2 at position H105 and one on the VL2 at position L42, and mutated into cysteine (Cys) residues to generate the VH2 of SEQ ID NO: 17 and VL2 of SEQ ID NO: 18. A flexible linker of sequence GGGSGGSGGCPPCGGSGG (SEQ ID NO: 2) comprising two Cys residues was used to conjugate the VL2 and the VH2 in the VL-linker-VH (LH) format. The distance and location of the Cys residues of the linker and the Cys residues of the VH2 and the VL2 is critical for the formation of the disulfide bonds between the Cys residues of the Linker and each anchor point of the VH2 and the VL2.

The stapled scFv in the VL-Linker-VH was further engineered into a Heavy Chain 2 (HC2) and expressed as IgG1. Additionally, the Fc silencing mutation L234A/L235A/D265S and the heterodimerization (“knob” mutation T366W) were engineered in the Fc domain of HC2.

HC2 comprising CD3_spFv-1 was then paired with HC1 and LC1 comprising the GUCY2C_Fab using the knob-in-hole heterodimerization technology to generate the stapled GUCY2C×CD3_biAb-1.

CDR sequences, VH2, VL2 and spFv sequence of CD3-spFv-1 are shown in Table 29-31.

TABLE 29
HCDRs and LCDRs of CD3-spFv-1
under the Kabat delineation.

| CDR | Sequence | SEQ ID NO |
|---|---|---|
| HCDR1 | RSTMH | 126 |
| HCDR2 | YINPSSAYTNYNQKFQG | 127 |
| HCDR3 | PQVHYDYAGFPY | 128 |
| LCDR1 | SASSSVSYMN | 129 |
| LCDR2 | DSSKLAS | 130 |
| LCDR3 | QQWSRNPPT | 131 |

TABLE 31
Nucleotide sequences of VH2, VL2 and
stapled VL-Linker-VH of CD3-spFv-1

| | | SEQ |
|---|---|---|
| | | ID |
| | sequence | NO: |
| VH2 | CAAGTACAACTTGTTCAATCTGGCGCTGAA | 135 |
| | GTCAAAAAACCCGGATCTAGTGTTAAAGTT | |
| | TCATGTAAGGCATCAGGCTATACTTTCACT | |
| | CGGTCTACAATGCACTGGGTCAAGCAGGCT | |
| | CCAGGGCAAGGTTTGGAATGGATAGGGTAC | |
| | ATAAATCCTTCTAGCGCCTATACTAATTAC | |
| | AACCAGAAGTTTCAGGGACGTGTTACTCTG | |
| | ACTGCTGACAAATCAACATCTACAGCCTAT | |
| | ATGGAACTGTCTTCATTGAGGAGTGAGGAC | |
| | ACTGCTGTGTATTACTGCGCTTCTCCTCAG | |
| | GTGCACTATGATTACGCTGGCTTTCCCTAC | |
| | TGGGGTTGTGGCACCCTCGTCACCGTTTCA | |
| | TCT | |
| VL2 | GAGATCGTCTTGACTCAGTCCCCTGCAACC | 136 |
| | CTCAGCGCAAGTCCCGGAGAGCGCGTTACA | |
| | TTGAGTTGTTCCGCATCCAGTTCAGTCAGC | |
| | TACATGAACTGGTACCAACAAAAGCCCGGT | |
| | TGTGCTCCAAGAAGGTGGATCTATGATTCC | |
| | AGTAAGCTGGCCTCCGGGGTCCCAGCAAGG | |
| | TTCAGCGGTTCCGGCAGCGGAAGGGACTAC | |
| | ACTCTGACCATAAGTTCCCTTGAACCAGAA | |
| | GACTTCGCCGTCTATTATTGTCAGCAGTGG | |
| | TCCCGAAATCCACCTACCTTCGGGGGCGGC | |
| | ACTAAAGTGGAGATTAAA | |
| Stapled | GAGATCGTCTTGACTCAGTCCCCTGCAACC | 137 |
| VL2- | CTCAGCGCAAGTCCCGGAGAGCGCGTTACA | |
| Linker- | TTGAGTTGTTCCGCATCCAGTTCAGTCAGC | |
| VH2 | TACATGAACTGGTACCAACAAAAGCCCGGT | |
| | TGTGCTCCAAGAAGGTGGATCTATGATTCC | |
| | AGTAAGCTGGCCTCCGGGGTCCCAGCAAGG | |
| | TTCAGCGGTTCCGGCAGCGGAAGGGACTAC | |
| | ACTCTGACCATAAGTTCCCTTGAACCAGAA | |
| | GACTTCGCCGTCTATTATTGTCAGCAGTGG | |
| | TCCCGAAATCCACCTACCTTCGGGGGCGGC | |
| | ACTAAAGTGGAGATTAAAGGAGGAGGTAGT | |
| | GGCGGCTCCGGTGGATGTCCTCCTTGCGGA | |
| | GGATCAGGAGGTCAAGTACAACTTGTTCAA | |
| | TCTGGCGCTGAAGTCAAAAAACCCGGATCT | |
| | AGTGTTAAAGTTTCATGTAAGGCATCAGGC | |
| | TATACTTTCACTCGGTCTACAATGCACTGG | |
| | GTCAAGCAGGCTCCAGGGCAAGGTTTGGAA | |
| | TGGATAGGGTACATAAATCCTTCTAGCGCC | |
| | TATACTAATTACAACCAGAAGTTTCAGGGA | |
| | CGTGTTACTCTGACTGCTGACAAATCAACA | |
| | TCTACAGCCTATATGGAACTGTCTTCATTG | |
| | AGGAGTGAGGACACTGCTGTGTATTACTGC | |
| | GCTTCTCCTCAGGTGCACTATGATTACGCT | |
| | GGCTTTCCCTACTGGGGTTGTGGCACCCTC | |
| | GTCACCGTTTCATCT | |

TABLE 30
Amino Acid sequences of VH2, VL2 and
stapled VL-Linker-VH of CD3-spFv-1

| | | SEQ |
|---|---|---|
| | | ID |
| | Sequence | NO: |
| VH2 | QVQLVQSGAEVKKPGSSVKVSCKASGYTFTRSTM | 132 |
| | HWVKQAPGQGLEWIGYINPSSAYTNYNQKFQGRV | |
| | TLTADKSTSTAYMELSSLRSEDTAVYYCASPQVH | |
| | YDYAGFPYWGCGTLVTVSS | |
| VL2 | EIVLTQSPATLSASPGERVTLSCSASSSVSYMNW | 133 |
| | YQQKPGCAPRRWIYDSSKLASGVPARFSGSGSGR | |
| | DYTLTISSLEPEDFAVYYCQQWSRNPPTFGGGTK | |
| | VEIK | |
| Stapled | EIVLTQSPATLSASPGERVTLSCSASSSVSYMNW | 134 |
| VL2- | YQQKPGCAPRRWIYDSSKLASGVPARFSGSGSGR | |
| Linker- | DYTLTISSLEPEDFAVYYCQQWSRNPPTFGGGTK | |
| VH2 | VEIKGGGSGGSGGCPPCGGSGGQVQLVQSGAEVK | |
| | KPGSSVKVSCKASGYTFTRSTMHWVKQAPGQGLE | |
| | WIGYINPSSAYTNYNQKFQGRVTLTADKSTSTAY | |
| | MELSSLRSEDTAVYYCASPQVHYDYAGFPYWGCG | |
| | TLVTVSS | |

The full-length amino acid sequences of GUCY2C-CD3-bisp-Ab1 are shown in Table 32-33. Table 32 shows the full-length amino acid sequences of the GUCY2C arm (HC1 and LC1) and of the CD3 arm (HC2). Table 33 shows the nucleic acid sequences of of the GUCY2C arm (HC1 and LC1) and of the CD3 arm (HC2).

TABLE 32
Full length amino acid sequence
of GUCY2CxCD3 bisp-Ab1

| | | Seq |
|---|---|---|
| | | ID |
| | Sequence | NO: |
| HC1 | QVQLQESGPGLVKPSETLSLTCTVSGASIS | 138 |
| (GUCY2C | HYYWSWIRRPAGKGLEWIGRIYTSGSTNYN | |
| binding | PSLKSRVTVSVDTSKNQFSLKLSSVTAADT | |
| arm) | AVYYCARDRYTGYFDLWGRGTLVTVSSAST | |
| | KGPSVFPLAPSSKSTSGGTAALGCLVKDYF | |
| | PEPVTVSWNSGALTSGVHTFPAVLQSSGLY | |
| | SLSSVVTVPSSSLGTQTYICNVNHKPSNTK | |
| | VDKKVEPKSCDKTHTCPPCPAPEAAGGPSV | |
| | FLFPPKPKDTLMISRTPEVTCVVVSVSHED | |
| | PEVKFNWYVDGVEVHNAKTKPREEQYNSTY | |
| | RVVSVLTVLHQDWLNGKEYKCKVSNKALPA | |
| | PIEKTISKAKGQPREPQVYTLPPSREEMTK | |
| | NQVSLSCAVKGFYPSDIAVEWESNGQPENN | |
| | YKTTPPVLDSDGSFFLVSKLTVDKSRWQQG | |
| | NVFSCSVMHEALHNRFTQKSLSLSPGK | |
| LC1 | EIVLTQSPGTLSLSPGERATLSCRASQSVS | 139 |
| (GUCY2C | SSYLAWYQQKPGQAPRLLIYGASSRATGIP | |
| binding | DRFSGSGSGTDFTLTISRLEPEDFAVYYCQ | |
| arm) | QYGSSPPTFGGGTKLEIKRTVAAPSVFIFP | |
| | PSDEQLKSGTASVVCLLNNFYPREAKVQWK | |
| | VDNALQSGNSQESVTEQDSKDSTYSLSSTL | |
| | TLSKADYEKHKVYACEVTHQGLSSPVTKSF | |
| | NRGEC | |
| HC2 | EIVLTQSPATLSASPGERVTLSCSASSSVS | 140 |
| (CD3 | YMNWYQQKPGCAPRRWIYDSSKLASGVPAR | |
| binding | FSGSGSGRDYTLTISSLEPEDFAVYYCQQW | |
| arm) | SRNPPTFGGGTKVEIKGGGSGGSGGCPPCG | |
| | GSGGQVQLVQSGAEVKKPGSSVKVSCKASG | |
| | YTFTRSTMHWVKQAPGQGLEWIGYINPSSA | |
| | YTNYNQKFQGRVTLTADKSTSTAYMELSSL | |
| | RSEDTAVYYCASPQVHYDYAGFPYWGCGTL | |
| | VTVSSEPKSSDKTHTCPPCPAPEAAGGPSV | |
| | FLFPPKPKDTLMISRTPEVTCVVVSVSHED | |
| | PEVKFNWYVDGVEVHNAKTKPREEQYNSTY | |
| | RVVSVLTVLHQDWLNGKEYKCKVSNKALPA | |
| | PIEKTISKAKGQPREPQVYTLPPSREEMTK | |
| | NQVSLWCLVKGFYPSDIAVEWESNGQPENN | |
| | YKTTPPVLDSDGSFFLYSKLTVDKSRWQQG | |
| | NVFSCSVMHEALHNHYTQKSLSLSPGK | |

TABLE 33
Full length nucleic acid sequence
of GUCY2CxCD3 bisp-Ab1

| | | Seq |
|---|---|---|
| | | ID |
| | Sequence | NO: |
| HC1 | CAGGTGCAGCTGCAGGAGTCGGGCCCAGGA | 141 |
| (GUCY2C | CTGGTGAAGCCTTCGGAGACCCTGTCCCTC | |
| binding | ACCTGCACTGTCTCTGGTGCCTCCATCAGT | |
| arm) | CATTACTACTGGAGCTGGATCCGGCGGCCC | |
| | GCCGGGAAGGGACTGGAGTGGATTGGGCGT | |
| | ATCTATACCAGTGGGAGCACCAACTACAAC | |
| | CCCTCCCTCAAGAGTCGAGTCACCGTGTCA | |
| | GTAGACACGTCCAAGAACCAGTTCTCCCTG | |
| | AAGCTGAGCTCTGTGACCGCCGCGGACACG | |
| | GCCGTATATTACTGTGCGAGAGATCGATAT | |
| | ACGGGGTACTTCGATCTCTGGGGCCGTGGC | |
| | ACCCTGGTCACTGTCTCCTCAGCCTCCACC | |
| | AAGGGCCCATCGGTCTTCCCCCTGGCACCC | |
| | TCCTCCAAGAGCACCTCTGGGGGCACAGCG | |
| | GCCCTGGGCTGCCTGGTCAAGGACTACTTC | |
| | CCCGAACCGGTGACGGTGTCGTGGAACTCA | |
| | GGCGCCCTGACCAGCGGCGTGCACACCTTC | |
| | CCGGCTGTCCTACAGTCCTCAGGACTCTAC | |
| | TCCCTCAGCAGCGTGGTGACCGTGCCCTCC | |
| | AGCAGCTTGGGCACCCAGACCTACATCTGC | |
| | AACGTGAATCACAAGCCCAGCAACACCAAG | |
| | GTGGACAAGAAAGTTGAGCCCAAATCTTGT | |
| | GACAAAACTCACACATGTCCACCGTGCCCA | |
| | GCACCTGAAGCAGCAGGGGGACCGTCAGTC | |
| | TTCCTCTTCCCCCCAAAACCCAAGGACACC | |
| | CTCATGATCTCCCGGACCCCTGAGGTCACA | |
| | TGCGTGGTGGTGAGCGTGAGCCACGAAGAC | |
| | CCTGAGGTCAAGTTCAACTGGTACGTGGAC | |
| | GGCGTGGAGGTGCATAATGCCAAGACAAAG | |
| | CCGCGGGAGGAGCAGTACAACAGCACGTAC | |
| | CGTGTGGTCAGCGTCCTCACCGTCCTGCAC | |
| | CAGGACTGGCTGAATGGCAAGGAGTACAAG | |
| | TGCAAGGTCTCCAACAAAGCCCTCCCAGCC | |
| | CCCATCGAGAAAACCATCTCCAAAGCCAAA | |
| | GGGCAGCCCCGAGAACCACAGGTGTACACC | |
| | CTGCCCCCATCCCGGGAGGAGATGACCAAG | |
| | AACCAGGTCAGCCTGTCCTGCGCGGTCAAA | |
| | GGCTTCTATCCCAGCGACATCGCCGTGGAG | |
| | TGGGAGAGCAATGGGCAGCCGGAGAACAAC | |
| | TACAAGACCACGCCTCCCGTGCTGGACTCC | |
| | GACGGCTCCTTCTTCCTCGTCAGCAAGCTC | |
| | ACCGTGGACAAGTCTAGATGGCAGCAGGGG | |
| | AACGTCTTCTCATGCTCCGTGATGCATGAG | |
| | GCTCTGCACAACCGGTTCACGCAGAAGAGC | |
| | CTCTCCCTGTCTCCGGGTAAA | |
| LC1 | GAAATTGTGTTGACGCAGTCTCCAGGCACC | 142 |
| (GUCY2C | CTGTCTTTGTCTCCAGGGGAAAGAGCCACC | |
| binding | CTCTCCTGCAGGGCCAGTCAGAGTGTTAGC | |
| arm) | AGCAGCTACTTAGCCTGGTACCAGCAGAAA | |
| | CCTGGCCAGGCTCCCAGGCTCCTCATCTAT | |
| | GGTGCATCCAGCAGGGCCACTGGCATCCCA | |
| | GACAGGTTCAGTGGCAGTGGGTCTGGGACA | |
| | GACTTCACTCTCACCATCAGCAGACTGGAG | |
| | CCTGAAGATTTTGCAGTTTATTACTGTCAG | |
| | CAGTATGGTAGCTCACCTCCCACTTTCGGC | |
| | GGAGGGACCAAGCTGGAGATCAAACGTACG | |
| | GTGGCTGCACCATCTGTCTTCATCTTCCCG | |
| | CCATCTGATGAGCAGTTGAAATCTGGAACT | |
| | GCCTCTGTTGTGTGCCTGCTGAATAACTTC | |
| | TATCCCAGAGAGGCCAAAGTACAGTGGAAG | |
| | GTGGATAACGCCCTCCAATCGGGTAACTCC | |
| | CAGGAGAGTGTCACAGAGCAGGACAGCAAG | |
| | GACAGCACCTACAGCCTCAGCAGCACCCTG | |
| | ACGCTGAGCAAAGCAGACTACGAGAAACAC | |
| | AAAGTCTACGCCTGCGAAGTCACCCATCAG | |
| | GGCCTGAGCTCGCCCGTCACAAAGAGCTTC | |
| | AACAGGGGAGAGTGT | |
| HC2 | GAGATCGTCTTGACTCAGTCCCCTGCAACC | 143 |
| (CD3 | CTCAGCGCAAGTCCCGGAGAGCGCGTTACA | |
| binding | TTGAGTTGTTCCGCATCCAGTTCAGTCAGC | |
| arm) | TACATGAACTGGTACCAACAAAAGCCCGGT | |
| | TGTGCTCCAAGAAGGTGGATCTATGATTCC | |
| | AGTAAGCTGGCCTCCGGGGTCCCAGCAAGG | |
| | TTCAGCGGTTCCGGCAGCGGAAGGGACTAC | |
| | ACTCTGACCATAAGTTCCCTTGAACCAGAA | |
| | GACTTCGCCGTCTATTATTGTCAGCAGTGG | |
| | TCCCGAAATCCACCTACCTTCGGGGGCGGC | |
| | ACTAAAGTGGAGATTAAAGGAGGAGGTAGT | |
| | GGCGGCTCCGGTGGATGTCCTCCTTGCGGA | |
| | GGATCAGGAGGTCAAGTACAACTTGTTCAA | |
| | TCTGGCGCTGAAGTCAAAAAACCCGGATCT | |
| | AGTGTTAAAGTTTCATGTAAGGCATCAGGC | |
| | TATACTTTCACTCGGTCTACAATGCACTGG | |
| | GTCAAGCAGGCTCCAGGGCAAGGTTTGGAA | |
| | TGGATAGGGTACATAAATCCTTCTAGCGCC | |
| | TATACTAATTACAACCAGAAGTTTCAGGGA | |
| | CGTGTTACTCTGACTGCTGACAAATCAACA | |
| | TCTACAGCCTATATGGAACTGTCTTCATTG | |
| | AGGAGTGAGGACACTGCTGTGTATTACTGC | |
| | GCTTCTCCTCAGGTGCACTATGATTACGCT | |
| | GGCTTTCCCTACTGGGGTTGTGGCACCCTC | |
| | GTCACCGTTTCATCTGAGCCCAAATCTAGC | |
| | GACAAAACTCACACATGTCCACCGTGCCCA | |
| | GCACCTGAAGCAGCAGGGGGACCGTCAGTC | |
| | TTCCTCTTCCCCCCAAAACCCAAGGACACC | |
| | CTCATGATCTCCCGGACCCCTGAGGTCACA | |
| | TGCGTGGTGGTGAGCGTGAGCCACGAAGAC | |
| | CCTGAGGTCAAGTTCAACTGGTACGTGGAC | |
| | GGCGTGGAGGTGCATAATGCCAAGACAAAG | |
| | CCGCGGGAGGAGCAGTACAACAGCACGTAC | |
| | CGTGTGGTCAGCGTCCTCACCGTCCTGCAC | |
| | CAGGACTGGCTGAATGGCAAGGAGTACAAG | |
| | TGCAAGGTCTCCAACAAAGCCCTCCCAGCC | |
| | CCCATCGAGAAAACCATCTCCAAAGCCAAA | |
| | GGGCAGCCCCGAGAACCACAGGTGTACACC | |
| | CTGCCCCCATCCCGGGAGGAGATGACCAAG | |
| | AACCAGGTCAGCCTGTGGTGCCTGGTCAAA | |
| | GGCTTCTATCCCAGCGACATCGCCGTGGAG | |
| | TGGGAGAGCAATGGGCAGCCGGAGAACAAC | |
| | TACAAGACCACGCCTCCCGTGCTGGACTCC | |
| | GACGGCTCCTTCTTCCTCTACAGCAAGCTC | |
| | ACCGTGGACAAGTCTAGATGGCAGCAGGGG | |
| | AACGTCTTCTCATGCTCCGTGATGCATGAG | |
| | GCTCTGCACAACCACTACACGCAGAAGAGC | |
| | CTCTCCCTGTCTCCGGGTAAA | |

### 7.8 Engineering of GUCY2C×CD3_biAb-2

The GUCY2C binding arm of GUCY2C×CD3_biAb-2 is the same as for GUCY2C×CD3_biAb-1 described above.

The CD3 binding arm of GUCY2C×CD3_biAb-2 is a high affinity CD3 binding arm in a spFv format, and will herein be referred to as anti-CD3 spFv-2 (CD3_spFv-2). The stapled scFv in the VL-Linker-VH was further engineered into a Heavy Chain 2 (HC2) and expressed as IgG1 comprising the Fc silencing mutation L234A/L235A/D265S and the heterodimerization (“knob” mutation T366W) in the Fc domain. HC2 comprising CD3_spFv-2 was then paired with the HC1 and the LC1 comprising the GUCY2C_Fab using the knob-in-hole heterodimerization technology to generate the stapled GUCY2C×CD3_biAb-2.

### 7.9 Expression of GUCY2C×CD3 Antibodies

The GUCY2C×CD3 bispecific antibodies were expressed in ExpiCHO-S cells (ThermoFisher; Cat #A29127) by transient transfection with purified plasmid DNAs encoding LC1, HC1, and HC2 using the ExpiFectamine CHO transfection kit (ThermoFisher; Cat #A29131). ExpiCHO-S cells were maintained in suspension in ExpiCHO Expression Medium (ThermoFisher; Cat #A2910001) in an orbital shaking incubator set to 37° C., 8% CO2 and 125 rpm. The cells were passaged into 4 sterile, vented, non-baffled Erlenmeyer flasks (Corning 431255) with 400 mL starting culture volume per flask.

On the day of transfection (Culture Day 0), the flask cultures were between 5.15-8.99×10\hat{ }6 viable cells/mL with a minimum viability of 98.2%. For the transfection, 200 μg of plasmid DNA encoding HC2, 200 pg plasmid DNA encoding HC1, and 400 pg plasmid DNA encoding LC1 (HC2:HC1:LC1 chain ratio at 1:1:2) were diluted in 64 mL of OptiPRO medium (ThermoFisher Cat #12309019). 5.12 mL of ExpiFectamine CHO transfection reagent was diluted in another 64 mL of OptiPRO medium. The plasmid DNA plus OptiPRO medium mixture and the ExpiFectamine CHO reagent plus OptiPRO medium mixture were combined and incubated at room temperature for one minute. This was then equivalently distributed across the 4 flasks of cells, ˜33 mL/flask. The transfected cells were returned to the orbital shaking incubator.

After overnight incubation (Culture Day 1), 240 mL of ExpiCHO Feed and 4.8 mL ExpiFectamine CHO Enhancer were combined and then distributed equivalently across the 4 flasks. Cells were further incubated until harvest (Culture Day 7). The culture supernatant from the transiently transfected cells (6.06×10\hat{ }66 viable cells/mL, 84.7% viability) was clarified by centrifugation (15 min, 5000 rpm (5316 RCF)) followed by 0.2 μm filtration.

### 7.10 Purification of GUCY2C×CD3 Antibodies

The GUCY2C×CD3 bispecific antibodies were purified as follows. The filtered cell culture supernatant as prepared above was loaded onto a pre-equilibrated (1×DPBS, pH 7.2) custom 60 mL MabSelect PrismA column at 20 mL/min using an AKTA Pure150. After loading, the column was washed with 1×DPBS, pH 7.2 until UV stabilized near baseline. The protein was eluted with 0.1 M sodium acetate, pH 3.45, and neutralized inline by the addition of 2.5 M Tris HC1, pH 7.5 to 10% (v/v) final volume, filtered (0.2 μm), and dialyzed into 20 mM MES, pH 5.5. The dialyzed eluate was further purified by cation exchange chromatography (CEX) using a custom Capto S ImpAct column (GE Healthcare, 2.6 cm×36 cm, CV=193 mL) pre-equilibrated into buffer A (20 mM MES, pH 6.5). The protein was loaded at 10 mL/min and then eluted from the column with a gradient of buffer B (20 mM MES, pH 6.5, 1 M NaCl: 0-5% buffer B over 10 min, 5-35% buffer B over 135 min). The peak fractions containing only monomeric protein were pooled, dialyzed into 1×dPBS, PH 7.2 and filtered (0.2 μm).

### Characterization of GUCY2C×CD3 Antibodies

### 7.11 Tumor Cell Binding Specificity and T-Cell Binding Specificity

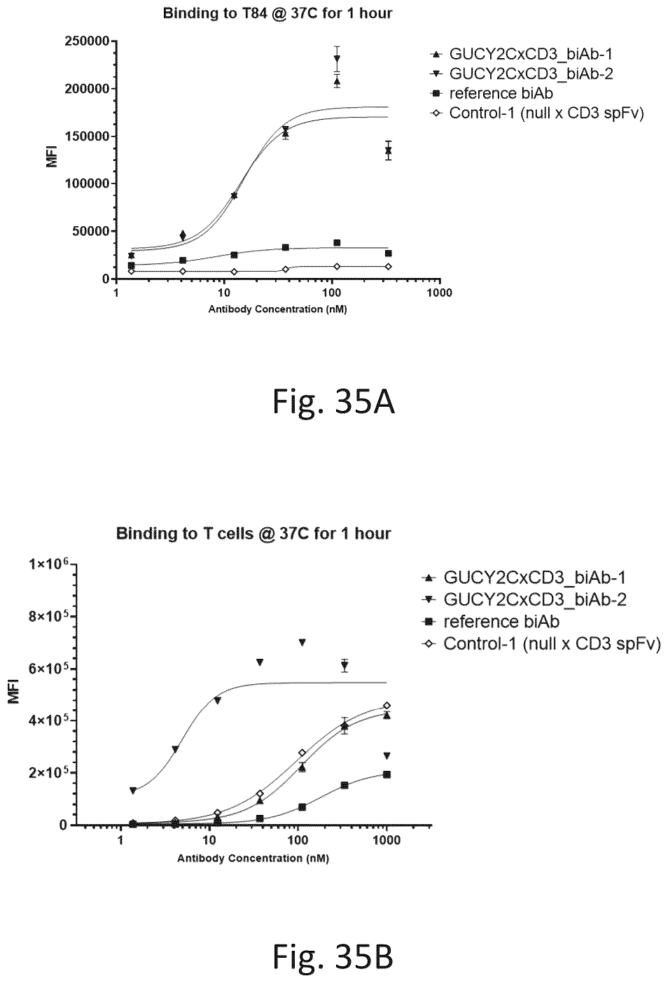

One hour cell binding studies by flow cytometry were carried out at physiologically relevant temperature (37° C.) on GUCY2C×CD3 bispecific antibodies (GUCY2C×CD3 biAb-1 and GUCY2C×CD3 biAb-2), known reference GUCY2C×CD3 bispecific antibody (biAb), and Null×CD3 control molecules. The goal was to characterize and quantify specific, cell binding for GUCY2C×CD3_biAb-1 and 2. As shown in FIG. 35A, GUCY2C×CD3_biAb-1 and 2 displayed, strong, specific binding to the GUCY2C endogenously expressing cell line T84. Maximum binding of GUCY2C×CD3_biAb-1 and 2 was higher than reference biAb. The Null×CD3 control did not bind to T84.

GUCY2C×CD3_biAb-1 bound to primary T cells to a similar extent as the Null×CD3, suggesting no impact of the GUCY2C Fab binding arm on T-cell engagement. GUCY2C×CD3 biAb-2, bearing a higher affinity CD3 binder, bound primary T cells with higher potency than GUCY2C×CD3-biAb-1 or Null×CD3 (FIG. 35B). Reference biAb also bound T cells as expected.

EC50 values for binding of GUCY2C×CD3 biAb-1 and 2 to T84 cells and T cells are summarized in Table 34.

TABLE 34

| Bispecific antibody | EC50 to T84 | EC50 to T cells |
|---|---|---|
| GUCY2CxCD3 biAb-1 | 12 nM | 108 nM |
| GUCY2CxCD3 biAb-2 | 12 nM | 4.9 nM |
| Reference biAb | 4.6 nM | 180 nM |

### 7.12 Epitope Comparison

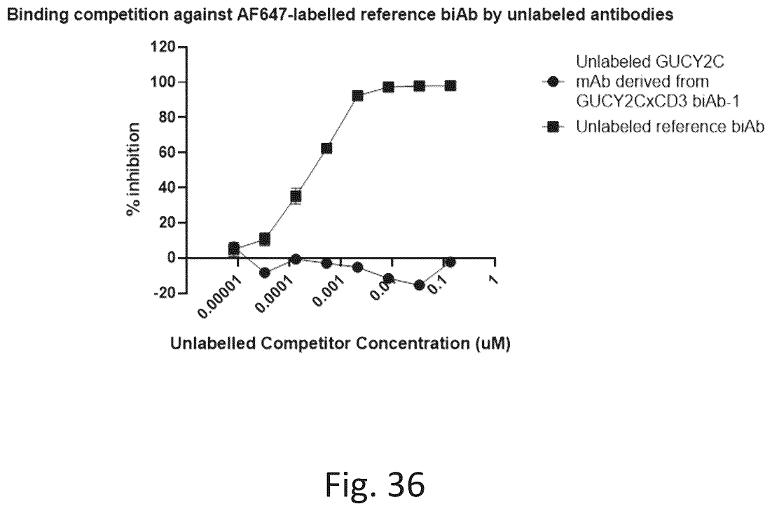

To determine whether the GUCY2C binder in GUCY2C×CD3_biAb-1 and 2 share the same epitope as reference biAb, competition flow cytometry experiments were performed using parental GUCY2C mAbs from which GUCY2C×CD3 biAb-1, GUCY2C×CD3_biAb-2 and reference Ab were derived. To that end, human GUCY2C transfected CHO cells were stained with AF647-labeled reference mAb in conjunction with a titration of unlabeled GUCY2C mAb from which GUCY2C×CD3 biAb-1 and 2 are derived. Plates were then washed and bound AF647-labeled reference mAb was then detected in a flow cytometer. As shown in FIG. 36, unlabeled GUCY2C mAb did not compete with AF647-labeled reference mAb for binding, while unlabeled reference biAb did. This indicates that the epitope of the GUCY2C binder in GUCY2C×CD3 biAb-1 and 2 is distinct from the epitope of the reference mAb.

### 7.13 In Vitro Killing and T-Cell Activation

Impedance-based cytotoxicity assays performed on the RTCA xCELLigence (Agilent) platform were the primary means used to characterize the in vitro potency of GUCY2C×CD3 bispecific antibodies (GUCY2C×CD3_biAb-1 and GUCY2C×CD3_biAb-2), reference GUCY2C×CD3 bispecific antibody (biAb), and Null×CD3 control molecules. Experiments conducted on the IncuCyte® S3 (Sartorius) real-time, live cell imaging system were investigated as an orthogonal readout. CRC cell lines HT55, LS1034, and T84 were tested. They express GUCY2C at a range of 15000-40000 receptors/cell, characterized as a medium level of GUCY2C. To best approximate physiological relevance in the tumor microenvironment (TME), the lowest E:T ratios where maximal cytotoxicity was reached were used for bispecific identification and characterization.

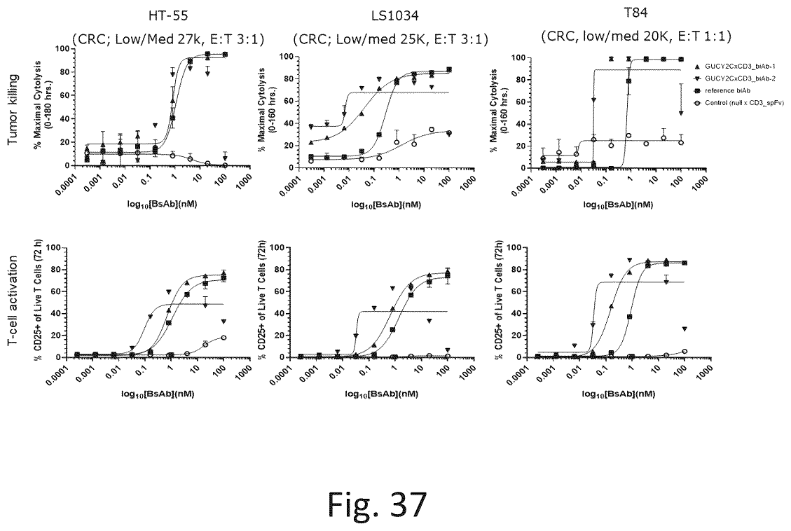

As shown in FIG. 37, GUCY2C×CD3_biAb-1 induced T-cell mediated cytotoxicity and T-cell activation at an E:T ratio of 1:1 for T84 and a E:T ratio of 3:1 for both HT55 and LS1034 cells. GUCY2C×CD3_biAb-2 also induced T-cell mediated cytotoxicity and T-cell activation against these cell lines, but showed a hook effect at high concentrations. The reference biAb showed weaker T-cell mediated cytotoxicity and T-cell activation than both GUCY2C×CD3 biAb-1 and 2. The control null×CD3 spFv did not show significant T-cell mediated cytotoxicity or T-cell activation against these cell lines.

EC50 values are shown in Table 35.

TABLE 35
Tumor killing EC50 (nM)

| Ab name | HT-55 | LS1034 | T84 |
|---|---|---|---|
| GUCY2CxCD3-biAb-1 | 0.8 | 0.04 | n/a |
| GUCY2CxCD3-biAb-2 | n/a | 0.007 | 0.03 |
| Reference biAb | 1.1 | 0.3 | 0.7 |

Abbreviations:
n/a = not applicable because curve fit was ambiguous and did not allow for accurate calculation of EC50 values

### 7.14 Anti-Tumor Efficacy in a T-Cell Humanized HT55 CDX Model

The antitumor activity and PD relationship of GUCY2C×CD3_biAb-1 was evaluated in the established SC human colorectal HT55 Cell-line Derived Xenograft (CDX) models in female immune-compromised NSG (ie, non-obese diabetic [NOD] severe combined immunodeficiency [scid] gamma or NOD.Cg-Prkdcscid Il2rgtm1Wj1/SzJ) mice humanized with human donor CD3+ pan-T cells.

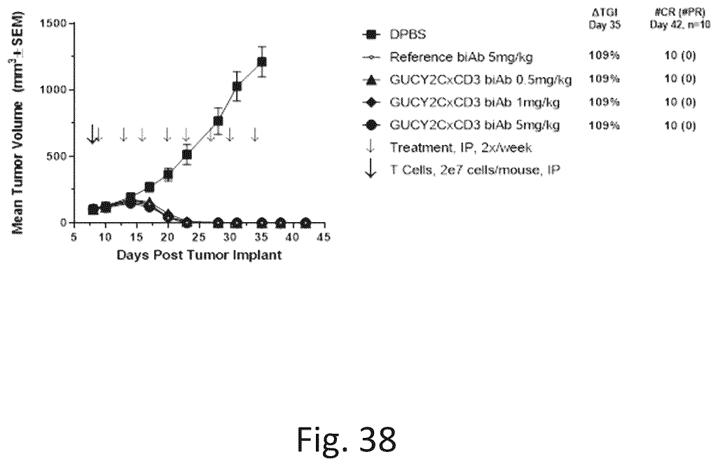

Mice bearing established SC HT55 xenografts were intraperitoneally (IP) dosed with GUCY2C×CD3 biAb-1 twice weekly at 0.5, 1 and 5 mg/kg or DPBS for a total of 8 doses (n=10/group). Significant antitumor efficacy was observed with GUCY2C×CD3 biAb-1 treatment at 0.5, 1, and 5 mg/kg with 109% A tumor growth inhibition (TGI) at all 3 dose levels (p<0.0001; FIG. 38), as compared to the DPBS-treated control group on Day 33 post tumor implantation. This resulted in 100% tumor regression (TR) and 10 of 10 complete regressions (CRs) in each treatment group by the end of the study.

### 7.15 Conformational Stability

The thermal stability of the GUCY2C×CD3_biAb-1 was characterized by capillary VP-DSC microcalorimeter (Malvern Panalytical Malvern, UK). Temperature scans were performed from 25 to 100° C. in duplicate. A buffer reference scan was subtracted from protein scan and the concentration of protein was normalized prior to thermodynamic analysis. The data were plotted and analyzed using MicroCal PEAQ software. The DSC curve was fitted using non-two-state model to obtain the enthalpy and apparent transition temperature (Tm) values. GUCY2C×CD3_biAb-1 showed good thermal stability (Table 36).

TABLE 36

| | |
|---|---|
| Tonset | 62.7° C. |
| Tms (Tm1, Tm2, Tm3) | 71.8, 76.3 and 90.7° C. |

### 7.16 Exploratory Toxicity Study

The GUCY2C arm of GUCY2C×CD3-biAb-1 is cynomolgus monkey cross reactive but its CD3 bind arm is human specific and lack cross reactive to monkey. A GUCY2C×CD3 bispecific (GUCY2C×CD3_biAb-3) with tissue cross-reactivity in cynomolgus monkey was therefore prepared for this study. GUCY2C×CD3-biAb-3 was prepared as described above for GUCY2C×CD3 biAb-1 and 2. GUCY2C×CD3 biAb-3, comprises the same GUCY2C Fab arm (HC1 and LC1) as GUCY2C×CD3 biAb-1 and 2 but comprises a different CD3 binding arm also in a spFv format but with cross reactivity to cyno CD3. The CD3 arm of GUCY2C×CD3-biAb-3 is referred herein as CD3_spFv-3. The binding affinity of the cynomolgus monkey cross reactive CD3 arm of CD3_spFv-3 is 2 to 5 times higher than that of the CD3_spFv-1 of GUCY2C×CD3-biAb-1. The higher CD3 binding affinity may result in higher level of cytokine release than would otherwise be observed with GUCY2C×CD3-biAb-1. GUCY2C×CD3-biAb-3 remains however an appropriate surrogate molecule in cynomolgus monkey to model the pharmacology and any associated potential toxicity resulting from GUCY2C×CD3-biAb-1 administration in humans.

Three (3) escalating dose levels (0.03, 0.3, and 1.2 mg/kg) were tested with GUCY2C×CD3_biAb-3 to assess the toxic dose level of GUCY2C×CD3_biAb-1. Each dose was administered twice (lx weekly) over 2 weeks to 1 monkey per sex per group.

The results show that all doses were tolerated (HNSTD=1.2 mg/kg). No mortality or moribundity was observed. Clinical signs included transient emesis in the highest treated group (1.2 mg/kg) after the first dose (Day 1) only, where no intervention is needed, and no emesis was observed after the second dose administration (Day 8). The GUCY2C×CD3_biAb-3 (containing the GUCY2C binder of the application) showed less undesired side effects (such as one or more from among emesis, high body temperature, decreased activity, hunched posture, reduced appetite; dehydration, body weight loss (<7%), and soft or liquid feces) than the reference biAb.

### Example 8: Claudin CD3 SpFv Antibodies

### 8.1 Engineering of CLDN18.2×CD3 Antibodies

The CD3 VH-Linker-VL or VL-linker-VH scFv binding molecules or VL-Linker-VH spFv molecules were further engineered as IgG1 into a scFv-hinge-CH2-CH3 format, or into a spFv-hinge-CH2-CH3 format comprising Fc silencing mutation (L234A/L235A/D265S) and dimerization mutations to promote heterodimerization of the CLDN18.2 and CD3 heavy chains and generation of CLDN18.2×CD3 antibodies.

The CLD18.2 specific VH and VL regions were engineered in VH-CH1-hinge-CH2-CH3 and VL-CL formats respectively and expressed as IgG1. The Fe silencing mutation L234A/L235A/D265S were introduced in the Fc region. Mutations designed to promote selective heterodimerization were also engineered in the CLDN18.2 Fc domain.

TABLE 37
Full length amino acid sequence of CD3
arm of CLDN18.2xCD3 bispecific Ab

| Name of | |
|---|---|
| CLDN18.2xCD3 | Sequence of CD3 arm |
| bispecific | (CD3B2030_N106A-spFv-LH) |
| CLDN18.2xCD3 | EIVLTQSPATLSASPGERVTLSCSASSSVS |
| bisp-Ab1 | YMNWYQQKPGCAPRRWIYDSSKLASGVPAR |
| | FSGSGSGRDYTLTISSLEPEDFAVYYCQQW |
| | SRNPPTFGGGTKVEIKGGGSGGSGGCPPCG |
| | GSGGQVQLVQSGAEVKKPGSSVKVSCKASG |
| | YTFTRSTMHWVKQAPGQGLEWIGYINPSSA |
| | YTNYNQKFQGRVTLTADKSTSTAYMELSSL |
| | RSEDTAVYYCASPQVHYDYAGFPYWGCGTL |
| | VTVSS |
| | (SEQ ID NO: 144) |
| CLDN18.2xCD3 | EIVLTQSPATLSASPGERVTLSCSASSSVS |
| bisp-Ab2 | YMNWYQQKPGCAPRRWIYDSSKLASGVPAR |
| | FSGSGSGRDYTLTISSLEPEDFAVYYCQQW |
| | SRNPPTFGGGTKVEIKGGGSGGSGGCPPCG |
| | GSGGQVQLVQSGAEVKKPGSSVKVSCKASG |
| | YTFTRSTMHWVKQAPGQGLEWIGYINPSSA |
| | YTNYNQKFQGRVTLTADKSTSTAYMELSSL |
| | RSEDTAVYYCASPQVHYDYAGFPYWGCGTL |
| | VTVSS |
| | (SEQ ID NO: 145) |
| CLDN18.2xCD3 | EIVLTQSPATLSASPGERVTLSCSASSSVS |
| bisp-Ab3 | YMNWYQQKPGCAPRRWIYDSSKLASGVPAR |
| | FSGSGSGRDYTLTISSLEPEDFAVYYCQQW |
| | SRNPPTFGGGTKVEIKGGGSGGSGGCPPCG |
| | GSGGQVQLVQSGAEVKKPGSSVKVSCKASG |
| | YTFTRSTMHWVKQAPGQGLEWIGYINPSSA |
| | YTNYNQKFQGRVTLTADKSTSTAYMELSSL |
| | RSEDTAVYYCASPQVHYDYAGFPYWGCGTL |
| | VTVSS |
| | (SEQ ID NO: 146) |
| CLDN18.2xCD3 | EIVLTQSPATLSASPGERVTLSCSASSSVS |
| bisp-Ab4 | YMNWYQQKPGCAPRRWIYDSSKLASGVPAR |
| | FSGSGSGRDYTLTISSLEPEDFAVYYCQQW |
| | SRNPPTFGGGTKVEIKGGGSGGSGGCPPCG |
| | GSGGQVQLVQSGAEVKKPGSSVKVSCKASG |
| | YTFTRSTMHWVKQAPGQGLEWIGYINPSSA |
| | YTNYNQKFQGRVTLTADKSTSTAYMELSSL |
| | RSEDTAVYYCASPQVHYDYAGFPYWGCGTL |
| | VTVSS |
| | (SEQ ID NO: 147) |

TABLE 38
Full length amino acid sequence of CLDN18.2
arm of CLDN18.2xCD3 bispecific sequences

| Name of | | |
|---|---|---|
| CLDN18.2xCD3 | | |
| bispecific | HC | LC |
| CLDN18.2xCD3 | EVQLLESGGGLVQPG | DIQMTQSPSSLSASV |
| bisp-Ab1 | GSLRLSCAASGFIFS | GDRVTITCRASQSVT |
| | NYYMAWVRLAPGKGL | ISSVDLMNWYQQKPG |
| | EWVSSISIGGGKSSY | KAPKLLIYRASNLAS |
| | ADSVKGRFTISRDNS | GIPSRFSGSGSGTDF |
| | KNTLYLQMNSLRAED | TLTISSLQPEDFATY |
| | TAVYYCVRHPQLGPN | YCQQSRESPYTFGQG |
| | PFDYWGQGTLVTVSS | TKLEIKRTVAAPSVF |
| | ASTKGPSVFPLAPSS | IFPPSDEQLKSGTAS |
| | KSTSGGTAALGCLVK | VVCLLNNFYPREAKV |
| | DYFPEPVTVSWNSGA | QWKVDNALQSGNSQE |
| | LTSGVHTFPAVLQSS | SVTEQDSKDSTYSLS |
| | GLYSLSSVVTVPSSS | STLTLSKADYEKHKV |
| | LGTQTYICNVNHKPS | YACEVTHQGLSSPVT |
| | NTKVDKKVEPKSCDK | KSFNRGEC |
| | THTCPPCPAPEAAGG | ((SEQ ID NO: 149) |
| | PSVFLFPPKPKDTLM | |
| | ISRTPEVTCVVVSVS | |
| | HEDPEVKFNWYVDGV | |
| | EVHNAKTKPREEQYN | |
| | STYRVVSVLTVLHQD | |
| | WLNGKEYKCKVSNKA | |
| | LPAPIEKTISKAKGQ | |
| | PREPQVYTLPPSREE | |
| | MTKNQVSLSCAVKGF | |
| | YPSDIAVEWESNGQP | |
| | ENNYKTTPPVLDSDG | |
| | SFFLVSKLTVDKSRW | |
| | QQGNVFSCSVMHEAL | |
| | HNRFTQKSLSLSPGK | |
| | (SEQ ID NO: 148) | |
| CLDN18.2xCD3 | EVQLLESGGGLVQPG | DIQMTQSPSSLSASV |
| bisp-Ab2 | GSLRLSCAASGFIFS | GDRVTITCRASQSVT |
| | NYYMAWVRLAPGKGL | ISSVDLMNWYQQKPG |
| | EWVSSISIGGGKSHY | KAPKLLIYRASNLAS |
| | ADSVKGRFTISRDNS | GIPSRFSGSGSGTDF |
| | KNTLYLQMNSLRAED | TLTISSLQPEDFATY |
| | TAVYYCVRHPSLGPN | YCQQSRESPYTFGQG |
| | PFDYWGQGTLVTVSS | TKLEIKRTVAAPSVF |
| | ASTKGPSVFPLAPSS | IFPPSDEQLKSGTAS |
| | KSTSGGTAALGCLVK | VVCLLNNFYPREAKV |
| | DYFPEPVTVSWNSGA | QWKVDNALQSGNSQE |
| | LTSGVHTFPAVLQSS | SVTEQDSKDSTYSLS |
| | GLYSLSSVVTVPSSS | STLTLSKADYEKHKV |
| | LGTQTYICNVNHKPS | YACEVTHQGLSSPVT |
| | NTKVDKKVEPKSCDK | KSFNRGEC |
| | THTCPPCPAPEAAGG | (SEQ ID NO: 151) |
| | PSVFLFPPKPKDTLM | |
| | ISRTPEVTCVVVSVS | |
| | HEDPEVKFNWYVDGV | |
| | EVHNAKTKPREEQYN | |
| | STYRVVSVLTVLHQD | |
| | WLNGKEYKCKVSNKA | |
| | LPAPIEKTISKAKGQ | |
| | PREPQVYTLPPSREE | |
| | MTKNQVSLSCAVKGF | |
| | YPSDIAVEWESNGQP | |
| | ENNYKTTPPVLDSDG | |
| | SFFLVSKLTVDKSRW | |
| | QQGNVFSCSVMHEAL | |
| | HNRFTQKSLSLSPGK | |
| | (SEQ ID NO: 150) | |
| CLDN18.2xCD3 | EVQLLESGGGLVQPG | DIQMTQSPSSLSASV |
| bisp-Ab3 | GSLRLSCAASGFIFS | GDRVTITCRASQSVT |
| | NYYMAWVRLAPGKGL | ISSVDLMNWYQQKPG |
| | EWVSSISIGGGKSHY | KAPKLLIYRASNLAS |
| | ADSVKGRFTISRDNS | GIPSRFSGSGSGTDF |
| | KNTLYLQMNSLRAED | TLTISSLQPEDFATY |
| | TAVYYCVRHPQSGPN | YCQQSRESPYTFGQG |
| | PFDYWGQGTLVTVSS | TKLEIKRTVAAPSVF |
| | ASTKGPSVFPLAPSS | IFPPSDEQLKSGTAS |
| | KSTSGGTAALGCLVK | VVCLLNNFYPREAKV |
| | DYFPEPVTVSWNSGA | QWKVDNALQSGNSQE |
| | LTSGVHTFPAVLQSS | SVTEQDSKDSTYSLS |
| | GLYSLSSVVTVPSSS | STLTLSKADYEKHKV |
| | LGTQTYICNVNHKPS | YACEVTHQGLSSPVT |
| | NTKVDKKVEPKSCDK | KSFNRGEC |
| | THTCPPCPAPEAAGG | (SEQ ID NO: 153) |
| | PSVFLFPPKPKDTLM | |
| | ISRTPEVTCVVVSVS | |
| | HEDPEVKFNWYVDGV | |
| | EVHNAKTKPREEQYN | |
| | STYRVVSVLTVLHQD | |
| | WLNGKEYKCKVSNKA | |
| | LPAPIEKTISKAKGQ | |
| | PREPQVYTLPPSREE | |
| | MTKNQVSLSCAVKGF | |
| | YPSDIAVEWESNGQP | |
| | ENNYKTTPPVLDSDG | |
| | SFFLVSKLTVDKSRW | |
| | QQGNVFSCSVMHEAL | |
| | HNRFTQKSLSLSPGK | |
| | (SEQ ID NO: 152) | |
| CLDN18.2xCD3 | EVQLLESGGGLVQPG | DIQMTQSPSSLSASV |
| bisp-Ab4 | GSLRLSCAASGFIFS | GDRVTITCRASQSVT |
| | NYYMAWVRLAPGKGL | ISSVDLMNWYQQKPG |
| | EWVSSISIGGGKSHY | KAPKLLIYRASNLAS |
| | ADSVKGRFTISRDNS | GIPSRFSGSGSGTDF |
| | KNTLYLQMNSLRAED | TLTISSLQPEDFATY |
| | TAVYYCVRHPQLGPN | YCQQSRESPYTFGQG |
| | PFDYWGQGTLVTVSS | TKLEIKRTVAAPSVF |
| | ASTKGPSVFPLAPSS | IFPPSDEQLKSGTAS |
| | KSTSGGTAALGCLVK | VVCLLNNFYPREAKV |
| | DYFPEPVTVSWNSGA | QWKVDNALQSGNSQE |
| | LTSGVHTFPAVLQSS | SVTEQDSKDSTYSLS |
| | GLYSLSSVVTVPSSS | STLTLSKADYEKHKV |
| | LGTQTYICNVNHKPS | YACEVTHQGLSSPVT |
| | NTKVDKKVEPKSCDK | KSFNRGEC |
| | THTCPPCPAPEAAGG | (SEQ ID NO: 155) |
| | PSVFLFPPKPKDTLM | |
| | ISRTPEVTCVVVSVS | |
| | HEDPEVKFNWYVDGV | |
| | EVHNAKTKPREEQYN | |
| | STYRVVSVLTVLHQD | |
| | WLNGKEYKCKVSNKA | |
| | LPAPIEKTISKAKGQ | |
| | PREPQVYTLPPSREE | |
| | MTKNQVSLSCAVKGF | |
| | YPSDIAVEWESNGQP | |
| | ENNYKTTPPVLDSDG | |
| | SFFLVSKLTVDKSRW | |
| | QQGNVFSCSVMHEAL | |
| | HNRFTQKSLSLSPGK | |
| | (SEQ ID NO: 154) | |

### 8.2 Binding of Bispecific to Cells by Flow Cytometry

Purified CLDN18.2×CD3 antibodies were assessed for their ability to bind to cell lines expressing endogenous CLDN18.2 (SK-CO-1). SK-CO-1 is a human colorectal adenocarcinoma cell line, NUGC-4 is a cell line derived from a signet ring cell carcinoma of stomach. DMS 454 is a cell line derived from a small cell lung carcinoma. NCI-H146 is a cell line derived from lung cancer. GSU is cell line derived from gastric carcinoma. All cell lines express human claudin 18.2 at varying levels.

SK-CO-1 cells and DMS 454 cells were suspended in 50 ml conical tubes at 20-22 million cells per 50 ml conical tube. In order to multiplex the flow cytometry analysis, the SK-CO-1 cells were stained with 0.02 uM CFSE, DMS 454 cell were stained with 0.2 uM CTV and the NUGC-4 cells were kept unstained. Cells were incubated for 10 minutes at room temperature protected from light, and centrifuged for 5 minutes at 400×g. The supernatant was removed and the cells were resuspended in HI FBS at ˜1.5×10\hat{ }6 cells/mL to quench the staining reactions. All cells were resuspended in fresh media at ˜1.5×10\hat{ }6 cells/mL. Equal volumes of CFSE stained SK-CO-1 cells, CTV stained DMS 454 cells and unstained SK-CO-1 cells were mixed and 50 μl of mixed cells were plated per well into assay plates (˜25k cells/each cell line; ˜75k/total mixed cells/well). Anti-human transferrin human mAb and human IgG1_AAS isotype (bivalent) were used as positive and negative controls, respectively. 50 μl of CLDN18.2×CD3 titrations at 2× concentration were added in each well containing cells and incubated for one hours at 37° C. 150 μl staining buffer was added to each well after one hour incubation. The plate was spun at 500×g for 5 minutes to pellet the cells. 150 μl staining buffer was added again to each well and the plate was spun at 500×g for 5 minutes to pellet the cells. Supernatant was removed and 50 μl of A647 conjugated anti human IgG Fc specific secondary detection antibody (Jackson ImmunoResearch) was the added at 2 μg/mL in staining buffer to each well. The plates were covered with foil and incubated for 30 minutes on ice. 150 μL of IntelliCyt running buffer was added to the wells, and the plate was spun at 500×g for 5 minutes to pellet cells and the supernatant was removed. Cells were then resuspended in 35 μL running buffer and the plates were run on an iQue Screener (Sartorius). Briefly, cells were gated on FCS vs. SCS dot plot to eliminate debris. Singlets were gated on SCS-A vs SCS-H dot plot. Cells were gated on BL1-H and VL1-H channels to separate the 3 populations of cells. Cells were assessed for binding of control mAbs or test panel sups by comparing to negative/isotype control binding by RL1-H (A647) Geomeans from the live cell population. Dose response curves were generated using 4 parameter fits to the calculate EC50 and max binding levels shown in Table 39.

TABLE 39
Binding of CLDN18.2xCD3 antibodies to SK-CO-1 cells

| CLDN18.2xCD3 antibody | EC50 to SK-CO-1 cells |
|---|---|
| name | (nM) |
| CLDN18.2xCD3 bisp-Ab1 | 12.7 |
| CLDN18.2xCD3 bisp-Ab4 | 41.3 |
| CLDN18.2xCD3 bisp-Ab2 | 193.3 |
| CLDN18.2xCD3 bisp-Ab3 | 228.7 |

The concentration-dependent binding of CLDN18.2×CD3 bisp-Ab4 to NCI-H146 and GSU cells was assessed at 37° C. after 1 hr incubation over a range of 0-1 uM (twelve 3-fold dilutions).

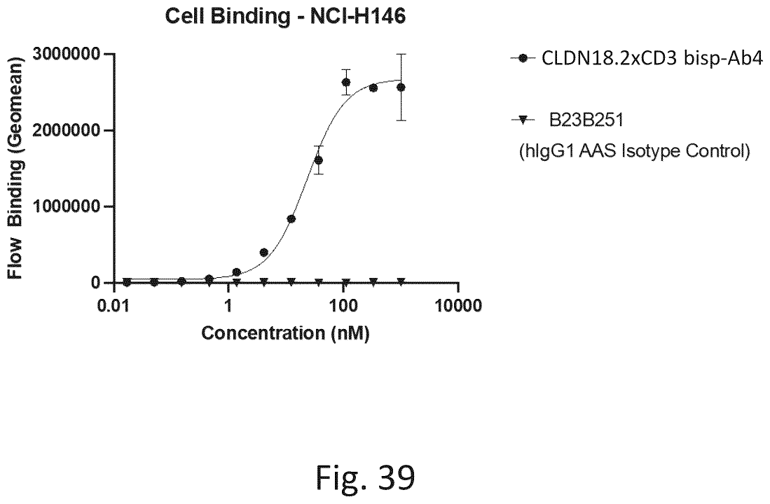

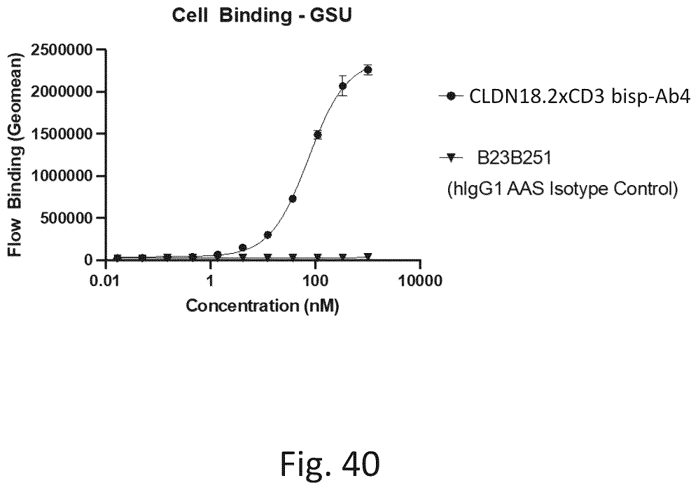

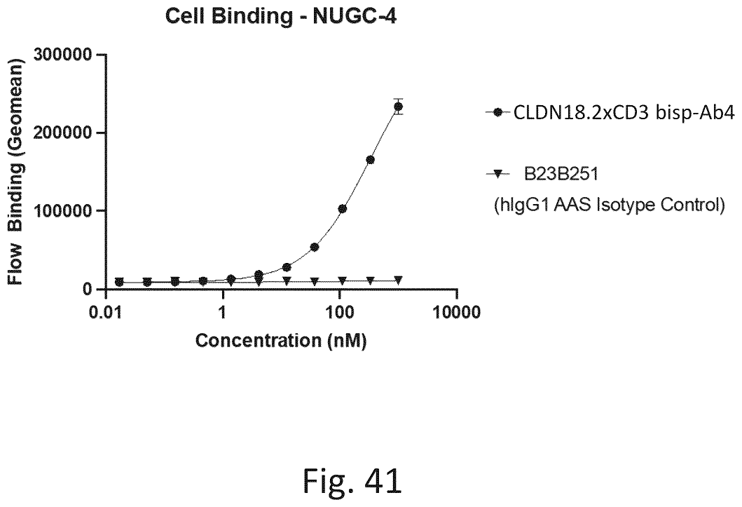

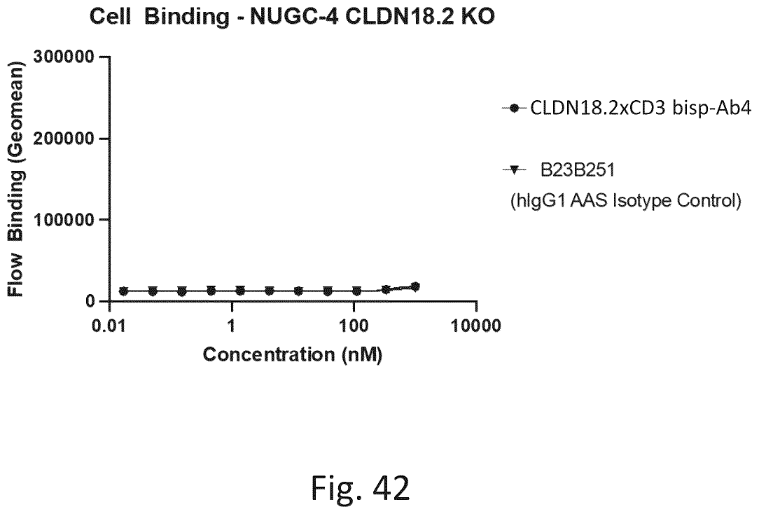

Cells were thawed in a 37° C. water bath and resuspended in complete assay media before being transferred to culture flasks. The cells used for this experiment were kept in active culture, passaged at least two times, and were >90% viable at assay initiation. The kinetic cell binding assay was initiated by adding 200 μL of cells and 200 μL of antibody into each well of a 2 mL 96-well deep plate and incubated at 37° C. At each time point, a 50 μL aliquot was removed and transferred to a round bottom 96-well plate containing cold FACS buffer. Cells were washed twice with FACS buffer and resuspended in 50 μL of FACS buffer containing secondary antibody AF 647 F(ab′)₂ Fragment Goat Anti-Human IgG at 6 μg/mL. The secondary antibody was incubated with cells for 30 min at 4° C. protected from light. After incubation, cells were washed three times with cold FACS buffer and then resuspended in FACS buffer containing 0.1% pluronic acid, 2 mM EDTA, and Sytox Green (diluted 1:1000) to label all dead cells. Staining was immediately analyzed on the Intellicyt iQue3 (Sartorius). NuGC-4 CLDN 18.2 KO cells served as a negative cell line in this experiment and confirmed the specificity for CLDN 18.2. Representative dose-response curves are exhibited in FIG. 39-42. The fitted EC50s for CLDN18.2×CD3 bisp-Ab4 binding to the tumor cell lines are also shown in Table 40. CLDN18.2×CD3 bisp-Ab4 displayed dose dependent specific binding that was consistent with receptor densities on each target cell lines as compared to CLDN 18.2 knockout cells.

TABLE 40
CLDN18.2xCD3 bisp-Ab4 Binding Parameters to
Endogenous Tumor Cell Lines

| | | | | NuGC-4 CLDN |
|---|---|---|---|---|
| | NCI-H146 | GSU | NuGC-4 | 18.2 KO |
| EC50 (nM) | 23 | 74 | N/A* | N/A* |

*Note:
EC50 values not assigned due to lack of saturation of dose response curves

### 8.3 Stability Analyses of CLDN18.2×CD3 Antibodies

The stability of C18B1068 and CLDN18.2×CD3 bisp-Ab4 was evaluated as described below.

### 8.3.1 Differential Scanning Calorimetry of C18B1068 and CLDN18.2×CD3 Bisp-Ab4

The thermal stability of the CLDN18.2×CD3 antibody C18B1068 and CLDN18.2×CD3 bisp-Ab4 was characterized by capillary VP-DSC microcalorimeter (Malvern Panalytical Malvern, UK). The concentration of protein was 1.0 mg/mL measured at a scan rate of 1° C./min with a cell volume of 0.450 mL in 10 mM histidine pH 6.5 buffer. Temperature scans were performed from 25 to 100° C. in duplicate. A buffer reference scan was subtracted from protein scan and the concentration of protein was normalized prior to thermodynamic analysis. The data were plotted and analyzed using MicroCal PEAQ software. The DSC curve was fitted using non-two-state model to obtain the enthalpy and apparent transition temperature (Tm) values.

CLDN18.2×CD3 bisp-Ab4 (stapled) showed increased thermal stability with ˜10 degrees higher Tonset relative to C18B1068 (Table 41).

TABLE 41

| | C18B1068 | CLDN18.2xCD3 bisp-Ab4 |
|---|---|---|
| Tonset | 56.3° C. | 65.8° C. |
| Tms (Tm1, Tm2, Tm3) | 67.9, 72.5 and 89.4° C. | 72.6, and 76.9° C. |

### 8.3.2 Stability of C18B1068 and CLDN18.2×CD3 Bisp-Ab4 Under HCLF and Stress Conditions

The stability of C18B1068 and CLDN18.2×CD3 bisp-Ab4 was also evaluated after incubation of the antibodies under High Concentration Liquid Formulation (HCLF) and stress conditions. C18B1068 and CLDN18.2×CD3 bisp-Ab4 were concentrated to ˜150 mg/ml in 10 mM Histidine buffer at pH6.5 and incubated for two weeks at 40 degree Celsius. The antibodies were then analyzed by analytical SEC (aSEC) and analytical ultracentrifugation (AUC).

### Analytical Size Exclusion Chromatography of C18B1068 and CLDN18.2×CD3 Bisp-Ab4

Stability of C18B1068 and CLDN18.2×CD3 bisp-Ab4 after incubation at HCLF and under stress conditions was analyzed by analytical size-exclusion (aSEC) to monitor for protein aggregates, dimer, monomer, and fragment. aSEC analysis was carried out using a TOSOH TSKgel BioAssist G3SW×L column (7.8 mm×30 cm, 5 μm, TOSOH) in 0.2 M Sodium Phosphate, pH 6.8 as the mobile phase, and at a flow rate of 0.7 mL/min on an Agilent 1200-series HPLC (Agilent Technologies, Santa Clara, CA, USA). In this described procedure, a group of reference standards with biologics size range of 2 to 600 kDa were injected onto the column. A target of 10-20 pg of total protein was injected per run. Peaks were monitored using absorbance at 280 nm. Data analysis of species found in each sample was performed using ChemStation software (Agilent Technologies) (Table 42).

TABLE 42

| | | CLDN18.2xCD3 |
|---|---|---|
| | C18B1068 | bisp-Ab4 |
| % monomer by aSEC after | 69.3 | 98.8-99.7% |
| submission to HCLF conditions | | |
| (150-152 mg/ml, at 40 degrees | | |
| C. for 2 weeks | | |

### Analytical Ultracentrifugation

Stability of C18B1068 and CLDN18.2×CD3 bisp-Ab4 was also evaluated by analytical ultracentrifugation by looking at the antibody sedimentation velocity using a Beckman Optima AUC. Samples were loaded (450 μL) into Beckman analytical ultracentrifuge cells equipped with 1.2 cm Beckman centerpieces (rated to 50k rpm), quartz windows, and torqued to 130 in-lbs. The centrifuge cells were placed into an An-50 (8 hole) rotor and placed in the chamber of the AUC. The temperature of the AUC was equilibrated to 20.5° C. for approximately 2 hours with the rotor in the chamber before initiating the run. Sedimentation velocity runs were performed at 40k rpm with 250 scans per antibody, a scan collection frequency of 90 seconds, and a 10 μM data resolution. Absorbance data was collected at 280 nm. Initially the data was analyzed using the software program DCDT+ in order to determine the meniscus position and to observe the sedimentation distribution profiles. The data was then analyzed using the analysis fitting software SEDFIT. The meniscus position was set at the value determined by DCDT+, the baseline was set at 7.2 cm, and manually choosing the fit range. The data was fit using the continuous c(s) distribution model with a sedimentation coefficient range of 0-20 s, resolution of 100, and a confidence interval of 0.49 for integration of peaks without regularization. To estimate the relative abundance of the different oligomeric species, present in each peak of the sedimentation of the samples, the integration function was used.

CLDN18.2×CD3 bisp-Ab4 (stapled) showed improved colloidal stability, maintaining >94.4% monomer at 150 mg/mL after 2 wks thermal stress relative to 69.7% for C18B1068 (Table 43).

TABLE 43

| | | CLDN18.2xCD3 |
|---|---|---|
| | C18B1068 | bisp-Ab4 |
| % monomer by AUC after 2 | 69.7% | 94.4% |
| weeks incubation at 150 mg/ml | | |
| and 40° C. | | |

### 8.3.3 Serum Stability of CLDN18.2×CD3 Antibodies of C18B1068 and CLDN18.2×CD3 Bisp-Ab4

Serum stability assay was developed to evaluate properties of protein molecules for non-specific or off-target binding to human serum components. The study may be predictive of poor pharmacokinetics and bio distribution properties. Binding and stability of the C18B1068 and CLDN18.2×CD3 bisp-Ab4 was evaluated in both buffer and human serum using a fluorescence-based chromatography method. C18B1068 and CLDN18.2×CD3 bisp-Ab4 were labeled with Alexa Fluor 488 conjugate, incubated in HBS-EP buffer (0.01 M HEPES pH 7.4, 0.15 M NaCl, 3 mM EDTA, 0.005% v/v Surfactant P20, 10× buffer, GE, cat #BR100669) and human serum ((BIOIVT/Bioreclamation, cat #—HUMANSRM) at 37° C. for 7 days and were analyzed by SEC-HPLC using Agilent HPLC system equipped with fluorescence detector.

100 μl of each antibody at 2.0 mg/mL in PBS was labeled with Alexa Fluor™ Antibody Labeling Kits, (ThermoFisher, Catalog number: A20181), according to the manufacturer's protocol. Serum stability assay was setup as following: for each labeled antibody, 500 μl samples were prepared samples to 300 nM final protein concentration in either 1×HBS-EP buffer or in human serum. Samples were taken for SEC-HPLC (t0). Samples were then divided into two, one set stored in 4° C. while the other set was stored in 37° C. After 7 days, samples evaluated by SEC-HPLC on a TOSOH TSKgel BioAssist G3SW×L column (7.8 mm×30 cm, 5 μm, TOSOH) preequilibrated with a mobile phase comprising 0.2 M Sodium Phosphate, pH 6.8, at a flow rate of 0.7 mL/min using an Agilent 1200-series HPLC (Agilent Technologies, Santa Clara, CA, USA). Peaks were monitored using absorbance at 494 nm. Data analysis of peaks found in each sample was performed using ChemStation software (Agilent Technologies).

CD3B1068 showed 90% monomer after 7 days incubation at 37° C. in serum and a 9.5% increase in High Molecular Weight proteins after 7 days, while CLDN18.2×CD3 bisp-Ab4 showed 93.7% monomer after 7 days incubation at 37° C. and a 3.3% increase in High Molecular Weight proteins after 7 days (Table 44).

TABLE 44
% monomer, High Molecular Weight Species (HMW S) and Low
Molecular Weight Species (LMW S) after 7 days at 37° C. in serum

| | | CLDN18.2xCD3 |
|---|---|---|
| | C18B1068 | bisp-Ab4 |
| % monomer | 90% | 93.7% |
| % HMW S | 9.5% | 5.9% |
| % LMW S | 0.5% | 0.4% |

1. A binding agent comprising an antigen binding region that binds to cluster of differentiation 3F (CD3ε), wherein the antigen binding region that binds to CD3ε comprises a stapled scFv (spFv) CDR sequences selected from the group consisting of:
a) the HCDR1 comprises the amino acid sequence of SEQ ID NO:7, the HCDR2 comprises the amino acid sequence of SEQ ID NO:8, the HCDR3 comprises the amino acid sequence of SEQ ID NO:9, the LCDR1 comprises the amino acid sequence of SEQ ID NO:10, the LCDR2 comprises the amino acid sequence of SEQ ID NO:11, and the LCDR3 comprises the amino acid sequence of SEQ ID NO:12;
b) the HCDR1 comprises the amino acid sequence of SEQ ID NO:13, the HCDR2 comprises the amino acid sequence of SEQ ID NO:14, the HCDR3 comprises the amino acid sequence of SEQ ID NO:9, the LCDR1 comprises the amino acid sequence of SEQ ID NO:10, the LCDR2 comprises the amino acid sequence of SEQ ID NO:11, and the LCDR3 comprises the amino acid sequence of SEQ ID NO:12;
c) the HCDR1 comprises the amino acid sequence of SEQ ID NO:15, the HCDR2 comprises the amino acid sequence of SEQ ID NO:16, the HCDR3 comprises the amino acid sequence of SEQ ID NO:9, the LCDR1 comprises the amino acid sequence of SEQ ID NO:10, the LCDR2 comprises the amino acid sequence of SEQ ID NO:11, and the LCDR3 comprises the amino acid sequence of SEQ ID NO:12;
d) the HCDR1 comprises the amino acid sequence of SEQ ID NO:17, the HCDR2 comprises the amino acid sequence of SEQ ID NO:18, the HCDR3 comprises the amino acid sequence of SEQ ID NO:19, the LCDR1 comprises the amino acid sequence of SEQ ID NO:20 the LCDR2 comprises the amino acid sequence of SEQ ID NO:21, and the LCDR3 comprises the amino acid sequence of SEQ ID NO:22; and
e) the HCDR1 comprises the amino acid sequence of SEQ ID NO:23, the HCDR2 comprises the amino acid sequence of SEQ ID NO:24, the HCDR3 comprises the amino acid sequence of SEQ ID NO:25, the LCDR1 comprises the amino acid sequence of SEQ ID NO:26, the LCDR2 comprises the amino acid sequence of DSS, and the LCDR3 comprises the amino acid sequence of SEQ ID NO:12.

2. The binding agent of claim 1, wherein in the antigen binding region that binds to CD3ε comprises a VH domain as set forth in SEQ ID NO:28, and a VL domain as set forth in SEQ ID NO:29.

3. The binding agent of claim 1, wherein in the antigen binding region that binds to CD3ε comprises a spFv as set forth in SEQ ID NO:30.

4. The binding agent of claim 1, wherein the binding agent is a bispecific antibody or a multi-specific antibody.

5. The binding agent of claim 1, further comprising an immunoglobulin (Ig) constant region, or a fragment of the Ig constant region, wherein optionally the fragment of the Ig constant region is an Fc region or an CH3 domain.

6. The binding agent of claim 5, wherein the Ig constant region, the fragment of the Ig constant region, the Fc region, or the CH3 domain comprises at least one mutation.

7. The binding agent of claim 6, wherein the at least one mutation is selected from the group consisting of L234A/L235A/D265S, F234A/L235A, L234A/L235A, V234A/G237A/P238S/H268A/V309L/A330S/P331S, F234A/L235A, S228P/F234A/L235A, N297A, V234A/G237A, K214T/E233P/L234V/L235A/G236-deleted/A327G/P331A/D365E/L358M, H268Q/V309L/A330S/P331S, S267E/L328F, L234F/L235E/D265A, L234A/L235A/G237A/P238S/H268A/A330S/P331S, S228P/F234A/L235A/G237A/P238S and S228P/F234A/L235A/G236-deleted/G237A/P238S, wherein residue numbering is according to the EU index.

8. The binding agent of claim 6, wherein the at least one mutation is selected from the group consisting of T366S/L368A/Y407V, T366W, T350V, L351Y, F405A, Y407V, T366Y, T366L, F405W, T394W, K392L, T394S, Y407T, Y407A, L351Y/F405A/Y407V, T366I/K392M/T394W, F405A/Y407V, T366L/K392M/T394W, T366L/K392L/T394W, L351Y/Y407A, L351Y/Y407V, T366A/K409F, T366V/K409F, T366A/K409F, T350V/L351Y/F405A/Y407V and T350V/T366L/K392L/T394W, wherein residue numbering is according to the EU index.

9. The binding agent of claim 6, wherein the binding agent comprises knob-in-hole mutations, wherein the knob mutations comprise T366S/L368A/Y407V, and the hole mutation comprises T366W.

10. The binding agent of claim 4, wherein the agent comprises a bispecific protein comprising an antigen binding region that binds a second antigen other than CD3R.

11. The binding agent of claim 10, wherein the second antigen is a tumor antigen.

12. A composition comprising the binding agent of claim 1, and a pharmaceutically acceptable carrier.

13. A polynucleotide comprising nucleotide sequences encoding a VH, a VL, or both a VH and a VL of the binding agent of claim 1.

14. A vector comprising the polynucleotide of claim 13.

15. A cell comprising the polynucleotide of claim 13.

16. A kit comprising the binding agent of claim 1.

17. A method of treating or slowing the progression of a disease or disorder in a subject, the method comprising administering to the subject at least one binding molecule of claim 1.

18. A method of directing a T cell to a target cell expressing a target antigen, comprising contacting the T cell with an effective amount of the binding agent of claim 10 or a composition comprising the binding agent and a pharmaceutically acceptable carrier, wherein the antigen binding region that binds to CD3ε binds the T cell and the antigen binding region that binds to a second antigen other than CD3ε binds to the target cell.


SEQUENCE LISTING (157 sequences)

SEQ ID NO: 1 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 2 - protein, 18 aa, synthetic construct
GGGSGGSGGCPPCGGSGG

SEQ ID NO: 3 - protein, 16 aa, synthetic construct
GGGSGGCPPCGGGSGG

SEQ ID NO: 4 - protein, 16 aa, synthetic construct
GGSGGSGGCPPCGSGG

SEQ ID NO: 5 - protein, 17 aa, synthetic construct
GGGSGGSGGCPPCGSGG

SEQ ID NO: 6 - protein, 17 aa, synthetic construct
GGGSGGGSGCPPCGGGG

SEQ ID NO: 7 - protein, 5 aa, synthetic construct
RSTMH

SEQ ID NO: 8 - protein, 17 aa, synthetic construct
YINPSSAYTNYNQKFQG

SEQ ID NO: 9 - protein, 12 aa, synthetic construct
PQVHYDYAGFPY

SEQ ID NO: 10 - protein, 10 aa, synthetic construct
SASSSVSYMN

SEQ ID NO: 11 - protein, 7 aa, synthetic construct
DSSKLAS

SEQ ID NO: 12 - protein, 9 aa, synthetic construct
QQWSRNPPT

SEQ ID NO: 13 - protein, 7 aa, synthetic construct
GYTFTRS

SEQ ID NO: 14 - protein, 6 aa, synthetic construct
NPSSAY

SEQ ID NO: 15 - protein, 10 aa, synthetic construct
GYTFTRSTMH

SEQ ID NO: 16 - protein, 10 aa, synthetic construct
YINPSSAYTN

SEQ ID NO: 17 - protein, 6 aa, synthetic construct
TRSTMH

SEQ ID NO: 18 - protein, 13 aa, synthetic construct
WIGYINPSSAYTN

SEQ ID NO: 19 - protein, 13 aa, synthetic construct
ASPQVHYDYAGFP

SEQ ID NO: 20 - protein, 6 aa, synthetic construct
SYMNWY

SEQ ID NO: 21 - protein, 10 aa, synthetic construct
RWIYDSSKLA

SEQ ID NO: 22 - protein, 8 aa, synthetic construct
QQWSRNPP

SEQ ID NO: 23 - protein, 8 aa, synthetic construct
GYTFTRST

SEQ ID NO: 24 - protein, 8 aa, synthetic construct
INPSSAYT

SEQ ID NO: 25 - protein, 14 aa, synthetic construct
ASPQVHYDYAGFPY

SEQ ID NO: 26 - protein, 5 aa, synthetic construct
SSVSY

SEQ ID NO: 27 - skipped

SEQ ID NO: 28 - protein, 121 aa, synthetic construct
QVQLVQSGAEVKKPGSSVKVSCKASGYTFTRSTMHWVKQAPGQGLEWIGYINPSSAYTNYNQKFQGRVTLTADKSTSTAYMELSSLRSEDTAVYYCASPQVHYDYAGFPYWGCGTLVTVSS

SEQ ID NO: 29 - protein, 106 aa, synthetic construct
EIVLTQSPATLSASPGERVTLSCSASSSVSYMNWYQQKPGCAPRRWIYDSSKLASGVPARFSGSGSGRDYTLTISSLEPEDFAVYYCQQWSRNPPTFGGGTKVEIK

SEQ ID NO: 30 - protein, 245 aa, synthetic construct
EIVLTQSPATLSASPGERVTLSCSASSSVSYMNWYQQKPGCAPRRWIYDSSKLASGVPARFSGSGSGRDYTLTISSLEPEDFAVYYCQQWSRNPPTFGGGTKVEIKGGGSGGSGGCPPCGGSGGQVQLVQSGAEVKKPGSSVKVSCKASGYTFTRSTMHWVKQAPGQGLEWIGYINPSSAYTNYNQKFQGRVTLTADKSTSTAYMELSSLRSEDTAVYYCASPQVHYDYAGFPYWGCGTLVTVSS

SEQ ID NO: 31 - protein, 4 aa, synthetic construct
CPPC

SEQ ID NO: 32 - protein, 107 aa, synthetic construct [sequence omitted]

SEQ ID NO: 33 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQDYGFPWTFGQGTKVEIK

SEQ ID NO: 34 - protein, 110 aa, synthetic construct
NFMLTQPHSVSESPGKTVTISCTRSSGSLANYYVQWYQQRPGSSPTIVIFANNQRPSGVPDRFSGSIDSSSNSASLTISGLKTEDEADYYCQTYDPYSVVFGGGTKLTVL

SEQ ID NO: 35 - protein, 110 aa, synthetic construct
NFMLTQPHSVSESPGKTVTISCTRSSGSLANYYVQWYQQRPGQSPTIVIFANNQRPSGVPDRFSGSIDSSSNSASLTISGLKTEDEADYYCQTYDPYSVVFGGGTKLTVL

SEQ ID NO: 36 - protein, 119 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKYDGIYGELDFWGQGTLVTVSS

SEQ ID NO: 37 - protein, 118 aa, synthetic construct
EVQLVQSGAEVKKPGESLKISCKGSGYSFTSYWISWVRQMPGKGLEWMGIIDPSDSDTRYSPSFQGQVTISADKSISTAYLQWSSLKASDTAMYYCARGDGSTDLDYWGQGTLVTVSS

SEQ ID NO: 38 - protein, 118 aa, synthetic construct
EVQLLESGGGLVQPGGSLRLSCAASGFGFSSYAMSWVRQAPGKGLEWVSAISGSGGSTYYADSVKGRFTISRDNSKNTLYLQMNSLRAEDTAVYYCARDLIHGVTRNWGQGTLVTVSS

SEQ ID NO: 39 - protein, 20 aa, synthetic construct
GGGGSGGGGSGGGGSGGGGS

SEQ ID NO: 40 - protein, 254 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIKRGGGGSGGGGSGGGGSGGGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKYDGIYGELDFWGQGTLVTVSSGHHHHHH

SEQ ID NO: 41 - protein, 252 aa, synthetic construct
DIQMTQSPSSLSASVGDRVTITCRASQSISSYLNWYQQKPGCAPKLLIYAASSLQSGVPSRFSGSGSGTDFTLTISSLQPEDFATYYCQQSYSTPLTFGQGTKVEIKRGGGSGGSGGCPPCGGSGGEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKYDGIYGELDFWGCGTLVTVSSGHHHHHH

SEQ ID NO: 42 - protein, 255 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKYDGIYGELDFWGQGTLVTVSSAGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIKRGHHHHHH

SEQ ID NO: 43 - protein, 252 aa, synthetic construct
EVQLLESGGGLVQPGGSLRLSCAASGFTFSSYAMSWVRQAPGCGLEWVSAISGSGGSTYYADSVKGRFTISRDNSKNTLYLQMNSLRAEDTAVYYCAKYDGIYGELDFWGQGTLVTVSSGGGSGGSGGCPPCGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGCGTKVEIKRGHHHHHH

SEQ ID NO: 44 - protein, 253 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQDYGFPWTFGQGTKVEIKGGGGSGGGGSGGGGSGGGGSEVQLVQSGAEVKKPGESLKISCKGSGYSFTSYWISWVRQMPGKGLEWMGIIDPSDSDTRYSPSFQGQVTISADKSISTAYLQWSSLKASDTAMYYCARGDGSTDLDYWGQGTLVTVSSGHHHHHH

SEQ ID NO: 45 - protein, 251 aa, synthetic construct
EIVLTQSPGTLSLSPGERATLSCRASQSVSSSYLAWYQQKPGCAPRLLIYGASSRATGIPDRFSGSGSGTDFTLTISRLEPEDFAVYYCQQDYGFPWTFGQGTKVEIKGGGSGGSGGCPPCGGSGGEVQLVQSGAEVKKPGESLKISCKGSGYSFTSYWISWVRQMPGKGLEWMGIIDPSDSDTRYSPSFQGQVTISADKSISTAYLQWSSLKASDTAMYYCARGDGSTDLDYWGCGTLVTVSSGHHHHHH

SEQ ID NO: 46 - protein, 253 aa, synthetic construct
EVQLVQSGAEVKKPGESLKISCKGSGYSFTSYWISWVRQMPGKGLEWMGIIDPSDSDTRYSPSFQGQVTISADKSISTAYLQWSSLKASDTAMYYCARGDGSTDLDYWGQGTLVTVSSGGGGSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQDYGFPWTFGQGTKVEIKGHHHHHH

SEQ ID NO: 47 - protein, 249 aa, synthetic construct
EVQLVQSGAEVKKPGESLKISCKGSGYSFTSYWISWVRQMPGCGLEWMGIIDPSDSDTRYSPSFQGQVTISADKSISTAYLQWSSLKASDTAMYYCARGDGSTDLDYWGQGTLVTVSSGGGSGGCPPCGGGSGGEIVLTQSPGTLSLSPGERATLSCRASQSVSSSYLAWYQQKPGQAPRLLIYGASSRATGIPDRFSGSGSGTDFTLTISRLEPEDFAVYYCQQDYGFPWTFGCGTKVEIKGHHHHHH

SEQ ID NO: 48 - protein, 255 aa, synthetic construct
NFMLTQPHSVSESPGKTVTISCTRSSGSLANYYVQWYQQRPGSSPTIVIFANNQRPSGVPDRFSGSIDSSSNSASLTISGLKTEDEADYYCQTYDPYSVVFGGGTKLTVLGGGGSGGGGSGGGGSGGGGSEVQLLESGGGLVQPGGSLRLSCAASGFGFSSYAMSWVRQAPGKGLEWVSAISGSGGSTYYADSVKGRFTISRDNSKNTLYLQMNSLRAEDTAVYYCARDLIHGVTRNWGQGTLVTVSSGHHHHHH

SEQ ID NO: 49 - protein, 251 aa, synthetic construct
NFMLTQPHSVSESPGKTVTISCTRSSGSLANYYVQWYQQRPGCSPTIVIFANNQRPSGVPDRFSGSIDSSSNSASLTISGLKTEDEADYYCQTYDPYSVVFGGGTKLTVLGGSGGSGGCPPCGSGGEVQLLESGGGLVQPGGSLRLSCAASGFGFSSYAMSWVRQAPGKGLEWVSAISGSGGSTYYADSVKGRFTISRDNSKNTLYLQMNSLRAEDTAVYYCARDLIHGVTRNWGCGTLVTVSSGHHHHHH

SEQ ID NO: 50 - protein, 255 aa, synthetic construct
NFMLTQPHSVSESPGKTVTISCTRSSGSLANYYVQWYQQRPGQSPTIVIFANNQRPSGVPDRFSGSIDSSSNSASLTISGLKTEDEADYYCQTYDPYSVVFGGGTKLTVLGGGGSGGGGSGGGGSGGGGSEVQLLESGGGLVQPGGSLRLSCAASGFGFSSYAMSWVRQAPGKGLEWVSAISGSGGSTYYADSVKGRFTISRDNSKNTLYLQMNSLRAEDTAVYYCARDLIHGVTRNWGQGTLVTVSSGHHHHHH

SEQ ID NO: 51 - protein, 252 aa, synthetic construct
NFMLTQPHSVSESPGKTVTISCTRSSGSLANYYVQWYQQRPGCSPTIVIFANNQRPSGVPDRFSGSIDSSSNSASLTISGLKTEDEADYYCQTYDPYSVVFGGGTKLTVLGGGSGGSGGCPPCGSGGEVQLLESGGGLVQPGGSLRLSCAASGFGFSSYAMSWVRQAPGKGLEWVSAISGSGGSTYYADSVKGRFTISRDNSKNTLYLQMNSLRAEDTAVYYCARDLIHGVTRNWGCGTLVTVSSGHHHHHH

SEQ ID NO: 52 - protein, 255 aa, synthetic construct
EVQLLESGGGLVQPGGSLRLSCAASGFGFSSYAMSWVRQAPGKGLEWVSAISGSGGSTYYADSVKGRFTISRDNSKNTLYLQMNSLRAEDTAVYYCARDLIHGVTRNWGQGTLVTVSSGGGGSGGGGSGGGGSGGGGSNFMLTQPHSVSESPGKTVTISCTRSSGSLANYYVQWYQQRPGSSPTIVIFANNQRPSGVPDRFSGSIDSSSNSASLTISGLKTEDEADYYCQTYDPYSVVFGGGTKLTVLGHHHHHH

SEQ ID NO: 53 - protein, 252 aa, synthetic construct
EVQLLESGGGLVQPGGSLRLSCAASGFGFSSYAMSWVRQAPGCGLEWVSAISGSGGSTYYADSVKGRFTISRDNSKNTLYLQMNSLRAEDTAVYYCARDLIHGVTRNWGQGTLVTVSSGGGSGGGSGCPPCGGGGNFMLTQPHSVSESPGKTVTISCTRSSGSLANYYVQWYQQRPGQSPTIVIFANNQRPSGVPDRFSGSIDSSSNSASLTISGLKTEDEADYYCQTYDPYSVVFGCGTKLTVLGHHHHHH

SEQ ID NO: 54 - protein, 5 aa, synthetic construct
SYAMS

SEQ ID NO: 55 - protein, 17 aa, synthetic construct
AISGGIGSTYYADSVKG

SEQ ID NO: 56 - protein, 12 aa, synthetic construct
DAVGATPYYFDY

SEQ ID NO: 57 - protein, 13 aa, synthetic construct
SGSSSNIGINYVS

SEQ ID NO: 58 - protein, 7 aa, synthetic construct
DNNKRPS

SEQ ID NO: 59 - protein, 11 aa, synthetic construct
GTWDSSLSAVV

SEQ ID NO: 60 - protein, 121 aa, synthetic construct
EVQLVESGGGLVQPGGSLRLSCAASGFTFSSYAMSWVRQAPGKGLEWVSAISGGIGSTYYADSVKGRFTISRDNSKNTLWLQMNSLRAEDTAVYYCAKDAVGATPYYFDYWGQGTLVTVSS

SEQ ID NO: 61 - protein, 110 aa, synthetic construct
QSVLTQPPSVSAAPGQKVTISCSGSSSNIGINYVSWYQQLPGTAPKLLIYDNNKRPSGIPDRFSGSKSGTSATLGITGLQTGDEADYYCGTWDSSLSAVVFGGGTKLTVL

SEQ ID NO: 62 - protein, 451 aa, synthetic construct
EVQLVESGGGLVQPGGSLRLSCAASGFTFSSYAMSWVRQAPGKGLEWVSAISGGIGSTYYADSVKGRFTISRDNSKNTLWLQMNSLRAEDTAVYYCAKDAVGATPYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 63 - protein, 216 aa, synthetic construct
QSVLTQPPSVSAAPGQKVTISCSGSSSNIGINYVSWYQQLPGTAPKLLIYDNNKRPSGIPDRFSGSKSGTSATLGITGLQTGDEADYYCGTWDSSLSAVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 64 - DNA, 1353 bp, synthetic construct
  source 1..1353: mol_type = other DNA
gaggtgcagctggttgaatctggcggaggattggttcagcctggcggttctctgagactgtcttgtgccgcttccggcttcaccttctccagctacgctatgtcctgggtccgacaggctcctggcaaaggactggaatgggtgtccgctatctctggcggcatcggctctacctactacgccgactctgtgaagggcagattcaccatcagccgggacaactccaagaacaccctgtggctgcagatgaactccctgagagccgaggataccgccgtgtactactgtgccaaggatgccgtgggcgctaccccttactacttcgattattggggccagggcaccctggtcaccgtttcttctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtcgaacaaagccctcccagcccccatcgagaaaaccatctccaaagccaaagggcagccccgagaaccacaggtgtacaccctgcccccatcccgggaggagatgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcagatggcagcaggggaacgtcttctcatgctccgtgatgcatgaggctctgcacaaccggttcacgcagaagtctctctccctgtctccgggaaaa

SEQ ID NO: 65 - DNA, 648 bp, synthetic construct
  source 1..648: mol_type = other DNA
cagtctgtgctgacccagcctccttctgtgtctgctgctcctggccagaaagtgaccatctcctgctccggctcctcctccaacatcggcatcaactacgtgtcctggtatcagcagctgcccggcacagctcccaaactgctgatctacgacaacaacaagcggcccagcggcatccctgacagattctccggatctaagtccggcacctctgctaccctgggcatcacaggattgcagacaggcgacgaggccgactactattgcggcacctgggactcttctctgtccgccgttgtttttggcggtggcaccaagctgacagtgctgggtcagcccaaggctgcacccagtgtcactctgttcccgccctcctctgaggagcttcaagccaacaaggccacactggtgtgtctcataagtgacttctacccgggagccgtgacagtggcctggaaggccgatagcagccccgtcaaggcgggagtcgaaaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 66 - protein, 477 aa, synthetic construct
EIVLTQSPATLSASPGERVTLSCSASSSVSYMNWYQQKPGCAPRRWIYDSSKLASGVPARFSGSGSGRDYTLTISSLEPEDFAVYYCQQWSRNPPTFGGGTKVEIKGGGSGGSGGCPPCGGSGGQVQLVQSGAEVKKPGSSVKVSCKASGYTFTRSTMHWVKQAPGQGLEWIGYINPSSAYTNYNQKFQGRVTLTADKSTSTAYMELSSLRSEDTAVYYCASPQVHYDYAGFPYWGCGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - DNA, 1431 bp, synthetic construct
  source 1..1431: mol_type = other DNA
gagatcgtgctgacccagtctcctgccacactgagtgcttctccaggcgagagagtgaccctgtcctgctccgcttcctcctccgtgtcctacatgaactggtatcagcagaagcccggctgcgcccctagaagatggatctacgactcctccaagctggcctctggcgtgcctgctagattttccggctctggctctggcagagactataccctgacaatctccagcctggaacctgaggacttcgccgtgtactactgccagcagtggtctaggaaccctcctacctttggcggaggcaccaaggtggaaatcaagggcggaggttctggtggatctggcggatgtcctccttgtggtggaagcggcggacaggttcagctggttcagtctggcgccgaagtgaagaaacctggctcctccgtgaaagtgtcctgcaaggcttccggctacacctttaccagatccaccatgcactgggtcaagcaggcccctggacaaggcttggagtggatcggctacatcaaccccagctccgcctacaccaactacaaccagaaattccagggcagagtgaccctgaccgccgacaagtctacctccaccgcctacatggaactgtccagcctgagatctgaggacaccgccgtgtactactgcgcctctcctcaggttcactacgactacgccggctttccttattggggctgtggcaccctggtcaccgtttcttctgagcccaaatctagcgacaaaactcacacatgcccaccgtgcccagcacctgaagccgccgggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtcgaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcatgaggctctgcacaaccactacacgcagaagtctctctccctgtctccgggaaaa

SEQ ID NO: 68 - DNA, 363 bp, synthetic construct
  source 1..363: mol_type = other DNA
caggttcagctggttcagtctggcgccgaagtgaagaaacctggctcctccgtgaaagtgtcctgcaaggcttccggctacacctttaccagatccaccatgcactgggtcaagcaggcccctggacaaggcttggagtggatcggctacatcaaccccagctccgcctacaccaactacaaccagaaattccagggcagagtgaccctgaccgccgacaagtctacctccaccgcctacatggaactgtccagcctgagatctgaggacaccgccgtgtactactgcgcctctcctcaggttcactacgactacgccggctttccttattggggctgtggcaccctggtcaccgtttcttct

SEQ ID NO: 69 - DNA, 318 bp, synthetic construct
  source 1..318: mol_type = other DNA
gagatcgtgctgacccagtctcctgccacactgagtgcttctccaggcgagagagtgaccctgtcctgctccgcttcctcctccgtgtcctacatgaactggtatcagcagaagcccggctgcgcccctagaagatggatctacgactcctccaagctggcctctggcgtgcctgctagattttccggctctggctctggcagagactataccctgacaatctccagcctggaacctgaggacttcgccgtgtactactgccagcagtggtctaggaaccctcctacctttggcggaggcaccaaggtggaaatcaag

SEQ ID NO: 70 - DNA, 1431 bp, synthetic construct; identical to SEQ ID NO: 67
  source 1..1431: mol_type = other DNA

SEQ ID NO: 71 - protein, 5 aa, synthetic construct
DYSMN

SEQ ID NO: 72 - protein, 17 aa, synthetic construct
SISSISSYVKYADSVKG

SEQ ID NO: 73 - protein, 6 aa, synthetic construct
GHYFDY

SEQ ID NO: 74 - protein, 11 aa, synthetic construct
RASQSVSSNLA

SEQ ID NO: 75 - protein, 7 aa, synthetic construct
GASTRAT

SEQ ID NO: 76 - protein, 9 aa, synthetic construct
QQYNNWPRT

SEQ ID NO: 77 - protein, 7 aa, synthetic construct
GFTFSDY

SEQ ID NO: 78 - protein, 6 aa, synthetic construct
SSISSY

SEQ ID NO: 79 - protein, 10 aa, synthetic construct
GFTFSDYSMN

SEQ ID NO: 80 - protein, 10 aa, synthetic construct
SISSISSYVK

SEQ ID NO: 81 - protein, 6 aa, synthetic construct
SDYSMN

SEQ ID NO: 82 - protein, 13 aa, synthetic construct
WVSSISSISSYVK

SEQ ID NO: 83 - protein, 7 aa, synthetic construct
ARGHYFD

SEQ ID NO: 84 - protein, 7 aa, synthetic construct
SSNLAWY

SEQ ID NO: 85 - protein, 10 aa, synthetic construct
LLIYGASTRA

SEQ ID NO: 86 - protein, 8 aa, synthetic construct
QQYNNWPR

SEQ ID NO: 87 - protein, 8 aa, synthetic construct
GFTFSDYS

SEQ ID NO: 88 - protein, 8 aa, synthetic construct
ISSISSYV

SEQ ID NO: 89 - protein, 8 aa, synthetic construct
ARGHYFDY

SEQ ID NO: 90 - protein, 6 aa, synthetic construct
QSVSSN

SEQ ID NO: 91 - skipped

SEQ ID NO: 92 - protein, 115 aa, synthetic construct
EVQLVESGGGRVKPGGSLRLSCAASGFTFSDYSMNWVRQAPGKGLEWVSSISSISSYVKYADSVKGRFTMSRDNAKNSLFLQMNSLRDEDMAVYYCARGHYFDYWGQGTLVTVSS

SEQ ID NO: 93 - DNA, 345 bp, synthetic construct
  source 1..345: mol_type = other DNA
gaggtgcagctggttgaatctggcggcggaagagtgaagcctggcggatctctgagactgtcttgtgccgcctctggcttcaccttctccgactactccatgaactgggtccgacaggctcctggcaaaggcctggaatgggtgtcctctatctcctccatctccagctacgtgaagtacgccgactccgtgaagggcagattcaccatgtccagagacaacgccaagaactccctgttcctgcagatgaacagcctgcgcgacgaggacatggccgtgtactattgtgccagaggccactacttcgactactggggacagggcacactggtcacagtctcttct

SEQ ID NO: 94 - protein, 107 aa, synthetic construct
EIVMTQSPATLSVSPGERATLSCRASQSVSSNLAWYQQKPGLAPRLLIYGASTRATGIPARFSGSGSGTEFTLTISSLQSEDFAVYYCQQYNNWPRTFGQGTKVEIK

SEQ ID NO: 95 - DNA, 321 bp, synthetic construct
  source 1..321: mol_type = other DNA
gagatcgtgatgacccagtctcctgccacactgtctgtgtctcccggcgagagagctaccctgtcttgtagagcctctcagtccgtgtcctccaacctggcctggtatcagcagaagcctggactggctccccggctgttgatctatggcgcttctaccagagccacaggcatccccgctagattctccggctctggctctggcacagagtttaccctgaccatctccagcctgcagtccgaggatttcgccgtgtactactgccagcagtacaacaactggccccggacctttggccagggcaccaaggtggaaatcaag

SEQ ID NO: 96 - protein, 477 aa, synthetic construct; identical to SEQ ID NO: 66

SEQ ID NO: 97 - protein, 445 aa, synthetic construct
EVQLVESGGGRVKPGGSLRLSCAASGFTFSDYSMNWVRQAPGKGLEWVSSISSISSYVKYADSVKGRFTMSRDNAKNSLFLQMNSLRDEDMAVYYCARGHYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 98 - protein, 214 aa, synthetic construct
EIVMTQSPATLSVSPGERATLSCRASQSVSSNLAWYQQKPGLAPRLLIYGASTRATGIPARFSGSGSGTEFTLTISSLQSEDFAVYYCQQYNNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 99 - DNA, 1431 bp, synthetic construct; identical to SEQ ID NO: 67
  source 1..1431: mol_type = other DNA

SEQ ID NO: 100 - DNA, 1335 bp, synthetic construct
  source 1..1335: mol_type = other DNA
gaggtgcagctggttgaatctggcggcggaagagtgaagcctggcggatctctgagactgtcttgtgccgcctctggcttcaccttctccgactactccatgaactgggtccgacaggctcctggcaaaggcctggaatgggtgtcctctatctcctccatctccagctacgtgaagtacgccgactccgtgaagggcagattcaccatgtccagagacaacgccaagaactccctgttcctgcagatgaacagcctgcgcgacgaggacatggccgtgtactattgtgccagaggccactacttcgactactggggacagggcacactggtcacagtctcttctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtcgaacaaagccctcccagcccccatcgagaaaaccatctccaaagccaaagggcagccccgagaaccacaggtgtacaccctgcccccatcccgggaggagatgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcagatggcagcaggggaacgtcttctcatgctccgtgatgcatgaggctctgcacaaccggttcacgcagaagtctctctccctgtctccgggaaaa

SEQ ID NO: 101 - DNA, 642 bp, synthetic construct
  source 1..642: mol_type = other DNA
gagatcgtgatgacccagtctcctgccacactgtctgtgtctcccggcgagagagctaccctgtcttgtagagcctctcagtccgtgtcctccaacctggcctggtatcagcagaagcctggactggctccccggctgttgatctatggcgcttctaccagagccacaggcatccccgctagattctccggctctggctctggcacagagtttaccctgaccatctccagcctgcagtccgaggatttcgccgtgtactactgccagcagtacaacaactggccccggacctttggccagggcaccaaggtggaaatcaagcgtactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 102 - protein, 5 aa, synthetic construct
HYYWS

SEQ ID NO: 103 - protein, 16 aa, synthetic construct
RIYTSGSTNYNPSLKS

SEQ ID NO: 104 - protein, 9 aa, synthetic construct
DRYTGYFDL

SEQ ID NO: 105 - protein, 12 aa, synthetic construct
RASQSVSSSYLA

SEQ ID NO: 106 - protein, 7 aa, synthetic construct
GASSRAT

SEQ ID NO: 107 - protein, 9 aa, synthetic construct
QQYGSSPPT

SEQ ID NO: 108 - protein, 7 aa, synthetic construct
GASISHY

SEQ ID NO: 109 - protein, 5 aa, synthetic construct
YTSGS

SEQ ID NO: 110 - protein, 10 aa, synthetic construct
GASISHYYWS

SEQ ID NO: 111 - protein, 9 aa, synthetic construct
RIYTSGSTN

SEQ ID NO: 112 - protein, 6 aa, synthetic construct
SHYYWS

SEQ ID NO: 113 - protein, 12 aa, synthetic construct
WIGRIYTSGSTN

SEQ ID NO: 114 - protein, 10 aa, synthetic construct
ARDRYTGYFD

SEQ ID NO: 115 - protein, 8 aa, synthetic construct
SSSYLAWY

SEQ ID NO: 116 - protein, 10 aa, synthetic construct
LLIYGASSRA

SEQ ID NO: 117 - protein, 8 aa, synthetic construct
QQYGSSPP

SEQ ID NO: 118 - protein, 8 aa, synthetic construct
GASISHYY

SEQ ID NO: 119 - protein, 7 aa, synthetic construct
IYTSGST

SEQ ID NO: 120 - protein, 11 aa, synthetic construct
ARDRYTGYFDL

SEQ ID NO: 121 - protein, 7 aa, synthetic construct
QSVSSSY

SEQ ID NO: 122 - protein, 117 aa, synthetic construct
QVQLQESGPGLVKPSETLSLTCTVSGASISHYYWSWIRRPAGKGLEWIGRIYTSGSTNYNPSLKSRVTVSVDTSKNQFSLKLSSVTAADTAVYYCARDRYTGYFDLWGRGTLVTVSS

SEQ ID NO: 123 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPPTFGGGTKLEIK

SEQ ID NO: 124 - DNA, 351 bp, synthetic construct
  source 1..351: mol_type = other DNA
caggtgcagctgcaggagtcgggcccaggactggtgaagccttcggagaccctgtccctcacctgcactgtctctggtgcctccatcagtcattactactggagctggatccggcggcccgccgggaagggactggagtggattgggcgtatctataccagtgggagcaccaactacaacccctccctcaagagtcgagtcaccgtgtcagtagacacgtccaagaaccagttctccctgaagctgagctctgtgaccgccgcggacacggccgtatattactgtgcgagagatcgatatacggggtacttcgatctctggggccgtggcaccctggtcactgtctcctca

SEQ ID NO: 125 - DNA, 324 bp, synthetic construct
  source 1..324: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtttattactgtcagcagtatggtagctcacctcccactttcggcggagggaccaagctggagatcaaa

SEQ ID NO: 126 - protein, 5 aa, synthetic construct
RSTMH

SEQ ID NO: 127 - protein, 17 aa, synthetic construct
YINPSSAYTNYNQKFQG

SEQ ID NO: 128 - protein, 12 aa, synthetic construct
PQVHYDYAGFPY

SEQ ID NO: 129 - protein, 10 aa, synthetic construct
SASSSVSYMN

SEQ ID NO: 130 - protein, 7 aa, synthetic construct
DSSKLAS

SEQ ID NO: 131 - protein, 9 aa, synthetic construct
QQWSRNPPT

SEQ ID NO: 132 - protein, 121 aa, synthetic construct; identical to SEQ ID NO: 28

SEQ ID NO: 133 - protein, 106 aa, synthetic construct; identical to SEQ ID NO: 29

SEQ ID NO: 134 - protein, 245 aa, synthetic construct; identical to SEQ ID NO: 30

SEQ ID NO: 135 - DNA, 363 bp, synthetic construct
  source 1..363: mol_type = other DNA
caagtacaacttgttcaatctggcgctgaagtcaaaaaacccggatctagtgttaaagtttcatgtaaggcatcaggctatactttcactcggtctacaatgcactgggtcaagcaggctccagggcaaggtttggaatggatagggtacataaatccttctagcgcctatactaattacaaccagaagtttcagggacgtgttactctgactgctgacaaatcaacatctacagcctatatggaactgtcttcattgaggagtgaggacactgctgtgtattactgcgcttctcctcaggtgcactatgattacgctggctttccctactggggttgtggcaccctcgtcaccgtttcatct

SEQ ID NO: 136 - DNA, 318 bp, synthetic construct
  source 1..318: mol_type = other DNA
gagatcgtcttgactcagtcccctgcaaccctcagcgcaagtcccggagagcgcgttacattgagttgttccgcatccagttcagtcagctacatgaactggtaccaacaaaagcccggttgtgctccaagaaggtggatctatgattccagtaagctggcctccggggtcccagcaaggttcagcggttccggcagcggaagggactacactctgaccataagttcccttgaaccagaagacttcgccgtctattattgtcagcagtggtcccgaaatccacctaccttcgggggcggcactaaagtggagattaaa

SEQ ID NO: 137 - DNA, 735 bp, synthetic construct
  source 1..735: mol_type = other DNA
gagatcgtcttgactcagtcccctgcaaccctcagcgcaagtcccggagagcgcgttacattgagttgttccgcatccagttcagtcagctacatgaactggtaccaacaaaagcccggttgtgctccaagaaggtggatctatgattccagtaagctggcctccggggtcccagcaaggttcagcggttccggcagcggaagggactacactctgaccataagttcccttgaaccagaagacttcgccgtctattattgtcagcagtggtcccgaaatccacctaccttcgggggcggcactaaagtggagattaaaggaggaggtagtggcggctccggtggatgtcctccttgcggaggatcaggaggtcaagtacaacttgttcaatctggcgctgaagtcaaaaaacccggatctagtgttaaagtttcatgtaaggcatcaggctatactttcactcggtctacaatgcactgggtcaagcaggctccagggcaaggtttggaatggatagggtacataaatccttctagcgcctatactaattacaaccagaagtttcagggacgtgttactctgactgctgacaaatcaacatctacagcctatatggaactgtcttcattgaggagtgaggacactgctgtgtattactgcgcttctcctcaggtgcactatgattacgctggctttccctactggggttgtggcaccctcgtcaccgtttcatct

SEQ ID NO: 138 - protein, 447 aa, synthetic construct
QVQLQESGPGLVKPSETLSLTCTVSGASISHYYWSWIRRPAGKGLEWIGRIYTSGSTNYNPSLKSRVTVSVDTSKNQFSLKLSSVTAADTAVYYCARDRYTGYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 139 - protein, 215 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 140 - protein, 477 aa, synthetic construct; identical to SEQ ID NO: 66

SEQ ID NO: 141 - DNA, 1341 bp, synthetic construct
  source 1..1341: mol_type = other DNA
caggtgcagctgcaggagtcgggcccaggactggtgaagccttcggagaccctgtccctcacctgcactgtctctggtgcctccatcagtcattactactggagctggatccggcggcccgccgggaagggactggagtggattgggcgtatctataccagtgggagcaccaactacaacccctccctcaagagtcgagtcaccgtgtcagtagacacgtccaagaaccagttctccctgaagctgagctctgtgaccgccgcggacacggccgtatattactgtgcgagagatcgatatacggggtacttcgatctctggggccgtggcaccctggtcactgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtcctgcgcggtcaaaggcttctatcccagcgacatcgccgtggagtgggagagcaatgggcagccggagaacaactacaagaccacgcctcccgtgctggactccgacggctccttcttcctcgtcagcaagctcaccgtggacaagtctagatggcagcaggggaacgtcttctcatgctccgtgatgcatgaggctctgcacaaccggttcacgcagaagagcctctccctgtctccgggtaaa

SEQ ID NO: 142 - DNA, 645 bp, synthetic construct
  source 1..645: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtttattactgtcagcagtatggtagctcacctcccactttcggcggagggaccaagctggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 143 - DNA, 1431 bp, synthetic construct
  source 1..1431: mol_type = other DNA
gagatcgtcttgactcagtcccctgcaaccctcagcgcaagtcccggagagcgcgttacattgagttgttccgcatccagttcagtcagctacatgaactggtaccaacaaaagcccggttgtgctccaagaaggtggatctatgattccagtaagctggcctccggggtcccagcaaggttcagcggttccggcagcggaagggactacactctgaccataagttcccttgaaccagaagacttcgccgtctattattgtcagcagtggtcccgaaatccacctaccttcgggggcggcactaaagtggagattaaaggaggaggtagtggcggctccggtggatgtcctccttgcggaggatcaggaggtcaagtacaacttgttcaatctggcgctgaagtcaaaaaacccggatctagtgttaaagtttcatgtaaggcatcaggctatactttcactcggtctacaatgcactgggtcaagcaggctccagggcaaggtttggaatggatagggtacataaatccttctagcgcctatactaattacaaccagaagtttcagggacgtgttactctgactgctgacaaatcaacatctacagcctatatggaactgtcttcattgaggagtgaggacactgctgtgtattactgcgcttctcctcaggtgcactatgattacgctggctttccctactggggttgtggcaccctcgtcaccgtttcatctgagcccaaatctagcgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagtctagatggcagcaggggaacgtcttctcatgctccgtgatgcatgaggctctgcacaaccactacacgcagaagagcctctccctgtctccgggtaaa

SEQ ID NO: 144 - protein, 245 aa, synthetic construct; identical to SEQ ID NO: 30

SEQ ID NO: 145 - protein, 245 aa, synthetic construct; identical to SEQ ID NO: 30

SEQ ID NO: 146 - protein, 245 aa, synthetic construct; identical to SEQ ID NO: 30

SEQ ID NO: 147 - protein, 245 aa, synthetic construct; identical to SEQ ID NO: 30

SEQ ID NO: 148 - protein, 450 aa, synthetic construct
EVQLLESGGGLVQPGGSLRLSCAASGFIFSNYYMAWVRLAPGKGLEWVSSISIGGGKSSYADSVKGRFTISRDNSKNTLYLQMNSLRAEDTAVYYCVRHPQLGPNPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 149 - protein, 218 aa, synthetic construct
DIQMTQSPSSLSASVGDRVTITCRASQSVTISSVDLMNWYQQKPGKAPKLLIYRASNLASGIPSRFSGSGSGTDFTLTISSLQPEDFATYYCQQSRESPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 150 - protein, 450 aa, synthetic construct
EVQLLESGGGLVQPGGSLRLSCAASGFIFSNYYMAWVRLAPGKGLEWVSSISIGGGKSHYADSVKGRFTISRDNSKNTLYLQMNSLRAEDTAVYYCVRHPSLGPNPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 151 - protein, 218 aa, synthetic construct; identical to SEQ ID NO: 149

SEQ ID NO: 152 - protein, 450 aa, synthetic construct
EVQLLESGGGLVQPGGSLRLSCAASGFIFSNYYMAWVRLAPGKGLEWVSSISIGGGKSHYADSVKGRFTISRDNSKNTLYLQMNSLRAEDTAVYYCVRHPQSGPNPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 153 - protein, 218 aa, synthetic construct; identical to SEQ ID NO: 149

SEQ ID NO: 154 - protein, 450 aa, synthetic construct
EVQLLESGGGLVQPGGSLRLSCAASGFIFSNYYMAWVRLAPGKGLEWVSSISIGGGKSHYADSVKGRFTISRDNSKNTLYLQMNSLRAEDTAVYYCVRHPQLGPNPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 155 - protein, 218 aa, synthetic construct; identical to SEQ ID NO: 149

SEQ ID NO: 156 - protein, 21 aa, synthetic construct
AGGGGSGGGGSGGGGSGGGGS

SEQ ID NO: 157 - protein, 4 aa, synthetic construct
  REGION 1: note = When part of an spFv VL domain, an intrachain disulfide bond will be formed
  REGION 4: note = When part of an spFv VH domain, an intrachain disulfide bond will be formed
CPPC